(19) United States
(12) Patent Application Publication
(10) Pub. No.: US 2025/0250230 A1
(43) Pub. Date: Aug. 7, 2025

(54) HETEROCYCLIC COMPOUND

(71) Applicant: TAKEDA PHARMACEUTICAL COMPANY LIMITED, Osaka (JP)

(72) Inventors: Shuhei IKEDA, Kanagawa (JP); Martin Alexander PAWLICZEK, Kanagawa (JP); Eiji KIMURA, Kanagawa (JP); Yasushi HATTORI, Kanagawa (JP); Marilena PIRA, Kanagawa (JP); Javier MIGUELEZ-RAMOS, Kanagawa (JP); Norihito TOKUNAGA, Kanagawa (JP); Yuhei MIYANOHANA, Kanagawa (JP); Yasutaka HOASHI, Kanagawa (JP); Yuichi KAJITA, Kanagawa (JP); Yoshiteru ITO, Kanagawa (JP); Tasuki KOIKE, Kanagawa (JP)

(21) Appl. No.: 18/855,917

(22) PCT Filed: Apr. 12, 2022
(86) PCT No.: PCT/IB2022/053399

§ 371 Date: Oct. 10, 2024

(51) Int. Cl. C07D 205/04; C07D 205/12; C07D 207/09; C07D 207/14; C07D 209/52; C07D 211/28; C07D 211/58; C07D 223/12; C07D 225/02; C07D 265/30; C07D 267/10; C07D 401/08; C07D 409/06; C07D 471/08; C07D 487/04

(52) U.S. Cl. CPC C07D 205/04; C07D 205/12; C07D 207/09; C07D 207/14; C07D 209/52; C07D 211/28; C07D 211/58; C07D 223/12; C07D 225/02; C07D 265/30; C07D 267/10; C07D 401/08; C07D 409/06; C07D 471/08; C07D 487/04

(57) ABSTRACT

The present invention provides a heterocyclic compound having an orexin type 2 receptor agonist activity. A compound represented by the formula (I) is useful as an agent for the prophylaxis or treatment of narcolepsy.

### TECHNICAL FIELD

The present invention relates to a heterocyclic compound, particularly, a heterocyclic compound having an orexin type 2 receptor agonist activity.

### BACKGROUND OF THE INVENTION

Orexin is a neuropeptide specifically produced in particular neurons located sparsely in the lateral hypothalamus and its surrounding area, and consists of two subtypes, orexin A and orexin B. Both orexin A and orexin B are endogenous ligands of the orexin receptors, which are G protein-coupled receptors mainly present in the brain, and two types of subtypes, type 1 and type 2, are known for the orexin receptors (non-patent document 1).

Since orexin-producing neurons (orexin neurons) are localized in the vicinity of the feeding center, and intraventricular administration of orexin peptide results in an increase in food intake, orexin initially attracted attention as a neuropeptide having a feeding behavioral regulation. Thereafter, however, it was reported that the cause of dog narcolepsy is genetic variation of orexin type 2 receptor (non-patent document 2), and the role of orexin in controlling sleep and wakefulness has been also attracted.

From the studies using a transgenic mouse having denatured orexin neurons and a double transgenic mouse obtained by crossing this mouse with orexin overexpressing transgenic mouse, it was clarified that narcolepsy-like symptoms that appear by degeneration of orexin neurons disappear due to sustained expression of orexin. Similarly, when orexin peptide was intraventricularly administered to a transgenic mouse having denatured orexin neuron, improvement of narcolepsy-like symptoms was also observed (non-patent document 3). Studies of orexin type 2 receptor knockout mice have suggested that orexin type 2 receptor is important for maintaining arousal (non-patent document 4, non-patent document 5). Such background suggests that orexin type 2 receptor agonists become therapeutic drugs for narcolepsy or therapeutic drugs for other sleep disorders exhibiting excessive sleepiness (non-patent document 6).

In addition, it is suggested that a peptidic agonist that selectively acts on the orexin type 2 receptor improves obesity due to high fat diet load in mice (non-patent document 7).

In addition, it is suggested that intraventricular administration of orexin peptide shortens the systemic anesthetic time of rat (non-patent document 8).

In addition, it is suggested that patients with sleep apnea syndrome show low orexin A concentration levels in plasma (non-patent document 9).

In addition, it is suggested that intraventricular administration of orexin peptide improves memory retention of senescence-accelerated model mouse (SAMP8) with cognitive dysfunction (non-patent document 10).

In addition, it is suggested that Orexin type 2 receptor agonist will be a therapeutic drug for cardiac failure (patent document 1, non-patent document 11).

In addition, it is suggested that the daytime sleepiness of Parkinson's disease patients is caused by orexin nerve fallout (non-patent document 12).

In addition, it is suggested that orexin regulates bone formation and bone loss, and orexin type 2 receptor agonist will be a therapeutic drug for diseases related to bone loss such as osteoporosis, rheumatoid arthritis and the like (patent document 2).

In addition, it is suggested that orexin receptor agonist is useful for the prophylaxis or treatment of sepsis, severe sepsis and septic shock, since the mortality was significantly improved by mere continuous administration of orexin from the periphery in septic shock model mouse (patent document 3).

Therefore, a compound having an orexin type 2 receptor agonist activity is expected to be useful as a novel therapeutic drug for narcolepsy, idiopathic hypersomnia, hypersomnia, sleep apnea syndrome, disturbance of consciousness such as coma and the like, narcolepsy syndrome accompanied by narcolepsy-like symptoms, hypersomnia syndrome accompanied by daytime hypersomnia (e.g., Parkinson's disease, Guillain-Barre syndrome and Kleine Levin syndrome), Alzheimer, obesity, insulin resistance syndrome, cardiac failure, diseases related to bone loss, sepsis and the like, further, anesthetic antagonist, a prophylactic or therapeutic drug for side effects and complications due to anesthesia.

As sulfonamide derivatives, a compound represented by the formula

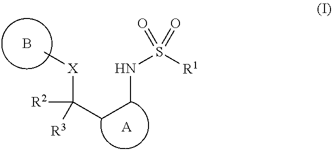

wherein each symbol is as described in the document (Patent Document 4) has been reported.

In addition, as compounds having an orexin type 2 receptor agonist activity, the following compounds have been reported.

A compound represented by the formula

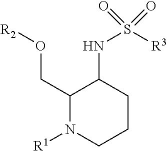

wherein each symbol is as described in the document (Patent Document 5).

A compound represented by the formula

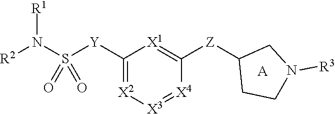

wherein each symbol is as described in the document (Patent Document 6).

A compound represented by the formula

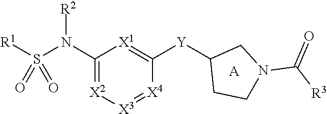

wherein each symbol is as described in the document (Patent Document 7).

A compound represented by the formula

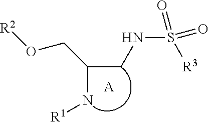

wherein each symbol is as described in the document (Patent Document 8).

A compound represented by the formula

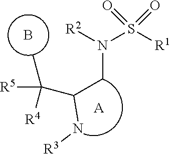

wherein each symbol is as described in the document (Patent Document 9).

A compound represented by the formula

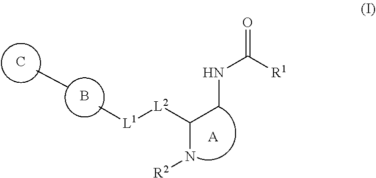

wherein each symbol is as described in the document (Patent Document 10).

A compound represented by the formula

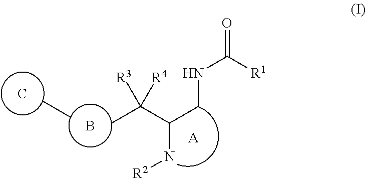

wherein each symbol is as described in the document (Patent Document 11).

A compound represented by the formula

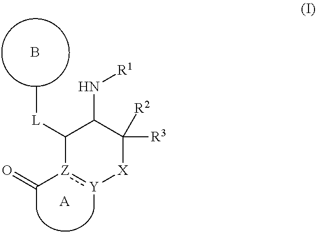

wherein each symbol is as described in the document (Patent Document 12).

A compound represented by the formula

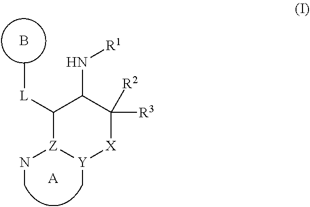

wherein each symbol is as described in the document (Patent Document 13).

A compound represented by the formula

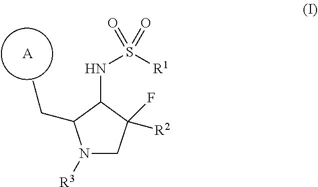

wherein each symbol is as described in the document (Patent Document 14).

A compound represented by the formula

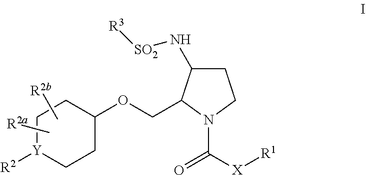

wherein each symbol is as described in the document (Patent Document 15).

A compound represented by the formula

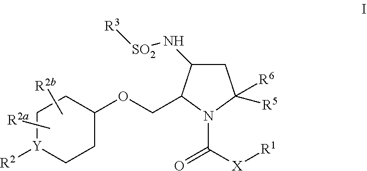

wherein each symbol is as described in the document (Patent Document 16).

A compound represented by the formula

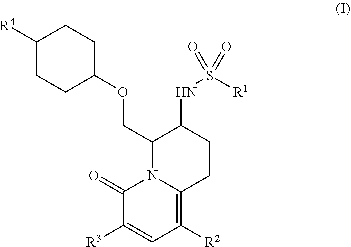

wherein each symbol is as described in the document (Patent Document 17).

However, these compounds are considered to be insufficient in terms of activity, pharmacokinetics or safety, and development of compounds compound having an orexin type 2 receptor agonist activity is still desired.

### DOCUMENT LIST

### [Patent Document]

[Patent Document 1] WO 2015/073707 A1
[Patent Document 2] WO 2015/048091 A1
[Patent Document 3] WO 2015/147240 A1
[Patent Document 4] WO 2012/137982 A9
[Patent Document 5] WO 2017/135306 A1
[Patent Document 6] WO 2018/164191 A1
[Patent Document 7] WO 2018/164192 A1
[Patent Document 8] WO 2019/027003 A1
[Patent Document 9] WO 2019/027058 A1
[Patent Document 10] WO 2020/004536 A1
[Patent Document 11] WO 2020/004537 A1
[Patent Document 12] WO 2020/122092 A1
[Patent Document 13] WO 2020/122093 A1
[Patent Document 14] WO 2020/158958 A1
[Patent Document 15] WO 2020/167701 A1
[Patent Document 16] WO 2020/167706 A1
[Patent Document 17] WO 2021/106975 A1

### Non-Patent Document

[Non-Patent Document 1] Cell, Vol. 92, 573-585, 1998)
[Non-Patent Document 2] Cell, Vol. 98, 365-376, 1999)
[Non-Patent Document 3] Proc. Natl. Acad. Sci. USA, Vol. 101, 4649-4654, 2004)
[Non-Patent Document 4] Cell, Vol. 98, 437-451, 1999)
[Non-Patent Document 5] Neuron, Vol. 38, 715-730, 2003)
[Non-Patent Document 6] CNS Drugs, Vol. 27, 83-90, 2013)
[Non-Patent Document 7] Cell Metabolism, Vol. 9, 64-76, 2009)
[Non-Patent Document 8] Neuroscience, Vol. 121, 855-863, 2003)
[Non-Patent Document 9] Respiration, Vol. 71, 575-579, 2004)
[Non-Patent Document 10] Peptides, Vol. 23, 1683-1688, 2002)
[Non-Patent Document 11] Journal of the American College of Cardiology. Vol. 66, 2015, Pages 2522-2533)
[Non-Patent Document 12] Brain. Vol. 130, 2007, Pages 1586-1595)

### SUMMARY OF THE INVENTION

### Problems to be Solved by the Invention

The present invention aims to provide a heterocyclic compound having an orexin type 2 receptor agonist activity.

### Means of Solving the Problems

The present inventors have found that a compound represented by the following formula (I) or a salt thereof (sometimes to be referred to as compound (I) in the present specification) has an orexin type 2 receptor agonist activity. As a result of further studies, they have completed the present invention.

Accordingly, the present invention provides the following.

[1] A compound represented by the formula:

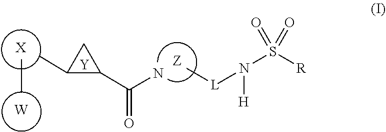

wherein
Ring W is an optionally further substituted ring;
Ring X is an optionally further substituted 5- or 6-membered aromatic ring;
Ring Y is an optionally further substituted cyclopropane ring;
Ring Z is an optionally further substituted nitrogen-containing heterocycle;
L is a bond, or an optionally substituted methylene; and
R is an optionally substituted C₁₋₆ alkyl group, an optionally substituted C₃₋₁₀ cycloalkyl group, or NR^{a}R^{b}; wherein
R^{a} is an optionally substituted C₁₋₆ alkyl group, or an optionally substituted C₃₋₁₀ cycloalkyl group;
R^{b} is a hydrogen atom, an optionally substituted C₁₋₆ alkyl group, or an optionally substituted C₃₋₁₀ cycloalkyl group; or
R^{a} and R^{b} in combination form an optionally further substituted nitrogen-containing heterocycle, together with adjacent nitrogen atom,
   or a salt thereof.
   [2] The compound or salt according to the above-mentioned [1], wherein
Ring W is an optionally further substituted ring;
Ring X is an optionally further substituted 5- or 6-membered aromatic ring;
Ring Y is an optionally further substituted cyclopropane ring;
Ring Z is an optionally further substituted nitrogen-containing heterocycle;
L is a bond, or an optionally substituted methylene; and
R is an optionally substituted C₁₋₆ alkyl, an optionally substituted C₃₋₁₀ cycloalkyl, or an optionally substituted di-C₁₋₆ alkyl amine.
   [3] The compound or salt according to the above-mentioned [1], wherein

### Ring W is

(1) a 3- to 8-membered monocyclic non-aromatic heterocycle,
(2) a 5- to 6-membered monocyclic aromatic heterocycle optionally further substituted by 1 to 3 halogen atoms,
(3) a C₆₋₁₄ aromatic hydrocarbon ring optionally further substituted by 1 to 3 substituents selected from
   (i) a halogen atom,
   (ii) a C₁₋₆ alkyl group optionally substituted by 1 to 3 C₁₋₆ alkoxy groups, and
   (iii) a C₁₋₆ alkoxy group, or
(4) a C₃₋₁₀ cycloalkane;

### Ring X is

(1) a benzene ring optionally further substituted by 1 to 3 substituents selected from
   (i) a halogen atom,
   (ii) an optionally halogenated C₁₋₆ alkyl group, and
   (iii) a C₁₋₆ alkoxy group, or
(2) a 5- or 6-membered monocyclic aromatic heterocycle optionally further substituted by 1 to 3 substituents selected from
   (i) a halogen atom, and
   (ii) C₁₋₆ alkyl groups;
      Ring Y is a cyclopropane ring optionally further substituted by 1 to 3 substituents selected from
(1) a halogen atom,
(2) a cyano group, and
(3) a C₁₋₆ alkyl group, optionally substituted by 1 to 3 substituents selected from a C₁₋₆ alkoxy group, cyano, and hydroxy;
   Ring Z is a 3- to 14-membered nitrogen-containing heterocycle optionally further substituted by 1 to 3 substituents selected from
(1) a halogen atom,
(2) a hydroxy group,
(3) a C₁₋₆ alkyl group, optionally substituted by 1 to 3 substituents selected from C₁₋₆ alkoxy group, halogen atom, and hydroxy,
(4) a C₁₋₆ alkoxy group,
(5) —C(O)—C₁₋₆ alkyl, and
(6) a 5-membered monocyclic aromatic heterocycle optionally substituted with a C₁₋₆ alkyl;
   L is a bond or a methylene group optionally substituted with 1 substituent selected from
(1) a C₁₋₆ alkyl group further optionally substituted by 1 to 3 substituents selected from a C₁₋₆ alkoxy group, and hydroxy, and
(2) a C₃s cycloalkyl group further optionally substituted with a hydroxy group;

### R is

(1) a C₁₋₆ alkyl group optionally substituted by 1 to 3 substituents selected from
   (i) a halogen atom,
   (ii) a C₁₋₆ alkoxy, and
   (iii) a cyclopropyl group,
(2) a C₃₋₁₀ cycloalkyl group further optionally substituted with a C₁₋₆ alkyl group, or
(3) NR^{a}R^{b}.
   wherein
R^{a} is a C₁₋₆ alkyl group, or a C₃₋₁₀ cycloalkyl group;
R^{b} is a hydrogen atom, or a C₁₋₆ alkyl group; or
R^{a} and R^{b} in combination form a nitrogen-containing heterocycle, together with adjacent nitrogen atom.
   [4] The compound or salt according to the above-mentioned [1]wherein
Ring W is a C₆₋₁₄ aromatic hydrocarbon ring optionally further substituted by 1 to 3 halogen atoms;
Ring X is a benzene ring optionally further substituted by 1 to 3 substituents selected from a halogen atom, and a C₁₋₆ alkyl group;
Ring Y is a cyclopropane ring optionally further substituted by 1 to 3 halogen atoms;
Ring Z is a 3- to 14-membered nitrogen-containing heterocycle ring optionally further substituted by 1 to 3 substituents selected from a halogen atom, a hydroxy group and a C₁₋₆ alkyl group;
L is a bond or a methylene group optionally substituted with a C₁₋₆ alkyl group;
R is
   (1) a C₁₋₆ alkyl group, or
   (2) NR^{a}R^{b};
      wherein R^{a} is a C₁₋₆ alkyl group; and
      R^{b} is a hydrogen atom, or a C₁₋₆ alkyl group.
      [5] The compound or salt according to the above-mentioned [1], wherein
Ring W is a benzene ring optionally substituted by 1 to 3 halogen atoms;
Ring X is a benzene ring optionally further substituted by 1 to 3 substitutents selected from a halogen atom, and a C₁₋₆ alkyl group;
Ring Y is a cyclopropane ring optionally further substituted by 1 to 3 halogen atoms;
Ring Z is an azetidine ring optionally further substituted by 1 to 3 substitutents selected from a halogen atom and a C₁₋₆ alkyl group;
L is a methylene group optionally substituted with a C₁₋₆ alkyl group; and
R is a C₁₋₆ alkyl group or NR^{a}R^{b};
   wherein R^{a} is a C₁₋₆ alkyl group; and
R^{b} is a hydrogen atom.
   [6] The compound or salt according to the above-mentioned [1], wherein
Ring W is a benzene ring optionally substituted by 1 to 3 halogen atoms;
Ring X is a benzene ring optionally further substituted by 1 to 3 halogen atoms;
Ring Y is a cyclopropane ring optionally further substituted by 1 to 3 halogen atoms;
Ring Z is an azetidine ring optionally further substituted by 1 to 3 halogen atoms;
L is a methylene group;
   R is a C₁₋₆ alkyl group, or NR^{a}R^{b};
   wherein R^{a} is a C₁₋₆ alkyl group; and
R¹ is a hydrogen atom.
   [7] A compound which is:

N-({(2R,3R)-3-fluoro-1-[(1S,2S)-2-fluoro-2-(2′,5,6′-trifluoro[1,1′-biphenyl]-2-yl)cyclopropane-1-carbonyl]azetidin-2-yl}methyl)ethanesulfonamide or a salt thereof.

[8] A compound which is:

N-({(2R,3R)-3-fluoro-1-[(1S,2S)-2-fluoro-2-(2′,5,6′-trifluoro[1,1′-biphenyl]-2-yl)cyclopropane-1-carbonyl]azetidin-2-yl}methyl)-N′-methylsulfuric diamide or a salt thereof.

[9] A compound which is:
N-({(2R,3R)-1-[(1S,2S)-2-(2′,6′-difluoro[1,1′-biphenyl]-2-yl)-2-fluorocyclopropane-1-carbonyl]-3-fluoroazetidin-2-yl}methyl)ethanesulfonamide or a salt thereof.
   [10] A compound of formula (II)

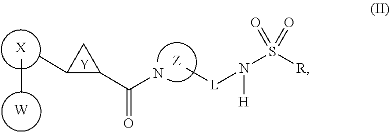

wherein:
Ring W is selected from benzene, 5- or 6-membered heteroaryl, 3-8-membered cycloalkyl, and 3-8-membered heterocyclyl, wherein each ring is optionally substituted by one, two, three, or four groups independently selected from halogen, (C₁-C₆)alkyl, and (C₁-C₆) alkoxy, wherein said (C₁-C₆)alkyl or (C₁-C₆)alkoxy is optionally substituted by one, two, three, or four halogen atoms;
Ring X is benzene or 5- or 6-membered heteroaryl, wherein each ring is optionally substituted with one, two, or three groups independently selected from halogen, (C₁-C₆)alkyl, and (C₁-C₆) alkoxy, wherein said (C₁-C₆)alkyl is optionally substituted by one, two, three, or four halogen atoms;
Ring Y is a cyclopropane ring optionally substituted by one, two, or three groups independently selected from halogen, (C₁-C₆)alkyl, (C₁-C₆) alkoxy, (C₁-C₆)alkyl-OH, (C₁-C₆)alkyl-O—(C₁-C₆)alkyl, (C₁-C₆)alkyl-CN, and —CN;
Ring Z is 3-9-membered nitrogen-containing monocyclic ring or 6-8-membered nitrogen-containing bicyclic ring, wherein each ring optionally contains one or two additional heteroatoms independently selected from oxygen, nitrogen, and sulfur; wherein said 3-9-membered nitrogen-containing monocyclic ring is optionally substituted by one, two, three, or four groups independently selected from halogen, (C₁-C₆)alkyl, halo(C₁-C₆)alkyl, (C₁-C₆) alkoxy, (C₁-C₆)alkyl-OH, (C₁-C₆)alkyl-O—(C₁-C₆)alkyl, —OH, and —C(O)—(C₁-C₆)alkyl and is also optionally substituted with a 5- or 6-membered heteroaryl ring optionally substituted with one, two, or three (C₁-C₆)alkyl groups; and wherein said 6-8-membered bicyclic ring is optionally substituted by one, two, three, or four groups independently selected from halogen, (C₁-C₆)alkyl, halo(C₁-C₆)alkyl, (C₁-C₆)alkoxy, (C₁-C₆)alkyl-OH, (C₁-C₆)alkyl-O—(C₁-C₆)alkyl, —OH, and —C(O)—(C₁-C₆)alkyl;
L is a bond, or —CH₂—, wherein said —CH₂— is optionally substituted by one or two groups independently selected from (C₁-C₆)alkyl, (C₁-C₆)alkoxy, and 3-6-membered cycloalkyl, wherein said (C₁-C₆)alkyl and 3-6-membered cycloalkyl ring are optionally substituted by one, two, or three groups independently selected from (C₁-C₆)alkyl, (C₁-C₆) alkoxy, and —OH; or, alternatively, wherein said —CH₂— is optionally substituted with two groups which, taken together with the carbon atom to which they are attached, form a 3-6 membered cycloalkyl or 3-6 membered heterocyclyl ring containing one or two oxygen atoms; and
R is (C₁-C₆)alkyl, —(C₁-C₆)alkyl-3-7 membered cycloalkyl, 3-7-membered cycloalkyl, (C₁-C₆) alkoxy, (C₁-C₆)alkyl-O—(C₁-C₆)alkyl, —NH₂, —NH((C₁-C₆)alkyl), —N((C₁-C₆)alkyl) 2, —NH(3-7-membered cycloalkyl ring), —N(3-7-membered cycloalkyl ring)₂, —N(3-7-membered cycloalkyl ring)(C₁-C₆)alkyl), and 3-7 membered heterocyclyl, wherein said (C₁-C₆)alkyl, —(C₁-C₆)alkyl-3-7 membered cycloalkyl, 3-7-membered cycloalkyl, (C₁-C₆) alkoxy, (C₁-C₆)alkyl-O—(C₁-C₆)alkyl, —NH((C₁-C₆)alkyl), N((C₁-C₆)alkyl)₂, —NH(3-7-membered cycloalkyl ring), —N(3-7-membered cycloalkyl ring)₂, —N(3-7-membered cycloalkyl ring)(C₁-C₆)alkyl, and 3-7-membered heterocyclyl are optionally substituted by one, two, or three groups independently selected from halogen and (C₁-C₆)alkyl;
   or a pharmaceutically acceptable salt thereof.
   [11] A compound of Formula (II′)

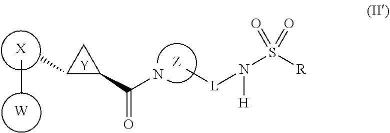

wherein:
Ring W is selected from benzene, 5- or 6-membered heteroaryl, 3-8-membered cycloalkyl, and 3-8-membered heterocyclyl, wherein each ring is optionally substituted by one, two, three, or four groups independently selected from halogen, (C₁-C₆)alkyl, and (C₁-C₆) alkoxy, wherein said (C₁-C₆)alkyl or (C₁-C₆)alkoxy is optionally substituted by one, two, three, or four halogen atoms;
Ring X is benzene or 5- or 6-membered heteroaryl, wherein each ring is optionally substituted with one, two, or three groups independently selected from halogen, (C₁-C₆)alkyl, and (C₁-C₆)alkoxy, wherein said (C₁-C₆)alkyl is optionally substituted by one, two, three, or four halogen atoms;
Ring Y is a cyclopropane ring optionally substituted by one, two, or three groups independently selected from halogen, (C₁-C₆)alkyl, (C₁-C₆) alkoxy, (C₁-C₆)alkyl-OH, (C₁-C₆)alkyl-O—(C₁-C₆)alkyl, (C₁-C₆)alkyl-CN, and —CN;
Ring Z is 3-9-membered nitrogen-containing monocyclic ring or 6-8-membered nitrogen-containing bicyclic ring, wherein each ring optionally contains one or two additional heteroatoms independently selected from oxygen, nitrogen, and sulfur; wherein said 3-9-membered nitrogen-containing monocyclic ring is optionally substituted by one, two, three, or four groups independently selected from halogen, (C₁-C₆)alkyl, halo(C₁-C₆)alkyl, (C₁-C₆) alkoxy, (C₁-C₆)alkyl-OH, (C₁-C₆)alkyl-O—(C₁-C₆)alkyl, —OH, and —C(O)—(C₁-C₆)alkyl and is also optionally substituted with a 5- or 6-membered heteroaryl ring optionally substituted with one, two, or three (C₁-C₆)alkyl groups; and wherein said 6-8-membered bicyclic ring is optionally substituted by one, two, three, or four groups independently selected from halogen, (C₁-C₆)alkyl, halo(C₁-C₆)alkyl, (C₁-C₆)alkoxy, (C₁-C₆)alkyl-OH, (C₁-C₆)alkyl-O—(C₁-C₆)alkyl, —OH, and —C(O)—(C₁-C₆)alkyl;
L is a bond, or —CH₂—, wherein said —CH₂— is optionally substituted by one or two groups independently selected from (C₁-C₆)alkyl, (C₁-C₆)alkoxy, and 3-6-membered cycloalkyl, wherein said (C₁-C₆)alkyl and 3-6-membered cycloalkyl ring are optionally substituted by one, two, or three groups independently selected from (C₁-C₆)alkyl, (C₁-C₆)alkoxy, and —OH; or, alternatively, wherein said —CH₂— is optionally substituted with two groups which, taken together with the carbon atom to which they are attached, form a 3-6 membered cycloalkyl or 3-6 membered heterocyclyl ring containing one or two oxygen atoms; and
R is (C₁-C₆)alkyl, —(C₁-C₆)alkyl-3-7 membered cycloalkyl, 3-7-membered cycloalkyl, (C₁-C₆)alkoxy, (C₁-C₆)alkyl-O—(C₁-C₆)alkyl, —NH₂, —NH((C₁-C₆)alkyl), —N((C₁-C₆)alkyl)₂, —NH(3-7-membered cycloalkyl ring), —N(3-7-membered cycloalkyl ring)₂, —N(3-7-membered cycloalkyl ring)(C₁-C₆)alkyl), and 3-7 membered heterocyclyl, wherein said (C₁-C₆)alkyl, —(C₁-C₆)alkyl-3-7 membered cycloalkyl, 3-7-membered cycloalkyl, (C₁-C₆)alkoxy, (C₁-C₆)alkyl-O—(C₁-C₆)alkyl, —NH((C₁-C₆)alkyl), N((C₁-C₆)alkyl)₂, —NH(3-7-membered cycloalkyl ring), —N(3-7-membered cycloalkyl ring)₂, —N(3-7-membered cycloalkyl ring)(C₁-C₆)alkyl, and 3-7-membered heterocyclyl are optionally substituted by one, two, or three groups independently selected from halogen and (C₁-C₆)alkyl,
   or a pharmaceutically acceptable salt thereof.
   [12] The compound or pharmaceutically acceptable salt thereof according to the above-mentioned [10] or [11] wherein
Ring W is benzene 5- or 6-membered heteroaryl, 3-8-membered cycloalkyl, and 6-membered heterocyclyl, wherein each ring is optionally substituted by one, two, three, or four groups independently selected from halogen, (C₁-C₆)alkyl, and (C₁-C₆)alkoxy, wherein said (C₁-C₆)alkyl or (C₁-C₆)alkoxy is optionally substituted by one, two, three, or four halogen atoms;
Ring X is benzene or 5- or 6-membered heteroaryl, wherein each ring is optionally substituted by one, two, or three groups independently selected from halogen, (C₁-C₆)alkyl, and (C₁-C₆)alkoxy, wherein said (C₁-C₆)alkyl is optionally substituted by one, two, three, or four halogen atoms;
Ring Y is a cyclopropane ring optionally substituted by one, two, or three groups independently selected from halogen, (C₁-C₆)alkyl, (C₁-C₆)alkyl-OH, (C₁-C₆)alkyl-O—(C₁-C₆)alkyl, (C₁-C₆)alkyl-CN, and —CN;
Ring Z is 3-9-membered nitrogen-containing monocyclic ring or 6-8-membered nitrogen-containing bicyclic ring, wherein each ring optionally contains one or two additional heteroatoms independently selected from oxygen, nitrogen, and sulfur; wherein said 3-9-membered nitrogen-containing monocyclic ring is optionally substituted by one, two, three, or four groups independently selected from halogen, (C₁-C₆)alkyl, halo(C₁-C₆)alkyl, ((C₁-C₆)alkyl)-OH, (C₁-C₆) alkoxy, (C₁-C₆)alkyl-O—(C₁-C₆)alkyl, —OH, and —C(O)—(C₁-C₆)alkyl and is also optionally substituted with a 5- or 6-membered heteroaryl ring optionally substituted with one, two, or three (C₁-C₆)alkyl groups; and wherein said 6-8-membered bicyclic ring is optionally substituted by one, two, three, or four groups independently selected from halogen, (C₁-C₆)alkyl, halo(C₁-C₆)alkyl, ((C₁-C₆)alkyl)-OH, (C₁-C₆)alkoxy, (C₁-C₆)alkyl-O—(C₁-C₆)alkyl, —OH, and —C(O)—(C₁-C₆)alkyl;
L is a bond or —CH₂—, wherein said —CH₂— is optionally substituted by one or two groups independently selected from (C₁-C₆)alkyl, (C₁-C₆)alkoxy, and 3-6-membered cycloalkyl, wherein said (C₁-C₆)alkyl and 3-6-membered cycloalkyl ring are optionally substituted by one, two, or three groups independently selected from (C₁-C₆)alkyl, (C₁-C₆) alkoxy, and —OH; and
R is (C₁-C₆)alkyl, —(C₁-C₆)alkyl-3-7 membered cycloalkyl, 3-7-membered cycloalkyl, (C₁-C₆)alkyl-O—(C₁-C₆)alkyl, —NH₂, —NH((C₁-C₆)alkyl), —N((C₁-C₆)alkyl)₂, —NH(3-7-membered cycloalkyl ring), —N(3-7-membered cycloalkyl ring)₂, —N(3-7-membered cycloalkyl ring)(C₁-C₆)alkyl), and 3-7 membered heterocyclyl, wherein said (C₁-C₆)alkyl, —(C₁-C₆)alkyl-3-7 membered cycloalkyl, 3-7-membered cycloalkyl, (C₁-C₆)alkoxy, (C₁-C₆)alkyl-O—(C₁-C₆)alkyl, —NH((C₁-C₆)alkyl), N((C₁-C₆)alkyl)₂, —NH(3-7-membered cycloalkyl ring), —N(3-7-membered cycloalkyl ring)₂, —N(3-7-membered cycloalkyl ring)(C₁-C₆)alkyl, and 3-7-membered heterocyclyl are optionally substituted by one, two, or three groups independently selected from halogen and (C₁-C₆)alkyl.
   [13] The compound or pharmaceutically acceptable salt thereof according to the above-mentioned [10] or [11], wherein
Ring W is benzene, 5- or 6-membered heteroaryl, 4-6-membered cycloalkyl, and 6-membered heterocyclyl, wherein each ring is optionally substituted by one, two, three, or four groups independently selected from halogen, (C₁-C₆)alkyl, and (C₁-C₆)alkoxy, wherein said (C₁-C₆)alkyl or (C₁-C₆)alkoxy is optionally substituted by one, two, three, or four halogen atoms;
Ring X is benzene or 5- or 6-membered heteroaryl, wherein each ring is optionally substituted by one, two, or three groups independently selected from halogen, (C₁-C₆)alkyl, and (C₁-C₆)alkoxy, wherein said (C₁-C₆)alkyl is optionally substituted by one, two, three, or four halogen atoms;
Ring Y is a cyclopropane ring optionally substituted by one, two, or three groups independently selected from halogen, (C₁-C₆)alkyl, (C₁-C₆)alkyl-OH, (C₁-C₆)alkyl-O—(C₁-C₆)alkyl, (C₁-C₆)alkyl-CN, and —CN;
Ring Z is 3-9-membered nitrogen-containing monocyclic ring or 6-8-membered nitrogen-containing bicyclic ring, wherein each ring optionally contains one or two additional heteroatoms independently selected from oxygen, nitrogen, and sulfur; wherein said 3-9-membered nitrogen-containing monocyclic ring is optionally substituted by one, two, or three groups independently selected from halogen, (C₁-C₆)alkyl, halo(C₁-C₆)alkyl, ((C₁-C₆)alkyl)-OH, (C₁-C₆) alkoxy, (C₁-C₆)alkyl-O—(C₁-C₆)alkyl, —OH, and —C(O)—(C₁-C₆)alkyl, and is also optionally substituted with a 5- or 6-membered heteroaryl ring optionally substituted with one, two, or three (C₁-C₆)alkyl groups; and wherein said 6-8-membered bicyclic ring is optionally substituted by one, two, three, or four groups independently selected from halogen, (C₁-C₆)alkyl, halo(C₁-C₆)alkyl, ((C₁-C₆)alkyl)-OH, (C₁-C₆)alkoxy, (C₁-C₆)alkyl-O—(C₁-C₆)alkyl, —OH, and —C(O)—(C₁-C₆)alkyl;
L is a bond or —CH₂—, wherein said —CH₂— is optionally substituted by one or two groups independently selected from (C₁-C₆)alkyl, (C₁-C₆)alkoxy, and 3-6-membered cycloalkyl, wherein said (C₁-C₆)alkyl and 3-6-membered cycloalkyl ring are optionally substituted by one, two, or three groups independently selected from (C₁-C₆)alkyl, (C₁-C₆)alkoxy, and —OH; and
R is (C₁-C₆)alkyl, —(C₁-C₆)alkyl-3-7 membered cycloalkyl, 3-7-membered cycloalkyl, (C₁-C₆)alkyl-O—(C₁-C₆)alkyl, —NH₂, —NH((C₁-C₆)alkyl), —N((C₁-C₆)alkyl)₂, —NH(3-7-membered cycloalkyl ring), —N(3-7-membered cycloalkyl ring)₂, —N(3-7-membered cycloalkyl ring)(C₁-C₆)alkyl), and 3-7 membered heterocyclyl, wherein said (C₁-C₆)alkyl, —(C₁-C₆)alkyl-3-7 membered cycloalkyl, 3-7-membered cycloalkyl, (C₁-C₆)alkoxy, (C₁-C₆)alkyl-O—(C₁-C₆)alkyl, —NH((C₁-C₆)alkyl), N((C₁-C₆)alkyl)₂, —NH(3-7-membered cycloalkyl ring), —N(3-7-membered cycloalkyl ring)₂, —N(3-7-membered cycloalkyl ring)(C₁-C₆)alkyl, and 3-7-membered heterocyclyl are optionally substituted by one, two, or three groups independently selected from halogen and (C₁-C₆)alkyl.
   [14] The compound or pharmaceutically acceptable salt thereof according to any one of the above-mentioned [10] to [13], wherein Ring W is benzene optionally further substituted by one, two, or three halogen atoms.
   [15] The compound or pharmaceutically acceptable salt of any one of the above-mentioned [10] to [14] wherein Ring W is benzene optionally substituted by one, two, or three halogen atoms independently selected from chloro and fluoro.
   [16] The compound or pharmaceutically acceptable salt thereof according to any one of the above-mentioned [10] to [15], wherein Ring X is benzene optionally substituted by one, two, or three groups independently selected from a halogen atom, and a C₁₋₆ alkyl group.
   [17] The compound or pharmaceutically acceptable salt thereof according to any one of the above-mentioned [10] to [16] wherein Ring Y is a cyclopropane ring optionally substituted by one, two, or three halogen atoms.
   [18] The compound or pharmaceutically acceptable salt thereof according to any one of the above-mentioned [10] to [17] wherein Ring Z is a 4- to 8-membered nitrogen-containing monocyclic ring optionally substituted by one, two, or three groups independently selected from a halogen atom and a C₁₋₆ alkyl group.
   [19] The compound or pharmaceutically acceptable salt of any one of the above-mentioned [10] to [18], wherein Ring Z is azetidine optionally substituted by one, two, or three groups independently selected from halogen atom and a (C₁-C₆)alkyl group.
   [20] The compound or pharmaceutically acceptable salt of any one of the above-mentioned [10] to [19], wherein Ring Z is azetidine optionally substituted by one fluoro or methyl group.
   [21] The compound or pharmaceutically acceptable salt thereof according to any one of the above-mentioned [10] to [20], wherein L is a bond or a —CH₂— linker optionally substituted with a C₁₋₆ alkyl group.
   [22] The compound or pharmaceutically acceptable salt thereof according to any one of [10] to the above-mentioned [21] wherein R is (C₁-C₆)alkyl, —NH((C₁-C₆)alkyl), or N((C₁-C₆)alkyl)₂.
   [23] A compound of Tables 1-1 to 1-80, or a pharmaceutically acceptable salt thereof.
   [24] The compound or pharmaceutically acceptable salt according to the above-mentioned [10] or [11], wherein
Ring W is benzene optionally substituted by one, two, or three halogen atoms;
Ring X is benzene optionally substituted by one, two, or three groups independently selected from a halogen atom and a C₁₋₆ alkyl;
Ring Y is a cyclopropane ring optionally substituted by one, two, or three halogen atoms;
Ring Z is a 4- to 8-membered nitrogen-containing monocyclic ring optionally substituted by one, two, or three groups independently selected from a halogen atom and a (C₁-C₆) alkyl group;
L is a bond or a —CH₂— group optionally substituted by a (C₁-C₆) alkyl group; and
R is a (C₁-C₆) alkyl group, —NH((C₁-C₆)alkyl), or —N((C₁-C₆)alkyl)₂.
   [25] The compound or pharmaceutically acceptable salt according to the above-mentioned [10] or [11], wherein
Ring W is benzene optionally substituted by one, two, or three halogen atoms;
Ring X is benzene optionally further substituted by one, two, or three groups independently selected from a halogen atom and a (C₁-C₆)alkyl group;
Ring Y is a cyclopropane ring optionally substituted by one, two, or three halogen atoms;
Ring Z is an azetidine ring optionally further substituted by one, two, or three groups independently selected from a halogen atom and a (C₁-C₆)alkyl group;
L is —CH₂— optionally substituted with a (C₁-C₆)alkyl group; and
R is (C₁-C₆)alkyl or —NH((C₁-C₆)alkyl).
   [26] The compound or pharmaceutically acceptable salt according to the above-mentioned [10] or [11], wherein
Ring W is benzene optionally substituted by one, two, or three halogen atoms;
Ring X is benzene optionally substituted by one, two, or three halogen atoms;
Ring Y is a cyclopropane ring optionally substituted by a one, two, or three halogen atoms;
Ring Z is azetidine optionally substituted by one, two, or three halogen atoms;
L is —CH₂—; and
R is a C₁₋₆ alkyl group, or —NH((C₁-C₆)alkyl).
   [27] A compound selected from
N-({(2R,3R)-1-[(1S,2S)-2-(2′,6′-difluoro[1,1′-biphenyl]-2-yl)-2-fluorocyclopropane-1-carbonyl]-3-fluoroazetidin-2-yl}methyl)ethanesulfonamide,
N-({(2R,3R)-3-fluoro-1-[(1S,2S)-2-fluoro-2-(2′,5,6′-trifluoro[1,1′-biphenyl]-2-yl)cyclopropane-1-carbonyl]azetidin-2-yl}methyl)ethanesulfonamide,
N-({(2R,3R)-3-fluoro-1-[(1S,2S)-2-fluoro-2-(2′,5,6′-trifluoro[1,1′-biphenyl]-2-yl)cyclopropane-1-carbonyl]azetidin-2-yl}methyl)-N′-methylsulfuric diamide,
N-[(1S)-1-{(2S)-1-[(1S,2S)-2-(2′,6′-difluoro [1,1′-biphenyl]-2-yl)-2-fluorocyclopropane-1-carbonyl]azetidin-2-yl}ethyl]methanesulfonamide,
N-({(2S,3R)-1-[(1S,2S)-2-(2′,6′-difluoro [1,1′-biphenyl]-2-yl)-2-fluorocyclopropane-1-carbonyl]-3-methylazetidin-2-yl}methyl)methanesulfonamide,
N-({(2R,3R)-1-[(1S,2S)-2-(2′,6′-difluoro-5-methyl[1,1′-biphenyl]-2-yl)-2-fluorocyclopropane-1-carbonyl]-3-fluoroazetidin-2-yl}methyl)methanesulfonamide,
N-({(2R,3R)-1-[(1R,2R)-2-(5-chloro-2′,6′-difluoro[1,1′-biphenyl]-2-yl)-2-fluorocyclopropane-1-carbonyl]-3-fluoroazetidin-2-yl}methyl)ethanesulfonamide, and
N-({(2R,3R)-1-[(1S,2S)-2-(5-chloro-2′,6′-difluoro[1,1′-biphenyl]-2-yl)-2-fluorocyclopropane-1-carbonyl]-3-fluoroazetidin-2-yl}methyl)ethanesulfonamide,
   or a pharmaceutically acceptable salt thereof.
   [28] A medicament comprising the compound or salt according to any one of the above-mentioned [1] to [27].
   [29] The medicament according to the above-mentioned [28], which is an orexin type 2 receptor agonist.
   [30] The medicament according to the above-mentioned [28], which is an agent for the prophylaxis or treatment of narcolepsy.
   [31] The compound or salt according to any one of the above-mentioned [1] to [27] for use in the prophylaxis or treatment of narcolepsy.
   [32] A method of activating an orexin type 2 receptor in a mammal, which comprises administering an effective amount of the compound or salt according to any one of the above-mentioned [1] to [27] to the mammal.
   [33] A method for the prophylaxis or treatment of narcolepsy in a mammal, which comprises administering an effective amount of the compound or salt according to any one of the above-mentioned [1] to [24] to the mammal.
   [34] A method of treating an orexin-mediated disease or disorder in a mammal in need thereof comprising administering to the mammal a therapeutically effective amount of the compound or a pharmaceutically acceptable salt according to any one of the above-mentioned [1] to [27].
   [35] The method according to the above-mentioned [34], wherein the orexin-mediated disease or disorder is narcolepsy.
   [36] Use of the compound or salt according to any one of the above-mentioned [1] to [27] for the manufacture of an agent for the prophylaxis or treatment of narcolepsy.
   [37] Use of the compound or salt according to any one of the above-mentioned [1] to [24] for the manufacture of a medicament in the treatment of an orexin-mediated disease or disorder.
   [38] A pharmaceutical composition comprising a compound or a pharmaceutically acceptable salt thereof according to any one of the above-mentioned [1] to [27].

### Effect of the Invention

The compound of the present invention has an orexin type 2 receptor agonist activity, and is useful as an agent for the prophylaxis or treatment of narcolepsy.

### DETAILED DESCRIPTION OF THE INVENTION

The definition of each substituent used in the present specification is described in detail in the following. Unless otherwise specified, each substituent has the following definition.

In the present specification, examples of the “halogen atom” include fluorine, chlorine, bromine and iodine.

As used herein, the term “alkyl” represents a saturated, straight or branched hydrocarbon moiety having the specified number of carbon atoms. The term “(C₁-C₆)alkyl” refers to an alkyl moiety containing from 1 to 6 carbon atoms. In the present specification, examples of the “C₁₋₆ alkyl group” include methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl and 2-ethylbutyl.

“Alkoxy” refers to a group containing an alkyl radical, defined herein above, attached through an oxygen linking atom. The term “(C₁-C₆)alkoxy” refers to a straight- or branched-chain hydrocarbon radical having at least 1 and up to 4 carbon atoms attached through an oxygen linking atom. Exemplary “(C₁-C₄)alkoxy” groups useful in the present invention include methoxy, ethoxy, n-propoxy, isopropoxy, n-butoxy, s-butoxy, isobutoxy, t-butoxy, n-pentyloxy, and n-hexyloxy.

In the present specification, examples of the “optionally halogenated C₁₋₆ alkyl group” include a C₁₋₆ alkyl group optionally having 1 to 7, preferably 1 to 5, halogen atoms.

Specific examples thereof include methyl, chloromethyl, difluoromethyl, trichloromethyl, trifluoromethyl, ethyl, 2-bromoethyl, 2,2,2-trifluoroethyl, tetrafluoroethyl, pentafluoroethyl, propyl, 2,2-difluoropropyl, 3,3,3-trifluoropropyl, isopropyl, butyl, 4,4,4-trifluorobutyl, isobutyl, sec-butyl, tert-butyl, pentyl, isopentyl, neopentyl, 5,5,5-trifluoropentyl, hexyl and 6,6,6-trifluorohexyl.

The term “halo(C₁-C₆)alkyl,” as used in the present specification, refers to a (C₁-C₆)alkyl group substituted with one, two, three, four, five, six, or seven halogen atoms.

The term “((C₁-C₆)alkyl)-OH,” as used in the present specification, refers to a (C₁-C₆)alkyl group substituted with a hydroxy group.

In the present specification, examples of the “C₂₋₆ alkenyl group” include ethenyl, 1-propenyl, 2-propenyl, 2-methyl-1-propenyl, 1-butenyl, 2-butenyl, 3-butenyl, 3-methyl-2-butenyl, 1-pentenyl, 2-pentenyl, 3-pentenyl, 4-pentenyl, 4-methyl-3-pentenyl, 1-hexenyl, 3-hexenyl and 5-hexenyl.

In the present specification, examples of the “C₂₋₆ alkynyl group” include ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and 4-methyl-2-pentynyl.

As used herein, “5- or 6-membered heteroaryl” represents a group or moiety comprising an aromatic monovalent monocyclic radical, containing 5 or 6 ring atoms, including at least one carbon atom and 1 to 4 heteroatoms independently selected from nitrogen, oxygen, and sulfur. Selected 5-membered heteroaryl groups contain one nitrogen, oxygen, or sulfur ring heteroatom, and optionally contain 1, 2, or 3 additional nitrogen ring atoms. Selected 6-membered heteroaryl groups contain 1, 2, or 3 nitrogen ring heteroatoms. Illustrative examples of 5- or 6-membered heteroaryl groups useful in the present invention include, but are not limited to furanyl, thienyl, pyrrolyl, imidazolyl, pyrazolyl, triazolyl, tetrazolyl, oxazolyl, thiazolyl, isoxazolyl, isothiazolyl, oxadiazolyl, thiadiazolyl, pyridinyl, pyridazinyl, pyrazinyl, pyrimidinyl, and triazinyl.

As used herein, the term “cycloalkyl” refers to a non-aromatic, saturated, cyclic hydrocarbon ring containing the specified number of carbon atoms. In the present specification, examples of the “C₃₋₁₀ cycloalkyl group” include cyclopropyl, cyclobutyl, cyclopentyl, cyclohexyl, cycloheptyl, cyclooctyl, bicyclo[2.2.1]heptyl, bicyclo[2.2.2]octyl, bicyclo[3.2.1]octyl and adamantyl.

In the present specification, examples of the “optionally halogenated C₃₋₁₀ cycloalkyl group” include a C₃₋₁₀ cycloalkyl group optionally having 1 to 7, preferably 1 to 5, halogen atoms. Specific examples thereof include cyclopropyl, 2,2-difluorocyclopropyl, 2,3-difluorocyclopropyl, cyclobutyl, difluorocyclobutyl, cyclopentyl, cyclohexyl, cycloheptyl and cyclooctyl.

In the present specification, examples of the “C₃₋₁₀ cycloalkenyl group” include cyclopropenyl, cyclobutenyl, cyclopentenyl, cyclohexenyl, cycloheptenyl and cyclooctenyl.

In the present specification, examples of the “C₆₋₁₄ aryl group” include phenyl, 1-naphthyl, 2-naphthyl, 1-anthryl, 2-anthryl and 9-anthryl.

In the present specification, examples of the “C₇₋₁₆ aralkyl group” include benzyl, phenethyl, naphthylmethyl and phenylpropyl.

In the present specification, examples of the “C₁₋₆ alkoxy group” include methoxy, ethoxy, propoxy, isopropoxy, butoxy, isobutoxy, sec-butoxy, tert-butoxy, pentyloxy and hexyloxy.

In the present specification, examples of the “optionally halogenated C₁₋₆ alkoxy group” include a C₁₋₆ alkoxy group optionally having 1 to 7, preferably 1 to 5, halogen atoms. Specific examples thereof include methoxy, difluoromethoxy, trifluoromethoxy, ethoxy, 2,2,2-trifluoroethoxy, propoxy, isopropoxy, butoxy, 4,4,4-trifluorobutoxy, isobutoxy, sec-butoxy, pentyloxy and hexyloxy.

In the present specification, examples of the “C₃₋₁₀ cycloalkyloxy group” include cyclopropyloxy, cyclobutyloxy, cyclopentyloxy, cyclohexyloxy, cycloheptyloxy and cyclooctyloxy.

In the present specification, examples of the “C₁₋₆ alkylthio group” include methylthio, ethylthio, propylthio, isopropylthio, butylthio, sec-butylthio, tert-butylthio, pentylthio and hexylthio.

In the present specification, examples of the “optionally halogenated C₁₋₆ alkylthio group” include a C₁₋₆ alkylthio group optionally having 1 to 7, preferably 1 to 5, halogen atoms. Specific examples thereof include methylthio, difluoromethylthio, trifluoromethylthio, ethylthio, propylthio, isopropylthio, butylthio, 4,4,4-trifluorobutylthio, pentylthio and hexylthio.

In the present specification, examples of the “C₁₋₆ alkyl-carbonyl group” include acetyl, propanoyl, butanoyl, 2-methylpropanoyl, pentanoyl, 3-methylbutanoyl, 2-methylbutanoyl, 2,2-dimethylpropanoyl, hexanoyl and heptanoyl.

In the present specification, examples of the “optionally halogenated C₁₋₆ alkyl-carbonyl group” include a C₁₋₆ alkyl-carbonyl group optionally having 1 to 7, preferably 1 to 5, halogen atoms. Specific examples thereof include acetyl, chloroacetyl, trifluoroacetyl, trichloroacetyl, propanoyl, butanoyl, pentanoyl and hexanoyl.

In the present specification, examples of the “C₁₋₆ alkoxy-carbonyl group” include methoxycarbonyl, ethoxycarbonyl, propoxycarbonyl, isopropoxycarbonyl, butoxycarbonyl, isobutoxycarbonyl, sec-butoxycarbonyl, tert-butoxycarbonyl, pentyloxycarbonyl and hexyloxycarbonyl.

In the present specification, examples of the “C₆₋₁₄ aryl-carbonyl group” include benzoyl, 1-naphthoyl and 2-naphthoyl.

In the present specification, examples of the “C₇₋₁₆ aralkyl-carbonyl group” include phenylacetyl and phenylpropionyl.

In the present specification, examples of the “5- to 14-membered aromatic heterocyclylcarbonyl group” include nicotinoyl, isonicotinoyl, thenoyl and furoyl.

In the present specification, examples of the “3- to 14-membered non-aromatic heterocyclylcarbonyl group” include morpholinylcarbonyl, piperidinylcarbonyl and pyrrolidinylcarbonyl.

In the present specification, examples of the “mono- or di-C₁₋₆ alkyl-carbamoyl group” include methylcarbamoyl, ethylcarbamoyl, dimethylcarbamoyl, diethylcarbamoyl and N-ethyl-N-methylcarbamoyl.

In the present specification, examples of the “mono- or di-C₇₋₁₆ aralkyl-carbamoyl group” include benzylcarbamoyl and phenethylcarbamoyl.

In the present specification, examples of the “C₁₋₆ alkylsulfonyl group” include methylsulfonyl, ethylsulfonyl, propylsulfonyl, isopropylsulfonyl, butylsulfonyl, sec-butylsulfonyl and tert-butylsulfonyl.

In the present specification, examples of the “optionally halogenated C₁₋₆ alkylsulfonyl group” include a C₁₋₆ alkylsulfonyl group optionally having 1 to 7, preferably 1 to 5, halogen atoms. Specific examples thereof include methylsulfonyl, difluoromethylsulfonyl, trifluoromethylsulfonyl, ethylsulfonyl, propylsulfonyl, isopropylsulfonyl, butylsulfonyl, 4,4,4-trifluorobutylsulfonyl, pentylsulfonyl and hexylsulfonyl.

In the present specification, examples of the “C₆₋₁₄ arylsulfonyl group” include phenylsulfonyl, 1-naphthylsulfonyl and 2-naphthylsulfonyl.

In the present specification, examples of the “substituent” include a halogen atom, a cyano group, a nitro group, an optionally substituted hydrocarbon group, an optionally substituted heterocyclic group, an acyl group, an optionally substituted amino group, an optionally substituted carbamoyl group, an optionally substituted thiocarbamoyl group, an optionally substituted sulfamoyl group, an optionally substituted hydroxy group, an optionally substituted sulfanyl (SH) group and an optionally substituted silyl group.

In the present specification, examples of the “hydrocarbon group” (including “hydrocarbon group” of “optionally substituted hydrocarbon group”) include a C₁₋₆ alkyl group, a C₂₋₆ alkenyl group, a C₂₋₆ alkynyl group, a C₃₋₁₀ cycloalkyl group, a C₃₋₁₀ cycloalkenyl group, a C₆₋₁₄ aryl group and a C₇₋₁₆ aralkyl group.

In the present specification, examples of the “optionally substituted hydrocarbon group” include a hydrocarbon group optionally having substituent(s) selected from the following Substituent group A.

### [Substituent Group A]

(1) a halogen atom,
(2) a nitro group,
(3) a cyano group,
(4) an oxo group,
(5) a hydroxy group,
(6) an optionally halogenated C₁₋₆ alkoxy group,
(7) a C₆₋₁₄ aryloxy group (e.g., phenoxy, naphthoxy),
(8) a C₇₋₁₆ aralkyloxy group (e.g., benzyloxy),
(9) a 5- to 14-membered aromatic heterocyclyloxy group (e.g., pyridyloxy),
(10) a 3- to 14-membered non-aromatic heterocyclyloxy group (e.g., tetrahydropyranyloxy, morpholinyloxy, piperidinyloxy),
(11) a C₁₋₆ alkyl-carbonyloxy group (e.g., acetoxy, propanoyloxy),
(12) a C₆₋₁₄ aryl-carbonyloxy group (e.g., benzoyloxy, 1-naphthoyloxy, 2-naphthoyloxy),
(13) a C₁₋₆ alkoxy-carbonyloxy group (e.g., methoxycarbonyloxy, ethoxycarbonyloxy, propoxycarbonyloxy, butoxycarbonyloxy),
(14) a mono- or di-C₁₋₆ alkyl-carbamoyloxy group (e.g., methylcarbamoyloxy, ethylcarbamoyloxy, dimethylcarbamoyloxy, diethylcarbamoyloxy),
(15) a C₆₋₁₄ aryl-carbamoyloxy group (e.g., phenylcarbamoyloxy, naphthylcarbamoyloxy),
(16) a 5- to 14-membered aromatic heterocyclylcarbonyloxy group (e.g., nicotinoyloxy),
(17) a 3- to 14-membered non-aromatic heterocyclylcarbonyloxy group (e.g., morpholinylcarbonyloxy, piperidinylcarbonyloxy),
(18) an optionally halogenated C₁₋₆ alkylsulfonyloxy group (e.g., methylsulfonyloxy, trifluoromethylsulfonyloxy),
(19) a C₆₋₁₄ arylsulfonyloxy group optionally substituted by a C₁₋₆ alkyl group (e.g., phenylsulfonyloxy, toluenesulfonyloxy),
(20) an optionally halogenated C₁₋₆ alkylthio group,
(21) a 5- to 14-membered aromatic heterocyclic group,
(22) a 3- to 14-membered non-aromatic heterocyclic group,
(23) a formyl group,
(24) a carboxy group,
(25) an optionally halogenated C₁₋₆ alkyl-carbonyl group,
(26) a C₆₋₁₄ aryl-carbonyl group,
(27) a 5- to 14-membered aromatic heterocyclylcarbonyl group,
(28) a 3- to 14-membered non-aromatic heterocyclylcarbonyl group,
(29) a C₁₋₆ alkoxy-carbonyl group,
(30) a C₆₋₁₄ aryloxy-carbonyl group (e.g., phenyloxycarbonyl, 1-naphthyloxycarbonyl, 2-naphthyloxycarbonyl),
(31) a C₇₋₁₆ aralkyloxy-carbonyl group (e.g., benzyloxycarbonyl, phenethyloxycarbonyl),
(32) a carbamoyl group,
(33) a thiocarbamoyl group,
(34) a mono- or di-C₁₋₆ alkyl-carbamoyl group,
(35) a C₆₋₁₄ aryl-carbamoyl group (e.g., phenylcarbamoyl),
(36) a 5- to 14-membered aromatic heterocyclylcarbamoyl group (e.g., pyridylcarbamoyl, thienylcarbamoyl),
(37) a 3- to 14-membered non-aromatic heterocyclylcarbamoyl group (e.g., morpholinylcarbamoyl, piperidinylcarbamoyl),
(38) an optionally halogenated C₁₋₆ alkylsulfonyl group,
(39) a C₆₋₁₄ arylsulfonyl group,
(40) a 5- to 14-membered aromatic heterocyclylsulfonyl group (e.g., pyridylsulfonyl, thienylsulfonyl),
(41) an optionally halogenated C₁₋₆ alkylsulfinyl group,
(42) a C₆₋₁₄ arylsulfinyl group (e.g., phenylsulfinyl, 1-naphthylsulfinyl, 2-naphthylsulfinyl),
(43) a 5- to 14-membered aromatic heterocyclylsulfinyl group (e.g., pyridylsulfinyl, thienylsulfinyl),
(44) an amino group,
(45) a mono- or di-C₁₋₆ alkylamino group (e.g., methylamino, ethylamino, propylamino, isopropylamino, butylamino, dimethylamino, diethylamino, dipropylamino, dibutylamino, N-ethyl-N-methylamino),
(46) a mono- or di-C₆₋₁₄ arylamino group (e.g., phenylamino),
(47) a 5- to 14-membered aromatic heterocyclylamino group (e.g., pyridylamino),
(48) a C₇₋₁₆ aralkylamino group (e.g., benzylamino),
(49) a formylamino group,
(50) a C₁₋₆ alkyl-carbonylamino group (e.g., acetylamino, propanoylamino, butanoylamino),
(51) a (C₁₋₆ alkyl)(C₁₋₆ alkyl-carbonyl) amino group (e.g., N-acetyl-N-methylamino),
(52) a C₆₋₁₄ aryl-carbonylamino group (e.g., phenylcarbonylamino, naphthylcarbonylamino),
(53) a C₁₋₆ alkoxy-carbonylamino group (e.g., methoxycarbonylamino, ethoxycarbonylamino, propoxycarbonylamino, butoxycarbonylamino, tert-butoxycarbonylamino),
(54) a C₇₋₁₆ aralkyloxy-carbonylamino group (e.g., benzyloxycarbonylamino),
(55) a C₁₋₆ alkylsulfonylamino group (e.g., methylsulfonylamino, ethylsulfonylamino),
(56) a C₆₋₁₄ arylsulfonylamino group optionally substituted by a C₁₋₆ alkyl group (e.g., phenylsulfonylamino, toluenesulfonylamino),
(57) an optionally halogenated C₁₋₆ alkyl group,
(58) a C₂₋₆ alkenyl group,
(59) a C₂₋₆ alkynyl group,
(60) a C₃₋₁₀ cycloalkyl group,
(61) a C₃₋₁₀ cycloalkenyl group, and
(62) a C₆₋₁₄ aryl group.

The number of the above-mentioned substituents in the “optionally substituted hydrocarbon group” is, for example, 1 to 5, preferably 1 to 3. When the number of the substituents is two or more, the respective substituents may be the same or different.

Unless otherwise specified, in the present specification, examples of the “heterocyclic group” (including “heterocyclic group” of “optionally substituted heterocyclic group”) include (i) an aromatic heterocyclic group, (ii) a non-aromatic heterocyclic group and (iii) a 7- to 10-membered bridged heterocyclic group, each containing, as a ring-constituting atom besides carbon atom, 1 to 4 heteroatoms selected from a nitrogen atom, a sulfur atom and an oxygen atom.

In the present specification, examples of the “aromatic heterocyclic group” (including “5- to 14-membered aromatic heterocyclic group”) include a 5- to 14-membered (preferably 5- to 10-membered) aromatic heterocyclic group containing, as a ring-constituting atom besides carbon atom, 1 to 4 heteroatoms selected from a nitrogen atom, a sulfur atom and an oxygen atom.

Preferable examples of the “aromatic heterocyclic group” include 5- or 6-membered monocyclic aromatic heterocyclic groups such as thienyl, furyl, pyrrolyl, imidazolyl, pyrazolyl, thiazolyl, isothiazolyl, oxazolyl, isoxazolyl, pyridyl, pyrazinyl, pyrimidinyl, pyridazinyl, 1,2,4-oxadiazolyl, 1,3,4-oxadiazolyl, 1,2,4-thiadiazolyl, 1,3,4-thiadiazolyl, triazolyl, tetrazolyl, triazinyl and the like; and 8- to 14-membered fused polycyclic (preferably bi- or tricyclic) aromatic heterocyclic groups such as benzothiophenyl, benzofuranyl, benzimidazolyl, benzoxazolyl, benzisoxazolyl, benzothiazolyl, benzisothiazolyl, benzotriazolyl, imidazopyridinyl, thienopyridinyl, furopyridinyl, pyrrolopyridinyl, pyrazolopyridinyl, oxazolopyridinyl, thiazolopyridinyl, imidazopyrazinyl, imidazopyrimidinyl, thienopyrimidinyl, furopyrimidinyl, pyrrolopyrimidinyl, pyrazolopyrimidinyl, oxazolopyrimidinyl, thiazolopyrimidinyl, pyrazolotriazinyl, naphtho[2,3-b]thienyl, phenoxathiinyl, indolyl, isoindolyl, 1H-indazolyl, purinyl, isoquinolyl, quinolyl, phthalazinyl, naphthyridinyl, quinoxalinyl, quinazolinyl, cinnolinyl, carbazolyl, β-carbolinyl, phenanthridinyl, acridinyl, phenazinyl, phenothiazinyl, phenoxazinyl and the like.

In the present specification, examples of the “non-aromatic heterocyclic group” (including “3- to 14-membered non-aromatic heterocyclic group”) include a 3- to 14-membered (preferably 4- to 10-membered) non-aromatic heterocyclic group containing, as a ring-constituting atom besides carbon atom, 1 to 4 heteroatoms selected from a nitrogen atom, a sulfur atom and an oxygen atom.

Preferable examples of the “non-aromatic heterocyclic group” include 3- to 8-membered monocyclic non-aromatic heterocyclic groups such as aziridinyl, oxiranyl, thiiranyl, azetidinyl, oxetanyl, thietanyl, tetrahydrothienyl, tetrahydrofuranyl, pyrrolinyl, pyrrolidinyl, imidazolinyl, imidazolidinyl, oxazolinyl, oxazolidinyl, pyrazolinyl, pyrazolidinyl, thiazolinyl, thiazolidinyl, tetrahydroisothiazolyl, tetrahydrooxazolyl, tetrahydroisooxazolyl, piperidinyl, piperazinyl, tetrahydropyridinyl, dihydropyridinyl, dihydrothiopyranyl, tetrahydropyrimidinyl, tetrahydropyridazinyl, dihydropyranyl, tetrahydropyranyl, tetrahydrothiopyranyl, morpholinyl, thiomorpholinyl, azepanyl, diazepanyl, azepinyl, oxepanyl, azocanyl, diazocanyl and the like; and 9- to 14-membered fused polycyclic (preferably bi- or tricyclic) non-aromatic heterocyclic groups such as dihydrobenzofuranyl, dihydrobenzimidazolyl, dihydrobenzoxazolyl, dihydrobenzothiazolyl, dihydrobenzisothiazolyl, dihydronaphtho[2,3-b]thienyl, tetrahydroisoquinolyl, tetrahydroquinolyl, 4H-quinolizinyl, indolinyl, isoindolinyl, tetrahydrothieno[2,3-c]pyridinyl, tetrahydrobenzazepinyl, tetrahydroquinoxalinyl, tetrahydrophenanthridinyl, hexahydrophenothiazinyl, hexahydrophenoxazinyl, tetrahydrophthalazinyl, tetrahydronaphthyridinyl, tetrahydroquinazolinyl, tetrahydrocinnolinyl, tetrahydrocarbazolyl, tetrahydro-β-carbolinyl, tetrahydroacrydinyl, tetrahydrophenazinyl, tetrahydrothioxanthenyl, octahydroisoquinolyl and the like.

In the present specification, preferable examples of the “7- to 10-membered bridged heterocyclic group” include quinuclidinyl and 7-azabicyclo[2.2.1]heptanyl.

In the present specification, examples of the “nitrogen-containing heterocyclic group” include a “heterocyclic group” containing at least one nitrogen atom as a ring-constituting atom.

In the present specification, “3-8-membered heterocyclyl” represents a group or moiety comprising a non aromatic, monovalent monocyclic radical, which is saturated or partially unsaturated, containing 3, 4, 5, 6, 7, or 8 ring atoms, which includes one or two heteroatoms selected independently from oxygen, sulfur, and nitrogen.

In the present specification, examples of the “optionally substituted heterocyclic group” include a heterocyclic group optionally having substituent(s) selected from the above-mentioned Substituent group A.

The number of the substituents in the “optionally substituted heterocyclic group” is, for example, 1 to 3. When the number of the substituents is two or more, the respective substituents may be the same or different.

In the present specification, examples of the “acyl group” include a formyl group, a carboxy group, a carbamoyl group, a thiocarbamoyl group, a sulfino group, a sulfo group, a sulfamoyl group and a phosphono group, each optionally having “1 or 2 substituents selected from a C₁₋₆ alkyl group, a C₂₋₆ alkenyl group, a C₃₋₁₀ cycloalkyl group, a C₃₋₁₀ cycloalkenyl group, a C₆₋₁₄ aryl group, a C₇₋₁₆ aralkyl group, a 5- to 14-membered aromatic heterocyclic group and a 3- to 14-membered non-aromatic heterocyclic group, each of which optionally has 1 to 3 substituents selected from a halogen atom, an optionally halogenated C₁₋₆ alkoxy group, a hydroxy group, a nitro group, a cyano group, an amino group and a carbamoyl group”.

Examples of the “acyl group” also include a hydrocarbon-sulfonyl group, a heterocyclylsulfonyl group, a hydrocarbon-sulfinyl group and a heterocyclylsulfinyl group.

Here, the hydrocarbon-sulfonyl group means a hydrocarbon group-bonded sulfonyl group, the heterocyclylsulfonyl group means a heterocyclic group-bonded sulfonyl group, the hydrocarbon-sulfinyl group means a hydrocarbon group-bonded sulfinyl group and the heterocyclylsulfinyl group means a heterocyclic group-bonded sulfinyl group.

Preferable examples of the “acyl group” include a formyl group, a carboxy group, a C₁₋₆ alkyl-carbonyl group, a C₂₋₆ alkenyl-carbonyl group (e.g., crotonoyl), a C₃₋₁₀ cycloalkyl-carbonyl group (e.g., cyclobutanecarbonyl, cyclopentanecarbonyl, cyclohexanecarbonyl, cycloheptanecarbonyl), a C₃₋₁₀ cycloalkenyl-carbonyl group (e.g., 2-cyclohexenecarbonyl), a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a C₆₋₁₄ aryloxy-carbonyl group (e.g., phenyloxycarbonyl, naphthyloxycarbonyl), a C₇₋₁₆ aralkyloxy-carbonyl group (e.g., benzyloxycarbonyl, phenethyloxycarbonyl), a carbamoyl group, a mono- or di-C₁₋₆ alkyl-carbamoyl group, a mono- or di-C₂₋₆ alkenyl-carbamoyl group (e.g., diallylcarbamoyl), a mono- or di-C₃₋₁₀ cycloalkyl-carbamoyl group (e.g., cyclopropylcarbamoyl), a mono- or di-C₆₋₁₄ aryl-carbamoyl group (e.g., phenylcarbamoyl), a mono- or di-C₇₋₁₆ aralkyl-carbamoyl group, a 5- to 14-membered aromatic heterocyclylcarbamoyl group (e.g., pyridylcarbamoyl), a thiocarbamoyl group, a mono- or di-C₁₋₆ alkyl-thiocarbamoyl group (e.g., methylthiocarbamoyl, N-ethyl-N-methylthiocarbamoyl), a mono- or di-C₂₋₆ alkenyl-thiocarbamoyl group (e.g., diallylthiocarbamoyl), a mono- or di-C₃₋₁₀ cycloalkyl-thiocarbamoyl group (e.g., cyclopropylthiocarbamoyl, cyclohexylthiocarbamoyl), a mono- or di-C₆₋₁₄ aryl-thiocarbamoyl group (e.g., phenylthiocarbamoyl), a mono- or di-C₇₋₁₆ aralkyl-thiocarbamoyl group (e.g., benzylthiocarbamoyl, phenethylthiocarbamoyl), a 5- to 14-membered aromatic heterocyclylthiocarbamoyl group (e.g., pyridylthiocarbamoyl), a sulfino group, a C₁₋₆ alkylsulfinyl group (e.g., methylsulfinyl, ethylsulfinyl), a sulfo group, a C₁₋₆ alkylsulfonyl group, a C₆₋₁₄ arylsulfonyl group, a phosphono group and a mono- or di-C₁₋₆ alkylphosphono group (e.g., dimethylphosphono, diethylphosphono, diisopropylphosphono, dibutylphosphono).

In the present specification, examples of the “optionally substituted amino group” include an amino group optionally having “1 or 2 substituents selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group, a mono- or di-C₇₋₁₆ aralkyl-carbamoyl group, a C₁₋₆ alkylsulfonyl group and a C₆₋₁₄ arylsulfonyl group, each of which optionally has 1 to 3 substituents selected from Substituent group A”.

Preferable examples of the optionally substituted amino group include an amino group, a mono- or di-(optionally halogenated C₁₋₆ alkyl) amino group (e.g., methylamino, trifluoromethylamino, dimethylamino, ethylamino, diethylamino, propylamino, dibutylamino), a mono- or di-C₂₋₆ alkenylamino group (e.g., diallylamino), a mono- or di-C₃₋₁₀ cycloalkylamino group (e.g., cyclopropylamino, cyclohexylamino), a mono- or di-C₆₋₁₄ arylamino group (e.g., phenylamino), a mono- or di-C₇₋₁₆ aralkylamino group (e.g., benzylamino, dibenzylamino), a mono- or di-(optionally halogenated C₁₋₆ alkyl)-carbonylamino group (e.g., acetylamino, propionylamino), a mono- or di-C₆₋₁₄ aryl-carbonylamino group (e.g., benzoylamino), a mono- or di-C₇₋₁₆ aralkyl-carbonylamino group (e.g., benzylcarbonylamino), a mono- or di-5- to 14-membered aromatic heterocyclylcarbonylamino group (e.g., nicotinoylamino, isonicotinoylamino), a mono- or di-3- to 14-membered non-aromatic heterocyclylcarbonylamino group (e.g., piperidinylcarbonylamino), a mono- or di-C₁₋₆ alkoxy-carbonylamino group (e.g., tert-butoxycarbonylamino), a 5- to 14-membered aromatic heterocyclylamino group (e.g., pyridylamino), a carbamoylamino group, a (mono- or di-C₁₋₆ alkyl-carbamoyl) amino group (e.g., methylcarbamoylamino), a (mono- or di-C₇₋₁₆ aralkyl-carbamoyl) amino group (e.g., benzylcarbamoylamino), a C₁₋₆ alkylsulfonylamino group (e.g., methylsulfonylamino, ethylsulfonylamino), a C₆₋₁₄ arylsulfonylamino group (e.g., phenylsulfonylamino), a (C₁₋₆ alkyl)(C₁₋₆ alkyl-carbonyl) amino group (e.g., N-acetyl-N-methylamino) and a (C₁₋₆ alkyl)(C₆₋₁₄ aryl-carbonyl) amino group (e.g., N-benzoyl-N-methylamino).

In the present specification, examples of the “optionally substituted carbamoyl group” include a carbamoyl group optionally having “1 or 2 substituents selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group and a mono- or di-C₇₋₁₆ aralkyl-carbamoyl group, each of which optionally has 1 to 3 substituents selected from Substituent group A”.

Preferable examples of the optionally substituted carbamoyl group include a carbamoyl group, a mono- or di-C₁₋₆ alkyl-carbamoyl group, a mono- or di-C₂₋₆ alkenyl-carbamoyl group (e.g., diallylcarbamoyl), a mono- or di-C₃₋₁₀ cycloalkyl-carbamoyl group (e.g., cyclopropylcarbamoyl, cyclohexylcarbamoyl), a mono- or di-C₆₋₁₄ aryl-carbamoyl group (e.g., phenylcarbamoyl), a mono- or di-C₇₋₁₆ aralkyl-carbamoyl group, a mono- or di-C₁₋₆ alkyl-carbonyl-carbamoyl group (e.g., acetylcarbamoyl, propionylcarbamoyl), a mono- or di-C₆₋₁₄ aryl-carbonyl-carbamoyl group (e.g., benzoylcarbamoyl) and a 5- to 14-membered aromatic heterocyclylcarbamoyl group (e.g., pyridylcarbamoyl).

In the present specification, examples of the “optionally substituted thiocarbamoyl group” include a thiocarbamoyl group optionally having “1 or 2 substituents selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group and a mono- or di-C₇₋₁₆ aralkyl-carbamoyl group, each of which optionally has 1 to 3 substituents selected from Substituent group A”.

Preferable examples of the optionally substituted thiocarbamoyl group include a thiocarbamoyl group, a mono- or di-C₁₋₆ alkyl-thiocarbamoyl group (e.g., methylthiocarbamoyl, ethylthiocarbamoyl, dimethylthiocarbamoyl, diethylthiocarbamoyl, N-ethyl-N-methylthiocarbamoyl), a mono- or di-C₂₋₆ alkenyl-thiocarbamoyl group (e.g., diallylthiocarbamoyl), a mono- or di-C₃₋₁₀ cycloalkyl-thiocarbamoyl group (e.g., cyclopropylthiocarbamoyl, cyclohexylthiocarbamoyl), a mono- or di-C₆₋₁₄ aryl-thiocarbamoyl group (e.g., phenylthiocarbamoyl), a mono- or di-C₇₋₁₆ aralkyl-thiocarbamoyl group (e.g., benzylthiocarbamoyl, phenethylthiocarbamoyl), a mono- or di-C₁₋₆ alkyl-carbonyl-thiocarbamoyl group (e.g., acetylthiocarbamoyl, propionylthiocarbamoyl), a mono- or di-C₆₋₁₄ aryl-carbonyl-thiocarbamoyl group (e.g., benzoylthiocarbamoyl) and a 5- to 14-membered aromatic heterocyclylthiocarbamoyl group (e.g., pyridylthiocarbamoyl).

In the present specification, examples of the “optionally substituted sulfamoyl group” include a sulfamoyl group optionally having “1 or 2 substituents selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group and a mono- or di-C₇₋₁₆ aralkyl-carbamoyl group, each of which optionally has 1 to 3 substituents selected from Substituent group A”.

Preferable examples of the optionally substituted sulfamoyl group include a sulfamoyl group, a mono- or di-C₁₋₆ alkyl-sulfamoyl group (e.g., methylsulfamoyl, ethylsulfamoyl, dimethylsulfamoyl, diethylsulfamoyl, N-ethyl-N-methylsulfamoyl), a mono- or di-C₂₋₆ alkenyl-sulfamoyl group (e.g., diallylsulfamoyl), a mono- or di-C₃₋₁₀ cycloalkyl-sulfamoyl group (e.g., cyclopropylsulfamoyl, cyclohexylsulfamoyl), a mono- or di-C₆₋₁₄ aryl-sulfamoyl group (e.g., phenylsulfamoyl), a mono- or di-C₇₋₁₆ aralkyl-sulfamoyl group (e.g., benzylsulfamoyl, phenethylsulfamoyl), a mono- or di-C₁₋₆ alkyl-carbonyl-sulfamoyl group (e.g., acetylsulfamoyl, propionylsulfamoyl), a mono- or di-C₆₋₁₄ aryl-carbonyl-sulfamoyl group (e.g., benzoylsulfamoyl) and a 5- to 14-membered aromatic heterocyclylsulfamoyl group (e.g., pyridylsulfamoyl).

In the present specification, examples of the “optionally substituted hydroxy group” include a hydroxy group optionally having “a substituent selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group, a mono- or di-C₇₋₁₆ aralkyl-carbamoyl group, a C₁₋₆ alkylsulfonyl group and a C₆₋₁₄ arylsulfonyl group, each of which optionally has 1 to 3 substituents selected from Substituent group A”.

Preferable examples of the optionally substituted hydroxy group include a hydroxy group, a C₁₋₆ alkoxy group, a C₂₋₆ alkenyloxy group (e.g., allyloxy, 2-butenyloxy, 2-pentenyloxy, 3-hexenyloxy), a C₃₋₁₀ cycloalkyloxy group (e.g., cyclohexyloxy), a C₆₋₁₄ aryloxy group (e.g., phenoxy, naphthyloxy), a C₇₋₁₆ aralkyloxy group (e.g., benzyloxy, phenethyloxy), a C₁₋₆ alkyl-carbonyloxy group (e.g., acetyloxy, propionyloxy, butyryloxy, isobutyryloxy, pivaloyloxy), a C₆₋₁₄ aryl-carbonyloxy group (e.g., benzoyloxy), a C₇₋₁₆ aralkyl-carbonyloxy group (e.g., benzylcarbonyloxy), a 5- to 14-membered aromatic heterocyclylcarbonyloxy group (e.g., nicotinoyloxy), a 3- to 14-membered non-aromatic heterocyclylcarbonyloxy group (e.g., piperidinylcarbonyloxy), a C₁₋₆ alkoxy-carbonyloxy group (e.g., tert-butoxycarbonyloxy), a 5- to 14-membered aromatic heterocyclyloxy group (e.g., pyridyloxy), a carbamoyloxy group, a C₁₋₆ alkyl-carbamoyloxy group (e.g., methylcarbamoyloxy), a C₇₋₁₆ aralkyl-carbamoyloxy group (e.g., benzylcarbamoyloxy), a C₁₋₆ alkylsulfonyloxy group (e.g., methylsulfonyloxy, ethylsulfonyloxy) and a C₆₋₁₄ arylsulfonyloxy group (e.g., phenylsulfonyloxy).

In the present specification, examples of the “optionally substituted sulfanyl group” include a sulfanyl group optionally having “a substituent selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group and a 5- to 14-membered aromatic heterocyclic group, each of which optionally has 1 to 3 substituents selected from Substituent group A” and a halogenated sulfanyl group.

Preferable examples of the optionally substituted sulfanyl group include a sulfanyl (—SH) group, a C₁₋₆ alkylthio group, a C₂₋₆ alkenylthio group (e.g., allylthio, 2-butenylthio, 2-pentenylthio, 3-hexenylthio), a C₃₋₁₀ cycloalkylthio group (e.g., cyclohexylthio), a C₆₋₁₄ arylthio group (e.g., phenylthio, naphthylthio), a C₇₋₁₆ aralkylthio group (e.g., benzylthio, phenethylthio), a C₁₋₆ alkyl-carbonylthio group (e.g., acetylthio, propionylthio, butyrylthio, isobutyrylthio, pivaloylthio), a C₆₋₁₄ aryl-carbonylthio group (e.g., benzoylthio), a 5- to 14-membered aromatic heterocyclylthio group (e.g., pyridylthio) and a halogenated thio group (e.g., pentafluorothio).

In the present specification, examples of the “optionally substituted silyl group” include a silyl group optionally having “1 to 3 substituents selected from a C₁₋₆ alkyl group, a C₂₋₆ alkenyl group, a C₃₋₁₀ cycloalkyl group, a C₆₋₁₄ aryl group and a C₇₋₁₆ aralkyl group, each of which optionally has 1 to 3 substituents selected from Substituent group A”.

Preferable examples of the optionally substituted silyl group include a tri-C₁₋₆ alkylsilyl group (e.g., trimethylsilyl, tert-butyl(dimethyl)silyl).

In the present specification, examples of the “hydrocarbon ring” include a C₆₋₁₄ aromatic hydrocarbon ring, C₃₋₁₀ cycloalkane and C₃₋₁₀ cycloalkene.

In the present specification, examples of the “C₆₋₁₄ aromatic hydrocarbon ring” include benzene and naphthalene.

In the present specification, examples of the “C₃₋₁₀ cycloalkane” include cyclopropane, cyclobutane, cyclopentane, cyclohexane, cycloheptane and cyclooctane.

In the present specification, examples of the “C₃₋₁₀ cycloalkene” include cyclopropene, cyclobutene, cyclopentene, cyclohexene, cycloheptene and cyclooctene.

In the present specification, examples of the “heterocycle” include an aromatic heterocycle and a non-aromatic heterocycle, each containing, as a ring-constituting atom besides carbon atom, 1 to 4 hetero atoms selected from a nitrogen atom, a sulfur atom and an oxygen atom.

In the present specification, examples of the “aromatic heterocycle” include a 5- to 14-membered (preferably 5- to 10-membered) aromatic heterocycle containing, as a ring-constituting atom besides carbon atom, 1 to 4 hetero atoms selected from a nitrogen atom, a sulfur atom and an oxygen atom. Preferable examples of the “aromatic heterocycle” include 5- or 6-membered monocyclic aromatic heterocycles such as thiophene, furan, pyrrole, imidazole, pyrazole, thiazole, isothiazole, oxazole, isoxazole, pyridine, pyrazine, pyrimidine, pyridazine, 1,2,4-oxadiazole, 1,3,4-oxadiazole, 1,2,4-thiadiazole, 1,3,4-thiadiazole, triazole, tetrazole, triazine and the like; and 8- to 14-membered fused polycyclic (preferably bi- or tricyclic) aromatic heterocycles such as benzothiophene, benzofuran, benzimidazole, benzoxazole, benzisoxazole, benzothiazole, benzisothiazole, benzotriazole, imidazopyridine, thienopyridine, furopyridine, pyrrolopyridine, pyrazolopyridine, oxazolopyridine, thiazolopyridine, imidazopyrazine, imidazopyrimidine, thienopyrimidine, furopyrimidine, pyrrolopyrimidine, pyrazolopyrimidine, oxazolopyrimidine, thiazolopyrimidine, pyrazolopyrimidine, pyrazolotriazine, naphtho[2,3-b]thiophene, phenoxathiin, indole, isoindole, 1H-indazole, purine, isoquinoline, quinoline, phthalazine, naphthyridine, quinoxaline, quinazoline, cinnoline, carbazole, β-carboline, phenanthridine, acridine, phenazine, phenothiazine, phenoxazine and the like.

In the present specification, examples of the “non-aromatic heterocycle” include a 3- to 14-membered (preferably 4- to 10-membered) non-aromatic heterocycle containing, as a ring-constituting atom besides carbon atom, 1 to 4 hetero atoms selected from a nitrogen atom, a sulfur atom and an oxygen atom. Preferable examples of the “non-aromatic heterocycle” include 3- to 8-membered monocyclic non-aromatic heterocycles such as aziridine, oxirane, thiirane, azetidine, oxetane, thietane, tetrahydrothiophene, tetrahydrofuran, pyrroline, pyrrolidine, imidazoline, imidazolidine, oxazoline, oxazolidine, pyrazoline, pyrazolidine, thiazoline, thiazolidine, tetrahydroisothiazole, tetrahydrooxazole, tetrahydroisoxazole, piperidine, piperazine, tetrahydropyridine, dihydropyridine, dihydrothiopyran, tetrahydropyrimidine, tetrahydropyridazine, dihydropyran, tetrahydropyran, tetrahydrothiopyran, morpholine, thiomorpholine, azepane, diazepane, azepine, azocane, diazocane, oxepane and the like; and 9- to 14-membered fused polycyclic (preferably bi- or tricyclic) non-aromatic heterocycles such as dihydrobenzofuran, dihydrobenzimidazole, dihydrobenzoxazole, dihydrobenzothiazole, dihydrobenzisothiazole, dihydronaphtho[2,3-b]thiophene, tetrahydroisoquinoline, tetrahydroquinoline, 4H-quinolizine, indoline, isoindoline, tetrahydrothieno[2,3-c]pyridine, tetrahydrobenzazepine, tetrahydroquinoxaline, tetrahydrophenanthridine, hexahydrophenothiazine, hexahydrophenoxazine, tetrahydrophthalazine, tetrahydronaphthyridine, tetrahydroquinazoline, tetrahydrocinnoline, tetrahydrocarbazole, tetrahydro-β-carboline, tetrahydroacridine, tetrahydrophenazine, tetrahydrothioxanthene, octahydroisoquinoline and the like.

In the present specification, examples of the “nitrogen-containing heterocycle” include a heterocycle containing at least one nitrogen atom as a ring-constituting atom, from among the “heterocycle”.

In the present specification, examples of the “4- to 6-membered heterocyclic group” include an aromatic or non-aromatic 4- to 6-membered heterocyclic group. Specific examples thereof include oxetanyl, furyl, pyrazolyl, pyridyl and pyrimidinyl.

In the present specification, examples of the “ring” include “hydrocarbon ring” and “heterocycle”.

The definition of each symbol in the formula (I) is explained in detail.

Ring W is an optionally further substituted ring.

Examples of the substituent of the above-mentioned “optionally further substituted ring” include substituents selected from Substituent group A. The number of the substituents is preferably 1 to 3. When the number of the substituents is 2 or more, the respective substituents may be the same or different.

Ring W is preferably
(1) a 3- to 8-membered monocyclic non-aromatic heterocycle (e.g., piperidine),
(2) a 5- to 6-membered monocyclic aromatic heterocycle (e.g., furan, pyridine) optionally further substituted by 1 to 3 halogen atoms (e.g., a fluorine atom),
(3) a C₆₋₁₄ aromatic hydrocarbon ring (e.g., benzene) optionally further substituted by 1 to 3 substituents selected from
   (i) a halogen atom (e.g., a fluorine atom, a chlorine atom),
   (ii) a C₁ alkyl group (e.g., methyl) optionally substituted by 1 to 3 C₁ alkoxy groups (e.g., methoxy), and
   (iii) a C₁ alkoxy group (e.g., methoxy), or
(4) a C₃₋₁₀ cycloalkane (e.g., cyclobutane, cyclopentane, cyclohexane).

Ring W is more preferably a C₆₋₁₄ aromatic hydrocarbon ring (e.g., benzene) optionally further substituted by 1 to 3 halogen atoms (e.g., a fluorine atom).

Ring W is particularly preferably a C₆₋₁₄ aromatic hydrocarbon ring (e.g., benzene) further substituted by 1 to 3 halogen atoms (e.g., a fluorine atom).

In one embodiment, Ring W is a benzene ring optionally further substituted with 2 fluorine atoms.

Ring X is an optionally further substituted 5- or 6-membered aromatic ring.

Examples of the substituent of the above-mentioned “optionally further substituted 5- or 6-membered aromatic ring” include substituents selected from Substituent group A. The number of the substituents is preferably 1 to 3. When the number of the substituents is 2 or more, the respective substituents may be the same or different.

Ring X is preferably
(1) a benzene ring optionally further substituted by 1 to 3 substituents selected from
   (i) a halogen atom (e.g., a fluorine atom, a chlorine atom),
   (ii) an optionally halogenated C₁₋₆ alkyl group (e.g., methyl, difluoromethyl), and
   (iii) a C₁₋₆ alkoxy group (e.g., methoxy), or
(2) a 5- or 6-membered monocyclic aromatic heterocycle (e.g., thiophene, thiazole, pyridine) optionally further substituted by 1 to 3 substituents selected from
   (i) a halogen atom (e.g., a fluorine atom), and
   (ii) C₁₋₆ alkyl groups (e.g., methyl).

Ring X is more preferably a benzene ring optionally further substituted by 1 to 3 substituents selected from
(i) a halogen atom (e.g., a fluorine atom, a chlorine atom), and
(ii) a C₁₋₆ alkyl group (e.g., methyl).

In one embodiment, Ring X is:

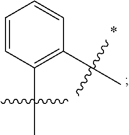 [SMILES: *c1ccccc1*]

further optionally substituted by 1 or 2 substituents independently selected from halogen (e.g., a fluorine atom, a chlorine atom) and methyl, wherein [symbol] is the point of attachment to Ring W and [symbol] is the point of attachment to Ring Y.

Ring Y is an optionally further substituted cyclopropane ring.

Examples of the substituent of the above-mentioned “optionally further substituted cyclopropane ring” include substituents selected from Substituent group A. The number of the substituents is preferably 1 to 3. When the number of the substituents is 2 or more, the respective substituents may be the same or different.

Ring Y is preferably cyclopropane ring optionally further substituted by 1 to 3 substituents selected from
(i) a halogen atom (e.g., a fluorine atom),
(ii) a cyano group, and
(iii) a C₁₋₆ alkyl group (e.g., methyl), optionally substituted by 1 to 3 substituents selected from C₁₋₆ alkoxy group (e.g., methoxy), cyano, and hydroxy.

Ring Y is more preferably cyclopropane ring optionally further substituted by 1 to 3 halogen atoms (e.g., a fluorine atom).

In one embodiment Ring Y is:

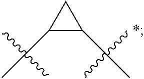 [SMILES: *C1CC1*]

further optionally substituted with one fluorine atom, wherein [symbol] is the point of attachment to Ring X and [symbol] is the point of attachment to Ring Z.

Ring Z is an optionally further substituted nitrogen-containing heterocycle.

Examples of the substituent of the above-mentioned “optionally further substituted nitrogen-containing heterocycle” include substituents selected from Substituent group A. The number of the substituents is preferably 1 to 3. When the number of the substituents is 2 or more, the respective substituents may be the same or different.

Ring Z is preferably a 3- to 14-membered nitrogen-containing heterocycle (e.g., azetidine, pyrrolidine, piperidine, morpholine, azepane, azabicyclo[3.1.0]hexane, azaspiro[3.3]heptane, azabicyclo[3.2.0]heptane, azabicyclo[3.2.1]octane, oxazepane, octahydrocyclopenta[c]pyrrole) optionally further substituted by 1 to 3 substituents selected from
(i) a halogen atom (e.g., a fluorine atom),
(ii) a hydroxy group,
(iii) a C₁₋₆ alkyl group (e.g., methyl, ethyl, isopropyl), optionally substituted by 1 to 3 substituents selected from C₁₋₆ alkoxy group (e.g., methoxy), halogen atom (e.g., a fluorine atom), and hydroxy,
(iv) a C₁₋₆ alkoxy group (e.g., methoxy),
(v) —C(O)—C₁₋₆ alkyl, and
(vi) a 5-membered monocyclic aromatic heterocycle (e.g., 1,2,4-oxadiazolyl, 1,3,4-oxadiazolyl) optionally substituted with a C₁₋₆ alkyl (e.g., methyl).

Ring Z is more preferably a 3- to 14-membered nitrogen-containing heterocycle (e.g., azetidine, pyrrolidine, piperidine, azepane, oxazepane, octahydrocyclopenta[c]pyrrole) optionally further substituted by 1 to 3 substituents selected from a halogen atom (e.g., a fluorine atom), a hydroxy group and a C₁₋₆ alkyl group (e.g., methyl).

In an embodiment, Ring Z is selected from the group consisting of:

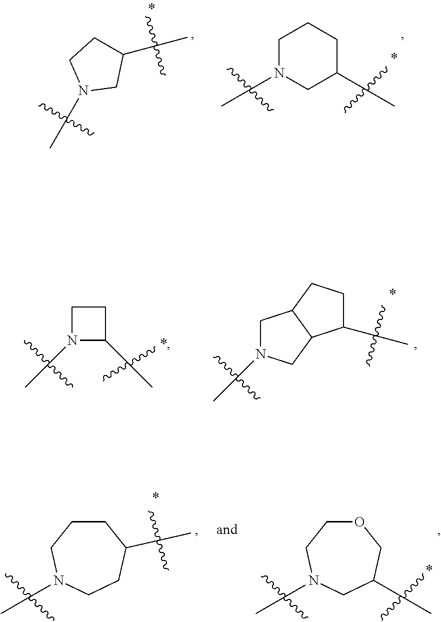 [SMILES: *C1CCN(*)C1 ; *C1CCCN(*)C1 ; *C1CCN1* ; *C1CCC2CN(*)CC12 ; *C1CCCN(*)CC1 ; *C1COCCN(*)C1]

further optionally substituted with 1 to 3 substituents independently selected from a halogen atom (e.g., a fluorine atom), a hydroxy group and a C₁₋₆ alkyl group (e.g., methyl), wherein [symbol] is the point of attachment to Ring Y—(C═O) and [symbol] is the point of attachment to L.

In one embodiment, Ring Z is:

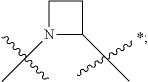 [SMILES: *C1CCN1*]

further optionally substituted with 1 to 3 substituents independently selected from a halogen atom (e.g., a fluorine atom) and a C₁₋₆ alkyl group (e.g., methyl), wherein [symbol] is the point of attachment to Ring Y—(C═O) and [symbol] is the point of attachment to L.

L is a bond, or an optionally substituted methylene group.

Examples of the substituent of the above-mentioned “optionally substituted methylene group” include substituents selected from Substituent group A. The number of the substituents is preferably 1 to 3. When the number of the substituents is 2 or more, the respective substituents may be the same or different.

L is a bond, or a methylene group optionally substituted with 1 substituent selected from
(1) a C₁₋₆ alkyl group (e.g., methyl, ethyl, isopropyl) further optionally substituted by 1 to 3 substituents selected from a C₁₋₆ alkoxy group (e.g., methoxy), and hydroxy, and
(2) a C₃₋₆ cycloalkyl group (e.g., cyclopropyl) further optionally substituted with a hydroxy group.

L is preferably a bond or a methylene group optionally substituted with a C₁₋₆ alkyl group (e.g., methyl).

L is preferably a methylene group optionally substituted with a C₁₋₆ alkyl group (e.g., methyl).

L is preferably a methylene group.

R is an optionally substituted C₁₋₆ alkyl group, an optionally substituted C₃₋₁₀ cycloalkyl group, or NR^{a}R^{b}.

wherein R^{a} is an optionally substituted C₁₋₆ alkyl group, or an optionally substituted C₃₋₁₀ cycloalkyl group; R^{b} is a hydrogen atom, an optionally substituted C₁₋₆ alkyl group, or an optionally substituted C₃₋₁₀ cycloalkyl group; or R^{a} and R^{b} in combination form an optionally further substituted nitrogen-containing heterocycle, together with adjacent nitrogen atom.

Example of “nitrogen-containing heterocycle” include a 3- to 8-membered (preferably 4-membered) monocyclic non-aromatic heterocycle containing, as a ring-constituting atom besides carbon atom, at least one nitrogen atom. Preferable examples of the “nitrogen-containing heterocycle” include azetidine.

Examples of the substituent of the above-mentioned “optionally substituted C₁₋₆ alkyl group”, “optionally substituted C₃₋₁₀ cycloalkyl group”, and “optionally further substituted nitrogen-containing heterocycle” include substituents selected from Substituent group A. The number of the substituents is preferably 1 to 3. When the number of the substituents is 2 or more, the respective substituents may be the same or different.

In one embodiment, R is an optionally substituted C₁₋₆ alkyl, an optionally substituted C₃₋₁₀ cycloalkyl, or an optionally substituted di-C₁₋₆ alkyl amine.

Examples of the substituent of the above-mentioned “optionally substituted C₁₋₆ alkyl group”, “optionally substituted C₃₋₁₀ cycloalkyl group” and “optionally substituted di-C₁₋₆ alkyl amine group” include substituents selected from Substituent group A. The number of the substituents is preferably 1 to 3. When the number of the substituents is 2 or more, the respective substituents may be the same or different.

R is preferably
(1) a C₁₋₆ alkyl group (e.g., methyl, ethyl, propyl, isopropyl) optionally substituted by 1 to 3 substituents selected from
   (i) a halogen atom (e.g., a fluorine atom, a bromine atom),
   (ii) a C₁₋₆ alkoxy (e.g., methoxy), and
   (iii) a cyclopropyl group (e.g., cyclopropyl).
(2) a C₃₋₁₀ cycloalkyl group (e.g., cyclopropyl) further optionally substituted with a C₁₋₆ alkyl group (e.g., methyl), or
(3) NR^{a}R^{b}; wherein R^{a} is a C₁₋₆ alkyl group (e.g., methyl, ethyl, or isopropyl), or a C₃₋₁₀ cycloalkyl group (e.g., cyclopropyl); R^{b} is a hydrogen atom, or a C₁₋₆ alkyl group (e.g., methyl); or R^{a} and R^{b} in combination form a nitrogen-containing heterocycle (e.g., azetidinyl), together with the adjacent nitrogen atom.

R is more preferably
(1) a C₁₋₆ alkyl group (e.g., methyl, ethyl), or
(2) NR^{a}R^{b};
   wherein R^{a} is a C₁₋₆ alkyl group (e.g., methyl); and R^{b} is a hydrogen atom, or a C₁₋₆ alkyl group (e.g., methyl).

R is more preferably
(1) a C₁₋₆ alkyl group (e.g., methyl, ethyl), or
(2) NR^{a}R^{b};
   wherein R^{a} is a C₁₋₆ alkyl group (e.g., methyl); and R^{b} is a hydrogen atom.

R is more preferably
(1) a C₁₋₆ alkyl group (e.g., methyl, ethyl), or
(2) a mono-C₁₋₆ alkylamino group (e.g., methylamino).

Compound (I) is preferably optically active, and more preferably a compound represented by the following formula (I′) having stereoisomerism on the Ring Y moiety.

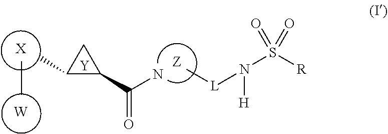

Preferable examples of compound (I) include the following compounds. These compounds are preferably represented by the above formula (I′).

### [Compound A]

Compound (I) is preferably compound (I) wherein Ring W is
(1) a 3- to 8-membered monocyclic non-aromatic heterocycle (e.g., piperidine),
(2) a 5- to 6-membered monocyclic aromatic heterocycle (e.g., furan, pyridine) optionally further substituted by 1 to 3 halogen atoms (e.g., a fluorine atom),
(3) a C₆₋₁₄ aromatic hydrocarbon ring (e.g., benzene) optionally further substituted by 1 to 3 substituents selected from
   (i) a halogen atom (e.g., a fluorine atom, a chlorine atom),
   (ii) a C₁₋₆ alkyl group (e.g., methyl) optionally substituted by 1 to 3 C₁₋₆ alkoxy groups (e.g., methoxy), and
   (iii) a C₁₋₆ alkoxy group (e.g., methoxy), or
(4) a C₃₋₁₀ cycloalkane (e.g., cyclobutane, cyclopentane, cyclohexane);

### Ring X is

(1) a benzene ring optionally further substituted by 1 to 3 substituents selected from
   (i) a halogen atom (e.g., a fluorine atom, a chlorine atom),
   (ii) an optionally halogenated C₁₋₆ alkyl group (e.g., methyl, difluoromethyl), and
   (iii) a C₁₋₆ alkoxy group (e.g., methoxy), or
(2) a 5- or 6-membered monocyclic aromatic heterocycle (e.g., thiophene, thiazole, pyridine) optionally further substituted by 1 to 3 C₁₋₆ alkyl groups (e.g., methyl);
Ring Y is cyclopropane optionally further substituted by 1 to 3 substituents selected from
   (i) a halogen atom (e.g., a fluorine atom),
   (ii) a cyano group, and
   (iii) a C₁₋₆ alkyl group (e.g., methyl);
Ring Z is a 3- to 14-membered nitrogen-containing heterocycle (e.g., azetidine, pyrrolidine, piperidine, morpholine, azepane, azabicyclo[3.1.0]hexane, azaspiro[3.3]heptane, azabicyclo[3.2.0]heptane, azabicyclo[3.2.1]octane, oxazepane, octahydrocyclopenta[c]pyrrole) optionally further substituted by 1 to 3 substituents selected from
   (i) a halogen atom (e.g., a fluorine atom),
   (ii) a hydroxy group,
   (iii) a C₁₋₆ alkyl group (e.g., methyl), and
   (iv) a C₁₋₆ alkoxy group (e.g., methoxy);
L is a bond or a methylene group; and
R is
(1) a C₁₋₆ alkyl group (e.g., methyl, ethyl) optionally substituted by 1 to 3 substituents selected from
   (i) a halogen atom (e.g., a fluorine atom, a bromine atom), and
   (ii) a C₁₋₆ alkoxy (e.g., methoxy),
(2) a C₃₋₁₀ cycloalkyl group (e.g., cyclopropyl), or
(3) a di-C₁₋₆ alkylamino group (e.g., dimethylamino).

### [Compound B]

Compound (I) is more preferably compound (I) wherein Ring W is a C₆₋₁₄ aromatic hydrocarbon ring (e.g., benzene) optionally further substituted by 1 to 3 halogen atoms (e.g., a fluorine atom);
Ring X is a benzene ring optionally further substituted by 1 to 3 substituents selected from
   (i) a halogen atom (e.g., a fluorine atom), and
   (ii) a C₁₋₆ alkyl group (e.g., methyl);
Ring Y is cyclopropane optionally further substituted by 1 to 3 halogen atoms (e.g., a fluorine atom);
Ring Z is a 3- to 14-membered nitrogen-containing heterocycle (e.g., azetidine, pyrrolidine, piperidine, azepane, oxazepane, octahydrocyclopenta[c]pyrrole) optionally further substituted by 1 to 3 substituents selected from a halogen atom (e.g., a fluorine atom), a hydroxy group and a C₁₋₆ alkyl group (e.g., methyl);
L is a bond or a methylene group; and
R is
   (1) a C₁₋₆ alkyl group (e.g., methyl, ethyl), or
   (2) a di-C₁₋₆ alkylamino group (e.g., dimethylamino).

### [Compound C]

Compound (I) is preferably compound (I) wherein Ring W is
(1) a 3- to 8-membered monocyclic non-aromatic heterocycle (e.g., piperidine),
(2) a 5- to 6-membered monocyclic aromatic heterocycle (e.g., furan, pyridine) optionally further substituted by 1 to 3 halogen atoms (e.g., a fluorine atom),
(3) a C₆₋₁₄ aromatic hydrocarbon ring (e.g., benzene) optionally further substituted by 1 to 3 substituents selected from
   (i) a halogen atom (e.g., a fluorine atom, a chlorine atom),
   (ii) a C₁₋₆ alkyl group (e.g., methyl) optionally substituted by 1 to 3 C₁₋₆ alkoxy groups (e.g., methoxy), and
   (iii) a C₁₋₆ alkoxy group (e.g., methoxy), or
(4) a C₃₋₁₀ cycloalkane (e.g., cyclobutane, cyclopentane, cyclohexane);
Ring X is
(1) a benzene ring optionally further substituted by 1 to 3 substituents selected from
   (i) a halogen atom (e.g., a fluorine atom, a chlorine atom),
   (ii) an optionally halogenated C₁₋₆ alkyl group (e.g., methyl, difluoromethyl), and
   (iii) a C₁₋₆ alkoxy group (e.g., methoxy), or
(2) a 5- or 6-membered monocyclic aromatic heterocycle (e.g., thiophene, thiazole, pyridine) optionally further substituted by 1 to 3 substituents selected from
   (i) a halogen atom (e.g., a fluorine atom), and
   (ii) C₁₋₆ alkyl groups (e.g., methyl);
Ring Y is cyclopropane ring optionally further substituted by 1 to 3 substituents selected from
   (i) a halogen atom (e.g., a fluorine atom),
   (ii) a cyano group, and
   (iii) a C₁₋₆ alkyl group (e.g., methyl) optionally substituted by 1 to 3 substituents selected from C₁₋₆ alkoxy group (e.g., methoxy), cyano, and hydroxy;
Ring Z is a 3- to 14-membered nitrogen-containing heterocycle (e.g., azetidine, pyrrolidine, piperidine, morpholine, azepane, azabicyclo[3.1.0]hexane, azaspiro[3.3]heptane, azabicyclo[3.2.0]heptane, azabicyclo[3.2.1]octane, oxazepane, octahydrocyclopenta[c]pyrrole) optionally further substituted by 1 to 3 substituents selected from
   (i) a halogen atom (e.g., a fluorine atom),
   (ii) a hydroxy group,
   (iii) a C₁₋₆ alkyl group (e.g., methyl, ethyl, isopropyl), optionally substituted by 1 to 3 substituents selected from C₁₋₆ alkoxy group (e.g., methoxy), halogen atom (e.g., a fluorine atom), and hydroxy,
   (iv) a C₁₋₆ alkoxy group (e.g., methoxy),
   (v) —C(O)—C₁₋₆alkyl, and
   (vi) a 5-membered monocyclic aromatic heterocycle (e.g., 1,2,4-oxadiazolyl, 1,3,4-oxadiazolyl) optionally substituted with a C₁₋₆ alkyl(e.g., methyl).
L is a bond or a methylene group optionally substituted with 1 substituent selected from
   (1) a C₁₋₆ alkyl group (e.g., methyl, ethyl, isopropyl) further optionally substituted by 1 to 3 substituents selected from a C₁₋₆ alkoxy group (e.g., methoxy), and hydroxy, and
   (2) a C₃s cycloalkyl group (e.g., cyclopropyl) further optionally substituted with a hydroxy group; and
R is
   (1) a C₁₋₆ alkyl group (e.g., methyl, ethyl, propyl, isopropyl) optionally substituted by 1 to 3 substituents selected from
      (i) a halogen atom (e.g., a fluorine atom, a bromine atom),
      (ii) a C₁₋₆ alkoxy (e.g., methoxy), and
      (iii) a cyclopropyl group (e.g., cyclopropyl).
   (2) a C₃₋₁₀ cycloalkyl group (e.g., cyclopropyl) further optionally substituted with a C₁₋₆ alkyl group (e.g., methyl), or
   (3) NR^{a}R^{b}.
wherein R^{a} is a C₁₋₆ alkyl group (e.g., methyl, ethyl, or isopropyl), or a C₃₋₁₀ cycloalkyl group (e.g., cyclopropyl);
R^{b} is a hydrogen atom, or a C₁₋₆ alkyl group (e.g., methyl); or
R^{a} and R^{b} in combination form a nitrogen-containing heterocycle (e.g., azetidinyl), together with the adjacent nitrogen atom.

### [Compound D]

Compound (I) is more preferably compound (I) wherein Ring W is a C₆₋₁₄ aromatic hydrocarbon ring (e.g., benzene) optionally further substituted by 1 to 3 halogen atoms (e.g., a fluorine atom);
Ring X is a benzene ring optionally further substituted by 1 to 3 substituents selected from
   (i) a halogen atom (e.g., a fluorine atom, a chlorine atom)), and
   (ii) a C₁₋₆ alkyl group (e.g., methyl);
Ring Y is cyclopropane ring optionally further substituted by 1 to 3 halogen atoms (e.g., a fluorine atom);
Ring Z is a 3- to 14-membered (more preferably 4- to 8-membered) nitrogen-containing heterocycle (e.g., azetidine, pyrrolidine, piperidine, azepane, oxazepane, octahydrocyclopenta[c]pyrrole) optionally further substituted by 1 to 3 substituents selected from a halogen atom (e.g., a fluorine atom), a hydroxy group and a C₁₋₆ alkyl group (e.g., methyl);
L is a bond or a methylene group optionally substituted with a C₁₋₆ alkyl group (e.g., methyl);
R is
   (1) a C₁₋₆ alkyl group (e.g., methyl, ethyl), or
   (2) NR^{a}R^{b};
wherein R^{a} is a C₁₋₆ alkyl group (e.g., methyl); and
R^{b} is a hydrogen atom, or a C₁₋₆ alkyl group (e.g., methyl).

### [Compound E]

In another embodiment, Compound (I) is a compound (I) wherein
Ring W is a benzene ring optionally further substituted with 2 fluorine atoms.
Ring X is

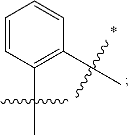 [SMILES: *c1ccccc1*]

further optionally substituted by 1 or 2 substituents independently selected from halogen (e.g., a fluorine atom, a chlorine atom) and methyl, wherein [symbol] is the point of attachment to Ring W and [symbol] is the point of attachment to Ring Y.
Ring Y is

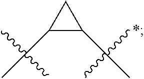 [SMILES: *C1CC1*]

further optionally substituted with one fluorine atom, wherein [symbol] is the point of attachment to Ring X and [symbol] is the point of attachment to Ring Z;
Ring Z is selected from the group consisting of:

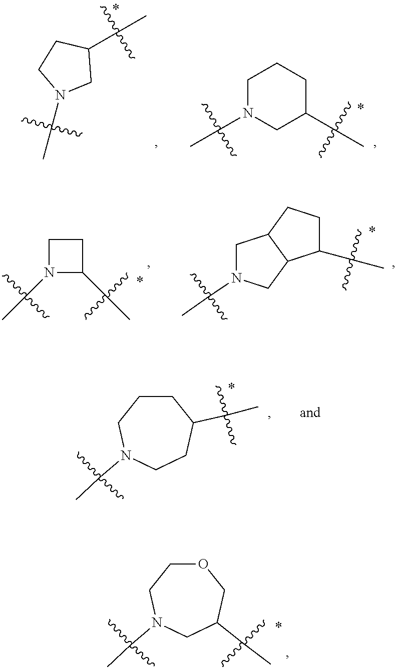 [SMILES: *C1CCN(*)C1 ; *C1CCCN(*)C1 ; *C1CCN1* ; *C1CCC2CN(*)CC12 ; *C1CCCN(*)CC1 ; *C1COCCN(*)C1]

further optionally substituted with 1 to 3 substituents independently selected from a halogen atom (e.g., a fluorine atom), a hydroxy group and a C₁₋₆ alkyl group (e.g., methyl), wherein [symbol] is the point of attachment to Ring Y and [symbol] is the point of attachment to L.
L is a methylene group optionally substituted with a C₁₋₆ alkyl group (e.g., methyl); and
R is
   (1) a C₁₋₆ alkyl group (e.g., methyl, ethyl), or
   (2) NR^{a}R^{b};
wherein R^{a} is a C₁₋₆ alkyl group (e.g., methyl); and
R^{b} is a hydrogen atom, or a C₁₋₆ alkyl group (e.g., methyl).

### [Compound F]

Compound (I) is more preferably compound (I) wherein
Ring W is a benzene ring optionally further substituted by 1 to 3 halogen atoms (e.g., a fluorine atom);
Ring X is a benzene ring optionally further substituted by 1 to 3 substituents selected from
   (i) a halogen atom (e.g., a fluorine atom, a chlorine atom), and
   (ii) a C₁₋₆ alkyl group (e.g., methyl);
Ring Y is cyclopropane ring optionally further substituted by 1 to 3 halogen atoms (e.g., a fluorine atom);
Ring Z is an azetidine ring optionally further substituted by 1 to 3 substituents selected from a halogen atom (e.g., a fluorine atom) and a C₁₋₆ alkyl group (e.g., methyl);
L is a methylene group optionally substituted with a C₁₋₆ alkyl group (e.g., methyl);
R is
   (1) a C₁₋₆ alkyl group (e.g., methyl, ethyl), or
   (2) NR^{a}R^{b};
wherein R^{a} is a C₁₋₆ alkyl group (e.g., methyl); and
R^{b} is a hydrogen atom.

### [Compound G]

Compound (I) is more preferably compound (I) wherein
Ring W is a benzene ring optionally further substituted by 1 to 3 halogen atoms (e.g., a fluorine atom);
Ring X is a benzene ring optionally further substituted by 1 to 3 halogen atoms (e.g., a fluorine atom);
Ring Y is cyclopropane ring optionally further substituted by 1 to 3 halogen atoms (e.g., a fluorine atom);
Ring Z is an azetidine ring optionally further substituted by 1 to 3 halogen atoms (e.g., a fluorine atom);
L is a methylene group;
R is a C₁ alkyl group (e.g., ethyl), or NR^{a}R^{b};
wherein R^{a} is a C₁ alkyl group (e.g., methyl); and
R^{b} is a hydrogen atom.

Specific examples of compound (I) include the compounds of the below Examples 1 to 12, 14 to 132, 134 to 138, 140 to 230, 232 to 353, 355 to 368, 370 to 450.

Specifically, compound (I) is preferably
N-[(1S)-1-{(2S)-1-[(1S,2S)-2-(2′,6′-difluoro [1,1′-biphenyl]-2-yl)-2-fluorocyclopropane-1-carbonyl]azetidin-2-yl}ethyl]methanesulfonamide or a salt thereof (Example 353); N-({(2S,3R)-1-[(1S,2S)-2-(2′,6′-difluoro[1,1′-biphenyl]-2-yl)-2-fluorocyclopropane-1-carbonyl]-3-methylazetidin-2-yl}methyl)methanesulfonamide or a salt thereof (Example 358); N-({(2R,3R)-1-[(1S,2S)-2-(2′,6′-difluoro [1,1′-biphenyl]-2-yl)-2-fluorocyclopropane-1-carbonyl]-3-fluoroazetidin-2-yl}methyl)ethanesulfonamide or a salt thereof (Example 393); N-({(2R,3R)-1-[(1S,2S)-2-(2′,6′-difluoro-5-methyl [1,1′-biphenyl]-2-yl)-2-fluorocyclopropane-1-carbonyl]-3-fluoroazetidin-2-yl}methyl)methanesulfonamide or a salt thereof (Example 411);
N-({(2R,3R)-3-fluoro-1-[(1S,2S)-2-fluoro-2-(2′,5,6′-trifluoro[1,1′-biphenyl]-2-yl)cyclopropane-1-carbonyl]azetidin-2-yl}methyl)ethanesulfonamide or a salt thereof (Example 412); N-({(2R,3R)-1-[(1R,2R)-2-(5-chloro-2′,6′-difluoro [1,1′-biphenyl]-2-yl)-2-fluorocyclopropane-1-carbonyl]-3-fluoroazetidin-2-yl}methyl)ethanesulfonamide, or N-({(2R,3R)-1-[(1S,2S)-2-(5-chloro-2′,6′-difluoro[1,1′-biphenyl]-2-yl)-2-fluorocyclopropane-1-carbonyl]-3-fluoroazetidin-2-yl}methyl)ethanesulfonamide or a salt thereof (Example 420); or
N-({(2R,3R)-3-fluoro-1-[(1S,2S)-2-fluoro-2-(2′,5,6′-trifluoro[1,1′-biphenyl]-2-yl)cyclopropane-1-carbonyl]azetidin-2-yl}methyl)-N′-methylsulfuric diamide or a salt thereof (Example 423).

Compound (I) is particularly preferably
N-({(2R,3R)-1-[(1S,2S)-2-(2′,6′-difluoro[1,1′-biphenyl]-2-yl)-2-fluorocyclopropane-1-carbonyl]-3-fluoroazetidin-2-yl}methyl)ethanesulfonamide or a salt thereof (Example 393);
N-({(2R,3R)-3-fluoro-1-[(1S,2S)-2-fluoro-2-(2′,5,6′-trifluoro[1,1′-biphenyl]-2-yl)cyclopropane-1-carbonyl]azetidin-2-yl}methyl)ethanesulfonamide or a salt thereof (Example 412); or
N-({(2R,3R)-3-fluoro-1-[(1S,2S)-2-fluoro-2-(2′,5,6′-trifluoro[1,1′-biphenyl]-2-yl)cyclopropane-1-carbonyl]azetidin-2-yl}methyl)-N′-methylsulfuric diamide or a salt thereof (Example 423).

The term “therapeutically effective amount” means any amount which, as compared to a corresponding subject who has not received such amount, results in improved treatment, healing, or amelioration of a disease, disorder, or side effect, or a decrease in the rate of advancement of a disease or disorder. The term also includes within its scope amounts effective to enhance normal physiological function. For use in therapy, therapeutically effective amounts of a compound of Formulae (I), as well as salts thereof, may be administered as the raw chemical. Additionally, the active ingredient may be presented as a pharmaceutical composition.

As a salt of a compound represented by the formula (I), a pharmacologically acceptable salt is preferable, and examples of such salt include a salt with inorganic base, a salt with organic base, a salt with inorganic acid, a salt with organic acid, a salt with basic or acidic amino acid and the like.

Preferable examples of the salt with inorganic base include alkali metal salts such as sodium salt, potassium salt and the like, alkaline earth metal salts such as calcium salt, magnesium salt and the like, aluminum salt, ammonium salt and the like.

Preferable examples of the salt with organic base include salts with trimethylamine, triethylamine, pyridine, picoline, ethanolamine, diethanolamine, triethanolamine, tromethamine[tris(hydroxymethyl)methylamine], tert-butylamine, cyclohexylamine, benzylamine, dicyclohexylamine, N,N-dibenzylethylenediamine and the like.

Preferable examples of the salt with inorganic acid include salts with hydrochloric acid, hydrobromic acid, nitric acid, sulfuric acid, phosphoric acid and the like.

Preferable examples of the salt with organic acid include salts with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid and the like.

Preferable examples of the salt with basic amino acid include salts with arginine, lysine, ornithine and the like. Preferable examples of the salt with acidic amino acid include salts with aspartic acid, glutamic acid and the like.

Because of its potential use in medicine, it will be appreciated that a salt of a compound of Formulae (II) and (II′) are ideally pharmaceutically acceptable. Pharmaceutically acceptable salts include, amongst others, those described in Berge, J. Pharm. Sci., 66, 1-19, (1977) or those listed in P. H. Stahl and C. G. Wermuth, editors, Handbook of Pharmaceutical Salts; Properties, Selection and Use, Second Edition Stahl/Wermuth: Wiley-VCH/VHCA (2011) (see http://www.wiley.com/WileyCDA/WileyTitle/productCd-3906390519.html).

Optionally the acid or base can be added to the compound of Formula (I) in a suitable solvent such as an organic solvent, to give the salt which can be isolated by a variety of methods, including crystallisation and filtration.

Representative pharmaceutically acceptable acid addition salts include, but are not limited to, 4-acetamidobenzoate, acetate, adipate, alginate, ascorbate, aspartate, benzenesulfonate (besylate), benzoate, bisulfate, bitartrate, butyrate, calcium edetate, camphorate, camphorsulfonate (camsylate), caprate (decanoate), caproate (hexanoate), caprylate (octanoate), cinnamate, citrate, cyclamate, digluconate, 2,5-dihydroxybenzoate, disuccinate, dodecylsulfate (estolate), edetate (ethylenediaminetetraacetate), estolate (lauryl sulfate), ethane-1,2-disulfonate (edisylate), ethanesulfonate (esylate), formate, fumarate, galactarate (mucate), gentisate (2,5-dihydroxybenzoate), glucoheptonate (gluceptate), gluconate, glucuronate, glutamate, glutarate, glycerophosphorate, glycolate, hexylresorcinate, hippurate, hydrabamine (N,N′-di(dehydroabietyl)-ethylenediamine), hydrobromide, hydrochloride, hydroiodide, hydroxynaphthoate, isobutyrate, lactate, lactobionate, laurate, malate, maleate, malonate, mandelate, methanesulfonate (mesylate), methylsulfate, mucate, naphthalene-1,5-disulfonate (napadisylate), naphthalene-2-sulfonate (napsylate), nicotinate, nitrate, oleate, palmitate, p-aminobenzenesulfonate, p-aminosalicyclate, pamoate (embonate), pantothenate, pectinate, persulfate, phenylacetate, phenylethylbarbiturate, phosphate, polygalacturonate, propionate, p-toluenesulfonate (tosylate), pyroglutamate, pyruvate, salicylate, sebacate, stearate, subacetate, succinate, sulfamate, sulfate, tannate, tartrate, teoclate (8-chlorotheophyllinate), thiocyanate, triethiodide, undecanoate, undecylenate, and valerate.

Representative pharmaceutically acceptable base addition salts include, but are not limited to, aluminium, 2-amino-2-(hydroxymethyl)-1,3-propanediol (TRIS), arginine, benethamine (N-benzylphenethylamine), benzathine (N,N′-dibenzylethylenediamine), bis-(2-hydroxyethyl)amine, bismuth, calcium, chloroprocaine, choline, clemizole (1-p chlorobenzyl-2-pyrrolildine-1′-ylmethylbenzimidazole), cyclohexylamine, dibenzylethylenediamine, diethylamine, diethyltriamine, dimethylamine, dimethylethanolamine, dopamine, ethanolamine, ethylenediamine, L-histidine, iron, isoquinoline, lepidine, lithium, lysine, magnesium, meglumine (N-methylglucamine), piperazine, piperidine, potassium, procaine, quinine, quinoline, sodium, strontium, t-butylamine, tromethamine (tris(hydroxymethyl)aminomethane), and zinc.

Salts may be prepared in situ during the final isolation and purification of a compound of Formulae (II) and (II′). If a basic compound of Formulae (II) and (II′) is isolated as a salt, the corresponding free base form of that compound may be prepared by any suitable method known to the art, including treatment of the salt with an inorganic or organic base. Similarly, if a compound of Formulae (II) and (II′) containing a carboxylic acid or other acidic functional group is isolated as a salt, the corresponding free acid form of that compound may be prepared by any suitable method known to the art, including treatment of the salt with an inorganic or organic acid.

It will be understood that if a compound of Formulae (II) and (II′) contains two or more basic moieties, the stoichiometry of salt formation may include 1, 2 or more equivalents of acid. Such salts would contain 1, 2 or more acid counterions, for example, a dihydrochloride salt.

Stoichiometric and non-stoichiometric forms of a pharmaceutically acceptable salt of a compound of Formulae (II) and (II′) are included within the scope of the invention, including sub-stoichiometric salts, for example where a counterion contains more than one acidic proton.

In certain embodiments the present disclosure provides a pharmaceutically acceptable salt of any of the above-mentioned embodiments [1]-[34].

The production method of the compound of the present invention is explained below.

The raw material compound and reagent used and the compound obtained in each step in the following production method may be each in a form of a salt, and examples of such salt include those similar to the salts of the compound represented by the formula (I), and the like.

When the compound obtained in each step is a free form, it can be converted to the objective salt according to a method known per se. When the compound obtained in each step is a salt, it can be converted to the objective free form or the other salt according to a method known per se.

The compound obtained in each step can be used directly as the reaction mixture or as a crude product for the next reaction. Alternatively, the compound obtained in each step can be isolated and purified from a reaction mixture according to a method known per se, for example, a separation means such as concentration, crystallization, recrystallization, distillation, solvent extraction, fractional distillation, column chromatography and the like.

When the raw material compound and reagent used in each step are commercially available, the commercially available product can also be used directly.

In the reaction in each step, while the reaction time varies depending on the kind of the reagent and solvent to be used, it is generally 1 min-48 hr, preferably 10 min-8 hr, unless otherwise specified.

In the reaction in each step, while the reaction temperature varies depending on the kind of the reagent and solvent to be used, it is generally −78° C.-300° C., preferably −78° C.-150° C., unless otherwise specified.

In the reaction in each step, while the pressure varies depending on the kind of the reagent and solvent to be used, it is generally 1 atm-20 atm, preferably 1 atm-3 atm, unless otherwise specified.

Microwave synthesizer such as Initiator manufactured by Biotage and the like may be used for the reaction in each step. While the reaction temperature varies depending on the kind of the reagent and solvent to be used, it is generally room temperature-300° C., preferably 50° C.-250° C., unless otherwise specified. While the reaction time varies depending on the kind of the reagent and solvent to be used, it is generally 1 min-48 hr, preferably 1 min-8 hr, unless otherwise specified.

In the reaction in each step, the reagent is used in an amount of 0.5 equivalents-20 equivalents, preferably 0.8 equivalents-5 equivalents, relative to the substrate, unless otherwise specified. When the reagent is used as a catalyst, the reagent is used in an amount of 0.001 equivalent-1 equivalent, preferably 0.01 equivalent-0.2 equivalent, relative to the substrate. When the reagent is used as a reaction solvent, the reagent is used in a solvent amount.

Unless otherwise specified, the reaction in each step is carried out without solvent, or by dissolving or suspending the raw material compound in a suitable solvent. Examples of the solvent include those described in Examples and the following solvents.
alcohols: methanol, ethanol, tert-butyl alcohol, 2-methoxyethanol and the like;
ethers: diethyl ether, diphenyl ether, tetrahydrofuran, 1,2-dimethoxyethane and the like;
aromatic hydrocarbons: chlorobenzene, toluene, xylene and the like;
saturated hydrocarbons: cyclohexane, hexane and the like; amides: N,N-dimethylformamide, N-methylpyrrolidone and the like;
halogenated hydrocarbons: dichloromethane, carbon tetrachloride and the like;
nitriles: acetonitrile and the like;
sulfoxides: dimethyl sulfoxide and the like;
aromatic organic bases: pyridine and the like;
anhydrides: acetic anhydride and the like;
organic acids: formic acid, acetic acid, trifluoroacetic acid and the like;
inorganic acids: hydrochloric acid, sulfuric acid and the like;
esters: ethyl acetate and the like;
ketones: acetone, methyl ethyl ketone and the like; water.

The above-mentioned solvent can be used in a mixture of two or more kinds thereof in an appropriate ratio.

When a base is used for the reaction in each step, examples thereof include those described in Examples and the following bases.
inorganic bases: sodium hydroxide, magnesium hydroxide, sodium carbonate, calcium carbonate, sodium hydrogen carbonate and the like;
organic bases: triethylamine, diethylamine, pyridine, 4-dimethylaminopyridine, N,N-dimethylaniline, 1,4-diazabicyclo[2.2.2]octane, 1,8-diazabicyclo[5.4.0]-7-undecene, imidazole, piperidine and the like;
metal alkoxides: sodium ethoxide, potassium tert-butoxide and the like;
alkali metal hydrides: sodium hydride and the like;
metal amides: sodium amide, lithium diisopropylamide, lithium hexamethyldisilazide and the like;
organic lithiums: n-butyllithium and the like.

When an acid or an acid catalyst is used for the reaction in each step, examples thereof include those described in Examples and the following acids and acid catalysts.
inorganic acids: hydrochloric acid, sulfuric acid, nitric acid, hydrobromic acid, phosphoric acid and the like;
organic acids: acetic acid, trifluoroacetic acid, citric acid, p-toluenesulfonic acid, 10-camphorsulfonic acid and the like; Lewis acid: boron trifluoride diethyl ether complex, zinc iodide, anhydrous aluminum chloride, anhydrous zinc chloride, anhydrous iron chloride and the like.

Unless otherwise specified, the reaction in each step is carried out according to a method known per se, for example, the method described in Jikken Kagaku Kouza, 5th Edition, vol. 13-19 (the Chemical Society of Japan ed.); Shin Jikken Kagaku Kouza, vol. 14-15 (the Chemical Society of Japan ed.); Fine Organic Chemistry, Revised 2nd Edition (L. F. Tietze, Th. Eicher, Nankodo); Organic Name Reactions, the Reaction Mechanism and Essence, Revised Edition (Hideo Togo, Kodansha); ORGANIC SYNTHESES Collective Volume I-VII (John Wiley & Sons Inc.); Modern Organic Synthesis in the Laboratory A Collection of Standard Experimental Procedures (Jie Jack Li, OXFORD UNIVERSITY); Comprehensive Heterocyclic Chemistry III, Vol. 1-Vol. 14 (Elsevier Japan); Strategic Applications of Named Reactions in Organic Synthesis (translated by Kiyoshi Tomioka, Kagakudojin); Comprehensive Organic Transformations (VCH Publishers Inc.), 1989, or the like, or the method described in Examples.

In each step, the protection or deprotection reaction of an functional group is carried out according to a method known per se, for example, the method described in “Protective Groups in Organic Synthesis, 4th Ed”, Wiley-Interscience, Inc., 2007 (Theodora W. Greene, Peter G. M. Wuts); “Protecting Groups 3rd Ed.” Thieme, 2004 (P. J. Kocienski), or the like, or the method described in Examples.

Examples of the protecting group for a hydroxy group of an alcohol and the like and a phenolic hydroxy group include ether-type protecting groups such as methoxymethyl ether, benzyl ether, tert-butyldimethylsilyl ether, tetrahydropyranyl ether and the like; carboxylate ester-type protecting groups such as acetate ester and the like; sulfonate ester-type protecting groups such as methanesulfonate ester and the like; carbonate ester-type protecting groups such as tert-butylcarbonate and the like, and the like.

Examples of the protecting group for a carbonyl group of an aldehyde include acetal-type protecting groups such as dimethylacetal and the like; cyclic acetal-type protecting groups such as 1,3-dioxane and the like, and the like.

Examples of the protecting group for a carbonyl group of a ketone include ketal-type protecting groups such as dimethylketal and the like; cyclic ketal-type protecting groups such as 1,3-dioxane and the like; oxime-type protecting groups such as O-methyloxime and the like; hydrazone-type protecting groups such as N,N-dimethylhydrazone and the like, and the like.

Examples of the protecting group for a carboxyl group include ester-type protecting groups such as methyl ester and the like; amide-type protecting groups such as N,N-dimethylamide and the like, and the like.

Examples of the protecting group for a thiol include ether-type protecting groups such as benzyl thioether and the like; ester-type protecting groups such as thioacetate ester, thiocarbonate, thiocarbamate and the like, and the like.

Examples of the protecting group for an amino group and an aromatic heterocycle such as imidazole, pyrrole, indole and the like include carbamate-type protecting groups such as benzyl carbamate and the like; amide-type protecting groups such as acetamide and the like; alkyl amine-type protecting groups such as N-triphenylmethylamine and the like; sulfonamide-type protecting groups such as methanesulfonamide and the like, and the like.

The protecting groups can be removed according to a method known per se, for example, by employing a method using acid, base, ultraviolet rays, hydrazine, phenylhydrazine, sodium N-methyldithiocarbamate, tetrabutylammonium fluoride, palladium acetate, trialkylsilyl halide (e.g., trimethylsilyl iodide, trimethylsilyl bromide) and the like, a reduction method, and the like.

When reduction reaction is carried out in each step, examples of the reducing agent to be used include metal hydrides such as lithium aluminum hydride, sodium triacetoxyborohydride, sodium cyanoborohydride, diisobutylaluminum hydride (DIBAL-H), sodium borohydride, tetramethylammonium triacetoxyborohydride and the like; boranes such as borane tetrahydrofuran complex and the like; Raney nickel; Raney cobalt; hydrogen; formic acid; triethylsilane and the like. When carbon-carbon double bond or triple bond is reduced, a method using a catalyst such as palladium-carbon, Lindlar's catalyst and the like may be employed.

When oxidation reaction is carried out in each step, examples of the oxidizing agent to be used include peroxides such as m-chloroperbenzoic acid (mCPBA), hydrogen peroxide, tert-butylhydroperoxide and the like; perchlorates such as tetrabutylammonium perchlorate and the like; chlorates such as sodium chlorate and the like; chlorites such as sodium chlorite and the like; periodates such as sodium periodate and the like; hypervalent iodine reagents such as iodosylbenzene and the like; reagents containing manganese such as manganese dioxide, potassium permanganate and the like; leads such as lead tetraacetate and the like; reagents containing chromium such as pyridinium chlorochromate (PCC), pyridinium dichromate (PDC), Jones reagent and the like; halogen compounds such as N-bromosuccinimide (NBS) and the like; oxygen; ozone; sulfur trioxide-pyridine complex; osmium tetroxide; selenium dioxide; 2,3-dichloro-5,6-dicyano-1,4-benzoquinone (DDQ) and the like.

When radical cyclization reaction is carried out in each step, examples of the radical initiator to be used include azo compounds such as azobisisobutyronitrile (AIBN) and the like; water-soluble radical initiators such as 4-4′-azobis-4-cyanopentanoic acid (ACPA) and the like; triethylboron in the presence of air or oxygen; benzoyl peroxide and the like. Examples of the radical reagent to be used include tributylstannane, tristrimethylsilylsilane, 1,1,2,2-tetraphenyldisilane, diphenylsilane, samarium iodide and the like.

When Wittig reaction is carried out in each step, examples of the Wittig reagent to be used include alkylidene phosphoranes and the like. The alkylidene phosphoranes can be prepared according to a method known per se, for example, by reacting a phosphonium salt with a strong base.

When Horner-Emmons reaction is carried out in each step, examples of the reagent to be used include phosphonoacetates such as methyl dimethylphosphonoacetate, ethyl diethylphosphonoacetate and the like; and bases such as alkali metal hydrides, organic lithiums and the like.

When Friedel-Crafts reaction is carried out in each step, a combination of a Lewis acid and an acid chloride or a combination of a Lewis acid and an alkylating agent (e.g., an alkyl halide, an alcohol, an olefin etc.) is used as a reagent. Alternatively, an organic acid or an inorganic acid can also be used instead of a Lewis acid, and an anhydride such as acetic anhydride and the like can also be used instead of an acid chloride.

When aromatic nucleophilic substitution reaction is carried out in each step, a nucleophile (e.g., an amine, imidazole etc.) and a base (e.g., an organic base etc.) are used as a reagent.

When nucleophilic addition reaction by a carbo anion, nucleophilic 1,4-addition reaction (Michael addition reaction) by a carbo anion or nucleophilic substitution reaction by a carbo anion is carried out in each step, and examples of the base to be used for generation of the carbo anion include organic lithiums, metal alkoxides, inorganic bases, organic bases and the like.

When Grignard reaction is carried out in each step, examples of the Grignard reagent to be used include arylmagnesium halides such as phenylmagnesium bromide and the like; and alkylmagnesium halides such as methylmagnesium bromide and the like. The Grignard reagent can be prepared according to a method known per se, for example, by reacting an alkyl halide or an aryl halide with a metal magnesium in an ether or tetrahydrofuran as a solvent.

When Knoevenagel condensation reaction is carried out in each step, a compound having an activated methylene group with two electron withdrawing groups (e.g., malonic acid, diethyl malonate, malononitrile etc.) and a base (e.g., an organic base, a metal alkoxide, an inorganic base) are used as a reagent.

When Vilsmeier-Haack reaction is carried out in each step, phosphoryl chloride and an amide derivative (e.g., N,N-dimethylformamide etc.) are used as a reagent.

When azidation reaction of an alcohol, an alkyl halide or a sulfonate is carried out in each step, examples of the azidating agent to be used include diphenylphosphorylazide (DPPA), trimethylsilylazide, sodium azide and the like. For example, for the azidation reaction of an alcohol, a method using diphenylphosphorylazide and 1,8-diazabicyclo[5.4.0]undec-7-ene (DBU), a method using trimethylsilylazide and a Lewis acid, and the like are employed.

When reductive amination reaction is carried out in each step, examples of the reducing agent to be used include sodium triacetoxyborohydride, sodium cyanoborohydride, hydrogen, formic acid and the like. When the substrate is an amine compound, examples of the carbonyl compound to be used include paraformaldehyde, aldehydes such as acetaldehyde and the like, and ketones such as cyclohexanone and the like. When the substrate is a carbonyl compound, examples of the amine to be used include ammonia, primary amines such as methylamine and the like; secondary amines such as dimethylamine and the like, and the like.

When Mitsunobu reaction is carried out in each step, an azodicarboxylate (e.g., diethyl azodicarboxylate (DEAD), diisopropyl azodicarboxylate (DIAD) etc.) and triphenylphosphine are used as a reagent.

When esterification reaction, amidation reaction or urea formation reaction is carried out in each step, examples of the reagent to be used include acyl halides such as acid chlorides, acid bromides and the like; activated carboxylic acids such as acid anhydrides, activated esters, sulfates and the like. Examples of the activating agent of the carboxylic acid include carbodiimide condensing agents such as 1-ethyl-3-(3-dimethylaminopropyl)carbodiimide hydrochloride (WSCD) and the like; triazine condensing agents such as 4-(4,6-dimethoxy-1,3,5-triazin-2-yl)-4-methylmorpholinium chloride n-hydrate (DMT-MM) and the like; carbonate condensing agents such as 1,1-carbonyldiimidazole (CDI) and the like; diphenylphosphoryl azide (DPPA); benzotriazol-1-yloxy-trisdimethylaminophosphonium salt (BOP reagent); 2-chloro-1-methyl-pyridinium iodide (Mukaiyama reagent); thionyl chloride; lower alkyl haloformates such as ethyl chloroformate and the like; O-(7-azabenzotriazol-1-yl)-N,N,N′,N′-tetramethyluronium hexafluorophosphorate (HATU); sulfuric acid; combinations thereof and the like. When carbodiimide condensing agent is used, an additive such as 1-hydroxybenzotriazole (HOBt), N-hydroxysuccinimide (HOSu), dimethylaminopyridine (DMAP) and the like may be added to the reaction system.

When coupling reaction is carried out in each step, examples of the metal catalyst to be used include palladium compounds such as palladium(II) acetate, tetrakis(triphenylphosphine)palladium(0), dichlorobis(triphenylphosphine)palladium(II), dichlorobis(triethylphosphine)palladium(II), tris(dibenzylideneacetone)dipalladium(0), 1,1′-bis(diphenylphosphino)ferrocene palladium(II) chloride and the like; nickel compounds such as tetrakis(triphenylphosphine)nickel(0) and the like; rhodium compounds such as tris(triphenylphosphine)rhodium(III) chloride and the like; cobalt compounds; copper compounds such as copper oxide, copper(I) iodide and the like; platinum compounds and the like. In addition, a base can be added to the reaction system, and examples thereof include inorganic bases and the like.

When thiocarbonylation reaction is carried out in each step, phosphorus pentasulfide is typically used as the thiocarbonylating agent. Alternatively, a reagent having a 1,3,2,4-dithiadiphosphetane-2,4-disulfide structure (e.g., 2,4-bis(4-methoxyphenyl)-1,3,2,4-dithiadiphosphetane-2,4-disulfide (Lawesson reagent) etc.) can also be used instead of phosphorus pentasulfide.

When Wohl-Ziegler reaction is carried out in each step, examples of the halogenating agent to be used include N-iodosuccinimide, N-bromosuccinimide (NBS), N-chlorosuccinimide (NCS), bromine, sulfuryl chloride and the like. In addition, the reaction can be accelerated by subjecting a radical initiator such as heat, light, benzoyl peroxide, azobisisobutyronitrile and the like to the reaction system reaction.

When halogenation reaction of a hydroxy group is carried out in each step, examples of the halogenating agent to be used include hydrohalic acids and acid halides of inorganic acids, specifically, hydrochloric acid, thionyl chloride, phosphorus oxychloride and the like for chlorination, 48% hydrobromic acid and the like for bromination. In addition, a method of producing an alkyl halide by reacting an alcohol with triphenylphosphine and carbon tetrachloride or carbon tetrabromide or the like can be employed. Alternatively, a method of producing an alkyl halide via two steps comprising converting an alcohol to the corresponding sulfonate, and then reacting the sulfonate with lithium bromide, lithium chloride or sodium iodide can also be employed.

When Arbuzov reaction is carried out in each step, examples of the reagent to be used include alkyl halides such as ethyl bromoacetate and the like; and phosphites such as triethyl phosphite, tri(isopropyl) phosphite and the like.

When sulfonate esterification reaction is carried out in each step, examples of the sulfonating agent to be used include methanesulfonyl chloride, p-toluenesulfonyl chloride, methanesulfonic anhydride, p-toluenesulfonic anhydride and the like.

When hydrolysis reaction is carried out in each step, an acid or a base is used as a reagent. For acid hydrolysis reaction of tert-butyl ester, formic acid, triethylsilane and the like may be added to reductively-trap tert-butyl cation which is by-produced.

When dehydration reaction is carried out in each step, examples of the dehydrating agent to be used include sulfuric acid, diphosphorus pentaoxide, phosphorus oxychloride, N,N′-dicyclohexylcarbodiimide, alumina, polyphosphoric acid and the like.

Among compound (3) used in the below-mentioned Scheme 3, the following compounds (3)-1 and (3)-2 can be produced from compound (1) according to the method shown in the following Scheme 1. In the scheme, R¹ is an optionally substituted C₁ alkyl group, R², R³ and R⁴ are each independently a hydrogen atom, a halogen atom or an optionally substituted C₁ alkyl group, and the other symbols are as defined above.

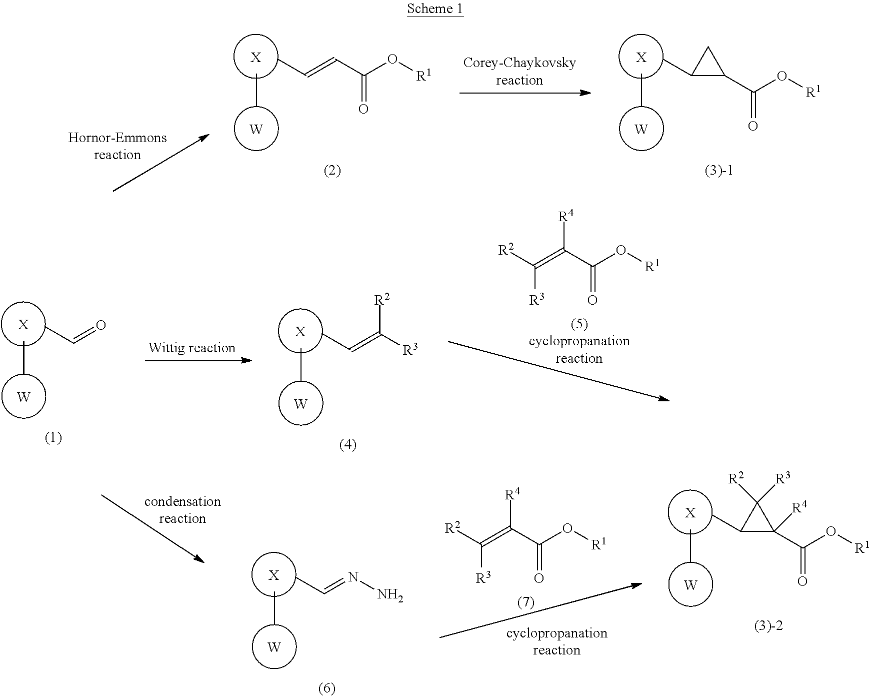

Examples of the “optionally substituted C₁₋₆ alkyl group” for R¹, R², R³ or R⁴ include a group wherein the hydrocarbon group is a C₁₋₆ alkyl group, from among the above-mentioned “optionally substituted hydrocarbon group”.

Compound (1) may be commercially easily available or can be produced according to a method known per se.

Compound (3)-1 can be produced by subjecting compound (2) to Corey-Chaykovsky reaction with a sulfonium salt in the presence of a base. Examples of the sulfonium salt to be used include trimethylsulfonium iodide and the like. Examples of the base to be used include inorganic bases, metal alkoxides, alkali metal hydrides and the like.

Compound (6) can be produced by subjecting compound (1) to a condensation reaction with hydrazine hydrate.

Compound (3)-2 can be produced by subjecting compound (4) to cyclopropanation reaction with compound (5) in the presence of a metal catalyst. Examples of the metal catalyst to be used include rhodium compounds such as rhodium(II) acetate dimer and the like, ruthenium compounds such as dichloro(p-cymene)ruthenium(II) dimer and the like, and the like.

Compound (3)-2 can also be produced subjecting compound (6) to a cyclopropanation reaction with compound (7) in the presence of an oxidant. Examples of the oxidant to be used include hypervalent iodine compounds such as iodosobenzene and the like, oxidation manganese (IV) and the like.

Among compound (3) used in the below-mentioned Scheme 3, the following compound (3)-3 can be produced from compound (8) according to the method shown in the following Scheme 2. In the scheme, LG¹ and LG² are each independently a leaving group, R⁵ is a hydrogen atom, a halogen atom or an optionally substituted C₁₋₆ alkyl group, and the other symbols are as defined above.

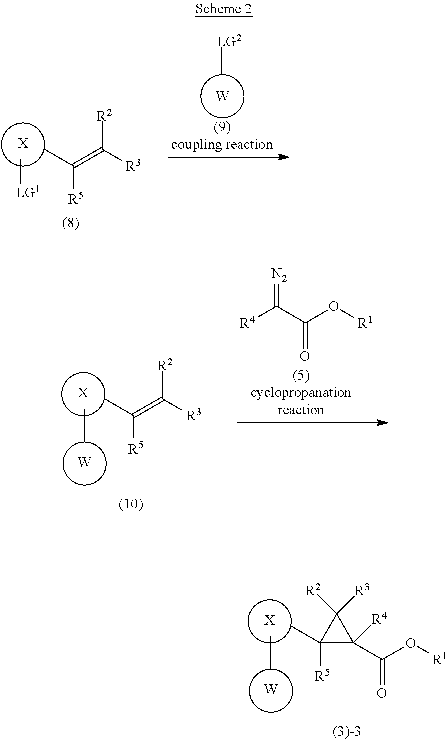

Examples of the “leaving group” for LG¹ include halogen atoms, optionally halogenated C₁₋₆ alkylsulfonyloxy (e.g., methanesulfonyloxy, ethanesulfonyloxy, trifluoromethanesulfonyloxy), C₆₋₁₄ arylsulfonyloxy optionally substituted by C₁₋₆ alkyl (e.g., benzenesulfonyloxy, toluenesulfonyloxy) and the like.

Examples of the “leaving group” for LG² include optionally substituted dihydroxyboryl (e.g., dihydroxyboryl, pinacolatoboryl) and the like.

Examples of the “optionally substituted C₁₋₆ alkyl group” for R⁵ include a group wherein the hydrocarbon group is a C₁₋₆ alkyl group, from among the above-mentioned “optionally substituted hydrocarbon group”.

Compound (8) and compound (9) may be commercially easily available or can be produced according to a method known per se.

Compound (3)-3 can be produced by subjecting compound (10) to a cyclopropanation reaction with compound (5) in the presence of a metal catalyst. Examples of the metal catalyst to be used include rhodium compounds such as rhodium(II) acetate dimer and the like, ruthenium compounds such as dichloro(p-cymene)ruthenium(II) dimer and the like, and the like.

Compound (11) can be produced from compound (3) according to the method shown in the following Scheme 3. In the scheme, each symbol is as defined above.

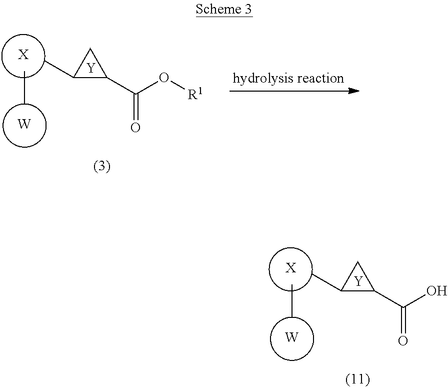

Compound (15) can be produced from compound (12) according to the method shown in the following Scheme 4. In the scheme, LG³ is a leaving group, P is a protecting group, and the other symbols are as defined above.

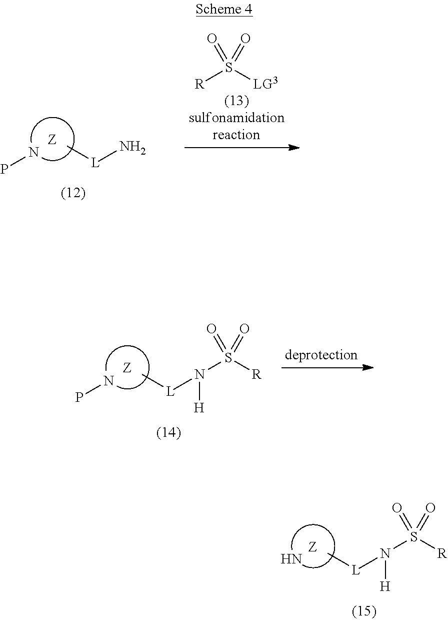

Examples of the “leaving group” for LG³ include halogen atoms, optionally halogenated C₁₋₆ alkylsulfonyloxy (e.g., methanesulfonyloxy, ethanesulfonyloxy, trifluoromethanesulfonyloxy), C₆₋₁₄ arylsulfonyloxy optionally substituted by C₁₋₆ alkyl (e.g., benzenesulfonyloxy, toluenesulfonyloxy) and the like.

Examples of the “protecting group” for P include those exemplified as the above-mentioned “protecting group for an amino group and an aromatic heterocycle such as imidazole, pyrrole, indole and the like”.

Compound (12) may be commercially easily available or can be produced according to a method known per se.

Compound (14) can be produced by subjecting compound (12) to a sulfonamidation reaction with compound (13). Example of compound (13) to be used include sulfonyl chloride, sulfamoyl chloride and the like. Compound (13) may be commercially easily available or can be produced according to a method known per se.

Compound (I) can be produced from compound (11) and compound (15) according to the method shown in the following Scheme 5. In the scheme, each symbol is as defined above.

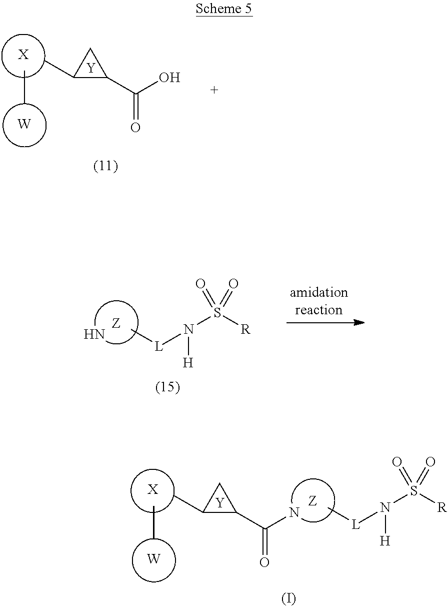

In the thus-obtained compound (I), an intramolecular functional group can also be converted to an object functional group by a combination of chemical reactions known per se. Examples of the chemical reaction include oxidation reaction, reduction reaction, alkylation reaction, acylation reaction, ureation reaction, hydrolysis reaction, amination reaction, esterification reaction, aryl coupling reaction, deprotection reaction and the like.

In the above-mentioned production method, when a starting compound has an amino group, a carboxyl group, a hydroxy group, a carbonyl group or a mercapto group as a substituent, a protecting group generally used in the peptide chemistry may be introduced into these groups, and the object compound can be obtained by removing the protecting group as necessary after the reaction.

Compound (I) obtained by the above-mentioned production method can be isolated and purified by a known means, such as solvent extraction, liquid conversion, phase transfer, crystallization, recrystallization, chromatography and the like.

When compound (I) contains optical isomer, stereoisomer, regio isomer and rotamer, these compounds are also included in compound (I), and each can be obtained as a single product by a synthesis method or a separation method known per se. For example, when an optical isomer exists in compound (I), an optical isomer resolved from the compound is also encompassed in compound (I).

Here, an optical isomer can be produced by a method known per se.

Compound (I) may be a crystal.

A crystal of compound (I) (hereinafter sometimes to be abbreviated as the crystal of the present invention) can be produced by crystallizing compound (I), by applying a crystallization method known per se.

In the present specification, the melting point means a melting point measured, for example, by micro melting point apparatus (Yanako, MP-500D or Buchi, B-545), DSC (differential scanning calorimetry analysis) apparatus (METTLER TOLEDO, DSC1) and the like.

Generally, the melting point sometimes varies depending on the measurement device, measurement condition and the like.

The crystal in the present specification may be a crystal showing a melting point different from the values described in the present specification as long as the difference is within a general error range.

The crystal of the present invention is superior in the physicochemical properties (e.g., melting point, solubility, stability) and biological properties (e.g., pharmacokinetics (absorbability, distribution, metabolism, excretion), efficacy expression), and is extremely useful as a medicament.

Compound (I) may be used as a prodrug. A prodrug of the compound (I) means a compound which is converted to the compound (I) of the present invention with a reaction due to an enzyme, an gastric acid, etc. under the physiological condition in the living body, that is, a compound which is converted to the compound (I) of the present invention with oxidation, reduction, hydrolysis, etc. according to an enzyme; a compound which is converted to the compound (I) of the present invention by hydrolysis etc. due to gastric acid, etc.

A prodrug of compound (I) may be
a compound obtained by subjecting an amino group in compound (I) to an acylation, alkylation or phosphorylation (e.g., a compound obtained by subjecting an amino group in compound (I) to an eicosanoylation, alanylation, pentylaminocarbonylation, (5-methyl-2-oxo-1,3-dioxolen-4-yl)methoxycarbonylation, tetrahydrofuranylation, pyrrolidylmethylation, pivaloyloxymethylation and tert-butylation, etc.);
a compound obtained by subjecting a hydroxy group in compound (I) to an acylation, alkylation, phosphorylation or boration (e.g., a compound obtained by subjecting an hydroxy group in compound (I) to an acetylation, palmitoylation, propanoylation, pivaloylation, succinylation, fumarylation, alanylation, dimethylaminomethylcarbonylation, etc.);
a compound obtained by subjecting a carboxyl group in compound (I) to an esterification or amidation (e.g., a compound obtained by subjecting a carboxyl group in compound (I) to an ethyl esterification, phenyl esterification, carboxymethyl esterification, dimethylaminomethyl esterification, pivaloyloxymethyl esterification, ethoxycarbonyloxyethyl esterification, phthalidyl esterification, (5-methyl-2-oxo-1,3-dioxolen-4-yl)methyl esterification, cyclohexyloxycarbonylethyl esterification and methylamidation, etc.) and the like. Any of these compounds can be produced from compound (I) by a method known per se.

A prodrug for compound (I) may also be one which is converted into compound (I) under a physiological condition, such as those described in IYAKUHIN no KAIHATSU (Development of Pharmaceuticals), Vol. 7, Design of Molecules, p. 163-198, Published by HIROKAWA SHOTEN (1990).

In the present specification, a prodrug may form a salt, and as such salt, those exemplified as a salt of the compound represented by the above-mentioned formula (I) can be mentioned.

Compound (I) may be labeled with an isotope (e.g., ³H, ¹³C, ¹⁴C, ¹⁸F, ³⁵S, ¹²⁵I) and the like.

Compound (I) labeled with or substituted by an isotope can be used, for example, as a tracer used for Positron Emission Tomography (PET) (PET tracer), and is useful in the field of medical diagnosis and the like.

Furthermore, compound (I) may be a hydrate or a non-hydrate, or a non-solvate (e.g., anhydride), or a solvate (e.g., hydrate).

Compound (I) also encompasses a deuterium conversion form wherein ¹H is converted to ²H(D).

Furthermore, compound (I) may be a pharmaceutically acceptable cocrystal or cocrystal salt. The cocrystal or cocrystal salt means a crystalline substance constituted with two or more special solids at room temperature, each having different physical properties (e.g., structure, melting point, melting heat, hygroscopicity, solubility and stability). The cocrystal or cocrystal salt can be produced by a cocrystallization method known per se.

Compound (I) or a prodrug thereof (hereinafter sometimes to be simply abbreviated as the compound of the present invention) can be used as it is or in the form of a pharmaceutical composition (also referred to as a medicament) by mixing with a pharmacologically acceptable carrier etc. to mammals (e.g., human, mouse, rat, rabbit, dog, cat, bovine, horse, swine, monkey) as an agent for the prophylaxis or treatment of various diseases mentioned below.

As pharmacologically acceptable carriers, various organic or inorganic carrier substances conventionally used as preparation materials can be used. These are incorporated as excipient, lubricant, binder and disintegrant for solid preparations; or solvent, solubilizing agent, suspending agent, isotonicity agent, buffer and soothing agent for liquid preparations; and the like; and preparation additives such as preservative, antioxidant, colorant, sweetening agent and the like can be added as necessary.

Preferable examples of the excipient include lactose, sucrose, D-mannitol, D-sorbitol, starch, gelatinated starch, dextrin, crystalline cellulose, low-substituted hydroxypropylcellulose, sodium carboxymethylcellulose, gum arabic, pullulan, light anhydrous silicic acid, synthetic aluminum silicate and magnesium alumino metasilicate.

Preferable examples of the lubricant include magnesium stearate, calcium stearate, talc and colloidal silica.

Preferable examples of the binder include gelatinated starch, sucrose, gelatin, gum arabic, methylcellulose, carboxymethylcellulose, sodium carboxymethylcellulose, crystalline cellulose, sucrose, D-mannitol, trehalose, dextrin, pullulan, hydroxypropylcellulose, hydroxypropylmethylcellulose and polyvinylpyrrolidone.

Preferable examples of the disintegrant include lactose, sucrose, starch, carboxymethylcellulose, calcium carboxymethylcellulose, croscarmellose sodium, sodium carboxymethyl starch, light anhydrous silicic acid and low-substituted hydroxypropylcellulose.

Preferable examples of the solvent include water for injection, physiological brine, Ringer's solution, alcohol, propylene glycol, polyethylene glycol, sesame oil, corn oil, olive oil and cottonseed oil.

Preferable examples of the solubilizing agent include polyethylene glycol, propylene glycol, D-mannitol, trehalose, benzyl benzoate, ethanol, trisaminomethane, cholesterol, triethanolamine, sodium carbonate, sodium citrate, sodium salicylate and sodium acetate.

Preferable examples of the suspending agent include surfactants such as stearyltriethanolamine, sodium lauryl sulfate, lauryl aminopropionate, lecithin, benzalkonium chloride, benzethonium chloride, glycerol monostearate and the like; hydrophilic polymers such as poly(vinyl alcohol), polyvinylpyrrolidone, carboxymethylcellulose sodium, methylcellulose, hydroxymethylcellulose, hydroxyethylcellulose, hydroxypropylcellulose and the like, polysorbates; and polyoxyethylene hydrogenated castor oil.

Preferable examples of the isotonicity agent include sodium chloride, glycerol, D-mannitol, D-sorbitol and glucose.

Preferable examples of the buffer include buffers of phosphate, acetate, carbonate, citrate etc.

Preferable examples of the soothing agent include benzyl alcohol.

Preferable examples of the preservative include p-oxybenzoate esters, chlorobutanol, benzyl alcohol, phenethyl alcohol, dehydroacetic acid and sorbic acid.

Preferable examples of the antioxidant include sulfite salts and ascorbate salts.

Preferable examples of the colorant include aqueous food tar colors (e.g., food colors such as Food Color Red Nos. 2 and 3, Food Color Yellow Nos. 4 and 5, Food Color Blue Nos. 1 and 2 and the like food colors), water insoluble lake dyes (e.g., aluminum salt of the above-mentioned aqueous food tar color), natural dyes (e.g., β-carotene, chlorophyll, red iron oxide) and the like.

Preferable examples of the sweetening agent include saccharin sodium, dipotassium glycyrrhizinate, aspartame and stevia.

Examples of the dosage form of the above-mentioned pharmaceutical composition include oral preparations such as tablet (including sugar-coated tablet, film-coated tablet, sublingual tablet, orally disintegrating tablet, buccal tablet), capsule (including soft capsule, microcapsule), pill, granule, powder, troche, syrup, liquid, emulsion, suspension, aerosol, films (e.g., orally disintegrable films, oral mucosa-adhesive film) and the like; and parenteral agents such as injection (e.g., subcutaneous injection, intravenous injection, intramuscular injection, intraperitoneal injection, drip infusion), external preparation (e.g., transdermal absorption type preparation, ointment, lotion, adhesive preparation), suppository (e.g., rectal suppository, vaginal suppository), pellet, nasal preparation, pulmonary preparation (inhalant), eye drop and the like. The compound and medicament of the present invention can be respectively safely administered orally or parenterally (e.g., intrarectal, intravenous, intraarterial, intramuscular, subcutaneous, intraorgan, intranasal, intradermal, instillation, intracerebral, intravaginal, intraperitoneal, intratumoral, proximal tumor administrations, and administration to the lesion).

These preparations may be a release control preparation (e.g., sustained-release microcapsule) such as an immediate-release preparation, a sustained-release preparation and the like.

The pharmaceutical composition can be produced according to a method conventionally used in the field of pharmaceutical formulation, for example, the method described in the Japanese Pharmacopoeia, and the like.

While the content of the compound of the present invention in the pharmaceutical composition of the present invention varies depending on the dosage form, dose of the compound of the present invention and the like, it is, for example, about 0.1 to 100 wt %.

When an oral preparation is produced, coating may be applied where necessary for the purpose of taste masking, enteric solubility or sustainability.

Examples of the coating base used for coating include sugar coating base, water-soluble film coating base, enteric film coating base, and sustained-release film coating base.

As the sugar coating base, sucrose is used, and one or more kinds selected from talc, and the precipitated calcium carbonate, gelatin, gum arabic, pullulan, carnauba wax and the like may be further used in combination.

Examples of the water-soluble film coating base include cellulose polymers such as hydroxypropylcellulose, hydroxypropylmethylcellulose, hydroxyethylcellulose, methylhydroxyethylcellulose and the like; synthetic polymers such as polyvinyl acetal diethylaminoacetate, aminoalkylmethacrylate copolymer E [Eudragit E (trade name)], polyvinylpyrrolidone and the like; and polysaccharides such as pullulan and the like.

Examples of the enteric film coating base include cellulose polymers such as hydroxypropylmethylcellulose phthalate, hydroxypropylmethylcellulose acetate succinate, carboxymethylethylcellulose, cellulose acetate phthalate and the like; acrylic acid polymers such as methacrylic acid copolymer L [Eudragit L (trade name)], methacrylic acid copolymer LD [Eudragit L-30D-55 (trade name)], methacrylic acid copolymer S [Eudragit S (trade name)] and the like; and naturally-occurring substances such as shellac and the like.

Examples of the sustained-release film coating base include cellulose polymers such as ethylcellulose and the like; and acrylic acid polymers such as aminoalkylmethacrylate copolymer RS [Eudragit RS (trade name)], ethyl acrylate-methyl methacrylate copolymer suspension [Eudragit NE (trade name)] and the like.

Two or more kinds of the above-mentioned coating bases may be used in a mixture at an appropriate ratio. In addition, for example, light shielding agents such as titanium oxide, red ferric oxide and the like may also be used during coating.

Since the compound of the present invention shows low toxicity (e.g., acute toxicity, chronic toxicity, genetic toxicity, reproductive toxicity, cardiotoxicity, carcinogenicity) and less side effects, it can be used as a prophylactic or therapeutic agent, or diagnostic agent for various diseases in mammals (e.g., human, bovine, horse, dog, cat, monkey, mouse, rat).

The compound of the present invention has an excellent an orexin type 2 receptor agonist activity, and may treat, prevent or ameliorate the risk of various neurological and psychiatric diseases associated with an orexin type 2 receptor. The compound of the present invention is useful as an agent for the prophylaxis or treatment of various diseases such as narcolepsy, idiopathic hypersomnia, hypersomnia, sleep apnea syndrome, narcolepsy syndrome accompanied by narcolepsy-like symptoms, hypersomnia syndrome accompanied by daytime hypersomnia (e.g., Kleine Levin syndrome, major depression with hypersomnia, Lewy body dementia, Parkinson's disease, progressive supranuclear paralysis, Prader-Willi syndrome, Moebius syndrome, hypoventilation syndrome, Niemann-Pick disease type C, brain contusion, cerebral infarction, brain tumor, muscular dystrophy, multiple sclerosis, acute disseminated encephalomyelitis, Guillain-Barre syndrome, Rasmussen's encephalitis, Wernicke's encephalitis, limbic encephalitis, Hashimoto's encephalopathy), coma, loss of consciousness, obesity (e.g., malignant mastocytosis, exogenous obesity, hyperinsulinar obesity, hyperplasmic obesity, hypophyseal adiposity, hypoplasmic obesity, hypothyroid obesity, hypothalamic obesity, symptomatic obesity, infantile obesity, upper body obesity, alimentary obesity, hypogonadal obesity, systemic mastocytosis, simple obesity, central obesity), insulin resistance syndrome, Alzheimer's disease, disturbance of consciousness such as coma and the like, side effects and complications due to anesthesia, sleep disturbance, sleep problem, insomnia, Intermittent sleep, nocturnal myoclonus, REM sleep interruption, jet lag, jet lag syndrome, sleep disorder of alternating worker, sleep disorder, night terror, depression, major depression, sleepwalking disease, enuresis, sleep disorder, Alzheimer's dusk, diseases associated with circadian rhythm, fibromyalgia, condition arising from decline in the quality of sleep, overeating, obsessive compulsive eating disorder, obesity-related disease, hypertension, diabetes, elevated plasma insulin concentration and insulin resistance, hyperlipidemia, hyperlipemia, endometrial cancer, breast cancer, prostate cancer, colorectal cancer, cancer, osteoarthritis, obstructive sleep apnea, cholelithiasis, gallstones, cardiac disease, abnormal heartbeat, arrhythmia, myocardial infarction, congestive cardiac failure, cardiac failure, coronary heart disease, cardiovascular disorder, sudden death, polycysticovarian disease, craniopharingioma, Froelich's syndrome, growth hormone deficient, normal mutant short stature, Turner's syndrome, children suffering from acute lymphoblastic leukemia, syndrome X, reproductive hormone abnormality, declining fertility, infertility, male gonadal function decline, sexual and reproductive dysfunction such as female male hirsutism, fetal defects associated with pregnant women obesity, gastrointestinal motility disorders such as obesity-related gastroesophageal reflux, obesity hypoventilation syndrome (Pickwick syndrome), respiratory diseases such as dyspnea, inflammation such as systemic inflammation of the vascular system, arteriosclerosis, hypercholesterolemia, hyperuricemia, lower back pain, gall bladder disease, gout, kidney cancer, risk of secondary outcomes of obesity such as lowering the risk of left ventricular hypertrophy, migraine pain, headache, neuropathic pain, Parkinson's disease, psychosis, schizophrenia, facial flushing, night sweats, diseases of the genital/urinary system, diseases related to sexual function or fertility, dysthymic disorder, bipolar disorder, bipolar I disorder, bipolar II disorder, cyclothymic disorder, acute stress disorder, agoraphobia, generalized anxiety disorder, obsessive disorder, panic attack, panic disorder, posttraumatic stress disorder, separation anxiety disorder, social phobia, anxiety disorder, acute neurological and psychiatric disorders such as cardiac bypass surgery and post-transplant cerebral deficit, stroke, ischemic stroke, cerebral ischemia, spinal cord trauma, head trauma, perinatal hypoxia, cardiac arrest, hypoglycemic nerve injury, Huntington's disease, amyotrophic lateral sclerosis, multiple sclerosis, eye damage, retinopathy, cognitive impairment, muscle spasm, tremor, epilepsy, disorders associated with muscle spasticity, delirium, amnestic disorder, age-related cognitive decline, schizoaffective disorder, delusional disorder, drug addiction, dyskinesia, chronic fatigue syndrome, fatigue, medication-induced Parkinsonism syndrome, Jill-do La Tourette's syndrome, chorea, myoclonus, tic, restless legs syndrome, dystonia, dyskinesia, attention deficit hyperactivity disorder (ADHD), behavior disorder, urinary incontinence, withdrawal symptoms, trigeminal neuralgia, hearing loss, tinnitus, nerve damage, retinopathy, macular degeneration, vomiting, cerebral edema, pain, bone pain, arthralgia, toothache, cataplexy, and traumatic brain injury.

Particularly, the compound of the present invention is useful as an agent for the prophylaxis or treatment of narcolepsy, idiopathic hypersomnia, hypersomnia, sleep apnea syndrome, narcolepsy syndrome accompanied by narcolepsy-like symptoms, hypersomnia syndrome accompanied by daytime hypersomnia (e.g., Parkinson's disease, Guillain-Barre syndrome and Kleine Levin syndrome), Alzheimer's disease, obesity, insulin resistance syndrome, cardiac failure, diseases related to bone loss, sepsis, disturbance of consciousness such as coma and the like, side effects and complications due to anesthesia, and the like, or anesthetic antagonist.

While the dose of the compound of the present invention varies depending on the subject of administration, administration route, target disease, symptom and the like, for example, when the compound of the present invention is administered orally or parenterally to an adult patient, its dose is for example, about 0.01 to 100 mg/kg body weight per dose, preferably 0.1 to 50 mg/kg body weight per dose and more preferably 0.5 to 20 mg/kg body weight per dose. This amount is desirably administered in one to 3 portions daily.

The compound of the present invention can be used in combination with other drugs (hereinafter to be abbreviated as concomitant drug).

By combining the compound of the present invention and a concomitant drug, a superior effect, for example,
(1) the dose can be reduced as compared to single administration of the compound of the present invention or a concomitant drug,
(2) the drug to be combined with the compound of the present invention can be selected according to the condition of patients (mild case, severe case and the like),
(3) the period of treatment can be set longer by selecting a concomitant drug having different action and mechanism from the compound of the present invention,
(4) a sustained treatment effect can be designed by selecting a concomitant drug having different action and mechanism from the compound of the present invention,
(5) a synergistic effect can be afforded by a combined use of the compound of the present invention and a concomitant drug, and the like, can be achieved.

In the present specification, the compound of the present invention and a concomitant drug used in combination are referred to as the “combination agent of the present invention”.

When using the combination agent of the present invention, the administration time of the compound of the present invention and the concomitant drug is not restricted, and the compound of the present invention or a pharmaceutical composition thereof, or the concomitant drug or a pharmaceutical composition thereof can be administered to an administration subject simultaneously, or may be administered at different times. The dosage of the concomitant drug may be determined according to the dose clinically used, and can be appropriately selected depending on an administration subject, administration route, disease, combination and the like.

The administration mode of the combination agent of the present invention and the concomitant drug is not particularly limited, and the compound of the present invention and the concomitant drug only need to be combined on administration.

Examples of such administration mode include the following: (1) administration of a single preparation obtained by simultaneously processing the compound of the present invention and the concomitant drug, (2) simultaneous administration of two kinds of preparations of the compound of the present invention and the concomitant drug, which have been separately produced, by the same administration route, (3) administration of two kinds of preparations of the compound of the present invention and the concomitant drug, which have been separately produced, by the same administration route in a staggered manner, (4) simultaneous administration of two kinds of preparations of the compound of the present invention and the concomitant drug, which have been separately produced, by different administration routes, (5) administration of two kinds of preparations of the compound of the present invention and the concomitant drug, which have been separately produced, by different administration routes in a staggered manner (e.g., administration in the order of the compound of the present invention and the concomitant drug, or in the reverse order) and the like.

The dose of the concomitant drug can be appropriately determined based on the dose employed in clinical situations. The mixing ratio of the compound of the present invention and a concomitant drug can be appropriately determined depending on the administration subject, administration route, target disease, symptom, combination and the like.

For example, the content of the compound of the present invention in the combination agent of the present invention differs depending on the form of a preparation, and usually from about 0.01 to about 100 wt %, preferably from about 0.1 to about 50 wt %, further preferably from about 0.5 to about 20 wt %, based on the whole preparation.

The content of the concomitant drug in the combination agent of the present invention differs depending on the form of a preparation, and usually from about 0.01 to about 100 wt %, preferably from about 0.1 to about 50 wt %, further preferably from about 0.5 to about 20 wt %, based on the whole preparation.

The content of additives such as a carrier and the like in the combination agent of the present invention differs depending on the form of a preparation, and usually from about 1 to about 99.99 wt %, preferably from about 10 to about 90 wt %, based on the preparation.

Similar contents may be employed even when the compound of the present invention and a concomitant drug are separately formulated into preparations.

Examples of the concomitant drug include the followings. A therapeutic drug for narcolepsy (e.g., methylphenidate, amphetamine, pemoline, phenelzine, protriptyline, sodium oxybate, modafinil, caffeine), antiobesity drug (amphetamine, benzfetamine, bromocriptine, bupropion, diethylpropion, exenatide, fenfluramine, liothyronine, liraglutide, mazindol, methamphetamine, octreotide, octreotide, orlistat, phendimetrazine, phendimetrazine, phenmetrazine, phentermine, Qnexa (registered trade mark), phenylpropanolamine, pramlintide, propylhexedrine, recombinant leptin, sibutramine, topiramate, zimelidine, zonisamide, Lorcaserin, metformin), acetylcholine esterase inhibitor (e.g., donepezil, rivastigmine, galanthamine, zanapezil, idebenone, tacrine), antidementia agent (e.g., memantine), inhibitor of β amyloid protein production, secretion, accumulation, aggregation and/or deposition, β secretase inhibitor (e.g., 6-(4-biphenylyl)methoxy-2-[2-(N,N-dimethylamino)ethyl]tetralin, 6-(4-biphenylyl)methoxy-2-(N,N-dimethylamino)methyltetralin, 6-(4-biphenylyl)methoxy-2-(N,N-dipropylamino)methyltetralin, 2-(N,N-dimethylamino)methyl-6-(4′-methoxybiphenyl-4-yl)methoxytetralin, 6-(4-biphenylyl)methoxy-2-[2-(N,N-diethylamino)ethyl]tetralin, 2-[2-(N,N-dimethylamino)ethyl]-6-(4′-methylbiphenyl-4-yl)methoxytetralin, 2-[2-(N,N-dimethylamino)ethyl]-6-(4′-methoxybiphenyl-4-yl)methoxytetralin, 6-(2′,4′-dimethoxybiphenyl-4-yl)methoxy-2-[2-(N,N-dimethylamino)ethyl]tetralin, 6-[4-(1,3-benzodioxol-5-yl)phenyl]methoxy-2-[2-(N,N-dimethylamino)ethyl]tetralin, 6-(3′,4′-dimethoxybiphenyl-4-yl)methoxy-2-[2-(N,N-dimethylamino)ethyl]tetralin, an optically active form thereof, a salt thereof and a hydrate thereof, OM99-2 (WO01/00663)), γ secretase inhibitor, β amyloid protein aggregation inhibitor (e.g., PTI-00703, ALZHEMED (NC-531), PPI-368 (National Publication of International Patent Application No. 11-514333), PPI-558 (National Publication of International Patent Application No. 2001-500852), SKF-74652 (Biochem. J. (1999), 340(1), 283-289)), β amyloid vaccine, β amyloid-degrading enzyme and the like, brain function enhancer (e.g., aniracetam, nicergoline), therapeutic drug for Parkinson's disease [(e.g., dopamine receptor agonist (e.g., L-DOPA, bromocriptine, pergolide, talipexole, pramipexole, cabergoline, amantadine), monoamine oxidase enzyme (MAO) inhibitor (e.g., deprenyl, selegiline, remacemide, riluzole), anticholinergic agent (e.g., trihexyphenidyl, biperiden), COMT inhibitor (e.g., entacapone)], therapeutic drug for amyotrophic lateral sclerosis (e.g., riluzole etc., neurotrophic factor), therapeutic drug for abnormal behavior accompanying progress of dementia, wandering and the like (e.g., sedative, anti-anxiety drug), apoptosis inhibitor (e.g., CPI-1189, IDN-6556, CEP-1347), neuronal differentiation-regenerate promoter (e.g., leteprinim, xaliproden; SR-57746-A), SB-216763, Y-128, VX-853, prosaptide, 5,6-dimethoxy-2-[2,2,4,6,7-pentamethyl-3-(4-methylphenyl)-2,3-dihydro-1-benzofuran-5-yl]isoindoline, 5,6-dimethoxy-2-[3-(4-isopropylphenyl)-2,2,4,6,7-pentamethyl-2,3-dihydro-1-benzofuran-5-yl]isoindoline, 6-[3-(4-isopropylphenyl)-2,2,4,6,7-pentamethyl-2,3-dihydro-1-benzofuran-5-yl]-6,7-dihydro-5H-[1,3]dioxolo[4,5-f]isoindole and an optically active form, salt or hydrate thereof), non-steroidal antiinflammatory agents (meloxicam, tenoxicam, indomethacin, ibuprofen, celecoxib, rofecoxib, aspirin etc.), steroid drug (dexamethasone, hexestrol, cortisone acetate etc.), disease-modifying anti-rheumatic drug (DMARDs), anti-cytokine drug (e.g., TNF inhibitor, MAP kinase inhibitor), therapeutic agent for incontinence, frequent urination (e.g., flavoxate hydrochloride, oxybutynin hydrochloride, propiverine hydrochloride), phosphodiesterase inhibitor (e.g., sildenafil(citrate)), dopamine agonist (e.g., apomorphine), antiarrhythmic drugs (e.g., mexiletine), sex hormone or a derivative thereof (e.g., progesterone, estradiol, estradiol benzoate), therapeutic agent for osteoporosis (e.g., alfacalcidol, calcitriol, elcatonin, calcitonin salmon, estriol, ipriflavone, pamidronate disodium, alendronate sodium hydrate, incadronate disodium), parathyroid hormone (PTH), calcium receptor antagonists, therapeutic drug for insomnia (e.g., benzodiazepines medicament, non-benzodiazepines medicament, melatonin agonist, orexin receptor antagonists), therapeutic drug for schizophrenia (e.g., typical antipsychotic agents such as haloperidol and the like; atypical antipsychotic agents such as clozapine, olanzapine, risperidone, aripiprazole and the like; medicament acting on metabotropic glutamate receptor or ion channel conjugated-type glutamate receptor; phosphodiesterase inhibitor), benzodiazepines medicament (chlordiazepoxide, diazepam, potassium clorazepate, lorazepam, clonazepam, alprazolam etc.), L-type calcium channel inhibitor (pregabalin etc.), tricyclic or tetracyclic antidepressant (imipramine hydrochloride, amitriptyline hydrochloride, desipramine hydrochloride, clomipramine hydrochloride etc.), selective serotonin reuptake inhibitor (fluvoxamine maleate, fluoxetine hydrochloride, citalopram hydrobromide, sertraline hydrochloride, paroxetine hydrochloride, escitalopram oxalate etc.), serotonin-noradrenaline reuptake inhibitor (venlafaxine hydrochloride, duloxetine hydrochloride, desvenlafaxine hydrochloride etc.), noradrenaline reuptake inhibitor (reboxetine mesylate etc.), mirtazapine, trazodone hydrochloride, nefazodone hydrochloride, bupropion hydrochloride, setiptiline maleate, 5-HT_{1A} agonist, (buspirone hydrochloride, tandospirone citrate, osemozotan hydrochloride etc.), 5-HT_{2A} antagonist, 5-HT_{2A} inverse agonist, 5-HT₃ antagonist (cyamemazine etc.), heart non-selective β inhibitor (propranolol hydrochloride, oxprenolol hydrochloride etc.), histamine H₁ antagonist (hydroxyzine hydrochloride etc.), CRF antagonist, other antianxiety drug (meprobamate etc.), tachykinin antagonist (MK-869, saredutant etc.), medicament that acts on metabotropic glutamate receptor, CCK antagonist, β3 adrenaline antagonist (amibegron hydrochloride etc.), GAT-1 inhibitor (tiagabine hydrochloride etc.), N-type calcium channel inhibitor, carbonic anhydrase II inhibitor, NMDA glycine moiety agonist, NMDA antagonist (memantine etc.), peripheral benzodiazepine receptor agonist, vasopressin antagonist, vasopressin V1b antagonist, vasopressin V1a antagonist, phosphodiesterase inhibitor, opioid antagonist, opioid agonist, uridine, nicotinic acid receptor agonist, thyroid hormone (T3, T4), TSH, TRH, MAO inhibitor (phenelzine sulfate, tranylcypromine sulfate, moclobemide etc.), therapeutic drug for bipolar disorder (lithium carbonate, sodium valproate, lamotrigine, riluzole, felbamate etc.), cannabinoid CB1 antagonist (rimonabant etc.), FAAH inhibitor, sodium channel inhibitor, anti-ADHD drug (methylphenidate hydrochloride, methamphetamine hydrochloride etc.), therapeutic drug for alcoholism, therapeutic drug for autism, therapeutic drug for chronic fatigue syndrome, therapeutic drug for spasm, therapeutic drug for fibromyalgia syndrome, therapeutic drug for headache, therapeutic drug for quitting smoking, therapeutic drug for myasthenia gravis, therapeutic drug for cerebral infarction, therapeutic drug for mania, therapeutic drug for hypersomnia, therapeutic drug for pain, therapeutic drug for dysthymia, therapeutic drug for autonomic ataxia, therapeutic drug for male and female sexual dysfunction, therapeutic drug for migraine, therapeutic drug for pathological gambler, therapeutic drug for restless legs syndrome, therapeutic drug for substance addiction, therapeutic drug for alcohol-related syndrome, therapeutic drug for irritable bowel syndrome, therapeutic drug for lipid abnormality such as cholesterol-lowering drug (statin series (pravastatin sodium, atorvastatin, simvastatin, rosuvastatin etc.), fibrate (clofibrate etc.), squalene synthetase inhibitor), therapeutic drug for abnormal behavior or suppressant of dromomania due to dementia (sedatives, antianxiety drug etc.), therapeutic drug for diabetes, therapeutic agent for diabetic complications, therapeutic drug for hypertension, therapeutic drug for hypotension, diuretic, chemotherapeutic agent, immunotherapeutic agent, antithrombotic agent, anti-cancer agent and the like.

Two or more kinds of the above-mentioned concomitant drug may be used in a mixture at an appropriate ratio.

When the compound of the present invention is applied to each of the above-mentioned diseases, it can also be used in combination with biologics (e.g., antibody drug, nucleic acid or nucleic acid derivative, aptamer drug, vaccine preparation), or can be used in combination with a gene therapy method and the like, or can also be used in combination with a treatment in psychiatric field without using drugs.

Examples of the antibody drug and vaccine preparation include vaccine preparation against angiotensin II, vaccine preparation against CETP, CETP antibody, antibody against TNFα antibody and other cytokines, amyloid β vaccine preparation, vaccine for type 1 diabetes (e.g., DIAPEP-277 of Peptor), anti-HIV antibody and HIV vaccine preparation, as well as antibodies or vaccine preparations against cytokines, renin-angiotensin type enzymes and products thereof, antibodies or vaccine preparations against enzymes or proteins involved in blood lipid metabolism, antibodies or vaccines relating to enzymes and proteins involved in blood coagulation or fibrinolysis system, antibodies or vaccine preparations against proteins involved in sugar metabolism and insulin resistance, and the like. In addition, it can be used in combination with biologics relating to growth factors such as GH, IGF and the like.

Examples of the gene therapy method include a treatment method using gene relating to cytokine, renin-angiotensin type enzyme and product thereof, G protein, G protein conjugated receptor and phosphorylating enzyme thereof, a treatment method using a DNA decoy such as NFκB decoy and the like, a treatment method using antisense, a treatment method using a gene relating to an enzyme or protein involved in blood lipid metabolism (e.g., a gene relating to metabolism, excretion and absorption of cholesterol or triglyceride or HDL-cholesterol or blood phospholipid), a treatment method using a gene relating to an enzyme or protein involved in angiogenesis therapy for peripheral vascular obstruction and the like (e.g., growth factors such as HGF, VEGF etc.), a treatment method using a gene relating to a protein involved in glucose metabolism and insulin resistance, antisense against cytokines such as TNF etc., and the like.

Examples of the treatment method in the psychiatric field without using drug include modified electroconvulsive therapy, deep brain stimulation therapy, repetitive transcranial magnetic stimulation therapy, psychotherapy including cognitive behavioral therapy and the like.

The compound of the present invention can also be used in combination with various organ regeneration methods such as cardiac regeneration, renal regeneration, pancreatic regeneration, revascularization and the like, cell transplantation therapy utilizing bone marrow cells (bone marrow-derived mononuclear cell, myelogenic stem cell), or artificial organ utilizing tissue engineering (e.g., artificial blood vessel, cardiomyocyte sheet).

### EXAMPLES

The present invention is explained in detail in the following by referring to Examples, Experimental Examples and Formulation Examples. However, the examples do not limit the present invention and the examples can be modified within the scope of the present invention.

The “room temperature” in the following Examples is generally about 10° C. to about 35° C. The ratio for mixed solvent is, unless otherwise specified, a volume mixing ratio and % means wt % unless otherwise specified.

The elution by column chromatography in the Examples was performed under the observation by TLC (Thin Layer Chromatography) unless otherwise specified. In the observation by TLC, 60 F₂₅₄ manufactured by Merck was used as a TLC plate, the solvent used as an elution solvent in column chromatography was used as an eluent, and UV detector was used for the detection. In silica gel column chromatography, the indication of NH means use of aminopropylsilane-bonded silica gel and the indication of DIOL means use of 3-(2,3-dihydroxypropoxy)propylsilane-bonded silica gel. Preparative HPLC (high performance liquid chromatography) was performed under the following conditions; column: Boston Prime C18 (150 mm×30 mm, 5 μm), Xtimate C18 (100 mm×30 mm, 3 μm), Gemini NX C18 (150 mm×30 mm, 5 μm), YMC Triart C18 (250 mm×50 mm, 7 μm), Exsil plus C18 (150 mm×50 mm, 5 μm), or Water Xbridge C18 (150 mm×30 mm, 5 μm), mobile phase: 0.05% aqueous ammonia/MeCN. The indication of C18 means use of octadecyl-bonded silica gel. The ratio for elution solvent is, unless otherwise specified, a volume mixing ratio.

For the analysis of ¹H NMR, ACD/SpecManager (trade name) software and the like were used. Peaks of a hydroxyl group, an amino group and the like, having very mild proton peak, are not sometimes described.

MS was measured by LC/MS. As the ionization method, ESI method, or APCI method was used. The data indicates actual measured value (found). While molecular ion peak is generally observed, a fragment ion is sometimes observed. In the case of a salt, a molecular ion peak or fragment ion peak of free form is generally observed.

In the following Examples, the following abbreviations are used.
MS: mass spectrum
M: mol concentration
N: normality
CDCl₃: deuterochloroform
DMSO-d₆: deuterodimethyl sulfoxide
¹H NMR: proton nuclear magnetic resonance
LC/MS: liquid chromatograph mass spectrometer
ESI: electrospray ionization,
APCI: atmospheric pressure chemical ionization
IPE: diisopropyl ether
IPA: 2-propanol
DMF: N,N-dimethylformamide
THF: tetrahydrofuran
MeOH: methanol
MeCN: acetonitrile
NMP: N-methylpyrrolidone
CPME: cyclopentyl methyl ether
DME: 1,2-dimethoxyethane
DMSO: dimethyl sulfoxide
DCM: dichloromethane
DCE: dichloroethane
EtOH: ethanol
XPhos Pd G3: (2-dicyclohexylphosphino-2′,4′,6′-triisopropyl-1,1′-biphenyl) [2-(2′-amino-1,1′-biphenyl)]palladium(II) methanesulfonate
SFC: supercritical fluid chromatography
HATU: 1-[bis(dimethylamino)methylene]-1H-1,2,3-triazolo[4,5-b]pyridinium 3-oxide hexafluorophosphate
HOBt: 1-hydroxybenzotriazole
EDCI: N-ethyl-N′-(3-dimethylaminopropyl)carbodiimide hydrochloride
TEA: triethylamine
DIPEA: N,N-diisopropylethylamine
NaH: sodium hydride
Boc: tert-butoxycarbonyl
DMAP: 4-dimethylaminopyridine
CO₂: carbon dioxide
Pd/C: palladium on carbon
Na2SO4: sodium sulfate, anhydrous
NaBH4: sodium borohydride
LiBH4: lithium borohydride
PPh3: triphenylphosphine
DIAD: diisopropyl azodicarboxylate
rt: room temperature
EtOAc: ethyl acetate
Tf2O: trifluoromethanesulfonic anhydride
TBAF: tetrabutylammonium fluoride
SPhos: 2-dicyclohexylphosphino-2′,6′-dimethoxybiphenyl
DBU: 1,8-diazabicyclo[5.4.0]undec-7-ene
(S,S)-pybox(iPr): (S,S)-2,6-bis(4-isopropyl-2-oxazoline-2-yl)pyridine
Pd(OH)2: palladium(II) hydroxide
Lindlar's catalyst: palladium on calcium carbonate
NaHCO₃: sodium bicarbonate
Boc2O: di-tert-butyl dicarbonate
Cs2CO3: cesium carbonate
CuCl: copper(I) chloride
Pd(OAc)2: palladium(II) acetate
MeMgBr: methylmagnesium bromide
MsCl: methanesulfonyl chloride
TsOH: p-toluenesulfonic acid
HCl: hydrogen chloride
BuLi: butyllithium

### Example 23

### N-{(3S)-1-[(1R,2R)-2-(2′,6′-difluoro[1,1′-biphenyl]-2-yl)cyclopropane-1-carbonyl]pyrrolidin-3-yl}methanesulfonamide

### A) tert-butyl (S)-3-(methylsulfonamido)pyrrolidine-1-carboxylate

To a mixture of tert-butyl (S)-3-aminopyrrolidine-1-carboxylate (4.93 g) and THF (20 mL) were added triethylamine (5.53 mL) and methanesulfonic anhydride (4.61 g) at 0° C., and the mixture was stirred at room temperature for 3 hr. The mixture was diluted with ethyl acetate and water at 0° C., and extracted with ethyl acetate. The organic layer was washed with brine, dried over sodium sulfate, passed through silica gel pad, and concentrated under reduced pressure to give the title compound (6.39 g).

¹H NMR (300 MHz, DMSO-d₆) δ 1.39 (9H, s), 1.69-1.88 (1H, m), 1.97-2.14 (1H, m), 2.94 (3H, s), 3.08 (1H, dd, J=10.9, 5.6 Hz), 3.14-3.27 (1H, m), 3.28-3.38 (1H, m), 3.43-3.53 (1H, m), 3.80-3.93 (1H, m), 7.34 (1H, s).

### B) (S)—N-(pyrrolidin-3-yl)methanesulfonamide Hydrochloride

A mixture of tert-butyl (S)-3-(methylsulfonamido)pyrrolidine-1-carboxylate (6.39 g) and 4 M hydrogen chloride-ethyl acetate solution (72.5 mL) was stirred overnight at room temperature. To the resulting suspension was added heptane (50 mL) with stirring, and the precipitate was collected, washed with ethyl acetate, and dried over at 50° C. to give the title compound (3.71 g).

¹H NMR (300 MHz, DMSO-d₆) δ 1.81-1.96 (1H, m), 2.09-2.23 (1H, m), 2.99 (3H, s), 3.05 (1H, dd, J=12.0, 5.3 Hz), 3.13-3.28 (2H, m), 3.33-3.39 (1H, m), 3.96-4.10 (1H, m), 7.48 (1H, br s), 8.93 (2H, br s).

### C) rac-ethyl (1R,2R)-2-(2-bromophenyl)cyclopropane-1-carboxylate

To a mixture of potassium tert-butoxide (42.2 g) and DMSO (400 mL) was added trimethylsulfoxonium iodide (82.8 g) at room temperature little by little. The mixture was stirred at the same temperature for 1 hr, a solution of ethyl (E)-3-(2-bromophenyl)acrylate (80.0 g) in DMSO (400 mL) was added thereto, and the mixture was stirred at the same temperature for 12 hr. The mixture was poured into ice-water, and extracted with ethyl acetate. The organic layer was washed with brine, dried over sodium sulfate, and concentrated under reduced pressure. The residue was purified by silica gel column chromatography (ethyl acetate/petroleum ether) to give the title compound (34.4 g).

¹H NMR (400 MHz, CDCl₃) δ 1.27-1.36 (4H, m), 1.61-1.67 (1H, m), 1.75-1.83 (1H, m), 2.66-2.75 (1H, m), 4.14-4.28 (2H, m), 6.98-7.05 (1H, m), 7.06-7.13 (1H, m), 7.19-7.26 (1H, m), 7.56 (1H, dd, J=8.0, 1.2 Hz).

### D) rac-ethyl (1R,2R)-2-(2′,6′-difluoro[1,1′-biphenyl]-2-yl)cyclopropane-1-carboxylate

To a mixture of rac-ethyl (1R,2R)-2-(2-bromophenyl)cyclopropane-1-carboxylate (5.00 g), 2,6-difluorophenylboronic acid (8.80 g), DME (60 mL) and water (50 mL) were added Xphos Pd G3 (1.57 g) and tripotassium phosphate (11.8 g), and the mixture was stirred under nitrogen atmosphere at 100° C. for 12 hr. The mixture was poured into water, and extracted with ethyl acetate. The organic layer was washed with brine, dried over sodium sulfate, and concentrated under reduced pressure. The residue was purified by silica gel column chromatography (ethyl acetate/petroleum ether) to give the title compound (3.73 g).

MS: [M+H]⁺ 303.3.

### E) rac-(1R,2R)-2-(2′,6′-difluoro[1,1′-biphenyl]-2-yl)cyclopropane-1-carboxylic Acid

To a mixture of rac-ethyl (1R,2R)-2-(2′,6′-difluoro[1,1′-biphenyl]-2-yl)cyclopropane-1-carboxylate (3.73 g) and EtOH (40 mL) was added aqueous sodium hydroxide solution (4.93 g/20 mL), and the mixture was stirred at room temperature for 12 hr. The mixture was concentrated under reduced pressure, diluted with water, adjusted to pH=4 with 2 M hydrochloric acid, and extracted with ethyl acetate. The organic layer was dried over sodium sulfate, and concentrated under reduced pressure to give the title compound (3.37 g).

¹H NMR (400 MHz, CDCl₃) δ 1.30-1.37 (1H, m), 1.40-1.47 (1H, m), 1.70-1.76 (1H, m), 2.39-2.46 (1H, m), 6.94-7.03 (2H, m), 7.15 (1H, t, J=7.2 Hz), 7.26-7.41 (4H, m)

### F) A Mixture of N-{(3S)-1-[(1R,2R)-2-(2′,6′-difluoro[1,1′-biphenyl]-2-yl)cyclopropane-1-carbonyl]pyrrolidin-3-yl}methanesulfonamide and N-{(3S)-1-[(1S,2S)-2-(2′,6′-difluoro[1,1′-biphenyl]-2-yl)cyclopropane-1-carbonyl]pyrrolidin-3-yl}methanesulfonamide

To a mixture of rac-(1R,2R)-2-(2′,6′-difluoro[1,1′-biphenyl]-2-yl)cyclopropane-1-carboxylic acid (2.00 g), (S)—N-(pyrrolidin-3-yl)methanesulfonamide hydrochloride (1.46 g), HATU (2.91 g) and DMF (15 mL) was added DIPEA (3.77 g), and the mixture was stirred at room temperature for 12 hr. The mixture was purified by reverse-phase silica gel column chromatography (C18, methanol/water) to give the title compound (1.53 g).

MS: [M+H]⁺ 421.1.

### G) N-{(3S)-1-[(1R,2R)-2-(2′,6′-difluoro[1,1′-biphenyl]-2-yl)cyclopropane-1-carbonyl]pyrrolidin-3-yl}methanesulfonamide

A mixture (4.10 g) of N-{(3S)-1-[(1R,2R)-2-(2′,6′-difluoro[1,1′-biphenyl]-2-yl)cyclopropane-1-carbonyl]pyrrolidin-3-yl}methanesulfonamide and N-{(3S)-1-[(1S,2S)-2-(2′,6′-difluoro[1,1′-biphenyl]-2-yl)cyclopropane-1-carbonyl]pyrrolidin-3-yl}methanesulfonamide was resolved by SFC (column: Phenomenex-Cellulose-2, 250 mm×50 mm, 10 μm, mobile phase: CO₂/EtOH containing 0.1% aqueous ammonia=55/45 v/v), and the fraction having a shorter retention time was concentrated. The residue was dissolved in MeCN (10 mL) and water (50 mL), and the solution was freeze-dried. The obtained solid was triturated with petroleum ether/ethyl acetate to give the title compound (1.31 g).

¹H NMR (400 MHz, CDCl₃) δ 1.24-1.32 (1H, m), 1.42-1.52 (1H, m), 1.58-1.72 (1H, m), 1.78-2.10 (1H, m), 2.10-2.30 (1H, m), 2.32-2.43 (1H, m), 2.98 (3H, d, J=12.0 Hz), 3.27-3.76 (4H, m), 3.94-4.12 (1H, m), 4.67-4.99 (1H, m), 6.91-7.05 (2H, m), 7.14 (1H, d, J=7.6 Hz), 7.22-7.41 (4H, m).

### Example 24

### N-{(3S)-1-[(1R,2R)-2-(2′,6′-difluoro [1,1′-biphenyl]-2-yl)cyclopropane-1-carbonyl]piperidin-3-yl}methanesulfonamide

### A) tert-butyl (S)-3-(methylsulfonamido)piperidine-1-carboxylate

To a mixture of tert-butyl (S)-3-aminopiperidine-1-carboxylate (11.4 g) and THF (50 mL) were added triethylamine (11.9 mL) and methanesulfonic anhydride (9.92 g) at 0° C., and the mixture was stirred overnight at room temperature. The mixture was diluted with ethyl acetate and water at 0° C., and extracted with ethyl acetate. The organic layer was washed with brine, dried over sodium sulfate, passed through silica gel pad, and concentrated under reduced pressure to give the title compound (14.9 g).

¹H NMR (300 MHz, CDCl₃) δ 1.40-1.80 (12H, m), 1.87-2.01 (1H, m), 3.01 (3H, s), 3.19-3.34 (2H, m), 3.35-3.57 (2H, m), 3.62-3.75 (1H, m), 4.54 (1H, br d, J=6.0 Hz).

### B) (S)—N-(piperidin-3-yl)methanesulfonamide Hydrochloride

A mixture of tert-butyl (S)-3-(methylsulfonamido)piperidine-1-carboxylate (14.9 g) and 4 M hydrogen chloride CPME solution (134 mL) was stirred at room temperature for 3 hr. The CPME was removed by decantation, and the remaining gum-like substance was dried to give the title compound (10.0 g).

¹H NMR (300 MHz, DMSO-d₆) δ 1.37-2.00 (4H, m), 2.62-2.85 (2H, m), 2.98 (3H, s), 3.04-3.17 (1H, m), 3.20-3.33 (1H, m), 3.47-3.61 (1H, m), 7.45 (1H, d, J=7.2 Hz), 8.87-9.26 (2H, m).

### C) A Mixture of N-{(3S)-1-[(1R,2R)-2-(2′,6′-difluoro[1,1′-biphenyl]-2-yl)cyclopropane-1-carbonyl]piperidin-3-yl}methanesulfonamide and N-{(3S)-1-[(1S,2S)-2-(2′,6′-difluoro[1,1′-biphenyl]-2-yl)cyclopropane-1-carbonyl]piperidin-3-yl}methanesulfonamide

To a mixture of rac-(1R,2R)-2-(2′,6′-difluoro[1,1′-biphenyl]-2-yl)cyclopropane-1-carboxylic acid (3.00 g), (S)—N-(piperidin-3-yl)methanesulfonamide hydrochloride (2.35 g), HATU (4.37 g) and DMF (30 mL) was added DIPEA (5.65 g), and the mixture was stirred at room temperature for 12 hr. The mixture was poured into water, and extracted with ethyl acetate. The organic layer was washed with brine, dried over sodium sulfate, and concentrated under reduced pressure. The residue was purified by silica gel column chromatography (ethyl acetate) to give the title compound (4.93 g).

MS: [M+H]⁺ 435.1.

### D) N-{(3S)-1-[(1R,2R)-2-(2′,6′-difluoro[1,1′-biphenyl]-2-yl)cyclopropane-1-carbonyl]piperidin-3-yl}methanesulfonamide

A mixture (4.93 g) of N-{(3S)-1-[(1R,2R)-2-(2′,6′-difluoro[1,1′-biphenyl]-2-yl)cyclopropane-1-carbonyl]piperidin-3-yl}methanesulfonamide and N-{(3S)-1-[(1S,2S)-2-(2′,6′-difluoro[1,1′-biphenyl]-2-yl)cyclopropane-1-carbonyl]piperidin-3-yl}methanesulfonamide was resolved by SFC (column: Phenomenex-Cellulose-2, 250 mm×50 mm, 10 μm, mobile phase: CO₂/EtOH containing 0.1% aqueous ammonia=55/45 v/v), and the fraction having a shorter retention time was concentrated. The residue was dissolved in MeCN (10 mL) and water (50 mL), and the solution was freeze-dried to give the title compound (1.56 g).

¹H NMR (400 MHz, CDCl₃) δ 1.22-1.36 (1H, m), 1.43-1.50 (1H, m), 1.51-1.61 (1H, m), 1.78-2.02 (4H, m), 2.20-2.42 (1H, m), 2.77-3.04 (3H, m), 3.27-3.92 (5H, m), 4.47-4.77 (1H, m), 6.92-7.42 (7H, m).

### Example 25

### N-{(4S)-1-[(1R,2R)-2-(2′,6′-difluoro[1,1′-biphenyl]-2-yl)cyclopropane-1-carbonyl]azepan-4-yl}methanesulfonamide

### A) tert-butyl 4-(methylsulfonamido)azepane-1-carboxylate

To a mixture of tert-butyl 4-aminoazepane-1-carboxylate (20.0 g) and DCM (180 mL) were added triethylamine (18.9 g) and methanesulfonyl chloride (13.9 g) at 0° C., and the mixture was stirred at room temperature for 16 hr. The mixture was diluted with water, and extracted with DCM. The organic layer was washed with brine, dried over sodium sulfate, and concentrated under reduced pressure to give the title compound (28.5 g).

¹H NMR (400 MHz, CDCl₃) δ 1.45 (9H, s), 1.65-2.15 (5H, m), 2.95 (3H, s), 3.06-3.67 (6H, m), 4.64 (1H, d, J=7.6 Hz).

### B) N-(azepan-4-yl)methanesulfonamide Hydrochloride

A mixture of tert-butyl 4-(methylsulfonamido)azepane-1-carboxylate (28.5 g) and 4 M hydrogen chloride dioxane solution (100 mL) was stirred at room temperature for 1 hr. The solvent was evaporated under reduced pressure to give the title compound (23.5 g).

¹H NMR (400 MHz, DMSO-d₆) δ 1.53-2.14 (6H, m), 2.90-3.21 (7H, m), 3.35-3.52 (1H, m), 7.23 (1H, d, J=6.8 Hz), 9.16 (2H, brs).

### C) a mixture of rac-N-{(4R)-1-[(1R,2R)-2-(2′,6′-difluoro[1,1′-biphenyl]-2-yl)cyclopropane-1-carbonyl]azepan-4-yl}methanesulfonamide and rac-N-{(4S)-1-[(1R,2R)-2-(2′,6′-difluoro[1,1′-biphenyl]-2-yl)cyclopropane-1-carbonyl]azepan-4-yl}methanesulfonamide

To a mixture of rac-(1R,2R)-2-(2′,6′-difluoro[1,1′-biphenyl]-2-yl)cyclopropane-1-carboxylic acid (150 mg), N-(azepan-4-yl)methanesulfonamide hydrochloride (150 mg), DIPEA (212 mg) and DMF (5.0 mL) was added HATU (312 mg) at 0° C., and the mixture was stirred at room temperature for 12 hr. The mixture was diluted with water, and extracted with ethyl acetate. The organic layer was washed with brine, dried over sodium sulfate, and concentrated under reduced pressure. The residue was purified by preparative HPLC (C18, 0.05% aqueous ammonia/MeCN), and freeze-dried to give the title compound (105 mg).

MS: [M+H]⁺ 449.3.

### D) N-{(4S)-1-[(1R,2R)-2-(2′,6′-difluoro[1,1′-biphenyl]-2-yl)cyclopropane-1-carbonyl]azepan-4-yl}methanesulfonamide

A mixture (105 mg) of rac-N-{(4R)-1-[(1R,2R)-2-(2′,6′-difluoro[1,1′-biphenyl]-2-yl)cyclopropane-1-carbonyl]azepan-4-yl}methanesulfonamide and rac-N-{(4S)-1-[(1R,2R)-2-(2′,6′-difluoro[1,1′-biphenyl]-2-yl)cyclopropane-1-carbonyl]azepan-4-yl}methanesulfonamide was resolved by SFC (column: Phenomenex-Cellulose-2, 250 mm×30 mm, 5 μm, mobile phase: CO₂/MeOH containing 0.1% aqueous ammonia=45/55 v/v), and the fraction having a shortest retention time was concentrated. The residue was dissolved in MeCN (3.0 mL) and water (3.0 mL), and the solution was freeze-dried to give a crude product (45 mg). This was again resolved by SFC (column: DAICEL CHIRALPAK IG, 250 mm×30 mm, 10 μm, mobile phase: CO₂/IPA containing 0.1% aqueous ammonia=60/40 v/v), and the fraction having a shorter retention time was concentrated. The residue was dissolved in MeCN (3.0 mL) and water (3.0 mL), and the solution was freeze-dried to give the title compound (13 mg).

¹H NMR (400 MHz, CDCl₃) δ 1.25-1.28 (1H, m), 1.46-1.53 (1H, m), 1.57-2.40 (8H, m), 2.95-2.97 (3H, m), 3.17-3.78 (5H, m), 4.15-4.19 (1H, m), 6.90-7.14 (3H, m), 7.26-7.41 (4H, m).

### Example 195

### N-{(3S)-1-[(1R,2R)-2-(2′,3,6′-trifluoro[1,1′-biphenyl]-2-yl)cyclopropane-1-carbonyl]pyrrolidin-3-yl}methanesulfonamide, or N-{(3S)-1-[(1S,2S)-2-(2′,3,6′-trifluoro[1,1′-biphenyl]-2-yl)cyclopropane-1-carbonyl]pyrrolidin-3-yl}methanesulfonamide

### A) ethyl (2E)-3-(2-bromo-6-fluorophenyl)prop-2-enoate

To a mixture of ethyl 2-diethoxyphosphorylacetate (64.6 g) and THF (450 mL) was added 60% NaH (10.6 g) at 0° C. The mixture was stirred at room temperature for 15 min, 2-bromo-6-fluorobenzaldehyde (45.0 g) was added thereto, and the mixture was stirred at the same temperature for 2 hr. The reaction was quenched with water, and extracted with ethyl acetate. The organic layer was dried over sodium sulfate, and concentrated under reduced pressure. The residue was purified by silica gel column chromatography (petroleum ether) to give the title compound (59.8 g).

¹H NMR (400 MHz, CDCl₃) δ 1.34 (3H, t, J=7.2 Hz), 4.28 (2H, q, J=7.2 Hz), 6.61-6.69 (1H, m), 7.04-7.11 (1H, m), 7.12-7.20 (1H, m), 7.43 (1H, d, J=8.0 Hz), 7.83 (1H, d, J=16.4 Hz).

### B) rac-ethyl (1R,2R)-2-(2-bromo-6-fluorophenyl)cyclopropane-1-carboxylate

To a mixture of potassium tert-butoxide (14.7 g) and DMSO (250 mL) was added trimethylsulfoxonium iodide (28.8 g) at room temperature. The mixture was stirred at the same temperature for 30 min, a solution of ethyl (2E)-3-(2-bromo-6-fluorophenyl)prop-2-enoate (29.8 g) in DMSO (250 mL) was added thereto, and the mixture was stirred at the same temperature for 6 hr. The reaction was quenched with saturated aqueous ammonium chloride solution, and extracted with ethyl acetate. The organic layer was washed with brine, dried over sodium sulfate, and concentrated under reduced pressure. The residue was purified by silica gel column chromatography (petroleum ether). The same reaction was performed in two batches to give the title compound (42.2 g).

¹H NMR (400 MHz, CDCl₃) δ 1.30 (3H, t, J=7.2 Hz), 1.44-1.52 (1H, m), 1.63-1.74 (1H, m), 1.92-2.02 (1H, m), 2.38-2.48 (1H, m), 4.16-4.28 (2H, m), 6.93-7.00 (1H, m), 7.02-7.11 (1H, m), 7.34-7.39 (1H, m).

### C) rac-ethyl (1R,2R)-2-(2′,3,6′-trifluoro[1,1′-biphenyl]-2-yl)cyclopropane-1-carboxylate

To a mixture of rac-ethyl (1R,2R)-2-(2-bromo-6-fluorophenyl)cyclopropane-1-carboxylate (15.0 g), 2,6-difluorophenylboronic acid (33.0 g) and DME (100 mL) were added aqueous tripotassium phosphate solution (33.3 g/100 mL) and Xphos Pd G3 (4.42 g), and the mixture was stirred at 100° C. for 16 hr. The mixture was diluted with water, and extracted with ethyl acetate. The organic layer was washed with brine, dried over sodium sulfate, and concentrated under reduced pressure. The residue was purified by preparative HPLC (C18, 0.05% aqueous ammonia/MeCN), and freeze-dried to give the title compound (2.00 g).

MS: [M+H]⁺ 321.0.

### D) rel-ethyl (1R,2R)-2-(2′,3,6′-trifluoro[1,1′-biphenyl]-2-yl)cyclopropane-1-carboxylate

rac-Ethyl (1R,2R)-2-(2′,3,6′-trifluoro[1,1′-biphenyl]-2-yl)cyclopropane-1-carboxylate (1.00 g) was resolved by SFC (column: DAICEL CHIRALCEL OD-H, 250 mm×30 mm, 5 μm, mobile phase: CO₂/IPA containing 0.1% aqueous ammonia=90/10 v/v) to give the title compound (320 mg) having a longer retention time.

MS: [M+H]⁺ 321.0.

### E) rel-(1R,2R)-2-(2′,3,6′-trifluoro[1,1′-biphenyl]-2-yl)cyclopropane-1-carboxylic Acid

A mixture of rel-ethyl (1R,2R)-2-(2′,3,6′-trifluoro[1,1′-biphenyl]-2-yl)cyclopropane-1-carboxylate (320 mg) synthesized in Step D), aqueous sodium hydroxide solution (400 mg/1.5 mL) and EtOH (4.5 mL) was stirred at room temperature for 16 hr. The mixture was adjusted to pH=5-6 with 2 M hydrochloric acid, diluted with water, and extracted with ethyl acetate. The organic layer was dried over sodium sulfate, and concentrated under reduced pressure to give the title compound (330 mg).

¹H NMR (400 MHz, CDCl₃) δ 1.15-1.30 (1H, m), 1.34-1.43 (1H, m), 1.64-1.71 (1H, m), 2.35-2.44 (1H, m), 6.94-7.14 (4H, m), 7.25-7.40 (2H, m).

### F) N-{(3S)-1-[(1R,2R)-2-(2′,3,6′-trifluoro[1,1′-biphenyl]-2-yl)cyclopropane-1-carbonyl]pyrrolidin-3-yl}methanesulfonamide, or N-{(3S)-1-[(1S,2S)-2-(2′,3,6′-trifluoro[1,1′-biphenyl]-2-yl)cyclopropane-1-carbonyl]pyrrolidin-3-yl}methanesulfonamide

To a mixture of (S)—N-(pyrrolidin-3-yl)methanesulfonamide hydrochloride (60.4 mg), rel-(1R,2R)-2-(2′,3,6′-trifluoro[1,1′-biphenyl]-2-yl)cyclopropane-1-carboxylic acid (80.0 mg) synthesized in Step E) and DMF (3 mL) were added HATU (125 mg) and DIPEA (106 mg), and the mixture was stirred at room temperature for 16 hr. The reaction was quenched with water, and extracted with ethyl acetate. The organic layer was washed with brine, dried over sodium sulfate, and concentrated under reduced pressure. The residue was purified by preparative HPLC (C18, 0.05% aqueous ammonia/MeCN), and freeze-dried to give the title compound (70.5 mg).

¹H NMR (400 MHz, CDCl₃) δ 0.94-1.04 (1H, m), 1.27-1.35 (1H, m), 1.68-2.12 (2H, m), 2.15-2.31 (1H, m), 2.33-2.40 (1H, m), 3.01 (3H, s), 3.38-3.76 (4H, m), 3.97-4.16 (1H, m), 4.45-4.75 (1H, m), 6.96-7.12 (4H, m), 7.27-7.43 (2H, m).

### Example 200

### N-{(3S)-1-[(1R,2R)-2-(2′,5,6′-trifluoro[1,1′-biphenyl]-2-yl)cyclopropane-1-carbonyl]pyrrolidin-3-yl}methanesulfonamide, or N-{(3S)-1-[(1S,2S)-2-(2′,5,6′-trifluoro[1,1′-biphenyl]-2-yl)cyclopropane-1-carbonyl]pyrrolidin-3-yl}methanesulfonamide

### A) ethyl (2E)-3-(2-bromo-4-fluorophenyl)prop-2-enoate

To a mixture of ethyl 2-diethoxyphosphorylacetate (35.9 g) and THF (250 mL) was added 60% NaH (5.91 g) at 0° C. The mixture was stirred at room temperature for 15 min, 2-bromo-4-fluorobenzaldehyde (25.0 g) was added thereto, and the mixture was stirred at the same temperature for 2 hr. The mixture was diluted with water, and extracted with ethyl acetate. The organic layer was dried over sodium sulfate, and concentrated under reduced pressure. The residue was purified by silica gel column chromatography (ethyl acetate/petroleum ether) to give the title compound (36.9 g).

¹H NMR (400 MHz, CDCl₃) δ 1.35 (3H, t, J=7.2 Hz), 4.29 (2H, q, J=7.2 Hz), 6.34 (1H, d, J=16.0 Hz), 7.02-7.13 (1H, m), 7.37 (1H, dd, J=8.0, 2.8 Hz), 7.60 (1H, dd, J=8.8, 6.0 Hz), 7.99 (1H, d, J=16.0 Hz).

### B) rac-ethyl (1R,2R)-2-(2-bromo-4-fluorophenyl)cyclopropane-1-carboxylate

To a mixture of potassium tert-butoxide (18.5 g) and DMSO (150 mL) was added trimethylsulfoxonium iodide (36.2 g) at room temperature. The mixture was stirred at the same temperature for 30 min, a mixture of ethyl (2E)-3-(2-bromo-4-fluorophenyl)prop-2-enoate (37.4 g) and DMSO (150 mL) was added thereto, and the mixture was stirred at the same temperature for 6 hr. The mixture was diluted with water, and extracted with ethyl acetate. The organic layer was dried over sodium sulfate, and concentrated under reduced pressure. The residue was purified by silica gel column chromatography (ethyl acetate/petroleum ether) to give the title compound (13.0 g).

¹H NMR (400 MHz, CDCl₃) δ 1.27-1.32 (4H, m), 1.59-1.66 (1H, m), 1.71-1.81 (1H, m), 2.60-2.68 (1H, m), 4.17-4.25 (2H, m), 6.91-7.04 (2H, m), 7.33 (1H, dd, J=8.4, 2.8 Hz).

### C) rac-ethyl (1R,2R)-2-[4-fluoro-2-(4,4,5,5-tetramethyl-1,3,2-dioxaborolan-2-yl)phenyl]cyclopropane-1-carboxylate

To a mixture of rac-ethyl (1R,2R)-2-(2-bromo-4-fluorophenyl)cyclopropane-1-carboxylate (5.00 g), bis(pinacolato)diboron (6.63 g) and DMSO (50 mL) were added [1,1′-bis(diphenylphosphino)ferrocene]dichloropalladium(II) dichloromethane complex (1.42 g) and potassium acetate (3.42 g), and the mixture was stirred under nitrogen atmosphere at 100° C. for 16 hr. The mixture was diluted with water, and extracted with ethyl acetate. The organic layer was washed with brine, dried over sodium sulfate, and concentrated under reduced pressure. The residue was purified by silica gel column chromatography (ethyl acetate/petroleum ether) to give the title compound (3.96 g).

¹H NMR (400 MHz, CDCl₃) δ 1.25-1.35 (16H, m), 1.49-1.58 (1H, m), 1.63-1.76 (1H, m), 3.01-3.19 (1H, m), 4.04-4.28 (2H, m), 6.90-6.96 (1H, m), 6.98-7.05 (1H, m), 7.45 (1H, dd, J=9.2, 2.8 Hz).

### D) rac-ethyl (1R,2R)-2-(2′,5,6′-trifluoro[1,1′-biphenyl]-2-yl)cyclopropane-1-carboxylate

To a mixture of rac-ethyl (1R,2R)-2-[4-fluoro-2-(4,4,5,5-tetramethyl-1,3,2-dioxaborolan-2-yl)phenyl]cyclopropane-1-carboxylate (3.96 g), 1,3-difluoro-2-iodobenzene (2.84 g) and DME (35 mL) were added Xphos Pd G3 (1.00 g) and tripotassium phosphate (7.55 g), and the mixture was stirred under nitrogen atmosphere at 100° C. for 16 hr. The mixture was diluted with water, and extracted with ethyl acetate. The organic layer was dried over sodium sulfate, and concentrated under reduced pressure. The residue was purified by silica gel column chromatography (ethyl acetate/petroleum ether) to give the title compound (2.32 g).

¹H NMR (400 MHz, CDCl₃) δ 1.17-1.26 (4H, m), 1.35-1.41 (1H, m), 1.61-1.68 (1H, m), 2.28-2.37 (1H, m), 3.91-4.08 (2H, m), 6.92-7.02 (3H, m), 7.03-7.09 (1H, m), 7.10-7.16 (1H, m), 7.31-7.39 (1H, m).

### E) rac-(1R,2R)-2-(2′,5,6′-trifluoro[1,1′-biphenyl]-2-yl)cyclopropane-1-carboxylic Acid

To a mixture of sodium hydroxide (578 mg), EtOH (20 mL) and water (5 mL) was added rac-ethyl (1R,2R)-2-(2′,5,6′-trifluoro[1,1′-biphenyl]-2-yl)cyclopropane-1-carboxylate (2.32 g), and the mixture was stirred at room temperature for 5 hr. The mixture was diluted with water, adjusted to pH=5-6 with 2 M hydrochloric acid, and extracted with ethyl acetate. The organic layer was dried over sodium sulfate, and concentrated under reduced pressure to give the title compound (1.99 g).

¹H NMR (400 MHz, CDCl₃) δ 1.25-1.31 (1H, m), 1.38-1.48 (1H, m), 1.58-1.71 (1H, m), 2.29-2.44 (1H, m), 6.97-7.16 (5H, m), 7.30-7.38 (1H, m).

### F) rel-(1R,2R)-2-(2′,5,6′-trifluoro[1,1′-biphenyl]-2-yl)cyclopropane-1-carboxylic Acid

rac-(1R,2R)-2-(2′,5,6′-Trifluoro [1,1′-biphenyl]-2-yl)cyclopropane-1-carboxylic acid (500 mg) was resolved by SFC (column: Phenomenex-Cellulose-2, 250 mm×30 mm, 10 μm, mobile phase: CO₂/MeOH containing 0.1% aqueous ammonia=80/20 v/v), and the fraction having a shorter retention time was freeze-dried to give the title compound (82.0 mg).

¹H NMR (400 MHz, DMSO-d₆) δ 1.07-1.21 (1H, m), 1.21-1.33 (1H, m), 1.56-1.69 (1H, m), 1.89-2.04 (1H, m), 7.14-7.30 (5H, m), 7.49-7.58 (1H, m).

### G) N-{(3S)-1-[(1R,2R)-2-(2′,5,6′-trifluoro[1,1′-biphenyl]-2-yl)cyclopropane-1-carbonyl]pyrrolidin-3-yl}methanesulfonamide, or N-{(3S)-1-[(1S,2S)-2-(2′,5,6′-trifluoro[1,1′-biphenyl]-2-yl)cyclopropane-1-carbonyl]pyrrolidin-3-yl}methanesulfonamide

To a mixture of rel-(1R,2R)-2-(2′,5,6′-trifluoro[1,1′-biphenyl]-2-yl)cyclopropane-1-carboxylic acid (30.0 mg) obtained in Step F, (S)—N-(pyrrolidin-3-yl)methanesulfonamide hydrochloride (20.6 mg) and DMF (1 mL) were added HATU (39.0 mg) and DIPEA (66.3 mg) at 0° C., and the mixture was stirred at room temperature for 16 hr. The mixture was diluted with water, and extracted with ethyl acetate. The organic layer was dried over sodium sulfate, and concentrated under reduced pressure. The residue was purified by preparative HPLC (C18, 0.05% aqueous ammonia/MeCN), and freeze-dried to give the title compound (18.0 mg).

¹H NMR (400 MHz, CDCl₃) δ 1.17-1.27 (1H, m), 1.40-1.48 (1H, m), 1.52-1.63 (1H, m), 1.80-2.38 (3H, m), 2.98-3.01 (3H, m), 3.28-3.72 (4H, m), 3.92-4.12 (1H, m), 4.50-5.00 (1H, m), 6.94-7.16 (5H, m), 7.30-7.45 (1H, m).

### Example 215

### N-{(3R)-1-[(1R,2R)-2-(2′,6′-difluoro [1,1′-biphenyl]-2-yl)cyclopropane-1-carbonyl]-4,4-difluoropiperidin-3-yl}methanesulfonamide, or N-{(3R)-1-[(1S,2S)-2-(2′,6′-difluoro[1,1′-biphenyl]-2-yl)cyclopropane-1-carbonyl]-4,4-difluoropiperidin-3-yl}methanesulfonamide

### A) A Mixture of tert-butyl {(3R)-1-[(1R,2R)-2-(2′,6′-difluoro[1,1′-biphenyl]-2-yl)cyclopropane-1-carbonyl]-4,4-difluoropiperidin-3-yl}carbamate and tert-butyl {(3R)-1-[(1S,2S)-2-(2′,6′-difluoro[1,1′-biphenyl]-2-yl)cyclopropane-1-carbonyl]-4,4-difluoropiperidin-3-yl}carbamate

To a mixture of rac-(1R,2R)-2-(2′,6′-difluoro[1,1′-biphenyl]-2-yl)cyclopropane-1-carboxylic acid (80 mg), tert-butyl N-[(3R)-4,4-difluoro-3-piperidyl]carbamate (83 mg) and DMF (5 mL) were added HOBt (39 mg), EDCI (84 mg) and TEA (118 mg), and the mixture was stirred at room temperature for 13 hr. The mixture was diluted with water, and extracted with ethyl acetate. The organic layer was washed with brine, dried over sodium sulfate, and concentrated under reduced pressure. The residue was purified by silica gel column chromatography (ethyl acetate/petroleum ether) to give the title compound (200 mg).

MS, found: 437.1.

### B) A Mixture of N-{(3R)-1-[(1R,2R)-2-(2′,6′-difluoro[1,1′-biphenyl]-2-yl)cyclopropane-1-carbonyl]-4,4-difluoropiperidin-3-yl}methanesulfonamide and N-{(3R)-1-[(1S,2S)-2-(2′,6′-difluoro[1,1′-biphenyl]-2-yl)cyclopropane-1-carbonyl]-4,4-difluoropiperidin-3-yl}methanesulfonamide

A mixture of a mixture (200 mg) of tert-butyl {(3R)-1-[(1R,2R)-2-(2′,6′-difluoro[1,1′-biphenyl]-2-yl)cyclopropane-1-carbonyl]-4,4-difluoropiperidin-3-yl}carbamate and tert-butyl {(3R)-1-[(1S,2S)-2-(2′,6′-difluoro [1,1′-biphenyl]-2-yl)cyclopropane-1-carbonyl]-4,4-difluoropiperidin-3-yl}carbamate, trifluoroacetic acid (3 mL) and DCM (5 mL) was stirred at room temperature for 13 hr. The mixture was concentrated under reduced pressure to give a mixture (201 mg) of [(3R)-3-amino-4,4-difluoropiperidin-1-yl][(1R,2R)-2-(2′,6′-difluoro [1,1′-biphenyl]-2-yl) cyclopropyl]methanone trifluoroacetate and [(3R)-3-amino-4,4-difluoropiperidin-1-yl][(1S,2S)-2-(2′,6′-difluoro[1,1′-biphenyl]-2-yl)cyclopropyl]methanone trifluoroacetate.

To a mixture of a mixture (201 mg) of [(3R)-3-amino-4,4-difluoropiperidin-1-yl][(1R,2R)-2-(2′,6′-difluoro[1,1′-biphenyl]-2-yl)cyclopropyl]methanone trifluoroacetate and [(3R)-3-amino-4,4-difluoropiperidin-1-yl][(1S,2S)-2-(2′,6′-difluoro[1,1′-biphenyl]-2-yl)cyclopropyl]methanone trifluoroacetate, and DCM (5 mL) were added methanesulfonyl chloride (68 mg) and TEA (161 mg), and the mixture was stirred at room temperature for 13 hr. The mixture was diluted with water, and extracted with ethyl acetate. The organic layer was washed with brine, dried over sodium sulfate, and concentrated under reduced pressure. The residue was purified by preparative HPLC (C18, 0.05% aqueous ammonia/MeCN), and freeze-dried to give the title compound (38.9 mg).

MS: [M+H]⁺ 471.2.

### C) N-{(3R)-1-[(1R,2R)-2-(2′,6′-difluoro[1,1′-biphenyl]-2-yl)cyclopropane-1-carbonyl]-4,4-difluoropiperidin-3-yl}methanesulfonamide, or N-{(3R)-1-[(1S,2S)-2-(2′,6′-difluoro[1,1′-biphenyl]-2-yl)cyclopropane-1-carbonyl]-4,4-difluoropiperidin-3-yl}methanesulfonamide

A mixture (37 mg) of N-{(3R)-1-[(1R,2R)-2-(2′,6′-difluoro[1,1′-biphenyl]-2-yl)cyclopropane-1-carbonyl]-4,4-difluoropiperidin-3-yl}methanesulfonamide and N-{(3R)-1-[(1S,2S)-2-(2′,6′-difluoro[1,1′-biphenyl]-2-yl)cyclopropane-1-carbonyl]-4,4-difluoropiperidin-3-yl}methanesulfonamide was resolved by SFC (column: DAICEL CHIRALPAK IG, 250 mm×30 mm, 10 μm, mobile phase: CO₂/EtOH containing 0.1% aqueous ammonia=70/30 v/v), and the fraction having a shorter retention time was concentrated. The residue was dissolved in MeCN (1 mL) and water (30 mL), and the solution was freeze-dried to give the title compound (10.6 mg).

¹H NMR (400 MHz, DMSO-d₆) δ 1.15-1.24 (2H, m), 1.98-2.30 (4H, m), 2.65-3.26 (5H, m), 3.41-3.93 (2H, m), 4.02-4.25 (1H, m), 7.15-7.27 (4H, m), 7.29-7.55 (3H, m), 7.80 (1H, brs).

### Example 216

### N-{(3R,4S)-1-[(1R,2R)-2-(2′,6′-difluoro[1,1′-biphenyl]-2-yl)cyclopropane-1-carbonyl]-4-fluoropiperidin-3-yl}methanesulfonamide, or N-{(3R,4S)-1-[(1S,2S)-2-(2′,6′-difluoro[1,1′-biphenyl]-2-yl)cyclopropane-1-carbonyl]-4-fluoropiperidin-3-yl}methanesulfonamide

### A) A Mixture of tert-butyl {(3R,4S)-1-[(1R,2R)-2-(2′,6′-difluoro[1,1′-biphenyl]-2-yl)cyclopropane-1-carbonyl]-4-fluoropiperidin-3-yl}carbamate and tert-butyl {(3R,4S)-1-[(1S,2S)-2-(2′,6′-difluoro[1,1′-biphenyl]-2-yl)cyclopropane-1-carbonyl]-4-fluoropiperidin-3-yl}carbamate

To a mixture of rac-(1R,2R)-2-(2′,6′-difluoro[1,1′-biphenyl]-2-yl)cyclopropane-1-carboxylic acid (100 mg), tert-butyl N-(3R,4S)-4-fluoro-3-piperidyl]carbamate (96 mg) and DMF (3 mL) were added HOBt (49 mg), EDCI (105 mg) and TEA (148 mg), and the mixture was stirred at room temperature for 13 hr. The mixture was diluted with water, and extracted with ethyl acetate. The organic layer was washed with brine, dried over sodium sulfate, and concentrated under reduced pressure. The residue was purified by silica gel column chromatography (ethyl acetate/petroleum ether) to give the title compound (215 mg).

MS, found: 375.1.

### B) a mixture of N-{(3R,4S)-1-[(1R,2R)-2-(2′,6′-difluoro[1,1′-biphenyl]-2-yl)cyclopropane-1-carbonyl]-4-fluoropiperidin-3-yl}methanesulfonamide and N-{(3R,4S)-1-[(1S,2S)-2-(2′,6′-difluoro[1,1′-biphenyl]-2-yl)cyclopropane-1-carbonyl]-4-fluoropiperidin-3-yl}methanesulfonamide

A mixture of a mixture (215 mg) of tert-butyl {(3R,4S)-1-[(1R,2R)-2-(2′,6′-difluoro[1,1′-biphenyl]-2-yl)cyclopropane-1-carbonyl]-4-fluoropiperidin-3-yl}carbamate and tert-butyl {(3R,4S)-1-[(1S,2S)-2-(2′,6′-difluoro [1,1′-biphenyl]-2-yl)cyclopropane-1-carbonyl]-4-fluoropiperidin-3-yl}carbamate, trifluoroacetic acid (3 mL) and DCM (5 mL) was stirred at room temperature for 13 hr. The mixture was concentrated under reduced pressure to give a mixture (250 mg) of [(3R,4S)-3-amino-4-fluoropiperidin-1-yl][(1R,2R)-2-(2′,6′-difluoro[1,1′-biphenyl]-2-yl)cyclopropyl]methanone trifluoroacetate and [(3R,4S)-3-amino-4-fluoropiperidin-1-yl][(1S,2S)-2-(2′,6′-difluoro [1,1′-biphenyl]-2-yl)cyclopropyl]methanone trifluoroacetate.

To a mixture of a mixture (250 mg) of [(3R,4S)-3-amino-4-fluoropiperidin-1-yl][(1R,2R)-2-(2′,6′-difluoro[1,1′-biphenyl]-2-yl)cyclopropyl]methanone trifluoroacetate and [(3R,4S)-3-amino-4-fluoropiperidin-1-yl][(1S,2S)-2-(2′,6′-difluoro[1,1′-biphenyl]-2-yl)cyclopropyl]methanone trifluoroacetate, and DCM (5 mL) were added methanesulfonyl chloride (115 mg) and TEA (270 mg), and the mixture was stirred at room temperature for 13 hr. The mixture was diluted with water, and extracted with ethyl acetate. The organic layer was washed with brine, dried over sodium sulfate, and concentrated under reduced pressure. The residue was purified by preparative HPLC (C18, 0.05% aqueous ammonia/MeCN), and freeze-dried to give the title compound (47.2 mg).

MS: [M+H]⁺ 453.2.

### C) N-{(3R,4S)-1-[(1R,2R)-2-(2′,6′-difluoro[1,1′-biphenyl]-2-yl)cyclopropane-1-carbonyl]-4-fluoropiperidin-3-yl}methanesulfonamide, or N-{(3R,4S)-1-[(1S,2S)-2-(2′,6′-difluoro[1,1′-biphenyl]-2-yl)cyclopropane-1-carbonyl]-4-fluoropiperidin-3-yl}methanesulfonamide

A mixture (44 mg) of N-{(3R,4S)-1-[(1R,2R)-2-(2′,6′-difluoro[1,1′-biphenyl]-2-yl)cyclopropane-1-carbonyl]-4-fluoropiperidin-3-yl}methanesulfonamide and N-{(3R,4S)-1-[(1S,2S)-2-(2′,6′-difluoro[1,1′-biphenyl]-2-yl)cyclopropane-1-carbonyl]-4-fluoropiperidin-3-yl}methanesulfonamide was resolved by SFC (column: Phenomenex-Cellulose-2, 250 mm×30 mm, 10 μm, mobile phase: CO₂/EtOH containing 0.1% aqueous ammonia=65/35 v/v), and the fraction having a shorter retention time was concentrated. The residue was dissolved in MeCN (1 mL) and water (30 mL), and the solution was freeze-dried to give the title compound (14.6 mg).

¹H NMR (400 MHz, DMSO-d₆) δ 1.11-1.23 (2H, m), 1.58-2.05 (3H, m), 2.12-2.26 (1H, m), 2.63-3.24 (5H, m), 3.39-3.81 (2H, m), 3.85-4.15 (1H, m), 4.79-5.01 (1H, m), 7.15-7.27 (4H, m), 7.29-7.55 (4H, m).

### Example 226

### N-{4,4-difluoro-1-[2-(2′,3,6′-trifluoro[1,1′-biphenyl]-2-yl)cyclopropane-1-carbonyl]pyrrolidin-3-yl}methanesulfonamide optical isomer

### A) 1-benzyl-4,4-difluoropyrrolidin-3-yl trifluoromethanesulfonate

To a mixture of 1-benzyl-4,4-difluoropyrrolidin-3-ol (1.50 g) and DCM (50 mL) was added dropwise a mixture of pyridine (2.78 g), trifluoromethanesulfonic anhydride (6.95 g) and DCM (5.0 mL) at −10° C., and the mixture was stirred at the same temperature for 2 hr. The mixture was poured into water, and extracted with DCM. The organic layer was washed with 5% aqueous citric acid solution and brine, dried over sodium sulfate, and concentrated under reduced pressure to give the title compound (2.00 g).

¹H NMR (400 MHz, CDCl₃) δ 2.83-2.88 (1H, m), 2.90-3.01 (1H, m), 3.07-3.16 (1H, m), 3.27-3.31 (1H, m), 3.69 (2H, s), 5.05-5.18 (1H, m), 7.27-7.39 (5H, m).

### B) 4-azido-1-benzyl-3,3-difluoropyrrolidine

To a mixture of 1-benzyl-4,4-difluoropyrrolidin-3-yl trifluoromethanesulfonate (1.8 g) and MeCN (30 mL) was added tetrabutylammonium azide (2.97 g), and the mixture was stirred at 80° C. for 2 hr. The mixture was diluted with water, and extracted with ethyl acetate. The organic layer was washed with brine, dried over sodium sulfate, and concentrated under reduced pressure. The residue was purified by silica gel column chromatography (ethyl acetate/petroleum ether) to give the title compound (1.10 g).

¹H NMR (400 MHz, CDCl₃) δ 2.59-2.64 (1H, m), 2.83-3.16 (3H, m), 3.55-3.72 (2H, m), 3.82-3.98 (1H, m), 7.27-7.37 (5H, m).

### C) rac-N-[(3S)-1-benzyl-4,4-difluoropyrrolidin-3-yl]methanesulfonamide

To a mixture of 4-azido-1-benzyl-3,3-difluoropyrrolidine (500 mg), THF (15 mL) and water (2.0 mL) was added triphenylphosphine (2.19 g, 20% purity in resin), and the mixture was stirred at 70° C. for 12 hr. The mixture was filtered, and the filtrate was concentrated under reduced pressure to give 1-benzyl-4,4-difluoropyrrolidin-3-amine (460 mg).

To a mixture of 1-benzyl-4,4-difluoropyrrolidin-3-amine (860 mg), TEA (820 mg) and DCM (10 mL) was added methanesulfonyl chloride (650 mg) at 0° C., and the mixture was stirred at room temperature for 1 hr. The mixture was diluted with water, and extracted with DCM. The organic layer was washed with brine, dried over sodium sulfate, and concentrated under reduced pressure. The residue was purified by silica gel column chromatography (ethyl acetate/petroleum ether) to give the title compound (1.01 g).

¹H NMR (400 MHz, DMSO-d₆) δ 2.32 (1H, t, J=9.0 Hz), 2.58-2.70 (1H, m), 2.93 (3H, s), 3.10-3.26 (2H, m), 3.49-3.71 (2H, m), 3.90-4.12 (1H, m), 7.23-7.40 (5H, m), 7.80 (1H, d, J=9.2 Hz).

### D) rel-N-[(3R)-1-benzyl-4,4-difluoropyrrolidin-3-yl]methanesulfonamide

rac-N-[(3S)-1-Benzyl-4,4-difluoropyrrolidin-3-yl]methanesulfonamide (800 mg) was resolved by SFC (column: DAICEL CHIRALPAK AD, 250 mm×30 mm, 10 μm, mobile phase: CO₂/EtOH containing 0.1% aqueous ammonia=85/15 v/v), and the fraction having a longer retention time was concentrated. The residue was dissolved in MeCN (3.0 mL) and water (3.0 mL), and the solution was freeze-dried to give the title compound (368 mg).

MS: [M+H]⁺ 291.3.

### E) N-{4,4-difluoro-1-[2-(2′,3,6′-trifluoro[1,1′-biphenyl]-2-yl)cyclopropane-1-carbonyl]pyrrolidin-3-yl}methanesulfonamide Optical Isomer

To a mixture of rel-N-[(3R)-1-benzyl-4,4-difluoropyrrolidin-3-yl]methanesulfonamide (368 mg) synthesized in Step D) and 12 M hydrogen chloride EtOH solution (5.0 mL) was added 10% Pd/C (50 mg), and the mixture was stirred under hydrogen atmosphere (15 psi) at room temperature for 3 hr. The mixture was filtered, and the filtrate was concentrated under reduced pressure to give rel-N-[(3R)-4,4-difluoropyrrolidin-3-yl]methanesulfonamide hydrochloride (305 mg).

To a mixture of rel-(1R,2R)-2-(2′,3,6′-trifluoro[1,1′-biphenyl]-2-yl)cyclopropane-1-carboxylic acid (30.0 mg) synthesized in Step E) of Example 195 and DMF (2 mL) were added HATU (59 mg), DIPEA (53 mg) and the above rel-N-[(3R)-4,4-difluoropyrrolidin-3-yl]methanesulfonamide hydrochloride (38 mg), and the mixture was stirred at room temperature for 14 hr. The mixture was diluted with water, and extracted with ethyl acetate. The organic layer was washed with brine, dried over sodium sulfate, and concentrated under reduced pressure. The residue was purified by preparative HPLC (C18, 0.05% aqueous ammonia/MeCN), and freeze-dried to give the title compound (23.9 mg).

¹H NMR (400 MHz, CDCl₃) δ 0.09-1.10 (1H, m), 1.32-1.41 (1H, m), 1.62-1.76 (1H, m), 2.30-2.44 (1H, m), 3.06-3.15 (3H, m), 3.20-3.40 (1H, m), 3.63-4.20 (3H, m), 4.20-4.38 (1H, m), 4.85-5.00 (1H, m), 6.95-7.16 (4H, m), 7.27-7.45 (2H, m).

### Example 232

### N-{(3S,5R)-1-[(1R,2R)-2-(2′,6′-difluoro[1,1′-biphenyl]-2-yl)cyclopropane-1-carbonyl]-5-fluoropiperidin-3-yl}methanesulfonamide

### A) A Mixture of ethyl (1R,2R)-2-(2-bromophenyl)cyclopropane-1-carboxylate and ethyl (1S,2S)-2-(2-bromophenyl)cyclopropane-1-carboxylate

To a mixture of dichloro(p-cymene)ruthenium(II) (dimer) (1.74 g), (S,S)-2,6-bis(4-isopropyl-2-oxazolin-2-yl)pyridine (1.71 g) and THF (200 mL) was added dropwise a mixture of 2-bromostyrene (20.0 g) and THF (100 mL) under nitrogen atmosphere at 20° C., and the mixture was warmed to 55° C. A mixture of ethyl diazoacetate (31.2 g) and toluene (200 mL) was added dropwise thereto over 3 hr under nitrogen atmosphere, and the mixture was stirred at 55° C. for 14 hr. The mixture was cooled, and the reaction was quenched with 10% acetic acid aqueous solution (50 mL). The organic layer was washed with water and brine, dried over sodium sulfate, and concentrated under reduced pressure. The residue was purified by silica gel column chromatography (ethyl acetate/petroleum ether) to the title mixture (18.6 g).

MS: [M+H]⁺ 268.9.

### B) a mixture of ethyl (1R,2R)-2-(2′,6′-difluoro[1,1′-biphenyl]-2-yl)cyclopropane-1-carboxylate and ethyl (1S,2S)-2-(2′,6′-difluoro[1,1′-biphenyl]-2-yl)cyclopropane-1-carboxylate

To a mixture of a mixture (10.0 g) of ethyl (1R,2R)-2-(2-bromophenyl)cyclopropane-1-carboxylate and ethyl (1S,2S)-2-(2-bromophenyl)cyclopropane-1-carboxylate, and DME (150 mL) were added 2,6-difluorophenylboronic acid (17.6 g), Xphos Pd G3 (3.15 g) and 1 M aqueous tripotassium phosphate solution (112 mL), and the mixture was stirred under nitrogen atmosphere at 100° C. for 14 hr. The mixture was cooled to room temperature, 2,6-difluorophenylboronic acid (17.6 g), Xphos Pd G3 (3.15 g), and 1 M aqueous tripotassium phosphate solution (112 mL) were added thereto, and the mixture was stirred under nitrogen atmosphere at 100° C. for 14 hr. The mixture was diluted with water, and extracted with ethyl acetate. The organic layer was dried over sodium sulfate, and concentrated under reduced pressure. The residue was purified by silica gel column chromatography (ethyl acetate/petroleum ether), followed by reverse-phase silica gel column chromatography (C18, methanol/water) to give the title compound (7.52 g).

MS: [M+H]⁺ 303.0.

### C) ethyl (1R,2R)-2-(2′,6′-difluoro[1,1′-biphenyl]-2-yl)cyclopropane-1-carboxylate

A mixture (7.52 g) of ethyl (1R,2R)-2-(2′,6′-difluoro[1,1′-biphenyl]-2-yl)cyclopropane-1-carboxylate and ethyl (1S,2S)-2-(2′,6′-difluoro[1,1′-biphenyl]-2-yl)cyclopropane-1-carboxylate was resolved by SFC (column: DAICEL CHIRALPAK IC, 250 mm×50 mm, 10 μm, mobile phase: CO₂/IPA containing 0.1% aqueous ammonia=85/15 v/v) to give the title compound (6.51 g) having a longer retention time.

MS: [M+H]⁺ 302.9.

### D) (1R,2R)-2-(2′,6′-difluoro[1,1′-biphenyl]-2-yl)cyclopropane-1-carboxylic Acid

To a mixture of ethyl (1R,2R)-2-(2′,6′-difluoro[1,1′-biphenyl]-2-yl)cyclopropane-1-carboxylate (6.51 g) and EtOH (100 mL) was added aqueous sodium hydroxide solution (8.61 g/50 mL), and the mixture was stirred at room temperature for 3 hr. The mixture was concentrated under reduced pressure, diluted with water, adjusted to pH 5-6 with 4 M hydrochloric acid, and extracted with ethyl acetate. The organic layer was dried over sodium sulfate, and concentrated under reduced pressure to give the title compound (5.90 g).

MS: [M−H]⁻ 272.9.

### E) tert-butyl {(3S,5R)-1-[(1R,2R)-2-(2′,6′-difluoro[1,1′-biphenyl]-2-yl)cyclopropane-1-carbonyl]-5-fluoropiperidin-3-yl}carbamate

To a mixture of (1R,2R)-2-(2′,6′-difluoro[1,1′-biphenyl]-2-yl)cyclopropane-1-carboxylic acid (40 mg), tert-butyl N-[(3S,5R)-5-fluoro-3-piperidyl]carbamate (35 mg) and DMF (2 mL) were added EDCI (42 mg), HOBt (20 mg) and TEA (59 mg), and the mixture was stirred at room temperature for 14 hr. The mixture was diluted with water, and extracted with ethyl acetate. The organic layer was washed with brine, dried over sodium sulfate, and concentrated under reduced pressure. The residue was purified by preparative thin layer chromatography (ethyl acetate/petroleum ether) to give the title compound (48 mg).

MS, found: 375.0.

### F) N-{(3S,5R)-1-[(1R,2R)-2-(2′,6′-difluoro[1,1′-biphenyl]-2-yl)cyclopropane-1-carbonyl]-5-fluoropiperidin-3-yl}methanesulfonamide

To a mixture of tert-butyl {(3S,5R)-1-[(1R,2R)-2-(2′,6′-difluoro[1,1′-biphenyl]-2-yl)cyclopropane-1-carbonyl]-5-fluoropiperidin-3-yl}carbamate (48 mg) and DCM (1 mL) was added trifluoroacetic acid (1 mL), and the mixture was stirred at room temperature for 2 hr. The mixture was concentrated under reduced pressure to give [(3S,5R)-3-amino-5-fluoropiperidin-1-yl][(1R,2R)-2-(2′,6′-difluoro[1,1′-biphenyl]-2-yl)cyclopropyl]methanone trifluoroacetate (58 mg).

To a mixture of [(3S,5R)-3-amino-5-fluoropiperidin-1-yl][(1R,2R)-2-(2′,6′-difluoro[1,1′-biphenyl]-2-yl)cyclopropyl]methanone trifluoroacetate (58 mg) and DCM (5 mL) were added TEA (48 mg) and methanesulfonyl chloride (20 mg) at 0° C., and the mixture was stirred at room temperature for 3 hr. The mixture was diluted with water, and extracted with DCM. The organic layer was washed with brine, dried over sodium sulfate, and concentrated under reduced pressure. The residue was purified by preparative HPLC (C18, 0.05% aqueous ammonia/MeCN), and freeze-dried to give the title compound (33.0 mg).

¹H NMR (400 MHz, CDCl₃) δ 1.23-1.42 (2H, m), 1.87-2.37 (3H, m), 2.40-3.04 (5H, m), 3.25-5.15 (6H, m), 6.87-7.26 (4H, m), 7.28-7.43 (3H, m).

### Example 238

### N-2-[(2-(2′,6′-difluoro[1,1′-biphenyl]-2-yl)cyclopropane-1-carbonyl]octahydrocyclopenta[c]pyrrol-4-yl}methanesulfonamide Optical Isomer

### A) rac-tert-butyl (3aR,4S,6aS)-4-[(methanesulfonyl)amino]hexahydrocyclopenta[c]pyrrole-2(1H)-carboxylate

To a mixture of rac-tert-butyl (3aR,4S,6aS)-4-aminohexahydrocyclopenta[c]pyrrole-2 (1H)-carboxylate (500 mg) and THF (8 mL) were added methanesulfonyl chloride (301 mg) and TEA (447 mg), and the mixture was stirred at room temperature for 2 hr. The mixture was poured into water, and extracted with ethyl acetate. The organic layer was washed with brine, dried over sodium sulfate, and concentrated under reduced pressure. The residue was purified by silica gel column chromatography (ethyl acetate/petroleum ether) to give the title compound (664 mg).

MS, found: 249.0.

### B) rac-N-[(3aR,4S,6aS)-octahydrocyclopenta[c]pyrrol-4-yl]methanesulfonamide Trifluoroacetate

To a mixture of rac-tert-butyl (3aR,4S,6aS)-4-[(methanesulfonyl)amino]hexahydrocyclopenta[c]pyrrole-2(1H)-carboxylate (664 mg) and DCM (12 mL) was added trifluoroacetic acid (6 mL), and the mixture was stirred at room temperature for 12 hr. The mixture was concentrated under reduced pressure to give the title compound (868 mg).

¹H NMR (400 MHz, DMSO-d₆) δ 1.28-1.43 (1H, m), 1.45-1.61 (1H, m), 1.85-2.04 (2H, m), 2.54-2.63 (1H, m), 2.73-2.86 (1H, m), 2.85-2.98 (4H, m), 3.04-3.14 (1H, m), 3.15-3.34 (2H, m), 3.39-3.58 (1H, m), 7.29 (1H, d, J=6.8 Hz), 8.69-9.05 (2H, m). C) a mixture of rac-N-{(3aR,4S,6aS)-2-[(1R,2R)-2-(2′,6′-difluoro[1,1′-biphenyl]-2-yl)cyclopropane-1-carbonyl]octahydrocyclopenta[c]pyrrol-4-yl}methanesulfonamide and rac-N-{(3aS,4R,6aR)-2-[(1R,2R)-2-(2′,6′-difluoro[1,1′-biphenyl]-2-yl)cyclopropane-1-carbonyl]octahydrocyclopenta[c]pyrrol-4-yl}methanesulfonamide

To a mixture of rac-(1R,2R)-2-(2′,6′-difluoro[1,1′-biphenyl]-2-yl)cyclopropane-1-carboxylic acid (43 mg), rac-N-[(3aR,4S,6aS)-octahydrocyclopenta[c]pyrrol-4-yl]methanesulfonamide trifluoroacetate (50 mg), HATU (63 mg) and DMF (3 mL) was added DIPEA (81 mg), and the mixture was stirred at room temperature for 12 hr. The mixture was poured into water, and extracted with ethyl acetate. The organic layer was washed with brine, dried over sodium sulfate, and concentrated under reduced pressure. The residue was purified by preparative HPLC (C18, 0.05% aqueous ammonia/MeCN), and freeze-dried to give the title compound (4.3 mg).

MS: [M+H]⁺ 461.2.

### D) N-2-[(2-(2′,6′-difluoro[1,1′-biphenyl]-2-yl)cyclopropane-1-carbonyl]octahydrocyclopenta[c]pyrrol-4-yl}methanesulfonamide Optical Isomer

A mixture (250 mg) of rac-N-{(3aR,4S,6aS)-2-[(1R,2R)-2-(2′,6′-difluoro[1,1′-biphenyl]-2-yl)cyclopropane-1-carbonyl]octahydrocyclopenta[c]pyrrol-4-yl}methanesulfonamide and rac-N-{(3aS,4R,6aR)-2-[(1R,2R)-2-(2′,6′-difluoro[1,1′-biphenyl]-2-yl)cyclopropane-1-carbonyl]octahydrocyclopenta[c]pyrrol-4-yl}methanesulfonamide was resolved by SFC (column: Phenomenex-Cellulose-2, 250 mm×30 mm, 10 μm, mobile phase: CO₂/EtOH containing 0.1% aqueous ammonia=55/45 v/v), and the mixture of the fraction having the first retention time and the fraction having the second retention time was concentrated. This was resolved by SFC (column: DAICEL CHIRALPAK IC, 250 mm×30 mm, 10 μm, mobile phase: CO₂/EtOH containing 0.1% aqueous ammonia=60/40 v/v), and the fraction having a longer retention time was concentrated. The residue was dissolved in MeCN (10 mL) and water (50 mL), and the solution was freeze-dried to give the title compound (36.7 mg).

¹H NMR (400 MHz, CDCl₃) δ 1.39-1.56 (3H, m), 1.60-1.86 (2H, m), 1.99-2.13 (1H, m), 2.18-2.39 (2H, m), 2.45-2.67 (1H, m), 2.68-2.86 (1H, m), 2.87-2.98 (3H, m), 3.32 (1H, dd, J=11.6, 4.4 Hz), 3.45-3.80 (4H, m), 4.24-4.34 (1H, m), 6.88-7.03 (2H, m), 7.12 (1H, d, J=7.6 Hz), 7.21-7.41 (4H, m).

### Example 259

### N′-({(2S)-1-[(1R,2R)-2-(2′,6′-difluoro[1,1′-biphenyl]-2-yl)cyclopropane-1-carbonyl]azetidin-2-yl}methyl)-N,N-dimethylsulfuric Diamide

### A) tert-butyl (S)-2-(((N,N-dimethylsulfamoyl)amino)methyl)azetidine-1-carboxylate

To a mixture of (S)-2-aminomethyl-1-Boc-azetidine (509 mg), DMAP (66.8 mg), TEA (1.14 mL) and THF (12 mL) was added N,N-dimethylsulfamoyl chloride (0.440 mL) at 0° C., and the mixture was stirred overnight at room temperature. The reaction was quenched with saturated aqueous sodium hydrogen carbonate solution at 0° C., and extracted with ethyl acetate. The organic layer was washed with water and brine, dried over magnesium sulfate, and concentrated under reduced pressure. The residue was purified by silica gel column chromatography (ethyl acetate/hexane) to give the title compound (498 mg).

¹H NMR (300 MHz, DMSO-d₆) δ 1.37 (9H, s), 1.94-2.25 (2H, m), 2.60-2.74 (6H, m), 3.08 (1 H, dt, J=13.3, 6.7 Hz), 3.24 (1H, ddd, J=13.3, 6.3, 4.1 Hz), 3.58-3.80 (2H, m), 4.09-4.23 (1H, m), 7.19-7.34 (1H, m).

### B) N′-({(2S)-1-[(1R,2R)-2-(2′,6′-difluoro[1,1′-biphenyl]-2-yl)cyclopropane-1-carbonyl]azetidin-2-yl}methyl)-N,N-dimethylsulfuric Diamide

A mixture of tert-butyl (S)-2-(((N,N-dimethylsulfamoyl)amino)methyl)azetidine-1-carboxylate (104 mg) and trifluoroacetic acid (0.500 mL) was stirred at room temperature for 1 hr. The mixture was concentrated under reduced pressure, and azeotroped with toluene. A mixture of the obtained residue (67.6 mg), (1R,2R)-2-(2′,6′-difluoro[1,1′-biphenyl]-2-yl)cyclopropane-1-carboxylic acid (106 mg), HATU (160 mg), TEA (0.146 mL) and DMF (2 mL) was stirred overnight at room temperature. The reaction was quenched with water, and extracted with ethyl acetate. The organic layer was washed with water and brine, dried over sodium sulfate, and concentrated under reduced pressure. The residue was purified by silica gel column chromatography (ethyl acetate/hexane), followed by silica gel column chromatography (NH, ethyl acetate/hexane) to give the title compound (140 mg).

¹H NMR (300 MHz, DMSO-d₆) δ 1.07-1.17 (1H, m), 1.21-1.31 (1H, m), 1.47-1.60 (1H, m), 1.72-1.80 (1H, m), 2.01-2.33 (2H, m), 2.61-2.66 (6H, m), 3.01-3.28 (2H, m), 3.56-3.74 (1H, m), 3.79-3.91 (1H, m), 4.04-4.18 (1H, m), 7.12-7.61 (8H, m).

### Example 262

### N-{(3S,5S)-1-[(1R,2R)-2-(2′,6′-difluoro[1,1′-biphenyl]-2-yl)cyclopropane-1-carbonyl]-5-methylpyrrolidin-3-yl}methanesulfonamide

### A) N-[(3S,5S)-5-methylpyrrolidin-3-yl]methanesulfonamide Trifluoroacetate

To a mixture of tert-butyl (2S,4S)-4-amino-2-methylpyrrolidine-1-carboxylate (50 mg), TEA (51 mg) and DCM (5 mL) was added methanesulfonyl chloride (43 mg) at 0° C., and the mixture was stirred at room temperature for 2 hr. The mixture was poured into ice-water, and extracted with DCM. The organic layer was washed with brine, dried over sodium sulfate, and concentrated under reduced pressure to give tert-butyl (2S,4S)-4-[(methanesulfonyl)amino]-2-methylpyrrolidine-1-carboxylate (83 mg).

To a mixture of tert-butyl (2S,4S)-4-[(methanesulfonyl)amino]-2-methylpyrrolidine-1-carboxylate (83 mg) and DCM (3 mL) was added trifluoroacetic acid (1 mL), and the mixture was stirred at room temperature for 2 hr. The mixture was concentrated under reduced pressure to give the title compound (93 mg).

MS: [M+H]⁺ 179.1.

### B) N-{(3S,5S)-1-[(1R,2R)-2-(2′,6′-difluoro[1,1′-biphenyl]-2-yl)cyclopropane-1-carbonyl]-5-methylpyrrolidin-3-yl}methanesulfonamide

To a mixture of N-[(3S,5S)-5-methylpyrrolidin-3-yl]methanesulfonamide trifluoroacetate (93 mg), (1R,2R)-2-(2′,6′-difluoro[1,1′-biphenyl]-2-yl)cyclopropane-1-carboxylic acid (87 mg) and DMF (5 mL) were added EDCI (73 mg), HOBt (52 mg) and TEA (129 mg), and the mixture was stirred at room temperature for 16 hr. The mixture was poured into water, and extracted with ethyl acetate. The organic layer was washed with brine, dried over sodium sulfate, and concentrated under reduced pressure. The residue was purified by preparative HPLC (C18, 0.05% aqueous ammonia/MeCN), and freeze-dried to give the title compound (34.2 mg).

¹H NMR (400 MHz, CDCl₃) δ 1.10-1.27 (3H, m), 1.29-1.56 (3H, m), 1.88-1.95 (1H, m), 2.00-2.17 (1H, m), 2.32-2.48 (1H, m), 2.99 (3H, s), 3.33-3.45 (1H, m), 3.61-3.82 (1H, m), 3.95-4.24 (2H, m), 4.42-4.60 (1H, m), 6.92-7.26 (4H, m), 7.27-7.42 (3H, m).

### Example 264

### N-{(3S,4R)-1-[(1R,2R)-2-(2′,6′-difluoro[1,1′-biphenyl]-2-yl)cyclopropane-1-carbonyl]-4-methylpyrrolidin-3-yl}methanesulfonamide

### A) N-[(3S,4R)-4-methylpyrrolidin-3-yl]methanesulfonamide Trifluoroacetate

To a mixture of tert-butyl (3S,4R)-3-amino-4-methylpyrrolidine-1-carboxylate (60 mg), TEA (61 mg) and DCM (5 mL) was added methanesulfonyl chloride (51 mg) at 0° C., and the mixture was stirred at room temperature for 2 hr. The mixture was poured into water, and extracted with ethyl acetate. The organic layer was washed with brine, dried over sodium sulfate, and concentrated under reduced pressure to give tert-butyl (3S,4R)-3-[(methanesulfonyl)amino]-4-methylpyrrolidine-1-carboxylate (67 mg).

To a mixture of tert-butyl (3S,4R)-3-[(methanesulfonyl)amino]-4-methylpyrrolidine-1-carboxylate (67 mg) and DCM (3 mL) was added trifluoroacetic acid (1 mL), and the mixture was stirred at room temperature for 2 hr. The mixture was concentrated under reduced pressure to give the title compound (97 mg).

MS: [M+H]⁺ 179.1.

### B) N-{(3S,4R)-1-[(1R,2R)-2-(2′,6′-difluoro[1,1′-biphenyl]-2-yl)cyclopropane-1-carbonyl]-4-methylpyrrolidin-3-yl}methanesulfonamide

To a mixture of N-[(3S,4R)-4-methylpyrrolidin-3-yl]methanesulfonamide trifluoroacetate (97 mg), (1R,2R)-2-(2′,6′-difluoro[1,1′-biphenyl]-2-yl)cyclopropane-1-carboxylic acid (91 mg) and DMF (5 mL) were added EDCI (76 mg), HOBt (54 mg) and TEA (134 mg), and the mixture was stirred at room temperature for 16 hr. The mixture was poured into water, and extracted with ethyl acetate. The organic layer was washed with brine, dried over sodium sulfate, and concentrated under reduced pressure. The residue was purified by preparative HPLC (C18, 0.05% aqueous ammonia/MeCN), and freeze-dried to give the title compound (20.2 mg).

¹H NMR (400 MHz, CDCl₃) δ 1.10-1.16 (3H, m), 1.24-1.30 (1H, m), 1.43-1.52 (1H, m), 1.61-1.71 (1H, m), 1.93-2.43 (2H, m), 2.84-3.33 (5H, m), 3.41-3.61 (1H, m), 3.66-3.79 (1H, m), 3.83-3.92 (1H, m), 4.45-4.63 (1H, m), 6.92-7.26 (4H, m), 7.29-7.41 (3H, m).

### Example 294

### N-{(6S)-4-[(1R,2R)-2-(2′,6′-difluoro [1,1′-biphenyl]-2-yl)cyclopropane-1-carbonyl]-1,4-oxazepan-6-yl}ethanesulfonamide, or N-{(6R)-4-[(1R,2R)-2-(2′,6′-difluoro[1,1′-biphenyl]-2-yl)cyclopropane-1-carbonyl]-1,4-oxazepan-6-yl}ethanesulfonamide

### A) tert-butyl 6-(ethylsulfonamido)-1,4-oxazepane-4-carboxylate

To a mixture of 4-Boc-6-amino-1,4-oxazepane (454 mg), DMAP (51.3 mg), TEA (0.878 mL) and THF (7 mL) was added ethanesulfonyl chloride (0.298 mL) at 0° C., and the mixture was stirred at room temperature for 2 hr. The reaction was quenched with saturated aqueous sodium hydrogen carbonate solution at 0° C., and extracted with ethyl acetate. The organic layer was washed with water and brine, dried over magnesium sulfate, and concentrated under reduced pressure. The residue was purified by silica gel column chromatography (ethyl acetate/hexane) to give the title compound (501 mg).

¹H NMR (300 MHz, DMSO-d₆) δ 1.13-1.25 (3H, m), 1.41 (9H, s), 2.91-3.26 (4H, m), 3.48-3.84 (7H, m), 7.22 (1H, br s).

### B) N-(1,4-oxazepan-6-yl)ethanesulfonamide Hydrochloride

To a mixture of tert-butyl 6-(ethylsulfonamido)-1,4-oxazepane-4-carboxylate (267 mg) and ethyl acetate (2 mL) was added 2 M hydrogen chloride CPME solution (4 mL), and the mixture was stirred overnight at room temperature. The resulting solid was collected by filtration to give the title compound (186 mg).

¹H NMR (300 MHz, DMSO-d₆) δ 1.20 (3H, t, J=7.3 Hz), 3.07-3.30 (5H, m), 3.35 (1H, d, J=3.8 Hz), 3.50-3.64 (1H, m), 3.79-3.95 (4H, m), 7.47 (1H, d, J=7.9 Hz), 9.46 (2H, br s).

### C) N-{(6S)-4-[(1R,2R)-2-(2′,6′-difluoro[1,1′-biphenyl]-2-yl)cyclopropane-1-carbonyl]-1,4-oxazepan-6-yl}ethanesulfonamide, or N-{(6R)-4-[(1R,2R)-2-(2′,6′-difluoro[1,1′-biphenyl]-2-yl)cyclopropane-1-carbonyl]-1,4-oxazepan-6-yl}ethanesulfonamide

To a mixture of (1R,2R)-2-(2′,6′-difluoro[1,1′-biphenyl]-2-yl)cyclopropane-1-carboxylic acid (166 mg), N-(1,4-oxazepan-6-yl)ethanesulfonamide hydrochloride (140 mg), TEA (0.319 mL) and DMF (2 mL) was added HATU (261 mg) at 0° C., and the mixture was stirred at the same temperature for 3 hr. The reaction was quenched with water, and extracted with ethyl acetate. The organic layer was washed with water and brine, dried over sodium sulfate, and concentrated under reduced pressure. The residue was purified by silica gel column chromatography (NH, ethyl acetate/hexane), followed by preparative HPLC (column: DAICEL CHIRALPAK IC, 250 mm×20 mm, 5 μm, mobile phase: hexane/ethanol/diethylamine=700/300/1 v/v/v) to give the title compound (94.1 mg) having a shorter retention time.

¹H NMR (400 MHz, CDCl₃) δ 0.93-1.42 (5H, m), 1.89-2.40 (2H, m), 2.73-3.09 (2H, m), 3.40-3.74 (8H, m), 3.77-4.23 (2H, m), 6.84-7.70 (7H, m).

### Example 304

### N-{(3S)-1-[(1S,2S)-2-(2′,6′-difluoro [1,1′-biphenyl]-2-yl)-2-fluorocyclopropane-1-carbonyl]pyrrolidin-3-yl}methanesulfonamide, or N-{(3S)-1-[(1R,2R)-2-(2′,6′-difluoro[1,1′-biphenyl]-2-yl)-2-fluorocyclopropane-1-carbonyl]pyrrolidin-3-yl}methanesulfonamide

### A) 2,6-difluoro-2′-(1-fluoroethenyl)-1,1′-biphenyl

To a mixture of 1-bromo-2-(1-fluoroethenyl)benzene (16.2 g), 2,6-difluorophenylboronic acid (63.6 g), DME (150 mL) and water (50 mL) were added Xphos Pd G3 (6.82 g) and tripotassium phosphate (51.3 g), and the mixture was stirred at 100° C. for 16 hr. The mixture was diluted with water, and extracted with ethyl acetate. The organic layer was washed with brine, dried over sodium sulfate, and concentrated under reduced pressure. The residue was purified by silica gel column chromatography (petroleum ether) to give the title compound (4.60 g).

¹H NMR (400 MHz, CDCl₃) δ 4.37-4.81 (2H, m), 6.94-7.01 (2H, m), 7.30-7.36 (2H, m), 7.45-7.51 (2H, m), 7.58-7.70 (1H, m).

### B) rac-ethyl (1S,2S)-2-(2′,6′-difluoro[1,1′-biphenyl]-2-yl)-2-fluorocyclopropane-1-carboxylate

To a mixture of 2,6-difluoro-2′-(1-fluoroethenyl)-1,1′-biphenyl (7.50 g) and DCE (100 mL) was added rhodium(II) acetate (dimer) (353 mg), and a mixture of ethyl diazoacetate (10.9 g) and DCE (100 mL) was added thereto at 70° C. over 6 hr. The mixture was stirred at the same temperature for 6 hr. The mixture was concentrated under reduced pressure. The residue was purified by silica gel column chromatography (ethyl acetate/petroleum ether) to give a crude product. This was purified by preparative HPLC (C18, 0.05% aqueous ammonia/MeCN), and the fraction having a shorter retention time was concentrated, and freeze-dried to give the title compound (1.60 g).

¹H NMR (400 MHz, CDCl₃) δ 1.22 (3H, t, J=7.2 Hz), 1.38-1.50 (1H, m), 1.87-1.99 (1H, m), 2.01-2.13 (1H, m), 3.93-4.21 (2H, m), 6.92-7.04 (2H, m), 7.29-7.64 (5H, m).

### C) rel-(1S,2S)-2-(2′,6′-difluoro[1,1′-biphenyl]-2-yl)-2-fluorocyclopropane-1-carboxylic Acid

rac-Ethyl (1S,2S)-2-(2′,6′-difluoro[1,1′-biphenyl]-2-yl)-2-fluorocyclopropane-1-carboxylate (800 mg) was resolved by SFC (column: DAICEL CHIRALPAK IC, 250 mm×30 mm, 10 μm, mobile phase: CO₂/EtOH containing 0.1% aqueous ammonia=80/20 v/v) to give rel-ethyl (1S,2S)-2-(2′,6′-difluoro[1,1′-biphenyl]-2-yl)-2-fluorocyclopropane-1-carboxylate (370 mg) having a longer retention time.

To a mixture of the rel-ethyl (1S,2S)-2-(2′,6′-difluoro[1,1′-biphenyl]-2-yl)-2-fluorocyclopropane-1-carboxylate (370 mg), MeOH (2 mL), THF (2 mL) and water (2 mL) was added sodium hydroxide (462 mg), and the mixture was stirred at room temperature for 12 hr. The mixture was diluted with water, adjusted to pH=5-6 with 2 M hydrochloric acid, and extracted with ethyl acetate. The organic layer was dried over sodium sulfate, and concentrated under reduced pressure to give the title mixture (330 mg).

¹H NMR (400 MHz, CDCl₃) δ 1.44-1.56 (1H, m), 1.58-1.72 (1H, m), 2.00-2.11 (1H, m), 7.17 (2H, t, J=8.4 Hz), 7.35-7.42 (1H, m), 7.46-7.69 (4H, m), 12.2 (1H, brs).

### D) N-{(3S)-1-[(1S,2S)-2-(2′,6′-difluoro[1,1′-biphenyl]-2-yl)-2-fluorocyclopropane-1-carbonyl]pyrrolidin-3-yl}methanesulfonamide, or N-{(3S)-1-[(1R,2R)-2-(2′,6′-difluoro[1,1′-biphenyl]-2-yl)-2-fluorocyclopropane-1-carbonyl]pyrrolidin-3-yl}methanesulfonamide

To a mixture of rel-(1S,2S)-2-(2′,6′-difluoro[1,1′-biphenyl]-2-yl)-2-fluorocyclopropane-1-carboxylic acid (50 mg) synthesized in Step C), (S)—N-(pyrrolidin-3-yl)methanesulfonamide hydrochloride (38 mg) and pyridine (2 mL) was added EDCI (49 mg), and the mixture was stirred at room temperature for 16 hr. The mixture was diluted with water, and extracted with ethyl acetate. The organic layer was washed with brine, dried over sodium sulfate, and concentrated under reduced pressure. The residue was purified by preparative HPLC (C18, 0.05% aqueous ammonia/MeCN), and freeze-dried to give the title compound (36 mg).

¹H NMR (400 MHz, CDCl₃) δ 1.13-1.30 (1H, m), 1.83-2.34 (4H, m), 2.88-3.02 (3H, m), 3.49-4.14 (5H, m), 4.74-5.83 (1H, m), 6.85-7.07 (2H, m), 7.26-7.43 (4H, m), 7.45-7.50 (1H, m).

### Example 306

### N-{(3S)-1-[(1S,2S)-2-(2′,6′-difluoro [1,1′-biphenyl]-2-yl)-2-fluorocyclopropane-1-carbonyl]piperidin-3-yl}methanesulfonamide, or N-{(3S)-1-[(1R,2R)-2-(2′,6′-difluoro[1,1′-biphenyl]-2-yl)-2-fluorocyclopropane-1-carbonyl]piperidin-3-yl}methanesulfonamide

To a mixture of rel-(1S,2S)-2-(2′,6′-difluoro[1,1′-biphenyl]-2-yl)-2-fluorocyclopropane-1-carboxylic acid (50.0 mg) synthesized in Step C) of Example 304, (S)—N-(piperidin-3-yl)methanesulfonamide hydrochloride (38.5 mg) and pyridine (3 mL) was added EDCI (65.6 mg), and the mixture was stirred at room temperature for 16 hr. The mixture was concentrated under reduced pressure. The residue was purified by preparative HPLC (C18, 0.05% aqueous ammonia/MeCN), and freeze-dried to give the title compound (28.8 mg).

¹H NMR (400 MHz, CDCl₃) δ 1.20-1.32 (1H, m), 1.60-1.73 (2H, m), 1.78-2.10 (3H, m), 2.23-2.67 (1H, m), 2.75-3.04 (3H, m), 3.30-4.11 (5H, m), 4.47 (1H, br d, J=7.2 Hz), 6.90-7.04 (2H, m), 7.30-7.64 (5H, m).

### Example 318

### N-{(3S)-1-[(1R,2R)-2-(2′,6′-difluoro-5-methyl [1,1′-biphenyl]-2-yl)cyclopropane-1-carbonyl]pyrrolidin-3-yl}methanesulfonamide, or N-{(3S)-1-[(1S,2S)-2-(2′,6′-difluoro-5-methyl[1,1′-biphenyl]-2-yl)cyclopropane-1-carbonyl]pyrrolidin-3-yl}methanesulfonamide

### A) ethyl (2E)-3-(2-bromo-4-methylphenyl)prop-2-enoate

To a mixture of ethyl 2-diethoxyphosphorylacetate (7.32 g) and THF (50 mL) was added 60% NaH (1.21 g) at 0° C. The mixture was stirred at room temperature for 30 min, 2-bromo-4-methylbenzaldehyde (5.00 g) was added thereto at room temperature, and the mixture was stirred at the same temperature for 2 hr. To a mixture of ethyl 2-diethoxyphosphorylacetate (7.32 g) and THF (50 mL) was added 60% NaH (400 mg), and to this mixture was added the previous reaction mixture. The mixture was stirred at room temperature for 12 hr, and the reaction was quenched with water at 0° C., and extracted with ethyl acetate. The organic layer was washed with brine, dried over sodium sulfate, and concentrated under reduced pressure. The residue was purified by silica gel column chromatography (ethyl acetate/petroleum ether) to give the title compound (6.50 g).

¹H NMR (400 MHz, CDCl₃) δ 1.34 (3H, t, J=7.6 Hz), 2.34 (3H, s), 4.19-4.37 (2H, q, J=7.2 Hz), 6.35 (1H, d, J=16.0 Hz), 7.12 (1H, d, J=8.4 Hz), 7.37-7.60 (2H, m), 8.02 (1H, d, J=16.0 Hz).

### B) rac-ethyl (1R,2R)-2-(2-bromo-4-methylphenyl)cyclopropane-1-carboxylate

To a mixture of 60% NaH (1.06 g) and DMSO (50 mL) was added trimethylsulfoxonium iodide (7.97 g) at 0° C., and the mixture was stirred at room temperature for 30 min. A mixture of ethyl (2E)-3-(2-bromo-4-methylphenyl)prop-2-enoate (6.50 g) and DMSO (40 mL) was added dropwise thereto, and the mixture was stirred at the same temperature for 14 hr. The reaction was quenched with water at 0° C., and extracted with ethyl acetate. The organic layer was washed with brine, dried over sodium sulfate, and concentrated under reduced pressure. The residue was purified by silica gel column chromatography (ethyl acetate/petroleum ether) to give the title compound (2.14 g).

¹H NMR (400 MHz, CDCl₃) δ 1.27-1.32 (4H, m), 1.59-1.63 (1H, m), 1.68-1.83 (1H, m), 2.29 (3H, s), 2.58-5.73 (1H, m), 4.13-4.27 (2H, m), 6.89 (1H, d, J=8.0 Hz), 7.02 (1H, d, J=8.0 Hz), 7.39 (1H, s).

### C) rac-ethyl (1R,2R)-2-(2′,6′-difluoro-5-methyl[1,1′-biphenyl]-2-yl)cyclopropane-1-carboxylate

To a mixture of rac-ethyl (1R,2R)-2-(2-bromo-4-methylphenyl)cyclopropane-1-carboxylate (500 mg) and DME (15 mL) were added 2,6-difluorophenylboronic acid (1.39 g), 1 M aqueous tripotassium phosphate solution (5.30 mL) and Xphos Pd G3 (150 mg), and the mixture was stirred under nitrogen atmosphere at 100° C. for 14 hr. The mixture was diluted with water, and extracted with ethyl acetate. The organic layer was washed with brine, dried over sodium sulfate, and concentrated under reduced pressure. The residue was purified by silica gel column chromatography (ethyl acetate/petroleum ether) to give the title compound (461 mg).

MS: [M+H]⁺ 317.0.

### D) rac-(1R,2R)-2-(2′,6′-difluoro-5-methyl[1,1′-biphenyl]-2-yl)cyclopropane-1-carboxylic Acid

To a mixture of sodium hydroxide (132 mg), EtOH (4 mL) and water (3 mL) was added a mixture of rac-ethyl (1R,2R)-2-(2′,6′-difluoro-5-methyl[1,1′-biphenyl]-2-yl)cyclopropane-1-carboxylate (87 mg) and EtOH (3 mL), and the mixture was stirred at room temperature for 14 hr. The mixture was concentrated under reduced pressure. The residue was diluted with water, and extracted with DCM. The aqueous layer was adjusted to pH=3-4 with 1 M hydrochloric acid, and extracted with DCM. The organic layer was dried over sodium sulfate, and concentrated under reduced pressure to give the title compound (85 mg).

¹H NMR (400 MHz, DMSO-d₆) δ 1.12-1.19 (1H, m), 1.25-1.33 (1H, m), 1.53-1.60 (1H, m), 1.95-2.04 (1H, m), 2.30 (3H, s), 6.92-7.28 (5H, m), 7.40-7.54 (1H, m), 12.05 (1H, br s).

### E) A Mixture of N-{(3S)-1-[(1R,2R)-2-(2′,6′-difluoro-5-methyl[1,1′-biphenyl]-2-yl)cyclopropane-1-carbonyl]pyrrolidin-3-yl}methanesulfonamide and N-{(3S)-1-[(1S,2S)-2-(2′,6′-difluoro-5-methyl[1,1′-biphenyl]-2-yl)cyclopropane-1-carbonyl]pyrrolidin-3-yl}methanesulfonamide

To a mixture of rac-(1R,2R)-2-(2′,6′-difluoro-5-methyl[1,1′-biphenyl]-2-yl)cyclopropane-1-carboxylic acid (28 mg) and DMF (1 mL) were added (S)—N-(pyrrolidin-3-yl)methanesulfonamide hydrochloride (18 mg), HATU (38 mg) and DIPEA (47 mg), and the mixture was stirred at room temperature for 14 hr. The mixture was diluted with water, and extracted with ethyl acetate. The organic layer was dried over sodium sulfate, and concentrated under reduced pressure. The residue was purified by preparative HPLC (C18, 0.05% aqueous ammonia/MeCN), and freeze-dried to give the title compound (16 mg).

MS: [M+H]⁺ 435.2.

### F) N-{(3S)-1-[(1R,2R)-2-(2′,6′-difluoro-5-methyl[1,1′-biphenyl]-2-yl)cyclopropane-1-carbonyl]pyrrolidin-3-yl}methanesulfonamide, or N-{(3S)-1-[(1S,2S)-2-(2′,6′-difluoro-5-methyl[1,1′-biphenyl]-2-yl)cyclopropane-1-carbonyl]pyrrolidin-3-yl}methanesulfonamide

A mixture (16 mg) of N-{(3S)-1-[(1R,2R)-2-(2′,6′-difluoro-5-methyl[1,1′-biphenyl]-2-yl)cyclopropane-1-carbonyl]pyrrolidin-3-yl}methanesulfonamide and N-{(3S)-1-[(1S,2S)-2-(2′,6′-difluoro-5-methyl[1,1′-biphenyl]-2-yl)cyclopropane-1-carbonyl]pyrrolidin-3-yl}methanesulfonamide was resolved by SFC (column: Phenomenex-Cellulose-2, 250 mm×30 mm, 10 μm, mobile phase: CO₂/MeOH containing 0.1% aqueous ammonia=60/40 v/v), and the fraction having a shorter retention time was concentrated. The residue was dissolved in MeCN (3 mL) and water (30 mL), and the solution was freeze-dried to give the title compound (5.2 mg).

¹H NMR (400 MHz, CDCl₃) δ 1.26-1.34 (1H, m), 1.39-1.50 (1H, m), 1.63-1.70 (1H, m), 1.76-2.10 (1H, m), 2.13-2.29 (1H, m), 2.29-2.39 (4H, m), 3.00 (3H, s), 3.27-3.81 (4H, m), 3.94-4.12 (1H, m), 4.42 (1H, s), 6.92-7.05 (3H, m), 7.07 (1H, s), 7.18 (1H, d, J=7.8 Hz), 7.27-7.39 (1H, m).

### Example 319

### N-{(3S,5S)-1-[(1R,2R)-2-(2′,6′-difluoro[1,1′-biphenyl]-2-yl)cyclopropane-1-carbonyl]-5-hydroxypiperidin-3-yl}methanesulfonamide

### A) tert-butyl (3S,5S)-3-hydroxy-5-[(methanesulfonyl)amino]piperidine-1-carboxylate

To a mixture of tert-butyl (3S,5S)-3-amino-5-hydroxypiperidine-1-carboxylate (50 mg) and DCM (3 mL) were added TEA (35 mg) and methanesulfonyl chloride (32 mg) at 0° C., and the mixture was stirred at room temperature for 2 hr. The mixture was diluted with water, and extracted with DCM. The organic layer was washed with brine, dried over sodium sulfate, and concentrated under reduced pressure. The residue was purified by silica gel column chromatography (ethyl acetate/petroleum ether) to give the title compound (54 mg).

¹H NMR (400 MHz, CDCl₃) δ 1.47 (9H, s), 1.77-2.08 (2H, m), 2.93-3.11 (4H, m), 3.28-3.57 (2H, m), 3.75-3.85 (1H, m), 3.95-4.10 (1H, m), 4.25-4.44 (1H, m).

### B) N-{(3S,5S)-1-[(1R,2R)-2-(2′,6′-difluoro[1,1′-biphenyl]-2-yl)cyclopropane-1-carbonyl]-5-hydroxypiperidin-3-yl}methanesulfonamide

To a mixture of tert-butyl (3S,5S)-3-hydroxy-5-[(methanesulfonyl)amino]piperidine-1-carboxylate (47 mg) and DCM (6 mL) was added trifluoroacetic acid (2 mL), and the mixture was stirred at room temperature for 2 hr. The mixture was concentrated under reduced pressure to give N-[(3S,5S)-5-hydroxypiperidin-3-yl]methanesulfonamide trifluoroacetate (75 mg).

To a mixture of (1R,2R)-2-(2′,6′-difluoro[1,1′-biphenyl]-2-yl)cyclopropane-1-carboxylic acid (50 mg), N-[(3S,5S)-5-hydroxypiperidin-3-yl]methanesulfonamide trifluoroacetate (67 mg) and DMF (2 mL) were added HOBt (25 mg), EDCI (52 mg) and TEA (74 mg), and the mixture was stirred at room temperature for 13 hr. The mixture was diluted with water, and extracted with ethyl acetate. The organic layer was washed with brine, dried over sodium sulfate, and concentrated under reduced pressure. The residue was purified by preparative HPLC (C18, 0.05% aqueous ammonia/MeCN), and freeze-dried to give the title compound (27 mg).

¹H NMR (400 MHz, CDCl₃) δ 1.20-1.50 (2H, m), 1.79-2.50 (5H, m), 2.75-3.00 (3H, m), 3.33-3.65 (3H, m), 3.67-4.05 (3H, m), 4.58-5.15 (1H, m), 6.91-7.24 (4H, m), 7.26-7.44 (3H, m).

### Example 353

### N-[(1S)-1-{(2S)-1-[(1S,2S)-2-(2′,6′-difluoro[1,1′-biphenyl]-2-yl)-2-fluorocyclopropane-1-carbonyl]azetidin-2-yl}ethyl]methanesulfonamide

### A) tert-butyl (S)-2-acetylazetidine-1-carboxylate

To a stirred solution of (2S)-1-tert-butoxycarbonylazetidine-2-carboxylic acid (4.50 g) and N,O-dimethylhydroxylamine (2.29 g, HCl salt) in DMF (40 mL) was added HATU (8.50 g) and then DIPEA (10.6 g) at 0° C., and the reaction mixture was stirred at 20° C. under N₂ atmosphere for 16 h. The reaction mixture was diluted with water and extracted with EtOAc. The combined organic layer was washed with brine, dried over Na₂SO₄, and concentrated in vacuo. The residue was purified by silica gel column chromatography (EtOAc) to afford tert-butyl (S)-2-(methoxy(methyl)carbamoyl)azetidine-1-carboxylate (5.46 g).

To a stirred solution of tert-butyl (S)-2-(methoxy(methyl)carbamoyl)azetidine-1-carboxylate (5.45 g). in THF (70 mL) was added MeMgBr (22.3 mL 3 M in THF) at −78° C., and the reaction mixture was stirred at 20° C. for 3 h under N₂ atmosphere. The reaction mixture was diluted with water and extracted with EtOAc. The combined organic layer was washed with brine, dried over Na₂SO₄, and concentrated in vacuo. The residue was purified by silica gel column chromatography (EtOAc/petroleum ether) to afford the title compound (4.40 g).

¹H NMR (400 MHz, CDCl₃) δ 1.45 (9H, s), 2.08-2.18 (1H, m), 2.27 (3H, s), 2.41-2.52 (1H, m), 3.84-4.00 (2H, m), 4.55-4.67 (1H, mi).

### B) tert-butyl (S)-2-((R*)-1-((methylsulfonyl)oxy)ethyl)azetidine-1-carboxylate

To a mixture of tert-butyl (S)-2-acetylazetidine-1-carboxylate (1.00 g) in MeOH (10 mL) was added NaBH₄ (380 mg) at 0° C. The mixture was stirred at 0° C. for 1 h. The mixture was diluted with water and it was extracted with EtOAc. The combined organic layer was dried over Na₂SO₄ and concentrated under reduced pressure to give a crude product. The crude product was purified by silica gel column chromatography (EtOAc/petroleum ether) to give tert-butyl (S)-2-((R*)-1-hydroxyethyl)azetidine-1-carboxylate (430 mg, more polar on the column).

To a mixture of tert-butyl (S)-2-((R*)-1-hydroxyethyl)azetidine-1-carboxylate (430 mg) and TEA (432 mg) in DCM (5 mL) at 0° C. was added MsCl (367 mg). The solution was stirred at 25° C. for 2 h under N₂ atmosphere. To this mixture was added water. It was extracted with DCM. The combined organic layer was washed with brine, dried over Na₂SO₄ and concentrated under reduced pressure. It was purified by silica gel column chromatography (EtOAc/petroleum ether) to give the title compound (591 mg).

¹H NMR (400 MHz, CDCl₃) δ 1.34 (3H, d, J=6.8 Hz), 1.43 (9H, s), 2.14-2.33 (2H, m), 3.00 (3H, s), 3.71-3.89 (2H, m), 4.16-4.24 (1H, m), 5.00-5.09 (1H, m).

### C) tert-butyl (S)-2-((S)-1-azidoethyl)azetidine-1-carboxylate

A solution of tert-butyl (S)-2-((R*)-1-((methylsulfonyl)oxy)ethyl)azetidine-1-carboxylate (5.66 g) synthesized in Step B) and tetrabutylammonium azide (19.0 g) in MeCN (150 mL) was stirred for 6 h at 80° C. The solvent was removed in vacuo and the residue was purified by silica gel column chromatography (EtOAc/hexane) to give the title compound (4.03 g).

¹H NMR (400 MHz, CDCl₃) δ 1.34 (3H, d, J=6.7 Hz), 1.45 (9H, s), 2.00-2.14 (1H, m), 2.19-2.34 (1H, m), 3.61-3.73 (1H, m), 3.74-3.90 (2H, m), 4.22-4.33 (1H, m).

### D) tert-butyl (S)-2-((S)-1-(methylsulfonamido)ethyl)azetidine-1-carboxylate

A mixture of tert-butyl (S)-2-((S)-1-azidoethyl)azetidine-1-carboxylate (4.03 g) and 20% Pd(OH)₂ on carbon (2.50 g) in MeOH (100 mL) was stirred under H₂ atmosphere (balloon) for 18 h at rt. The mixture was filtered through a pad of celite and the solvent was removed in vacuo to yield tert-butyl (S)-2-((S)-1-aminoethyl)azetidine-1-carboxylate (4.03 g) which was used without further purification for the next step.

To a solution of tert-butyl (S)-2-((S)-1-aminoethyl)azetidine-1-carboxylate (3.57 g) and TEA (9.94 mL) was added methanesulfonic anhydride (4.66 g) at 0° C. and the resulting solution was stirred for 1 h at rt. The solvent was removed in vacuo and the residue was purified by silica gel column chromatography (EtOAc/hexane) and by silica gel column chromatography (NH, EtOAc/hexane) to give the title compound (4.16 g).

¹H NMR (400 MHz, CDCl₃) δ 1.20 (3H, d, J=6.2 Hz), 1.46 (9H, s), 1.93-2.02 (1H, m), 2.30-2.41 (1H, m), 2.94 (3H, s), 3.48-3.58 (1H, m), 3.76-3.90 (2H, m), 4.05-4.15 (1H, m), 6.18-6.75 (1H, m).

### E) N-[(1S)-1-{(2S)-1-[(1S,2S)-2-(2′,6′-difluoro[1,1′-biphenyl]-2-yl)-2-fluorocyclopropane-1-carbonyl]azetidin-2-yl}ethyl]methanesulfonamide

To tert-butyl (S)-2-((S)-1-(methylsulfonamido)ethyl)azetidine-1-carboxylate (3.27 g) in EtOAc (120 mL) was added TsOH·H₂O (2.68 g) at rt and the mixture was stirred at 65° C. for 16 h and then the solvent was removed in vacuo to yield crude N—((S)-1-((S)-azetidin-2-yl)ethyl)methanesulfonamide 4-methylbenzenesulfonate.

To the crude N—((S)-1-((S)-azetidin-2-yl)ethyl)methanesulfonamide 4-methylbenzenesulfonate were added DMF (120 mL), rel-(1S,2S)-2-(2′,6′-difluoro[1,1′-biphenyl]-2-yl)-2-fluorocyclopropane-1-carboxylic acid (3.78 g) synthesized in Step C) of Example 304, TEA (6.55 mL), and HATU (6.70 g) at 0° C. and the resulting solution was stirred for 1 h at that temperature. The mixture was quenched with sat. NH₄Cl aq. at 0° C. and extracted with EtOAc. The organic layer was separated, washed with brine, dried over MgSO₄ and concentrated in vacuo. The residue was purified by silica gel column chromatography (EtOAc/hexane) and by silica gel column chromatography (NH, EtOAc/hexane) to give the title compound (2.31 g) as a white amorphous solid.

The amorphous solid of N-[(1S)-1-{(2S)-1-[(1S,2S)-2-(2′,6′-difluoro[1,1′-biphenyl]-2-yl)-2-fluorocyclopropane-1-carbonyl]azetidin-2-yl}ethyl]methanesulfonamide (2.30 g) was dissolved in EtOAc (6.00 mL) and the solution was stirred at rt. After 5 min, white precipitates were formed. A small amount of seed crystal and heptane (1.00 mL) were successively added thereto. The mixture was stirred at rt overnight. The resultant precipitates were collected and dried in vacuo at 50° C. to give the title compound (1.85 g) as a white solid.

¹H NMR (400 MHz, CDCl₃) δ 1.18-1.32 (4H, m), 1.87-1.99 (1H, m), 2.01-2.18 (2H, m), 2.39-2.52 (1H, m), 2.90 (3H, s), 3.58-3.69 (1H, m), 4.18-4.36 (3H, m), 6.82 (1H, br s), 6.94-7.02 (2H, m), 7.31-7.55 (5H, m).

### Example 358

### N-({(2S,3R)-1-[(1S,2S)-2-(2′,6′-difluoro [1,1′-biphenyl]-2-yl)-2-fluorocyclopropane-1-carbonyl]-3-methylazetidin-2-yl}methyl)methanesulfonamide

### A) A Mixture of [(2R,3R)-1-benzyl-3-methylazetidin-2-yl]methanol and [(2S,3R)-1-benzyl-3-methylazetidin-2-yl]methanol

To a mixture of a mixture (10.0 g) of tert-butyl (2R,3R)-1-benzyl-3-methylazetidine-2-carboxylate and tert-butyl (2S,3R)-1-benzyl-3-methylazetidine-2-carboxylate, and THF (300 mL) were added LiBH₄ (3.33 g) and EtOH (8.81 g) at 20° C., and the reaction mixture was stirred at 60° C. for 60 h. The reaction mixture was diluted with sat. NH₄Cl aq. (100 mL) and extracted with EtOAc (50 mL×2). The aqueous phase was concentrated in vacuum and then DCM (50 mL×10) was added. Then the mixture was filtered and the combined organic solution was concentrated under reduced pressure to give the title compound (4.80 g).

¹H NMR (400 MHz, CD₃OD) δ1.22-1.29 (3H, m), 2.81-3.14 (1H, m), 3.54-3.89 (3H, m), 4.13-4.57 (4H, m), 7.39-7.58 (5H, m).

### B) tert-butyl (2S,3R)-2-{[(tert-butoxycarbonyl)(methanesulfonyl)amino]methyl}-3-methylazetidine-1-carboxylate

To a mixture of a mixture (12.0 g) of [(2R,3R)-1-benzyl-3-methylazetidin-2-yl]methanol and [(2S,3R)-1-benzyl-3-methylazetidin-2-yl]methanol, and MeOH (100 mL) was added 20% Pd(OH)₂ on carbon (7.00 g) under N₂ atmosphere. The suspension was degassed under vacuum and purged with H₂ three times. The mixture was stirred under H₂ (15 psi) atmosphere at 15° C. for 16 h. The mixture was filtered to give a filtrate and it was concentrated under reduced pressure to give a mixture (6.30 g) of [(2R,3R)-3-methylazetidin-2-yl]methanol and [(2S,3R)-3-methylazetidin-2-yl]methanol.

To a mixture of the mixture (6.30 g) of [(2R,3R)-3-methylazetidin-2-yl]methanol and [(2S,3R)-3-methylazetidin-2-yl]methanol, dioxane (30 mL), and water (40 mL) were added NaOH (6.23 g) and Boc₂O (20.4 g). The mixture was stirred at 15° C. for 16 h. The mixture was quenched with water (100 mL) and extracted with EtOAc (100 mL×3). The combined organic layer was concentrated under reduced pressure to give a mixture (17.5 g) of tert-butyl (2R,3R)-2-(hydroxymethyl)-3-methylazetidine-1-carboxylate and tert-butyl (2S,3R)-2-(hydroxymethyl)-3-methylazetidine-1-carboxylate.

To a mixture of the mixture (17.5 g) of tert-butyl (2R,3R)-2-(hydroxymethyl)-3-methylazetidine-1-carboxylate and tert-butyl (2S,3R)-2-(hydroxymethyl)-3-methylazetidine-1-carboxylate, tert-butyl N-methylsulfonylcarbamate (20.4 g), PPh₃ (34.2 g), and THF (150 mL) was added DIAD (26.4 g, 95% purity) at 0° C. under N₂ atmosphere. The mixture was stirred at 15° C. for 16 h. The mixture was quenched with water and it was extracted with EtOAc. The combined organic layer was concentrated under reduced pressure to give a residue. The residue was purified by silica gel column chromatography (EtOAc/petroleum ether) to give the title compound (5.10 g).

¹H NMR (400 MHz, CDCl₃) δ 1.15 (3H, d, J=7.2 Hz), 1.40 (9H, s), 1.53 (9H, s), 2.74-2.84 (1H, m), 3.30-3.57 (4H, m), 3.69-3.79 (1H, m), 3.98 (1H, t, J=8.8 Hz), 4.23-4.35 (1H, m), 4.40-4.50 (1H, m).

### C) N-({(2S,3R)-1-[(1S,2S)-2-(2′,6′-difluoro[1,1′-biphenyl]-2-yl)-2-fluorocyclopropane-1-carbonyl]-3-methylazetidin-2-yl}methyl)methanesulfonamide

To a mixture of tert-butyl (2S,3R)-2-{[(tert-butoxycarbonyl)(methanesulfonyl)amino]methyl}-3-methylazetidine-1-carboxylate (5.00 g) and EtOAc (100 mL) was added TsOH·H₂O (3.30 g) at rt. The mixture was stirred at 70° C. under N₂ atmosphere for 5 h. The mixture was concentrated in vacuo to give a crude N-(((2S,3R)-3-methylazetidin-2-yl)methyl)methanesulfonamide 4-methylbenzenesulfonate. To a mixture of rel-(1S,2S)-2-(2′,6′-difluoro[1,1′-biphenyl]-2-yl)-2-fluorocyclopropane-1-carboxylic acid (3.87 g) synthesized in Step C) of Example 304, EDCI (2.57 g), HOBt (1.84 g), and DMF (80 mL) was added TEA (5.6 mL) at 0° C. After being stirred at 0° C. for 5 min, to the mixture was added a mixture of the crude N-(((2S,3R)-3-methylazetidin-2-yl)methyl)methanesulfonamide 4-methylbenzenesulfonate and DMF (20 mL) at 0° C. The mixture was stirred at 0° C. to rt under N₂ atmosphere overnight. To the mixture were successively added EtOAc (100 mL) and water (200 mL) at 0° C. and extracted with EtOAc. The organic layer was separated, washed with sat. NaHCO₃ aq., water and brine, dried over Na₂SO₄, and concentrated in vacuo. The residue was purified by silica gel column chromatography (EtOAc/hexane) to give the title compound (3.84 g) as a white amorphous solid.

The amorphous solid of N-({(2S,3R)-1-[(1S,2S)-2-(2′,6′-difluoro[1,1′-biphenyl]-2-yl)-2-fluorocyclopropane-1-carbonyl]-3-methylazetidin-2-yl}methyl)methanesulfonamide (4.64 g) was dissolved in EtOAc (10 mL). To the stirred solution was added hexane (3 mL) and seeds of the compound crystal at rt. After 30 min, hexane (3 mL) was added. After 30 min, hexane (6 mL) was added. After 1 h, the resulting solid was collected and washed with 30% AcOEt in hexane to give the title compound (4.09 g) as colorless crystal.

¹H NMR (400 MHz, CDCl₃) δ 1.18-1.36 (4H, m), 1.85-1.98 (1H, m), 1.98-2.06 (1H, m), 2.84-2.97 (4H, m), 3.31-3.49 (2H, m), 3.74-3.87 (1H, m), 4.42-4.59 (2H, m), 6.75-6.85 (1H, m), 6.94-7.03 (2H, m), 7.32-7.40 (2H, m), 7.45-7.58 (3H, m).

### Example 393

### N-({(2R,3R)-1-[(1S,2S)-2-(2′,6′-difluoro [1,1′-biphenyl]-2-yl)-2-fluorocyclopropane-1-carbonyl]-3-fluoroazetidin-2-yl}methyl)ethanesulfonamide

### A) ((2R,3S)-1-benzyl-3-(benzyloxy)azetidin-2-yl)methanol

To a mixture of (2R,3S)-1-benzyl-3-(benzyloxy)-2-(((tert-butyldiphenylsilyl)oxy)methyl)azetidine (30.9 g) and THF (50 mL) was added 1 M TBAF in THF (89 mL) at 0° C. and the mixture was stirred at 60° C. for 30 min. The reaction mixture was concentrated in vacuo. The residue was purified by silica gel column chromatography (EtOAc/hexane) to give the crude title compound. This material was dissolved in EtOAc, filtered through a NH silica gel pad eluted with EtOAc, and concentrated in vacuo to give the title compound (15.2 g).

¹H NMR (400 MHz, CDCl₃) δ 2.56 (1H, br s), 2.83-2.90 (1H, m), 3.22-3.38 (3H, m), 3.54-3.60 (1H, m), 3.61-3.74 (2H, m), 4.11-4.21 (1H, m), 4.45 (2H, s), 7.22-7.40 (10H, m).

### B) tert-butyl (2R,3S)-3-hydroxy-2-(hydroxymethyl)azetidine-1-carboxylate

A mixture of ((2R,3S)-1-benzyl-3-(benzyloxy)azetidin-2-yl)methanol (15.2 g), 20% Pd(OH)₂ on carbon (3.05 g), Boc₂O (13.7 mL), and MeOH (130 mL) was hydrogenated under balloon pressure at rt for 4.5 h. After that, the balloon was replaced with new one and the mixture was hydrogenated under balloon pressure at rt overnight. After that, 20% Pd(OH)₂ on carbon (3.05 g) and MeOH (70 mL) were added thereto, the balloon was replaced with new one, and the mixture was hydrogenated under balloon pressure at rt for further 24 h. The catalyst was removed by filtration through a silica gel/NH silica gel/Celite pad eluted with EtOAc and the filtrate was concentrated in vacuo. The residue was purified by silica gel column chromatography (EtOAc/hexane; MeOH/EtOAc) to give the title compound (9.53 g).

¹H NMR (400 MHz, CDCl₃) δ 1.45 (9H, s), 2.87 (1H, br d, J=5.7 Hz), 3.69-3.90 (3H, m), 3.99-4.06 (1H, m), 4.16-4.36 (2H, m).

### C) tert-butyl (2R,3S)-2-(azidomethyl)-3-(((trifluoromethyl)sulfonyl)oxy)azetidine-1-carboxylate

To a cooled (0° C.) mixture of tert-butyl (2R,3S)-3-hydroxy-2-(hydroxymethyl)azetidine-1-carboxylate (3.83 g), 2,6-lutidine (26.3 mL), and MeCN (64 mL) was added Tf₂O (9.27 mL) dropwise via syringe and the mixture was stirred at the same temperature for 1 h. After that, tetra-n-butylammonium azide (5.90 g) was added in one portion, and then the mixture was gradually warmed up to rt overnight. The mixture was diluted with EtOAc (100 mL) and water (100 mL) at 0° C., stirred at the same temperature for 5 min, and then extracted with EtOAc. The organic layer was washed with water and brine, dried over Na₂SO₄, filtered through a silica gel/NH silica gel pad eluted with EtOAc, and concentrated in vacuo. This crude material was purified by silica gel column chromatography (EtOAc/hexane) to give the title compound (5.19 g).

¹H NMR (400 MHz, CDCl₃) δ 1.47 (9H, s), 3.45-3.51 (1H, m), 3.86-3.97 (1H, m), 4.03-4.09 (1H, m), 4.20-4.27 (1H, m), 4.48-4.54 (1H, m), 5.23-5.31 (1H, m).

### D) tert-butyl (2R,3R)-2-(azidomethyl)-3-fluoroazetidine-1-carboxylate

To a cooled (0° C.) mixture of tert-butyl (2R,3S)-2-(azidomethyl)-3-(((trifluoromethyl)sulfonyl)oxy)azetidine-1-carboxylate (10.6 g) and THF (58.6 mL) was added 1M TBAF in THF (58.6 mL) dropwise via syringe and the mixture was stirred at the same temperature for 0.5 h. After that, the mixture was stirred at rt for 2 h. The mixture was diluted with EtOAc (200 mL) and added dropwise aq. NH₄Cl (100 mL) at 0° C. and the mixture was stirred at the same temperature for 5 min. The organic layer was separated, washed with brine. The combined aqueous layer was extracted with EtOAc. The combined organic layer was concentrated in vacuo and the residue was purified by silica gel column chromatography (EtOAc/hexane) to give the title compound (6.16 g).

¹H NMR (300 MHz, CDCl₃) δ 1.45 (9H, s), 3.54-3.65 (1H, m), 3.71-3.81 (1H, m), 3.92-4.07 (1H, m), 4.18 (1H, ddd, J=19.7, 10.7, 6.3 Hz), 4.36-4.49 (1H, m), 5.18-5.45 (1H, m).

### E) tert-butyl (2R,3R)-2-(ethylsulfonamidomethyl)-3-fluoroazetidine-1-carboxylate

A mixture of tert-butyl (2R,3R)-2-(azidomethyl)-3-fluoroazetidine-1-carboxylate (5.27 g) and Lindlar's catalyst (2.64 g) in EtOAc (80 mL) was hydrogenated under balloon pressure at rt overnight. The catalyst was removed by filtration through a Celite pad eluted with EtOAc and the filtrate was concentrated in vacuo. The residue was dissolved in THF (80 mL), and TEA (4.79 mL) was added, and ethanesulfonyl chloride (2.39 mL) was added dropwise thereto via syringe over 2 min at 0° C. The mixture was stirred at the same temperature for 3.5 h. The mixture was diluted with EtOAc and water at 0° C. and the mixture was stirred at the same temperature for 5 min. The organic layer was separated, washed with brine, dried over Na₂SO₄, filtered through a silica gel/NH silica gel pad, and concentrated in vacuo. The residue was purified by silica gel column chromatography (EtOAc/hexane) to give the title compound (6.17 g).

¹H NMR (400 MHz, CDCl₃) δ 1.37 (3H, t, J=7.4 Hz), 1.46 (9H, s), 3.06 (2H, q, J=7.3 Hz), 3.44-3.58 (2H, m), 3.91-4.05 (1H, m), 4.13-4.28 (1H, m), 4.45-4.57 (1H, m), 5.17-5.42 (1H, m), 5.85 (1H, br s).

### F) N-({(2R,3R)-1-[(1S,2S)-2-(2′,6′-difluoro[1,1′-biphenyl]-2-yl)-2-fluorocyclopropane-1-carbonyl]-3-fluoroazetidin-2-yl}methyl)ethanesulfonamide

A mixture of tert-butyl (2R,3R)-2-(ethylsulfonamidomethyl)-3-fluoroazetidine-1-carboxylate (1.39 g), p-toluenesulfonic acid monohydrate (1.78 g), and EtOAc (20 mL) was heated at 80° C. for 16 h. The mixture was concentrated in vacuo to give N-(((2R,3R)-3-fluoroazetidin-2-yl)methyl)ethanesulfonamide 4-methylbenzenesulfonate (1.73 g) as a crude material which was used in the next step.

To a cooled (0° C.) mixture of N-(((2R,3R)-3-fluoroazetidin-2-yl)methyl)ethanesulfonamide 4-methylbenzenesulfonate (1.73 g) and DMF (30 mL) were successively added rel-(1S,2S)-2-(2′,6′-difluoro[1,1′-biphenyl]-2-yl)-2-fluorocyclopropane-1-carboxylic acid (1.37 g) synthesized in Step C) of Example 304, EDCI (1.08 g), HOBt (0.760 g), and DIPEA (3.28 mL) and the mixture was gradually warmed up to rt overnight. The mixture was diluted with EtOAc and aq. NaHCO₃ at 0° C. and the mixture was stirred at the same temperature for 5 min. The organic layer was separated, washed with brine, dried over Na₂SO₄, filtered through a silica gel/NH silica gel pad, and concentrated in vacuo. The residue was purified by silica gel column chromatography (EtOAc/hexane) to give the title compound (1.57 g).

¹H NMR (400 MHz, CDCl₃) δ 1.29-1.40 (4H, m), 1.89-2.04 (2H, m), 2.97-3.07 (2H, m), 3.44-3.53 (1H, m), 3.55-3.64 (1H, m), 4.29-4.42 (1H, m), 4.52-4.73 (2H, m), 5.26-5.48 (1H, m), 6.09 (1H, br d, J=8.1 Hz), 6.92-7.03 (2H, m), 7.31-7.55 (5H, m).

### Example 411

### N-({(2R,3R)-1-[(1S,2S)-2-(2′,6′-difluoro-5-methyl [1,1′-biphenyl]-2-yl)-2-fluorocyclopropane-1-carbonyl]-3-fluoroazetidin-2-yl}methyl)methanesulfonamide

### A) 2-bromo-1-ethenyl-4-methylbenzene

To a solution of methyltriphenylphosphonium iodide (36.5 g) in THF (360 mL) was added n-butyllithium (33.2 mL, 2.5 M in hexane) dropwise at 0° C. under N₂ atmosphere. The mixture was stirred for 1 h at 0° C. and then a solution of 2-bromo-4-methylbenzaldehyde (15.0 g) in THF (100 mL) was added slowly at −78° C. Then it was allowed to warm to 25° C. and stirred at 25° C. for 16 h. The mixture was diluted with water and extracted with EtOAc. The combined organic layer was washed with brine, dried over Na₂SO₄ and then concentrated under reduced pressure. The residue was purified by silica gel column chromatography (petroleum ether) to give title compound (12.0 g).

¹H NMR (400 MHz, CDCl₃) δ 2.32 (3H, s), 5.30 (1H, d, J=12.0 Hz), 5.62 (1H, d, J=18.4 Hz), 6.98-7.11 (2H, m), 7.36-7.46 (2H, m).

### B) 2-bromo-1-(2-bromo-1-fluoroethyl)-4-methylbenzene

To a mixture of 2-bromo-1-ethenyl-4-methylbenzene (12.0 g) in DCM (150 mL) were added triethylamine trihydrofluoride (19.6 g) and N-bromosuccinimide (13.0 g) at 0° C., then the mixture was stirred at 25° C. for 16 h. The mixture was concentrated under reduced pressure. The residue was purified by silica gel column chromatography (petroleum ether) to give title compound (14.5 g).

¹H NMR (400 MHz, CDCl₃) δ 2.34 (3H, s), 3.51-3.83 (2H, m), 5.80-6.02 (1H, m), 7.17-7.22 (1H, m), 7.37-7.43 (2H, m).

### C) 2-bromo-1-(1-fluoroethenyl)-4-methylbenzene

A mixture of 2-bromo-1-(2-bromo-1-fluoroethyl)-4-methylbenzene (34.0 g) in DCM (150 mL) was added DBU (21.0 g), then the mixture was stirred at 50° C. for 16 h. The mixture was concentrated under reduced pressure. The residue was purified by silica gel column chromatography (petroleum ether) to give title compound (22.0 g).

¹H NMR (400 MHz, CDCl₃) δ 2.35 (3H, s), 4.87-5.09 (2H, m), 7.10-7.15 (1H, m), 7.34-7.38 (1H, m), 7.46 (1H, s).

### D) 2′,6′-difluoro-2-(1-fluoroethenyl)-5-methyl-1,1′-biphenyl

A mixture of 2-(2,6-difluorophenyl)-4,4,5,5-tetramethyl-1,3,2-dioxaborolane (24.6 g), 2-bromo-1-(1-fluoroethenyl)-4-methylbenzene (11.0 g), cesium carbonate (66.7 g), copper (I) chloride (5.06 g), SPhos (4.20 g) and palladium (II) acetate (459 mg) in DMF (150 mL) was stirred at 100° C. for 20 min under N₂ atmosphere. The mixture was filtered and washed with EtOAc. Then the organic layer was washed with water, dried over Na₂SO₄ and concentrated under vacuum. The residue was purified by silica gel column chromatography (petroleum ether) to give title compound (20.0 g).

¹H NMR (400 MHz, CDCl₃) δ 2.41 (3H, s), 4.20-4.83 (2H, m), 6.90-7.00 (2H, m), 7.13 (1H, s), 7.26-7.35 (2H, m), 7.49-7.58 (1H, m).

### E) ethyl (1S,2S)-2-(2′,6′-difluoro-5-methyl[1,1′-biphenyl]-2-yl)-2-fluorocyclopropane-1-carboxylate

To a mixture of dichloro(p-cymene)ruthenium(II) (949 mg) and (S,S)-pybox(iPr) (934 mg) in THF (50 mL) was added a solution of 2′,6′-difluoro-2-(1-fluoroethenyl)-5-methyl-1,1′-biphenyl (14.8 g) in THF (150 mL) dropwise at 20° C. under N₂ atmosphere. After the mixture was heated at 55° C., to the mixture was added a solution of ethyl 2-diazoacetate (17.0 g) in toluene (150 mL) dropwise over 3 h under N₂ atmosphere. And then it was stirred at 55° C. for 14 h under N₂ atmosphere. The reaction mixture was concentrated under reduced pressure. The residue was purified by silica gel column chromatography (EtOAc/petroleum ether) and reversed column chromatography (C18, MeOH/water) to give crude ethyl 2-(2′,6′-difluoro-5-methyl[1,1′-biphenyl]-2-yl)-2-fluorocyclopropane-1-carboxylate (16.0 g). The crude ethyl 2-(2′,6′-difluoro-5-methyl[1,1′-biphenyl]-2-yl)-2-fluorocyclopropane-1-carboxylate (9.00 g) was purified by SFC (column: DAICEL CHIRALPAK IC, 250 mm×50 mm, 10 μm, mobile phase: CO₂/EtOH containing 0.1% aqueous ammonia=80/20 v/v).

The fractions of the 3^{rd} peak with the longest retention time were concentrated under reduced pressure and lyophilized to give the title compound (6.00 g).

¹H NMR (400 MHz, CDCl₃) δ 1.17-1.24 (3H, m), 1.32-1.44 (1H, m), 1.84-1.96 (1H, m), 2.01-2.09 (1H, m), 2.41 (3H, s), 3.93-4.15 (2H, m), 6.90-7.03 (2H, m), 7.13 (1H, s), 7.27-7.37 (2H, m), 7.45-7.52 (1H, m).

### F) (1S,2S)-2-(2′,6′-difluoro-5-methyl[1,1′-biphenyl]-2-yl)-2-fluorocyclopropane-1-carboxylic Acid

To a mixture of ethyl (1S,2S)-2-(2′,6′-difluoro-5-methyl[1,1′-biphenyl]-2-yl)-2-fluorocyclopropane-1-carboxylate (6.00 g) in MeOH (20 mL), THF (20 mL) and water (20 mL) was added NaOH (7.18 g), and then the mixture was stirred at 60° C. for 16 h. The mixture was acidified by 1 M HCl aq. until pH=3 and then extracted with EtOAc. The combined organic layer was washed with brine, dried over Na₂SO₄ and then concentrated under reduced pressure to give title compound (5.60 g).

¹H NMR (400 MHz, DMSO-d₆) δ 1.41-1.51 (1H, m), 1.55-1.66 (1H, m), 1.96-2.06 (1H, m), 2.37 (3H, s), 7.11-7.20 (3H, m), 7.32-7.38 (1H, m), 7.43-7.54 (2H, m), 12.25 (1H, br s).

### G) tert-butyl (2R,3R)-3-fluoro-2-(methylsulfonamidomethyl)azetidine-1-carboxylate

A mixture of tert-butyl (2R,3R)-2-(azidomethyl)-3-fluoroazetidine-1-carboxylate (0.701 g) and Lindlar's catalyst (0.701 g) in EtOAc (10 mL) was hydrogenated under balloon pressure at rt overnight. The catalyst was removed by filtration through a Celite pad eluted with EtOAc and the filtrate was concentrated in vacuo. The residue was dissolved in THF (10 mL), and then TEA (1.27 mL) and methanesulfonic anhydride (0.796 g) were successively added thereto at 0° C. The mixture was stirred at the same temperature for 5 min. After that, the mixture was stirred at rt for 4 h. The mixture was diluted with EtOAc and aq. NaHCO₃ at 0° C. and the mixture was stirred at the same temperature for 5 min. The organic layer was separated, washed with brine, dried over Na₂SO₄, filtered through a silica gel/NH silica gel pad, and concentrated in vacuo. The residue was purified by silica gel column chromatography (EtOAc/hexane) to give the title compound (0.789 g).

¹H NMR (400 MHz, CDCl₃) δ 1.46 (9H, s), 2.97 (3H, s), 3.47-3.61 (2H, m), 3.92-4.05 (1H, m), 4.15-4.27 (1H, m), 4.44-4.58 (1H, m), 5.18-5.42 (1H, m), 5.88 (1H, br s).

### H) N-({(2R,3R)-1-[(1S,2S)-2-(2′,6′-difluoro-5-methyl[1,1′-biphenyl]-2-yl)-2-fluorocyclopropane-1-carbonyl]-3-fluoroazetidin-2-yl}methyl)methanesulfonamide

A mixture of tert-butyl (2R,3R)-3-fluoro-2-(methylsulfonamidomethyl)azetidine-1-carboxylate (0.327 g), p-toluenesulfonic acid monohydrate (0.441 g), and EtOAc (5 mL) was refluxed for 3.5 h. The mixture was concentrated in vacuo to give N-(((2R,3R)-3-fluoroazetidin-2-yl)methyl)methanesulfonamide 4-methylbenzenesulfonate as a crude material which was used in the next step.

To a cooled (0° C.) mixture of the crude N-(((2R,3R)-3-fluoroazetidin-2-yl)methyl)methanesulfonamide 4-methylbenzenesulfonate (0.410 g) and DMF (5 mL) were successively added (1S,2S)-2-(2′,6′-difluoro-5-methyl[1,1′-biphenyl]-2-yl)-2-fluorocyclopropane-1-carboxylic acid (0.354 g), EDCI (0.288 g), HOBt (0.203 g), and DIPEA (0.808 mL) and the mixture was gradually warmed up to rt overnight. The mixture was diluted with EtOAc and aq. NaHCO₃ at 0° C. and the mixture was stirred at the same temperature for 5 min. The organic layer was separated, washed with brine, dried over Na₂SO₄, filtered through a silica gel/NH silica gel pad, and concentrated in vacuo. The residue was purified by silica gel column chromatography (EtOAc/hexane) to give the title compound as a white solid. This material (0.333 g) was dissolved in EtOAc (5 mL) at 80° C. with stirring and heptane (4 mL) was added dropwise thereto. The resultant suspension was cooled to rt and stirred overnight to give the title compound (0.305 g) as a white solid.

¹H NMR (400 MHz, CDCl₃) δ 1.26-1.37 (1H, m), 1.84-2.03 (2H, m), 2.41 (3H, s), 2.93 (3H, s), 3.45-3.56 (1H, m), 3.56-3.67 (1H, m), 4.27-4.42 (1H, m), 4.51-4.73 (2H, m), 5.24-5.49 (1H, m), 6.17 (1H, br d, J=8.2 Hz), 6.91-7.04 (2H, m), 7.12-7.18 (1H, m), 7.27-7.42 (3H, m).

### Example 412

### N-({(2R,3R)-3-fluoro-1-[(1S,2S)-2-fluoro-2-(2′,5,6′-trifluoro[1,1′-biphenyl]-2-yl)cyclopropane-1-carbonyl]azetidin-2-yl}methyl)ethanesulfonamide

### A) 2-bromo-1-ethenyl-4-fluorobenzene

To a solution of methyltriphenylphosphonium iodide (120 g) in THF (800 mL) was added n-butyllithium (108 mL, 2.5 M in hexane) dropwise at 0° C. under N₂ atmosphere. The reaction mixture was stirred for 1 h at 0° C. and a solution of 2-bromo-4-fluorobenzaldehyde (50.0 g) in THF (300 mL) was added slowly at −78° C. Then it was allowed to warm to 25° C. and stirred at 25° C. for 16 h. The mixture was diluted with water and extracted with EtOAc. The organic layer was concentrated under reduced pressure to give a residue. The residue was purified by silica gel column chromatography (petroleum ether) to give the title compound (33.6 g).

¹H NMR (400 MHz, CDCl₃) δ 5.34 (1H, d, J=11.2 Hz), 5.63 (1H, d, J=17.2 Hz), 6.90-7.05 (2H, m), 7.27-7.32 (1H, m), 7.49-7.56 (1H, m).

### B) 2-bromo-1-(2-bromo-1-fluoroethyl)-4-fluorobenzene

To a mixture of 2-bromo-1-ethenyl-4-fluorobenzene (33.6 g) in DCM (400 mL) were added triethylamine trihydrofluoride (80.8 g) and N-bromosuccinimide (59.5 g) at 0° C., then the mixture was stirred at 20° C. for 16 h. The mixture was concentrated under reduced pressure. The residue was purified by silica gel column chromatography (petroleum ether) to give the title compound (40.2 g).

¹H NMR (400 MHz, CDCl₃) δ 3.56-3.65 (1H, m), 3.71-3.87 (1H, m), 5.83-6.05 (1H, m), 7.11-7.20 (1H, m), 7.31-7.40 (1H, m), 7.50-7.58 (1H, m).

### C) 2-bromo-4-fluoro-1-(1-fluoroethenyl)benzene

To a mixture of 2-bromo-1-(2-bromo-1-fluoroethyl)-4-fluorobenzene (30.3 g) in DCM (300 mL) was added DBU (23.1 g), then the mixture was stirred at 50° C. for 16 h. The mixture was concentrated under reduced pressure to give a residue. The residue was purified by silica gel column chromatography (petroleum ether) to give the title compound (18.5 g).

¹H NMR (400 MHz, CDCl₃) δ 4.86-5.01 (1H, m), 5.05-5.13 (1H, m), 7.00-7.10 (1H, m), 7.35-7.41 (1H, m), 7.43-7.52 (1H, m).

### D) 2′,5,6′-trifluoro-2-(1-fluoroethenyl)-1,1′-biphenyl

This reaction was conducted in three batches. A mixture of 2-bromo-4-fluoro-1-(1-fluoroethenyl)benzene (13.8 g×3), 2-(2,6-difluorophenyl)-4,4,5,5-tetramethyl-1,3,2-dioxaborolane (30.3 g×3), cesium carbonate (82.1 g×3), copper (I) chloride (6.24 g×3), palladium (II) acetate (566 mg×3) and SPhos (5.17 g×3) in DMF (200 mL×3) were stirred at 100° C. for 20 min under N₂ atmosphere. The three reaction mixtures were combined and filtered. The filtrate was diluted with water, and then extracted with EtOAc. The combined organic layer was concentrated under reduced pressure to afford a crude product. The crude product was purified by silica gel column chromatography (petroleum ether) to give the title compound (22.9 g).

¹H NMR (400 MHz, CDCl₃) δ 4.24-4.49 (1H, m), 4.71-4.81 (1H, m), 6.94-7.07 (3H, m), 7.11-7.20 (1H, m), 7.29-7.41 (1H, m), 7.58-7.65 (1H, m).

### E) ethyl (1S,2S)-2-fluoro-2-(2′,5,6′-trifluoro[1,1′-biphenyl]-2-yl)cyclopropane-1-carboxylate

This reaction was conducted in four batches. To a mixture of dichloro(p-cymene)ruthenium(II) (1.10 g x 4) and (S,S)-pybox(iPr) (1.09 g x 4) in THF (20 mL×4) was added a solution of 2′,5,6′-trifluoro-2-(1-fluoroethenyl)-1,1′-biphenyl (17.5 g×4) in THF (120 mL×4) at 20° C. under N₂ atmosphere. After the mixture was heated at 60° C., and to the mixture was added a solution of ethyl 2-diazoacetate (19.8 g x 4) in toluene (130 mL×4) dropwise over 5 h under N₂ atmosphere. And then it was stirred at 60° C. for 12 h. The four reaction mixtures were combined and concentrated under reduced pressure to afford a residue. The residue was purified by silica gel column chromatography (petroleum ether, then EtOAc/petroleum ether) and reversed column (C18, MeOH/water) to give crude ethyl 2-fluoro-2-(2′,5,6′-trifluoro[1,1′-biphenyl]-2-yl)cyclopropane-1-carboxylate (35.3 g).

The crude ethyl 2-fluoro-2-(2′,5,6′-trifluoro[1,1′-biphenyl]-2-yl)cyclopropane-1-carboxylate (750 mg) was purified by SFC (column: DAICEL CHIRALPAK IC, 250 mm×30 mm, 10 μm, mobile phase: CO₂/IPA containing 0.1% aqueous ammonia=75/25 v/v) The fractions of the 3^{rd} peak with the longest retention time were concentrated under reduced pressure to give the title compound (660 mg).

¹H NMR (400 MHz, CDCl₃) δ 1.17-1.23 (3H, m), 1.33-1.43 (1H, m), 1.87-1.98 (1H, m), 2.01-2.09 (1H, m), 3.94-4.04 (1H, m), 4.05-4.17 (1H, m), 6.92-7.08 (3H, m), 7.12-7.21 (1H, m), 7.32-7.42 (1H, m), 7.56-7.64 (1H, m).

### F) (1S,2S)-2-fluoro-2-(2′,5,6′-trifluoro[1,1′-biphenyl]-2-yl)cyclopropane-1-carboxylic Acid

To a mixture of ethyl (1S,2S)-2-fluoro-2-(2′,5,6′-trifluoro[1,1′-biphenyl]-2-yl)cyclopropane-1-carboxylate (3.30 g) in THF (15 mL), MeOH (15 mL) and water (15 mL) was added NaOH (3.90 g), and then the mixture was stirred at 20° C. for 16 h. The reaction mixture was acidified with 4M HCl aq. to pH=6-7. The mixture was extracted with EtOAc. The combined organic layer was concentrated under reduced pressure to afford the title compound (3.30 g).

¹H NMR (400 MHz, DMSO-d₆) δ 1.40-1.50 (1H, m), 1.56-1.63 (1H, m), 2.04-2.11 (1H, m), 7.15-7.22 (2H, m), 7.32-7.43 (2H, m), 7.48-7.58 (1H, m), 7.68-7.74 (1H, m).

### G) N-({(2R,3R)-3-fluoro-1-[(1S,2S)-2-fluoro-2-(2′,5,6′-trifluoro[1,1′-biphenyl]-2-yl)cyclopropane-1-carbonyl]azetidin-2-yl}methyl)ethanesulfonamide

To a cooled (0° C.) mixture of tert-butyl (2R,3R)-2-(ethylsulfonamidomethyl)-3-fluoroazetidine-1-carboxylate (5.87 g), toluene (30 mL), and water (1.5 mL) was added TFA (15 mL) and the mixture was stirred at rt for 2.5 h. The mixture was concentrated in vacuo, followed by being azeotroped with toluene (20 mL×2), to give N-(((2R,3R)-3-fluoroazetidin-2-yl)methyl)ethanesulfonamide 2,2,2-trifluoroacetate (6.15 g) as a crude material which was used in the next step.

To a cooled (0° C.) mixture of (1S,2S)-2-fluoro-2-(2′,5,6′-trifluoro[1,1′-biphenyl]-2-yl)cyclopropane-1-carboxylic acid (6.15 g) and DMF (20 mL) were successively added EDCI (3.99 g), HOBt (2.81 g), and DIPEA (13.9 mL) and the mixture was stirred rt for 1 h. To the mixture was added N-(((2R,3R)-3-fluoroazetidin-2-yl)methyl)ethanesulfonamide 2,2,2-trifluoroacetate (6.15 g) in DMF (20 mL) at 0° C. and the mixture was stirred at rt overnight. The mixture was diluted with EtOAc and aq. NaHCO₃ at 0° C. and the mixture was stirred at the same temperature for 5 min. The organic layer was separated, washed with brine, dried over Na₂SO₄, filtered through a silica gel/NH silica gel pad, and concentrated in vacuo. The residue was purified by silica gel column chromatography (EtOAc/hexane) to give the title compound (8.81 g) as a white amorphous solid.

The amorphous solid of N-({(2R,3R)-3-fluoro-1-[(1S,2S)-2-fluoro-2-(2′,5,6′-trifluoro[1,1′-biphenyl]-2-yl)cyclopropane-1-carbonyl]azetidin-2-yl}methyl)ethanesulfonamide (1.25 g) was suspended in EtOAc/hexane and a seed crystal was added to the mixture. After crystallized, the mixture was concentrated in vacuo. The residual solid (1.25 g) was dissolved in EtOAc and hexane was added thereto dropwise at 50° C. to make a saturated solution. Then, the seed crystal was added thereto and the mixture was stirred at the same temperature for 30 min to form a precipitate, and then cooled to rt and stirred at the same temperature overnight. The resulting precipitate was collected by filtration to give the title compound (1.13 g) as a white solid.

¹H NMR (400 MHz, CDCl₃) δ 1.25-1.38 (4H, m), 1.86-2.01 (2H, m), 3.01 (2H, q, J=7.4 Hz), 3.42-3.68 (2H, m), 4.27-4.42 (1H, m), 4.47-4.72 (2H, m), 5.25-5.48 (1H, m), 6.06 (1H, br d, J=8.9 Hz), 6.95-7.03 (2H, m), 7.05-7.09 (1H, m), 7.12-7.21 (1H, m), 7.33-7.46 (2H, m).

### Example 420

### N-({(2R,3R)-1-[(1R,2R)-2-(5-chloro-2′,6′-difluoro [1,1′-biphenyl]-2-yl)-2-fluorocyclopropane-1-carbonyl]-3-fluoroazetidin-2-yl}methyl)ethanesulfonamide, or N-({(2R,3R)-1-[(1S,2S)-2-(5-chloro-2′,6′-difluoro[1,1′-biphenyl]-2-yl)-2-fluorocyclopropane-1-carbonyl]-3-fluoroazetidin-2-yl}methyl)ethanesulfonamide

### A) 2-bromo-4-chloro-1-ethenylbenzene

To a solution of methyltriphenylphosphonium iodide (55.3 g) in THF (300 mL) was added n-BuLi (50.1 mL, 2.5 M in hexane) dropwise at 0° C. under N₂ atmosphere. The resulting orange reaction mixture was stirred for 1 h at 0° C. and 2-bromo-4-chlorobenzaldehyde (25.0 g) in THF (150 mL) was added slowly at −78° C. Then it was allowed to warm to 25° C. and stirred at 25° C. for 16 h. The reaction mixture was diluted with water and extracted with EtOAc. The combined organic layer was washed with brine, dried over Na₂SO₄, filtrated, and concentrated in vacuum. The residue was purified by silica gel column chromatography (petroleum ether) to give the title compound (15.9 g).

¹H NMR (400 MHz, CDCl₃) δ 5.38 (1H, d, J=11.2 Hz), 5.69 (1H, d, J=17.2 Hz), 6.94-7.04 (1H, m), 7.24-7.29 (1H, m), 7.45-7.50 (1H, d, J=8.4 Hz), 7.55-7.58 (1H, m)

### B) 2-bromo-4-chloro-1-(1-fluoroethenyl)benzene

To a mixture of 2-bromo-1-ethenyl-4-chlorobenzene (15.9 g) in DCM (150 mL) were added triethylamine trihydrofluoride (23.6 g) and NBS (15.6 g) at 0° C., then the mixture was stirred at 20° C. for 16 h. The mixture was concentrated under reduced pressure. The residue was purified by silica gel column chromatography (petroleum ether) to give crude 2-bromo-1-(2-bromo-1-fluoroethyl)-4-chlorobenzene (14.4 g).

To a mixture of the crude 2-bromo-1-(2-bromo-1-fluoroethyl)-4-chlorobenzene (14.4 g) in DCM (150 mL) was added DBU (8.31 g), then the mixture was stirred at 50° C. for 12 h. The mixture was concentrated under reduced pressure. The residue was purified by silica gel column chromatography (petroleum ether) to give title compound (9.60 g).

¹H NMR (400 MHz, CDCl₃) δ 4.78-5.32 (2H, m), 7.26-7.36 (1H, m), 7.38-7.47 (1H, m), 7.56-7.67 (1H, m).

### C) 5-chloro-2′,6′-difluoro-2-(1-fluoroethenyl)-1,1′-biphenyl

A mixture of 2-(2,6-difluorophenyl)-4,4,5,5-tetramethyl-1,3,2-dioxaborolane (18.4 g), 2-bromo-4-chloro-1-(1-fluoroethenyl)benzene (9.00 g), Cs₂CO₃ (49.8 g), CuCl (3.78 g), Pd(OAc)₂ (343 mg) and SPhos (3.14 g) in DMF (100 mL) was stirred at 100° C. for 20 min under N₂ atmosphere. The mixture was diluted with EtOAc and then filtered. The filtrate was washed by water, dried over Na₂SO₄, and concentrated in vacuo. The residue was purified by silica gel column chromatography (petroleum ether) to give title compound (4.40 g).

¹H NMR (400 MHz, CDCl₃) δ 4.46 (1H, dd, J=48.8, 3.6 Hz), 4.80 (1H, dd, J=17.6, 3.6 Hz), 6.94-7.01 (2H, m), 7.32-7.45 (3H, m), 7.53-7.59 (1H, m).

### D) ethyl (1R*,2R*)-2-(5-chloro-2′,6′-difluoro[1,1′-biphenyl]-2-yl)-2-fluorocyclopropane-1-carboxylate

To a mixture of dichloro(p-cymene)ruthenium(II) (261 mg) and (S,S)-pybox (257 mg) in THF (20 mL) was added a solution of 5-chloro-2′,6′-difluoro-2-(1-fluoroethenyl)-1,1′-biphenyl (4.40 g) in THF (50 mL) dropwise at 20° C. under N₂ atmosphere, and the mixture was heated at 55° C., and to the mixture was added a solution ethyl 2-diazoacetate (4.67 g) in toluene (50 mL) dropwise over 3 h under N2 atmosphere. And it was stirred at 55° C. for 14 h under N₂ atmosphere. It was concentrated under vacuum. The residue was purified by silica gel column chromatography (EtOAc/petroleum ether) and reversed column chromatography (C18, MeOH/water) to give crude ethyl 2-(5-chloro-2′,6′-difluoro[1,1′-biphenyl]-2-yl)-2-fluorocyclopropane-1-carboxylate (2.00 g).

The crude ethyl 2-(5-chloro-2′,6′-difluoro[1,1′-biphenyl]-2-yl)-2-fluorocyclopropane-1-carboxylate (2.00 g) was purified by SFC (column: DAICEL CHIRALPAK IC, 250 mm×50 mm, 10 μm, mobile phase: CO₂/EtOH containing 0.1% aqueous ammonia=85/15 v/v). The fractions of the 3^{rd} peak with the longest retention time were concentrated under reduced pressure and lyophilized to give the title compound (1.10 g).

¹H NMR (400 MHz, CDCl₃) δ 1.20 (3H, t, J=6.8 Hz), 1.34-1.44 (1H, m), 1.85-2.10 (2H, m), 3.90-4.15 (2H, m), 6.90-7.05 (2H, m), 7.30-7.60 (4H, m).

### E) (1R*,2R*)-2-(5-chloro-2′,6′-difluoro[1,1′-biphenyl]-2-yl)-2-fluorocyclopropane-1-carboxylic Acid

To a mixture of ethyl (1R*,2R*)-2-(5-chloro-2′,6′-difluoro[1,1′-biphenyl]-2-yl)-2-fluorocyclopropane-1-carboxylate (1.10 g) synthesized in Step D) in MeOH (3 mL), THF (3 mL) and water (3 mL) was added NaOH (1.24 g), and then the mixture was stirred at 25° C. for 16 h. The reaction mixture was acidified with 1 M HCl aq. to pH=3. The mixture was extracted with EtOAc. The combined organic layer was washed with brine, dried over Na₂SO₄ and then concentrated under reduced pressure to give the title compound (978 mg).

¹H NMR (400 MHz, CDCl₃) δ 1.41-1.52 (1H, m), 1.87-2.11 (2H, m), 6.91-7.03 (2H, m), 7.29-7.40 (2H, m), 7.43-7.57 (2H, m).

### F) N-({(2R,3R)-1-[(1R,2R)-2-(5-chloro-2′,6′-difluoro[1,1′-biphenyl]-2-yl)-2-fluorocyclopropane-1-carbonyl]-3-fluoroazetidin-2-yl}methyl)ethanesulfonamide, or N-({(2R,3R)-1-[(1S,2S)-2-(5-chloro-2′,6′-difluoro[1,1′-biphenyl]-2-yl)-2-fluorocyclopropane-1-carbonyl]-3-fluoroazetidin-2-yl}methyl)ethanesulfonamide

To a solution of tert-butyl (2R,3R)-2-(ethylsulfonamidomethyl)-3-fluoroazetidine-1-carboxylate (422 mg) in toluene (12 mL) was added TFA (4 mL) at rt and the resulting mixture was stirred for 1 h. The solvent was removed in vacuo to yield crude N-(((2R,3R)-3-fluoroazetidin-2-yl)methyl)ethanesulfonamide 2,2,2-trifluoroacetate (442 mg) which was used without further purification.

To a solution of (1R*,2R*)-2-(5-chloro-2′,6′-difluoro[1,1′-biphenyl]-2-yl)-2-fluorocyclopropane-1-carboxylic acid (558 mg) synthesized in Step E), the crude N-(((2R,3R)-3-fluoroazetidin-2-yl)methyl)ethanesulfonamide 2,2,2-trifluoroacetate (442 mg) and TEA (0.993 mL) in DMF(10 mL) was added HATU (1.09 g) at rt and the resulting solution was stirred for 1 h. The mixture was quenched with sat. NH₄Cl aq. at rt and extracted with EtOAc. The organic layer was separated, washed with brine, dried over MgSO₄ and concentrated in vacuo. The residue was purified by silica gel column chromatography (EtOAc/hexane) to give the title compound (656 mg).

¹H NMR (400 MHz, CDCl₃) δ 1.29-1.40 (4H, m), 1.88-2.02 (2H, m), 2.95-3.08 (2H, m), 3.44-3.64 (2H, m), 4.26-4.41 (1H, m), 4.49-4.73 (2H, m), 5.27-5.47 (1H, m), 6.00-6.09 (1H, m), 6.92-7.05 (2H, m), 7.31-7.48 (4H, m).

### Example 423

### N-({(2R,3R)-3-fluoro-1-[(1S,2S)-2-fluoro-2-(2′,5,6′-trifluoro[1,1′-biphenyl]-2-yl)cyclopropane-1-carbonyl]azetidin-2-yl}methyl)-N′-methylsulfuric Diamide

### A) tert-butyl (2R,3R)-3-fluoro-2-(((N-methylsulfamoyl)amino)methyl)azetidine-1-carboxylate

A mixture of tert-butyl (2R,3R)-2-(azidomethyl)-3-fluoroazetidine-1-carboxylate (2.50 g) and 20% Pd(OH)₂ on carbon (0.723 g) in EtOAc (35 mL) was hydrogenated under balloon pressure at rt for 4.5 h. The catalyst was removed by filtration through a Celite pad eluted with EtOAc and the filtrate was concentrated in vacuo. The residue was dissolved in THF (30 mL), and then TEA (4.54 mL), DMAP (0.133 g), and methylsulfamoyl chloride (1.09 mL) in THF (5 mL) were successively added thereto at 0° C. The mixture was stirred at rt overnight. The mixture was diluted with EtOAc and aq. NaHCO₃ at 0° C. and the mixture was stirred at the same temperature for 5 min. The organic layer was separated, washed with brine, dried over Na₂SO₄, filtered through a silica gel/NH silica gel pad, and concentrated in vacuo. The residue was purified by silica gel column chromatography (EtOAc/hexane) to give the title compound (2.11 g).

¹H NMR (400 MHz, CDCl₃) δ 1.46 (9H, s), 2.68-2.78 (3H, m), 3.35-3.54 (2H, m), 3.90-4.05 (1H, m), 4.10-4.29 (2H, m), 4.48-4.63 (1H, m), 5.15-5.44 (1H, m), 5.81 (1H, br s).

### B) N-({(2R,3R)-3-fluoro-1-[(1S,2S)-2-fluoro-2-(2′,5,6′-trifluoro[1,1′-biphenyl]-2-yl)cyclopropane-1-carbonyl]azetidin-2-yl}methyl)-N′-methylsulfuric Diamide

A mixture of tert-butyl (2R,3R)-3-fluoro-2-(((N-methylsulfamoyl)amino)methyl)azetidine-1-carboxylate (2.08 g), p-toluenesulfonic acid monohydrate (2.66 g), and EtOAc (20 mL) was refluxed for 1 h. The mixture was concentrated in vacuo to give N-{[(2R,3R)-3-fluoroazetidin-2-yl]methyl}-N′-methylsulfuric diamide 4-methylbenzenesulfonate (2.58 g) as a crude material which was used in the next step.

To a cooled (0° C.) mixture of the crude N-{[(2R,3R)-3-fluoroazetidin-2-yl]methyl}-N′-methylsulfuric diamide 4-methylbenzenesulfonate (2.58 g) and DMF (20 mL) were successively added (1S,2S)-2-fluoro-2-(2′,5,6′-trifluoro[1,1′-biphenyl]-2-yl)cyclopropane-1-carboxylic acid (2.17 g), EDCI (1.61 g), HOBt (1.13 g), and DIPEA (4.88 mL) and the mixture was stirred at rt overnight. The mixture was diluted with EtOAc and aq. NaHCO₃ at 0° C. and the mixture was stirred at the same temperature for 5 min. The organic layer was separated, washed with brine, dried over Na₂SO₄, filtered through a silica gel/NH silica gel pad, and concentrated in vacuo. A part of the residue was purified by silica gel column chromatography (EtOAc/hexane) to give the title compound as a white amorphous solid. This material was suspended in EtOAc (10 mL)/heptane (20 mL) with sonication and a seed crystal was added thereto and the suspension was further sonicated for 15 min until the solid was formed. The mixture was concentrated in vacuo and the residual solid (1.75 g) was dissolved in EtOAc (16 mL) at 80° C. with stirring and heptane (8 mL) was added dropwise thereto to form a suspension. After addition of a seed crystal, the resultant suspension was stirred at 80° C. for 5 min and cooled to rt and stirred overnight. To the suspension was added heptane (4 mL) and stirred at rt for further 30 min. The precipitates were collected and dried in vacuo at 60° C. to give the title compound (1.66 g) as a white solid.

¹H NMR (400 MHz, CDCl₃) δ 1.24-1.37 (1H, m), 1.86-2.05 (2H, m), 2.69 (3H, d, J=5.5 Hz), 3.40-3.56 (2H, m), 4.11-4.21 (1H, m), 4.29-4.43 (1H, m), 4.50-4.63 (1H, m), 4.66-4.78 (1H, m), 5.25-5.48 (1H, m), 5.95-6.04 (1H, m), 6.95-7.03 (2H, m), 7.04-7.10 (1H, m), 7.12-7.20 (1H, m), 7.34-7.54 (2H, m).

The compounds of Examples are shown in the following tables. MS in the tables means actual measured value. The compounds of Examples 1 to 12, 14 to 22, 26 to 132, 134 to 138, 140 to 194, 196 to 199, 201 to 214, 217 to 225, 227 to 230, 233 to 237, 239 to 258, 260, 261, 263, 265 to 293, 295 to 303, 305, 307 to 317, 320-352, 355-357, 359-368, 370-392, 394-410, 413-419, 421, 422, and 424-450 in the following tables were produced according to the methods described in the above-mentioned Examples, or methods analogous thereto.

TABLE 1

| Ex. No. | IUPAC Name | Structure | MS |
|---|---|---|---|
| 1 | a mixture of rac-N-{(4R)-1-[(1R,2R)-2-([1,1′- biphenyl]-2-yl)cyclopropane-1- carbonyl]azepan-4-yl}methanesulfonamide and rac-N-{(4S)-1-[(1R,2R)-2-([1,1′- biphenyl]-2-yl)cyclopropane-1- carbonyl]azepan-4-yl}methanesulfonamide | 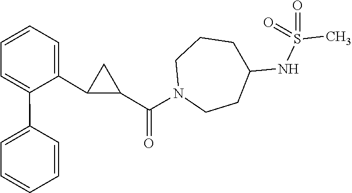 [SMILES: CS(=O)(=O)NC1CCCN(C(=O)C2CC2c2ccccc2-c2ccccc2)CC1] | 413.3 |
| 2 | rac-N-{1-[(1R,2R)-2-([1,1′-biphenyl]-2- yl)cyclopropane-1-carbonyl]piperidin-4- yl}methanesulfonamide | 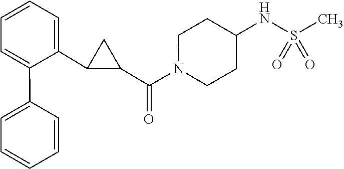 [SMILES: CS(=O)(=O)NC1CCN(C(=O)C2CC2c2ccccc2-c2ccccc2)CC1] | 399.1 |
| 3 | a mixture of rac-N-{(3R)-1-[(1R,2R)-2-(2′- fluoro-6′-methyl[1,1′-biphenyl]-2- yl)cyclopropane-1-carbonyl]pyrrolidin-3- yl}methanesulfonamide and rac-N-{(3S)-1- [(1R,2R)-2-(2′-fluoro-6′-methyl[1,1′-biphenyl]- 2-yl)cyclopropane-1-carbonyl]pyrrolidin-3- yl}methanesulfonamide | 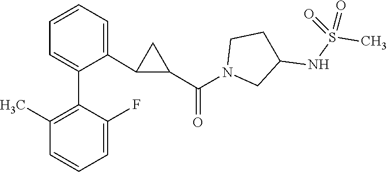 | 417.2 |
| 4 | a mixture of rac-N-{(3R)-1-[(1R,2R)-2-(2′,6′- difluoro[1,1′-biphenyl]-2-yl)cyclopropane-1- carbonyl]pyrrolidin-3-yl}methanesulfonamide and rac-N-{(3S)-1-[(1R,2R)-2-(2′,6′- difluoro[1,1′-biphenyl]-2-yl)cyclopropane-1- carbonyl]pyrrolidin-3-yl}methanesulfonamide | 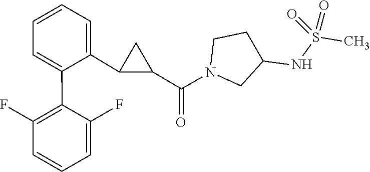 [SMILES: CS(=O)(=O)NC1CCN(C(=O)C2CC2c2ccccc2-c2c(F)cccc2F)C1] | 421.1 |
| 5 | rac-N-{2-[(1R,2R)-2-(2′,6′-difluoro[1,1′- biphenyl]-2-yl)cyclopropane-1- carbonyl]octahydrocyclopenta[c]pyrrol-5- yl}methanesulfonamide | 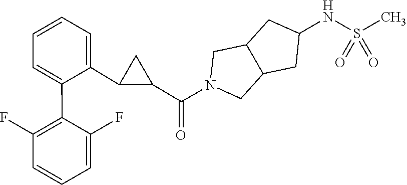 [SMILES: CS(=O)(=O)NC1CC2CN(C(=O)C3CC3c3ccccc3-c3c(F)cccc3F)CC2C1] | 461.2 |
| 6 | a mixture of rac-N-{(3R)-1-[(1R,2R)-2-(2′- fluoro-6′-methoxy[1,1′-biphenyl]-2- yl)cyclopropane-1-carbonyl]pyrrolidin-3- yl}methanesulfonamide and rac-N-{(3S)-1- [(1R,2R)-2-(2′-fluoro-6′-methoxy[1,1′- biphenyl]-2-yl)cyclopropane-1- carbonyl]pyrrolidin-3-yl}methanesulfonamide | 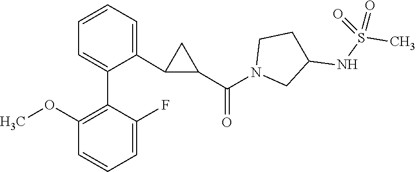 | 433.2 |
| 7 | a mixture of rac-N-{(3R)-1-[(1R,2R)-2-(4′- fluoro[1,1′-biphenyl]-2-yl)cyclopropane-1- carbonyl]pyrrolidin-3-yl}methanesulfonamide and rac-N-{(3S)-1-[(1R,2R)-2-(4′-fluoro[1,1′- biphenyl]-2-yl)cyclopropane-1- carbonyl]pyrrolidin-3-yl}methanesulfonamide | 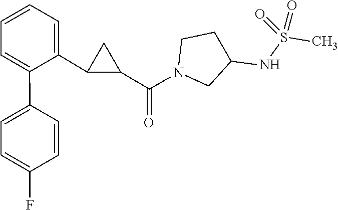 [SMILES: CS(=O)(=O)NC1CCN(C(=O)C2CC2c2ccccc2-c2ccc(F)cc2)C1] | 403.2 |
| 8 | a mixture of rac-N-{(3R)-1-[(1R,2R)-2-(3′- fluoro[1,1′-biphenyl]-2-yl)cyclopropane-1- carbonyl]pyrrolidin-3-yl}methanesulfonamide and rac-N-{(3S)-1-[(1R,2R)-2-(3′-fluoro[1,1′- biphenyl]-2-yl)cyclopropane-1- carbonyl]pyrrolidin-3-yl}methanesulfonamide | 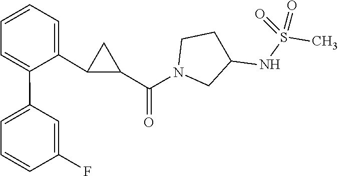 [SMILES: CS(=O)(=O)NC1CCN(C(=O)C2CC2c2ccccc2-c2cccc(F)c2)C1] | 403.2 |
| 9 | a mixture of N-{(3S)-1-[(1R,2R)-2-([1,1′- biphenyl]-2-yl)cyclopropane-1- carbonyl]piperidin-3-yl}methanesulfonamide and N-{(3S)-1-[(1S,2S)-2-([1,1′-biphenyl]-2- yl)cyclopropane-1-carbonyl]piperidin-3- yl}methanesulfonamide | 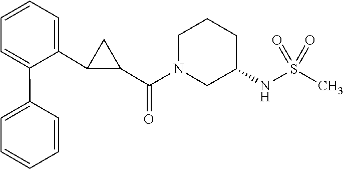 [SMILES: CS(=O)(=O)N[C@H]1CCCN(C(=O)C2CC2c2ccccc2-c2ccccc2)C1] | 399.2 |
| 10 | a mixture of rac-N-{(3R)-1-[(1R,2R)-2-(2′,4′- difluoro[1,1′-biphenyl]-2-yl)cyclopropane-1- carbonyl]pyrrolidin-3-yl}methanesulfonamide and rac-N-{(3S)-1-[(1R,2R)-2-(2′,4′- difluoro[1,1′-biphenyl]-2-yl)cyclopropane-1- carbonyl]pyrrolidin-3-yl}methanesulfonamide | 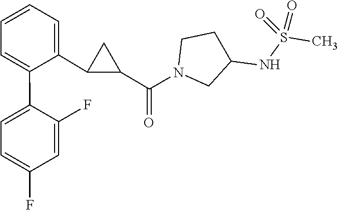 [SMILES: CS(=O)(=O)NC1CCN(C(=O)C2CC2c2ccccc2-c2ccc(F)cc2F)C1] | 421.2 |
| 11 | a mixture of rac-N-{(3R)-1-[(1R,2R)-2-([1,1′- biphenyl]-2-yl)cyclopropane-1- carbonyl]pyrrolidin-3-yl}methanesulfonamide and rac-N-{(3S)-1-[(1R,2R)-2-([1,1′- biphenyl]-2-yl)cyclopropane-1- carbonyl]pyrrolidin-3-yl}methanesulfonamide | 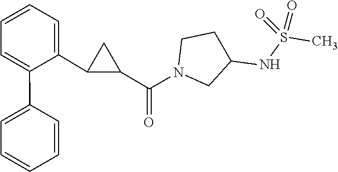 [SMILES: CS(=O)(=O)NC1CCN(C(=O)C2CC2c2ccccc2-c2ccccc2)C1] | 385.2 |
| 12 | a mixture of rac-N-{(3R)-1-[(1R,2R)-2-(2′- fluoro[1,1′-biphenyl]-2-yl)cyclopropane-1- carbonyl]pyrrolidin-3-yl}methanesulfonamide and rac-N-{(3S)-1-[(1R,2R)-2-(2′-fluoro[1,1′- biphenyl]-2-yl)cyclopropane-1- carbonyl]pyrrolidin-3-yl}methanesulfonamide | 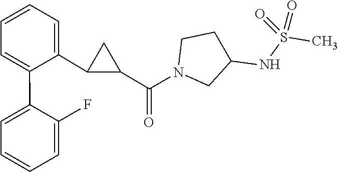 [SMILES: CS(=O)(=O)NC1CCN(C(=O)C2CC2c2ccccc2-c2ccccc2F)C1] | 403.3 |
| 14 | a mixture of N-{(3R)-1-[(1R,2R)-2-([1,1′- biphenyl]-2-yl)cyclopropane-1- carbonyl]pyrrolidin-3-yl}methanesulfonamide and N-{(3R)-1-[(1S,2S)-2-([1,1′-biphenyl]-2- yl)cyclopropane-1-carbonyl]pyrrolidin-3- yl}methanesulfonamide | 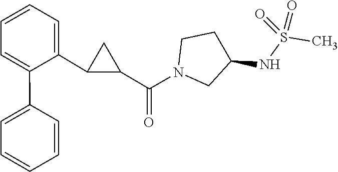 [SMILES: CS(=O)(=O)N[C@@H]1CCN(C(=O)C2CC2c2ccccc2-c2ccccc2)C1] | 385.2 |
| 15 | a mixture of rac-N-{(3R)-1-[(1R,2R)-2-(2′- chloro-6′-fluoro[1,1′-biphenyl]-2- yl)cyclopropane-1-carbonyl]pyrrolidin-3- yl}methanesulfonamide and rac-N-{(3S)-1- [(1R,2R)-2-(2′-chloro-6′-fluoro[1,1′-biphenyl]- 2-yl)cyclopropane-1-carbonyl]pyrrolidin-3- yl}methanesulfonamide | 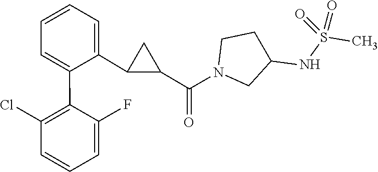 [SMILES: CS(=O)(=O)NC1CCN(C(=O)C2CC2c2ccccc2-c2c(F)cccc2Cl)C1] | 437.1 |
| 16 | rac-N-{1-[(1R,2R)-2-([1,1′-biphenyl]-2- yl)cyclopropane-1-carbonyl]azetidin-3- yl}methanesulfonamide | 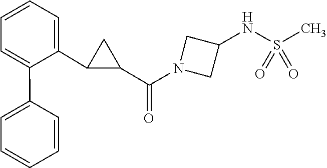 [SMILES: CS(=O)(=O)NC1CN(C(=O)C2CC2c2ccccc2-c2ccccc2)C1] | 371.1 |
| 17 | a mixture of rac-N-{(3R)-1-[(1R,2R)-2-([1,1′- biphenyl]-2-yl)cyclopropane-1- carbonyl]piperidin-3-yl}methanesulfonamide and rac-N-{(3S)-1-[(1R,2R)-2-([1,1′- biphenyl]-2-yl)cyclopropane-1- carbonyl]piperidin-3-yl}methanesulfonamide | 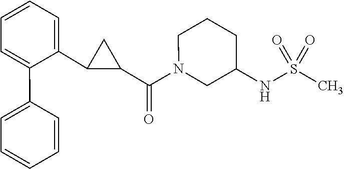 [SMILES: CS(=O)(=O)NC1CCCN(C(=O)C2CC2c2ccccc2-c2ccccc2)C1] | 399.2 |
| 18 | a mixture of N-{(3S)-1-[(1R,2R)-2-([1,1′- biphenyl]-2-yl)cyclopropane-1- carbonyl]pyrrolidin-3-yl}methanesulfonamide and N-[(3S)-1-[(1S,2S)-2-([1,1′-biphenyl]-2- yl)cyclopropane-1-carbonyl]pyrrolidin-3- yl]methanesulfonamide | 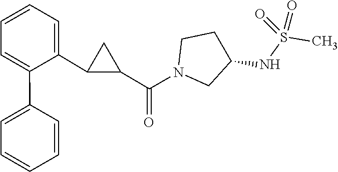 [SMILES: CS(=O)(=O)N[C@H]1CCN(C(=O)C2CC2c2ccccc2-c2ccccc2)C1] | 385.1 |
| 19 | a mixture of rac-N-{(3R)-1-[(1R,2R)-2-(6- fluoro[1,1′-biphenyl]-2-yl)cyclopropane-1- carbonyl]pyrrolidin-3-yl}methanesulfonamide and rac-N-{(3S)-1-[(1R,2R)-2-(6-fluoro[1,1′- biphenyl]-2-yl)cyclopropane-1- carbonyl]pyrrolidin-3-yl}methanesulfonamide | 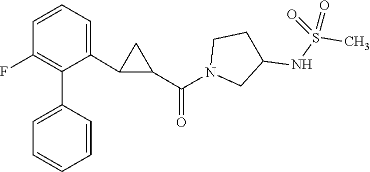 [SMILES: CS(=O)(=O)NC1CCN(C(=O)C2CC2c2cccc(F)c2-c2ccccc2)C1] | 403.2 |
| 20 | a mixture of rac-N-{(3R)-1-[(1R,2R)-2-(5- fluoro[1,1′-biphenyl]-2-yl)cyclopropane-1- carbonyl]pyrrolidin-3-yl}methanesulfonamide and rac-N-{(3S)-1-[(1R,2R)-2-(5-fluoro[1,1′- biphenyl]-2-yl)cyclopropane-1- carbonyl]pyrrolidin-3-yl}methanesulfonamide | 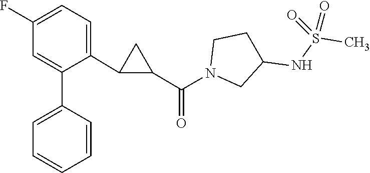 [SMILES: CS(=O)(=O)NC1CCN(C(=O)C2CC2c2ccc(F)cc2-c2ccccc2)C1] | 403.2 |
| 21 | a mixture of rac-N-{(3R)-1-[(1R,2R)-2-(4- fluoro[1,1′-biphenyl]-2-yl)cyclopropane-1- carbonyl]pyrrolidin-3-yl}methanesulfonamide and rac-N-{(3S)-1-[(1R,2R)-2-(4-fluoro[1,1′- biphenyl]-2-yl)cyclopropane-1- carbonyl]pyrrolidin-3-yl}methanesulfonamide | 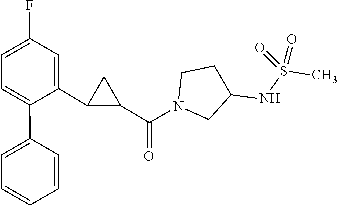 [SMILES: CS(=O)(=O)NC1CCN(C(=O)C2CC2c2cc(F)ccc2-c2ccccc2)C1] | 403.2 |
| 22 | a mixture of rac-N-{(3R)-1-[(1R,2R)-2-(3- fluoro[1,1′-biphenyl]-2-yl)cyclopropane-1- carbonyl]pyrrolidin-3-yl}methanesulfonamide and rac-N-{(3S)-1-[(1R,2R)-2-(3-fluoro[1,1′- biphenyl]-2-yl)cyclopropane-1- carbonyl]pyrrolidin-3-yl}methanesulfonamide | 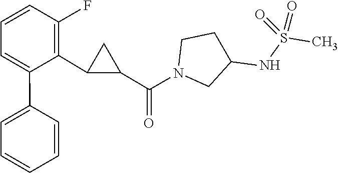 [SMILES: CS(=O)(=O)NC1CCN(C(=O)C2CC2c2c(F)cccc2-c2ccccc2)C1] | 403.2 |
| 23 | N-{(3S)-1-[(1R,2R)-2-(2′,6′-difluoro[1,1′- biphenyl]-2-yl)cyclopropane-1- carbonyl]pyrrolidin-3-yl}methanesulfonamide | 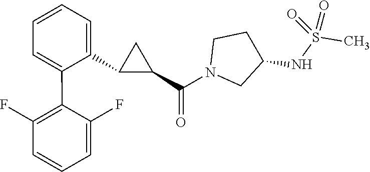 [SMILES: CS(=O)(=O)N[C@H]1CCN(C(=O)[C@@H]2C[C@H]2c2ccccc2-c2c(F)cccc2F)C1] | 421.2 |
| 24 | N-{(3S)-1-[(1R,2R)-2-(2′,6′-difluoro[1,1′- biphenyl]-2-yl)cyclopropane-1- carbonyl]piperidin-3-yl}methanesulfonamide | 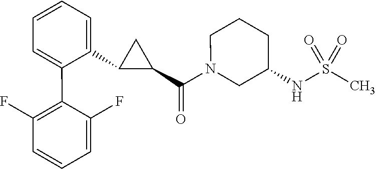 [SMILES: CS(=O)(=O)N[C@H]1CCCN(C(=O)[C@@H]2C[C@H]2c2ccccc2-c2c(F)cccc2F)C1] | 435.3 |
| 25 | N-{(4S)-1-[(1R,2R)-2-(2′,6′-difluoro[1,1′- biphenyl]-2-yl)cyclopropane-1- carbonyl]azepan-4-yl}methanesulfonamide | 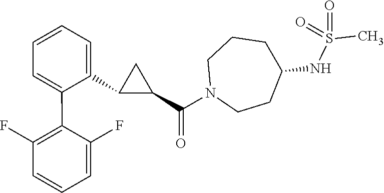 [SMILES: CS(=O)(=O)N[C@H]1CCCN(C(=O)[C@@H]2C[C@H]2c2ccccc2-c2c(F)cccc2F)CC1] | 449.2 |
| 26 | a mixture of rac-N-{(3R)-1-[(1R,2R)-2-(2′,3,6′- trifluoro[1,1′-biphenyl]-2-yl)cyclopropane-1- carbonyl]pyrrolidin-3-yl}methanesulfonamide and rac-N-{(3S)-1-[(1R,2R)-2-(2′,3,6′- trifluoro[1,1′-biphenyl]-2-yl)cyclopropane-1- carbonyl]pyrrolidin-3-yl}methanesulfonamide | 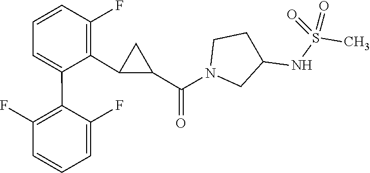 [SMILES: CS(=O)(=O)NC1CCN(C(=O)C2CC2c2c(F)cccc2-c2c(F)cccc2F)C1] | 439.1 |
| 27 | a mixture of rac-N-{(3R)-1-[(1R,2R)-2-(2′,6′- difluoro[1,1′-biphenyl]-2-yl)cyclopropane-1- carbonyl]pyrrolidin-3-yl}ethanesulfonamide and rac-N-{(3S)-1-[(1R,2R)-2-(2′,6′- difluoro[1,1′-biphenyl]-2-yl)cyclopropane-1- carbonyl]pyrrolidin-3-yl}ethanesulfonamide | 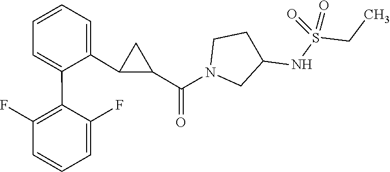 [SMILES: CCS(=O)(=O)NC1CCN(C(=O)C2CC2c2ccccc2-c2c(F)cccc2F)C1] | 435.2 |
| 28 | a mixture of rac-N-{(3R)-1-[(1R,2R)-2-(2′,6′- difluoro[1,1′-biphenyl]-2-yl)cyclopropane-1- carbonyl]piperidin-3-yl}ethanesulfonamide and rac-N-{(3S)-1-[(1R,2R)-2-(2′,6′- difluoro[1,1′-biphenyl]-2-yl)cyclopropane-1- carbonyl]piperidin-3-yl}ethanesulfonamide | 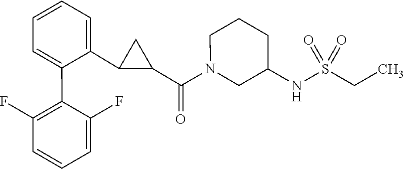 [SMILES: CCS(=O)(=O)NC1CCCN(C(=O)C2CC2c2ccccc2-c2c(F)cccc2F)C1] | 449.2 |
| 29 | a mixture of rac-N-{(4R)-1-[(1S,3S)-3-([1,1′- biphenyl]-2-yl)-2,2-dimethylcyclopropane-1- carbonyl]azepan-4-yl}methanesulfonamide and rac-N-{(4S)-1-[(1S,3S)-3-([1,1′- biphenyl]-2-yl)-2,2-dimethylcyclopropane-1- carbonyl]azepan-4-yl}methanesulfonamide | 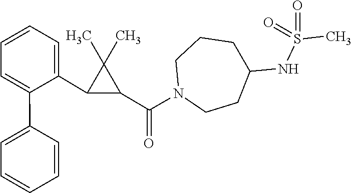 [SMILES: CC1(C)C(C(=O)N2CCCC(NS(C)(=O)=O)CC2)C1c1ccccc1-c1ccccc1] | 441.2 |
| 30 | a mixture of rac-N-({(2S)-1-[(1R,2R)-2-(2′,6′- difluoro[1,1′-biphenyl]-2-yl)cyclopropane-1- carbonyl]pyrrolidin-2- yl}methyl)methanesulfonamide and rac-N- ({(2R)-1-[(1R,2R)-2-(2′,6′-difluoro[1,1′- biphenyl]-2-yl)cyclopropane-1- carbonyl]pyrrolidin-2- yl}methyl)methanesulfonamide | 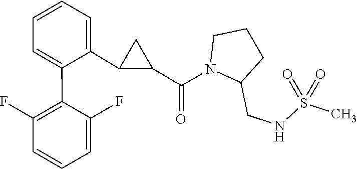 [SMILES: CS(=O)(=O)NCC1CCCN1C(=O)C1CC1c1ccccc1-c1c(F)cccc1F] | 435.2 |
| 31 | a mixture of rac-N-{(3R)-1-[(1R,2R)-2-(2′,3,6′- trifluoro[1,1′-biphenyl]-2-yl)cyclopropane-1- carbonyl]piperidin-3-yl}methanesulfonamide and rac-N-{(3S)-1-[(1R,2R)-2-(2′,3,6′- trifluoro[1,1′-biphenyl]-2-yl)cyclopropane-1- carbonyl]piperidin-3-yl}methanesulfonamide | 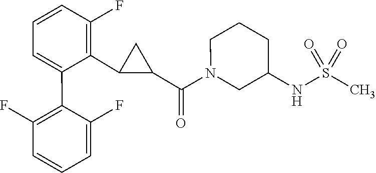 [SMILES: CS(=O)(=O)NC1CCCN(C(=O)C2CC2c2c(F)cccc2-c2c(F)cccc2F)C1] | 453.1 |
| 32 | a mixture of rac-N-{(4R)-1-[(1R,2R)-2-(2′,3,6′- trifluoro[1,1′-biphenyl]-2-yl)cyclopropane-1- carbonyl]azepan-4-yl}methanesulfonamide and rac-N-{(4S)-1-[(1R,2R)-2-(2′,3,6′- trifluoro[1,1′-biphenyl]-2-yl)cyclopropane-1- carbonyl]azepan-4-yl}methanesulfonamide | 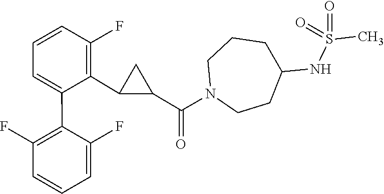 [SMILES: CS(=O)(=O)NC1CCCN(C(=O)C2CC2c2c(F)cccc2-c2c(F)cccc2F)CC1] | 467.1 |
| 33 | a mixture of rac-N-{(3R)-1-[(1R,2R)-2-(2′,6- difluoro[1,1′-biphenyl]-2-yl)cyclopropane-1- carbonyl]pyrrolidin-3-yl}methanesulfonamide and rac-N-{(3S)-1-[(1R,2R)-2-(2′,6- difluoro[1,1′-biphenyl]-2-yl)cyclopropane-1- carbonyl]pyrrolidin-3-yl}methanesulfonamide | 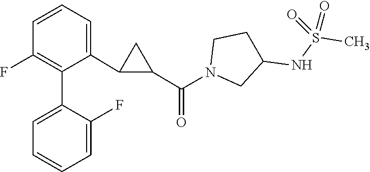 [SMILES: CS(=O)(=O)NC1CCN(C(=O)C2CC2c2cccc(F)c2-c2ccccc2F)C1] | 421.2 |
| 34 | a mixture of rac-N-{(3R)-1-[(1R,2R)-2-(5- methyl[1,1′-biphenyl]-2-yl)cyclopropane-1- carbonyl]pyrrolidin-3-yl}methanesulfonamide and rac-N-{(3S)-1-[(1R,2R)-2-(5-methyl[1,1′- biphenyl]-2-yl)cyclopropane-1- carbonyl]pyrrolidin-3-yl}methanesulfonamide | 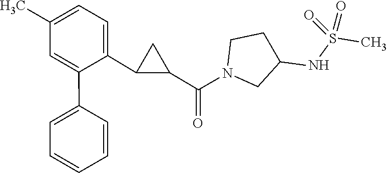 | 399.2 |
| 35 | a mixture of rac-N-{(3R)-1-[(1R,2R)-2-(6- methyl[1,1′-biphenyl]-2-yl)cyclopropane-1- carbonyl]pyrrolidin-3-yl}methanesulfonamide and rac-N-{(3S)-1-[(1R,2R)-2-(6-methyl[1,1′- biphenyl]-2-yl)cyclopropane-1- carbonyl]pyrrolidin-3-yl}methanesulfonamide | 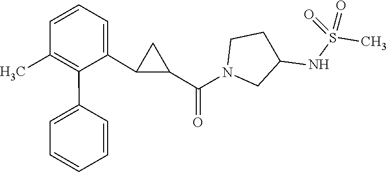 | 399.2 |
| 36 | a mixture of rac-N-{(3R)-1-[(1R,2R)-2-(3- methyl[1,1′-biphenyl]-2-yl)cyclopropane-1- carbonyl]pyrrolidin-3-yl}methanesulfonamide and rac-N-{(3S)-1-[(1R,2R)-2-(3-methyl[1,1′- biphenyl]-2-yl)cyclopropane-1- carbonyl]pyrrolidin-3-yl}methanesulfonamide | 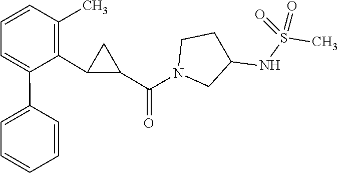 [SMILES: Cc1cccc(-c2ccccc2)c1C1CC1C(=O)N1CCC(NS(C)(=O)=O)C1] | 399.2 |
| 37 | a mixture of rac-N-{(3R)-1-[(1R,2R)-2-(2′,6′- difluoro[1,1′-biphenyl]-2-yl)cyclopropane-1- carbonyl]pyrrolidin-3- yl}cyclopropanesulfonamide and rac-N-{(3S)- 1-[(1R,2R)-2-(2′,6′-difluoro[1,1′-biphenyl]-2- yl)cyclopropane-1-carbonyl]pyrrolidin-3- yl}cyclopropanesulfonamide | 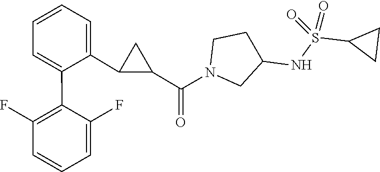 [SMILES: O=C(C1CC1c1ccccc1-c1c(F)cccc1F)N1CCC(NS(=O)(=O)C2CC2)C1] | 447.2 |
| 38 | a mixture of rac-N-{(4R)-1-[(1R,2R)-2-(2′,6′- difluoro[1,1′-biphenyl]-2-yl)cyclopropane-1- carbonyl]azepan-4-yl}methanesulfonamide and rac-N-{(4S)-1-[(1R,2R)-2-(2′,6′- difluoro[1,1′-biphenyl]-2-yl)cyclopropane-1- carbonyl]azepan-4-yl}methanesulfonamide | 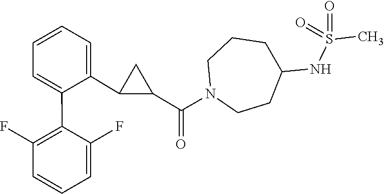 [SMILES: CS(=O)(=O)NC1CCCN(C(=O)C2CC2c2ccccc2-c2c(F)cccc2F)CC1] | 449.2 |
| 39 | a mixture of rac-N-{(3R)-1-[(1R,2R)-2-(2′,6′- difluoro[1,1′-biphenyl]-2-yl)cyclopropane-1- carbonyl]piperidin-3-yl}methanesulfonamide and rac-N-{(3S)-1-[(1R,2R)-2-(2′,6′- difluoro[1,1′-biphenyl]-2-yl)cyclopropane-1- carbonyl]piperidin-3-yl}methanesulfonamide | 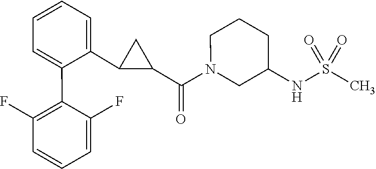 [SMILES: CS(=O)(=O)NC1CCCN(C(=O)C2CC2c2ccccc2-c2c(F)cccc2F)C1] | 435.2 |
| 40 | a mixture of rac-N-({(3R)-1-[(1R,2R)-2-(2′,6′- difluoro[1,1′-biphenyl]-2-yl)cyclopropane-1- carbonyl]pyrrolidin-3- yl}methyl)methanesulfonamide and rac-N- ({(3S)-1-[(1R,2R)-2-(2′,6′-difluoro[1,1′- biphenyl]-2-yl)cyclopropane-1- carbonyl]pyrrolidin-3- yl}methyl)methanesulfonamide | 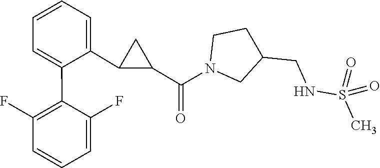 [SMILES: CS(=O)(=O)NCC1CCN(C(=O)C2CC2c2ccccc2-c2c(F)cccc2F)C1] | 435.2 |
| 41 | a mixture of rac-N-{(3R)-1-[(1R,2R)-2-(2′,6′- difluoro[1,1′-biphenyl]-2-yl)cyclopropane-1- carbonyl]azepan-3-yl}methanesulfonamide and rac-N-{(3S)-1-[(1R,2R)-2-(2′,6′- difluoro[1,1′-biphenyl]-2-yl)cyclopropane-1- carbonyl]azepan-3-yl}methanesulfonamide | 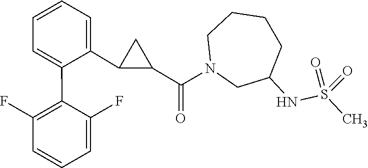 [SMILES: CS(=O)(=O)NC1CCCCN(C(=O)C2CC2c2ccccc2-c2c(F)cccc2F)C1] | 449.2 |
| 42 | a mixture of rac-N-{(4R)-1-[(1R,2R)-2-(3′- methyl[1,1′-biphenyl]-2-yl)cyclopropane-1- carbonyl]azepan-4-yl}methanesulfonamide and rac-N-{(4S)-1-[(1R,2R)-2-(3′-methyl[1,1′- biphenyl]-2-yl)cyclopropane-1- carbonyl]azepan-4-yl}methanesulfonamide | 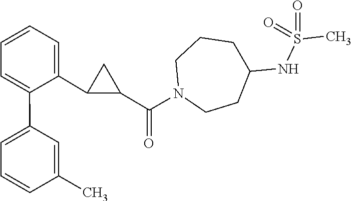 [SMILES: Cc1cccc(-c2ccccc2C2CC2C(=O)N2CCCC(NS(C)(=O)=O)CC2)c1] | 427.2 |
| 43 | a mixture of rac-N-{(4R)-1-[(1R,2R)-2-(2′- chloro[1,1′-biphenyl]-2-yl)cyclopropane-1- carbonyl]azepan-4-yl}methanesulfonamide and rac-N-{(4S)-1-[(1R,2R)-2-(2′-chloro[1,1′- biphenyl]-2-yl)cyclopropane-1- carbonyl]azepan-4-yl}methanesulfonamide | 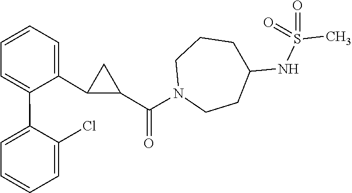 [SMILES: CS(=O)(=O)NC1CCCN(C(=O)C2CC2c2ccccc2-c2ccccc2Cl)CC1] | 447.2 |
| 44 | a mixture of rac-N-[(4R)-1-{(1R,2R)-2-[2- (furan-3-yl)phenyl]cyclopropane-1- carbonyl}azepan-4-yl]methanesulfonamide and rac-N-[(4S)-1-{(1R,2R)-2-[2-(furan-3- yl)phenyl]cyclopropane-1-carbonyl}azepan-4- yl]methanesulfonamide | 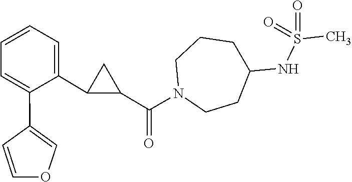 [SMILES: CS(=O)(=O)NC1CCCN(C(=O)C2CC2c2ccccc2-c2ccoc2)CC1] | 403.2 |
| 45 | a mixture of rac-N-[(4R)-1-{(1R,2R)-2-[2- (furan-2-yl)phenyl]cyclopropane-1- carbony}azepan-4-yl]methanesulfonamide and rac-N-[(4S)-1-{(1R,2R)-2-[2-(furan-2- yl)phenyl]cyclopropane-1-carbonyl}azepan-4- yl]methanesulfonamide | 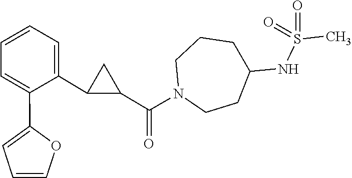 [SMILES: CS(=O)(=O)NC1CCCN(C(=O)C2CC2c2ccccc2-c2ccco2)CC1] | 403.2 |
| 46 | a mixture of rac-N-{(4R)-1-[(1R,2R)-2-(4′- chloro[1,1′-biphenyl]-2-yl)cyclopropane-1- carbonyl]azepan-4-yl}methanesulfonamide and rac-N-{(4S)-1-[(1R,2R)-2-(4′-chloro[1,1′- biphenyl]-2-yl)cyclopropane-1- carbonyl]azepan-4-yl}methanesulfonamide | 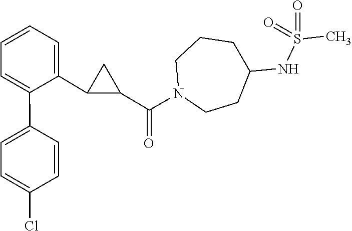 [SMILES: CS(=O)(=O)NC1CCCN(C(=O)C2CC2c2ccccc2-c2ccc(Cl)cc2)CC1] | 447.2 |
| 47 | a mixture of rac-N-{(4R)-1-[(1R,2R)-2-(2′- methoxy[1,1′-biphenyl]-2-yl)cyclopropane-1- carbonyl]azepan-4-yl}methanesulfonamide and rac-N-{(4S)-1-[(1R,2R)-2-(2′- methoxy[1,1′-biphenyl]-2-yl)cyclopropane-1- carbonyl]azepan-4-yl}methanesulfonamide | 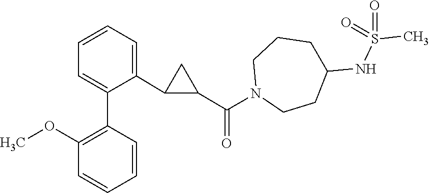 | 443.3 |
| 48 | a mixture of rac-N-[(4R)-1-{(1R,2R)-2-[4′- (methoxymethyl)[1,1′-biphenyl]-2- yl]cyclopropane-1-carbonyl}azepan-4- yl]methanesulfonamide and rac-N-[(4S)-1- {(1R,2R)-2-[4′-(methoxymethyl)[1,1′-biphenyl]- 2-yl]cyclopropane-1-carbonyl}azepan-4- yl]methanesulfonamide | 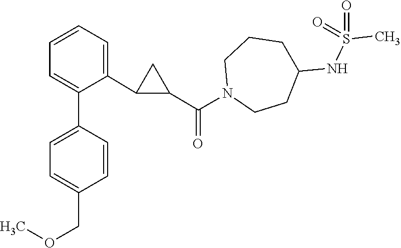 | 457.3 |
| 49 | a mixture of rac-N-{(4R)-1-[(1R,2R)-2-(4′- methyl[1,1′-biphenyl]-2-yl)cyclopropane-1- carbonyl]azepan-4-yl}methanesulfonamide and rac-N-{(4S)-1-[(1R,2R)-2-(4′-methyl[1,1′- biphenyl]-2-yl)cyclopropane-1- carbonyl]azepan-4-yl}methanesulfonamide | 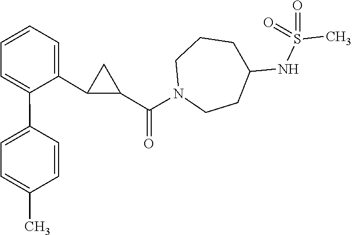 [SMILES: Cc1ccc(-c2ccccc2C2CC2C(=O)N2CCCC(NS(C)(=O)=O)CC2)cc1] | 427.2 |
| 50 | a mixture of rac-N-{(4R)-1-[(1R,2R)-2-(3′- chloro[1,1′-biphenyl]-2-yl)cyclopropane-1- carbonyl]azepan-4-yl}methanesulfonamide and rac-N-{(4S)-1-[(1R,2R)-2-(3′-chloro[1,1′- biphenyl]-2-yl)cyclopropane-1- carbonyl]azepan-4-yl}methanesulfonamide | 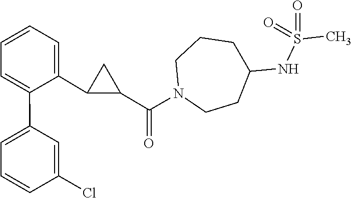 [SMILES: CS(=O)(=O)NC1CCCN(C(=O)C2CC2c2ccccc2-c2cccc(Cl)c2)CC1] | 447.2 |
| 51 | a mixture of rac-N-{(4R)-1-[(1R,2R)-2-(2′,5′- difluoro[1,1′-biphenyl]-2-yl)cyclopropane-1- carbonyl]azepan-4-yl}methanesulfonamide and rac-N-{(4S)-1-[(1R,2R)-2-(2′,5′- difluoro[1,1′-biphenyl]-2-yl)cyclopropane-1- carbonyl]azepan-4-yl}methanesulfonamide | 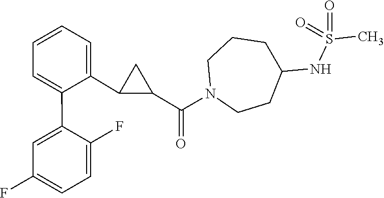 [SMILES: CS(=O)(=O)NC1CCCN(C(=O)C2CC2c2ccccc2-c2cc(F)ccc2F)CC1] | 449.2 |
| 52 | a mixture of rac-N-{(4R)-1-[(1R,2R)-2-(2′- methyl[1,1′-biphenyl]-2-yl)cyclopropane-1- carbonyl]azepan-4-yl}methanesulfonamide and rac-N-{(4S)-1-[(1R,2R)-2-(2′-methyl[1,1′- biphenyl]-2-yl)cyclopropane-1- carbonyl]azepan-4-yl}methanesulfonamide | 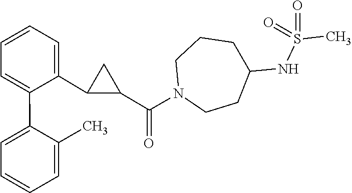 [SMILES: Cc1ccccc1-c1ccccc1C1CC1C(=O)N1CCCC(NS(C)(=O)=O)CC1] | 427.2 |
| 53 | a mixture of rac-N-{(4R)-1-[(1R,2R)-2-(3′- methoxy[1,1′-biphenyl]-2-yl)cyclopropane-1- carbonyl]azepan-4-yl}methanesulfonamide and rac-N-{(4S)-1-[(1R,2R)-2-(3′- methoxy[1,1′-biphenyl]-2-yl)cyclopropane-1- carbonyl]azepan-4-yl}methanesulfonamide | 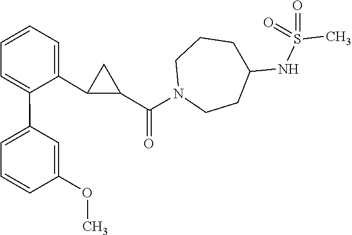 [SMILES: COc1cccc(-c2ccccc2C2CC2C(=O)N2CCCC(NS(C)(=O)=O)CC2)c1] | 443.3 |
| 54 | a mixture of rac-N-{(4R)-1-[(1R,2R)-2-(2′,3′- difluoro[1,1′-biphenyl]-2-yl)cyclopropane-1- carbonyl]azepan-4-yl}methanesulfonamide and rac-N-{(4S)-1-[(1R,2R)-2-(2′,3′- difluoro[1,1′-biphenyl]-2-yl)cyclopropane-1- carbonyl]azepan-4-yl}methanesulfonamide | 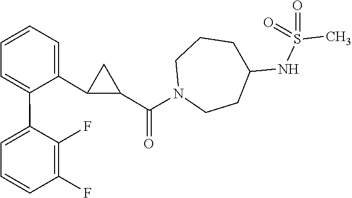 [SMILES: CS(=O)(=O)NC1CCCN(C(=O)C2CC2c2ccccc2-c2cccc(F)c2F)CC1] | 449.2 |
| 55 | a mixture of rac-N-{(4R)-1-[(1R,2R)-2-(3′,4′- difluoro[1,1′-biphenyl]-2-yl)cyclopropane-1- carbonyl]azepan-4-yl}methanesulfonamide and rac-N-{(4S)-1-[(1R,2R)-2-(3′,4′- difluoro[1,1′-biphenyl]-2-yl)cyclopropane-1- carbonyl]azepan-4-yl}methanesulfonamide | 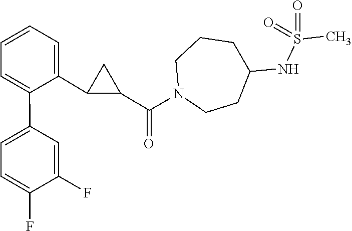 [SMILES: CS(=O)(=O)NC1CCCN(C(=O)C2CC2c2ccccc2-c2ccc(F)c(F)c2)CC1] | 449.2 |
| 56 | a mixture of rac-N-{(4R)-1-[(1R,2R)-2-(2′,4′- difluoro[1,1′-biphenyl]-2-yl)cyclopropane-1- carbonyl]azepan-4-yl}methanesulfonamide and rac-N-{(4S)-1-[(1R,2R)-2-(2′,4′- difluoro[1,1′-biphenyl]-2-yl)cyclopropane-1- carbonyl]azepan-4-yl}methanesulfonamide | 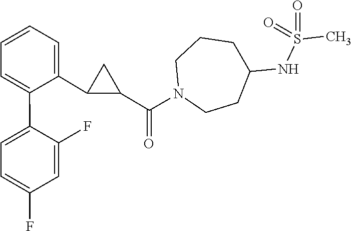 [SMILES: CS(=O)(=O)NC1CCCN(C(=O)C2CC2c2ccccc2-c2ccc(F)cc2F)CC1] | 449.2 |
| 57 | a mixture of rac-N-{(4R)-1-[(1R,2R)-2-(3′,5′- difluoro[1,1′-biphenyl]-2-yl)cyclopropane-1- carbonyl]azepan-4-yl}methanesulfonamide and rac-N-{(4S)-1-[(1R,2R)-2-(3′,5′- difluoro[1,1′-biphenyl]-2-yl)cyclopropane-1- carbonyl]azepan-4-yl}methanesulfonamide | 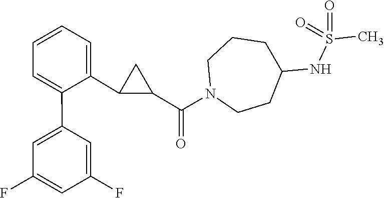 [SMILES: CS(=O)(=O)NC1CCCN(C(=O)C2CC2c2ccccc2-c2cc(F)cc(F)c2)CC1] | 449.2 |
| 58 | a mixture of rac-N-{(4R)-1-[(1R,2R)-2-(4′- fluoro[1,1′-biphenyl]-2-yl)cyclopropane-1- carbonyl]azepan-4-yl}methanesulfonamide and rac-N-{(4R)-1-[(1R,2R)-2-(4′-fluoro[1,1′- biphenyl]-2-yl)cyclopropane-1- carbonyl]azepan-4-yl}methanesulfonamide | 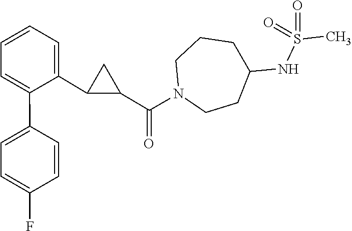 [SMILES: CS(=O)(=O)NC1CCCN(C(=O)C2CC2c2ccccc2-c2ccc(F)cc2)CC1] | 431.2 |
| 59 | a mixture of rac-N-{(4R)-1-[(1R,2R)-2-(2′- fluoro[1,1′-biphenyl]-2-yl)cyclopropane-1- carbonyl]azepan-4-yl}methanesulfonamide and rac-N-{(4S)-1-[(1R,2R)-2-(2′-fluoro[1,1′- biphenyl]-2-yl)cyclopropane-1- carbonyl]azepan-4-yl}methanesulfonamide | 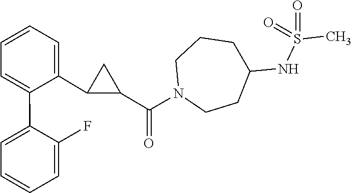 [SMILES: CS(=O)(=O)NC1CCCN(C(=O)C2CC2c2ccccc2-c2ccccc2F)CC1] | 431.2 |
| 60 | a mixture of rac-N-{(4R)-1-[(1R,2R)-2-(3′- fluoro[1,1′-biphenyl]-2-yl)cyclopropane-1- carbonyl]azepan-4-yl}methanesulfonamide and rac-N-{(4S)-1-[(1R,2R)-2-(3′-fluoro[1,1′- biphenyl]-2-yl)cyclopropane-1- carbonyl]azepan-4-yl}methanesulfonamide | 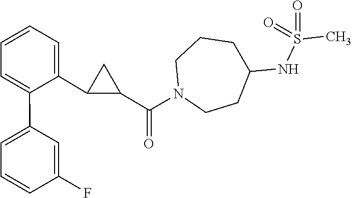 [SMILES: CS(=O)(=O)NC1CCCN(C(=O)C2CC2c2ccccc2-c2cccc(F)c2)CC1] | 431.2 |
| 61 | a mixture of rac-N-{(3R)-1-[(1R,2R)-2-(4′- methyl[1,1′-biphenyl]-2-yl)cyclopropane-1- carbonyl]piperidin-3-yl}methanesulfonamide and rac-N-{(3S)-1-[(1R,2R)-2-(4′-methyl[1,1′- biphenyl]-2-yl)cyclopropane-1- carbonyl]piperidin-3-yl}methanesulfonamide | 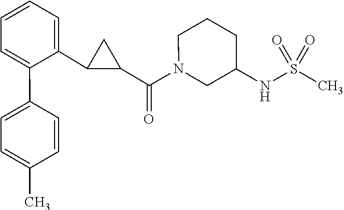 [SMILES: Cc1ccc(-c2ccccc2C2CC2C(=O)N2CCCC(NS(C)(=O)=O)C2)cc1] | 413.2 |
| 62 | a mixture of rac-N-{(3R)-1-[(1R,2R)-2-(2′- methoxy[1,1′-biphenyl]-2-yl)cyclopropane-1- carbonyl]piperidin-3-yl}methanesulfonamide and rac-N-{(3S)-1-[(1R,2R)-2-(2′- methoxy[1,1′-biphenyl]-2-yl)cyclopropane-1- carbonyl]piperidin-3-yl}methanesulfonamide | 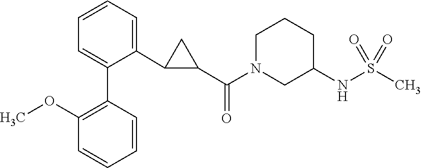 | 429.2 |
| 63 | a mixture of rac-N-{(3R)-1-[(1R,2R)-2-(4′- chloro[1,1′-biphenyl]-2-yl)cyclopropane-1- carbonyl]piperidin-3-yl}methanesulfonamide and rac-N-{(3S)-1-[(1R,2R)-2-(4′-chloro[1,1′- biphenyl]-2-yl)cyclopropane-1- carbonyl]piperidin-3-yl}methanesulfonamide | 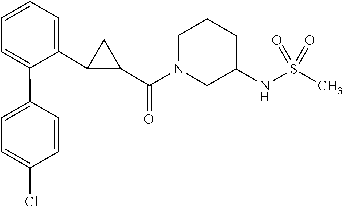 [SMILES: CS(=O)(=O)NC1CCCN(C(=O)C2CC2c2ccccc2-c2ccc(Cl)cc2)C1] | 433.1 |
| 64 | a mixture of rac-N-{(3R)-1-[(1R,2R)-2-(3′- chloro[1,1′-biphenyl]-2-yl)cyclopropane-1- carbonyl]piperidin-3-yl}methanesulfonamide and rac-N-{(3S)-1-[(1R,2R)-2-(3′-chloro[1,1′- biphenyl]-2-yl)cyclopropane-1- carbonyl]piperidin-3-yl}methanesulfonamide | 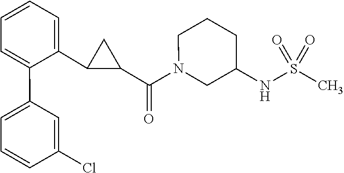 [SMILES: CS(=O)(=O)NC1CCCN(C(=O)C2CC2c2ccccc2-c2cccc(Cl)c2)C1] | 433.2 |
| 65 | a mixture of rac-N-{(3R)-1-[(1R,2R)-2-(2′,5′- difluoro[1,1′-biphenyl]-2-yl)cyclopropane-1- carbonyl]piperidin-3-yl}methanesulfonamide and rac-N-{(3S)-1-[(1R,2R)-2-(2′,5′- difluoro[1,1′-biphenyl]-2-yl)cyclopropane-1- carbonyl]piperidin-3-yl}methanesulfonamide | 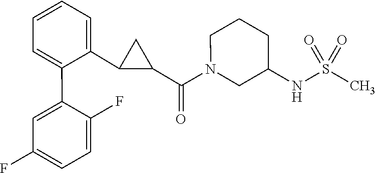 [SMILES: CS(=O)(=O)NC1CCCN(C(=O)C2CC2c2ccccc2-c2cc(F)ccc2F)C1] | 435.2 |
| 66 | a mixture of rac-N-{(3R)-1-[(1R,2R)-2-(2′- methyl[1,1′-biphenyl]-2-yl)cyclopropane-1- carbonyl]piperidin-3-yl}methanesulfonamide and rac-N-{(3S)-1-[(1R,2R)-2-(2′-methyl[1,1′- biphenyl]-2-yl)cyclopropane-1- carbonyl]piperidin-3-yl}methanesulfonamide | 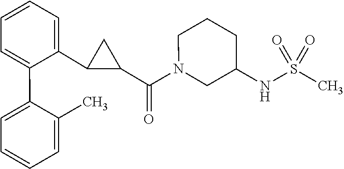 [SMILES: Cc1ccccc1-c1ccccc1C1CC1C(=O)N1CCCC(NS(C)(=O)=O)C1] | 413.3 |
| 67 | a mixture of rac-N-{(3R)-1-[(1R,2R)-2-(2′,3′- difluoro[1,1′-biphenyl]-2-yl)cyclopropane-1- carbonyl]piperidin-3-yl}methanesulfonamide and rac-N-{(3S)-1-[(1R,2R)-2-(2′,3′- difluoro[1,1′-biphenyl]-2-yl)cyclopropane-1- carbonyl]piperidin-3-yl}methanesulfonamide | 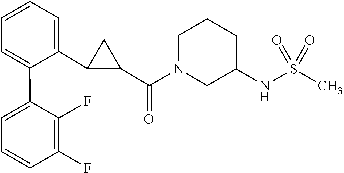 [SMILES: CS(=O)(=O)NC1CCCN(C(=O)C2CC2c2ccccc2-c2cccc(F)c2F)C1] | 435.2 |
| 68 | a mixture of rac-N-{(3R)-1-[(1R,2R)-2-(2′,4′- difluoro[1,1′-biphenyl]-2-yl)cyclopropane-1- carbonyl]piperidin-3-yl}methanesulfonamide and rac-N-{(3S)-1-[(1R,2R)-2-(2′,4′- difluoro[1,1′-biphenyl]-2-yl)cyclopropane-1- carbonyl]piperidin-3-yl}methanesulfonamide | 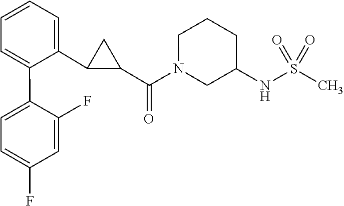 [SMILES: CS(=O)(=O)NC1CCCN(C(=O)C2CC2c2ccccc2-c2ccc(F)cc2F)C1] | 435.2 |
| 69 | a mixture of rac-N-{(3R)-1-[(1R,2R)-2-(3′- methyl[1,1′-biphenyl]-2-yl)cyclopropane-1- carbonyl]piperidin-3-yl}methanesulfonamide and rac-N-{(3S)-1-[(1R,2R)-2-(3′-methyl[1,1′- biphenyl]-2-yl)cyclopropane-1- carbonyl]piperidin-3-yl}methanesulfonamide | 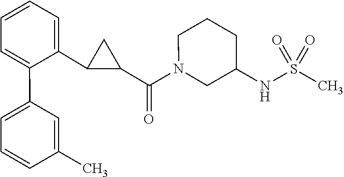 [SMILES: Cc1cccc(-c2ccccc2C2CC2C(=O)N2CCCC(NS(C)(=O)=O)C2)c1] | 413.3 |
| 70 | a mixture of rac-N-{(3R)-1-[(1R,2R)-2-(4′- fluoro[1,1′-biphenyl]-2-yl)cyclopropane-1- carbonyl]piperidin-3-yl}methanesulfonamide and rac-N-{(3S)-1-[(1R,2R)-2-(4′-fluoro[1,1′- biphenyl]-2-yl)cyclopropane-1- carbonyl]piperidin-3-yl}methanesulfonamide | 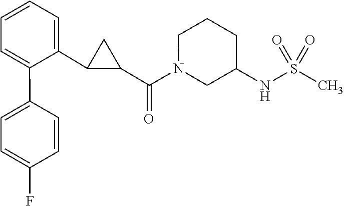 [SMILES: CS(=O)(=O)NC1CCCN(C(=O)C2CC2c2ccccc2-c2ccc(F)cc2)C1] | 417.2 |
| 71 | a mixture of rac-N-{(3R)-1-[(1R,2R)-2-(3′,4′- difluoro[1,1′-biphenyl]-2-yl)cyclopropane-1- carbonyl]piperidin-3-yl}methanesulfonamide and rac-N-{(3S)-1-[(1R,2R)-2-(3′,4′- difluoro[1,1′-biphenyl]-2-yl)cyclopropane-1- carbonyl]piperidin-3-yl}methanesulfonamide | 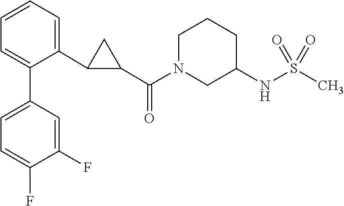 [SMILES: CS(=O)(=O)NC1CCCN(C(=O)C2CC2c2ccccc2-c2ccc(F)c(F)c2)C1] | 435.2 |
| 72 | a mixture of rac-N-{(3R)-1-[(1R,2R)-2-(2′- fluoro[1,1′-biphenyl]-2-yl)cyclopropane-1- carbonyl]piperidin-3-yl}methanesulfonamide and rac-N-{(3S)-1-[(1R,2R)-2-(2′-fluoro[1,1′- biphenyl]-2-yl)cyclopropane-1- carbonyl]piperidin-3-yl}methanesulfonamide | 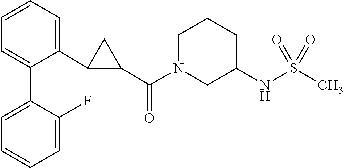 [SMILES: CS(=O)(=O)NC1CCCN(C(=O)C2CC2c2ccccc2-c2ccccc2F)C1] | 417.2 |
| 73 | a mixture of rac-N-{(3R)-1-[(1R,2R)-2-(3′,5′- difluoro[1,1′-biphenyl]-2-yl)cyclopropane-1- carbonyl]piperidin-3-yl}methanesulfonamide and rac-N-{(3S)-1-[(1R,2R)-2-(3′,5′- difluoro[1,1′-biphenyl]-2-yl)cyclopropane-1- carbonyl]piperidin-3-yl}methanesulfonamide | 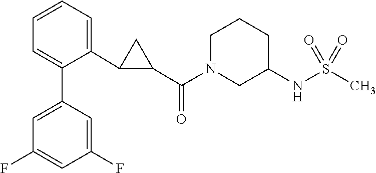 [SMILES: CS(=O)(=O)NC1CCCN(C(=O)C2CC2c2ccccc2-c2cc(F)cc(F)c2)C1] | 435.2 |
| 74 | a mixture of rac-N-{(3R)-1-[(1R,2R)-2-(2′- chloro[1,1′-biphenyl]-2-yl)cyclopropane-1- carbonyl]piperidin-3-yl}methanesulfonamide and rac-N-{(3S)-1-[(1R,2R)-2-(2′-chloro[1,1′- biphenyl]-2-yl)cyclopropane-1- carbonyl]piperidin-3-yl}methanesulfonamide | 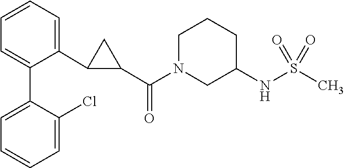 [SMILES: CS(=O)(=O)NC1CCCN(C(=O)C2CC2c2ccccc2-c2ccccc2Cl)C1] | 433.2 |
| 75 | a mixture of rac-N-{(3R)-1-[(1R,2R)-2-(3′- fluoro[1,1′-biphenyl]-2-yl)cyclopropane-1- carbonyl]piperidin-3-yl}methanesulfonamide and rac-N-{(3S)-1-[(1R,2R)-2-(3′-fluoro[1,1′- biphenyl]-2-yl)cyclopropane-1- carbonyl]piperidin-3-yl}methanesulfonamide | 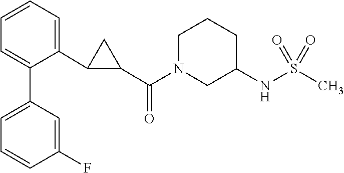 [SMILES: CS(=O)(=O)NC1CCCN(C(=O)C2CC2c2ccccc2-c2cccc(F)c2)C1] | 417.2 |
| 76 | a mixture of rac-N-{(3R)-1-[(1R,2R)-2-(2′- methoxy[1,1′-biphenyl]-2-yl)cyclopropane-1- carbonyl]pyrrolidin-3-yl}methanesulfonamide and rac-N-{(3S)-1-[(1R,2R)-2-(2′- methoxy[1,1′-biphenyl]-2-yl)cyclopropane-1- carbonyl]pyrrolidin-3-yl}methanesulfonamide | 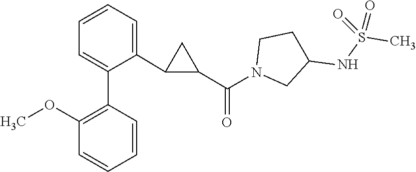 | 415.2 |
| 77 | a mixture of rac-N-{(3R)-1-[(1R,2R)-2-(4′- chloro[1,1′-biphenyl]-2-yl)cyclopropane-1- carbonyl]pyrrolidin-3-yl}methanesulfonamide and rac-N-{(3S)-1-[(1R,2R)-2-(4′-chloro[1,1′- biphenyl]-2-yl)cyclopropane-1- carbonyl]pyrrolidin-3-yl}methanesulfonamide | 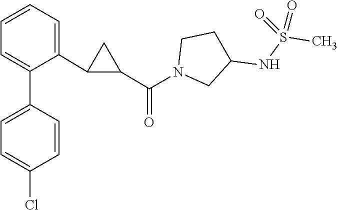 [SMILES: CS(=O)(=O)NC1CCN(C(=O)C2CC2c2ccccc2-c2ccc(Cl)cc2)C1] | 419.1 |
| 78 | a mixture of rac-N-{(3R)-1-[(1R,2R)-2-(2′- methyl[1,1′-biphenyl]-2-yl)cyclopropane-1- carbonyl]pyrrolidin-3-yl}methanesulfonamide and rac-N-{(3S)-1-[(1R,2R)-2-(2′-methyl[1,1′- biphenyl]-2-yl)cyclopropane-1- carbonyl]pyrrolidin-3-yl}methanesulfonamide | 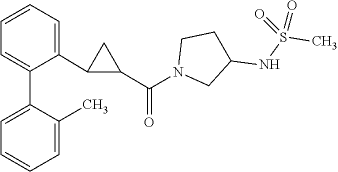 [SMILES: Cc1ccccc1-c1ccccc1C1CC1C(=O)N1CCC(NS(C)(=O)=O)C1] | 399.2 |
| 79 | a mixture of rac-N-{(3R)-1-[(1R,2R)-2-(3′- chloro[1,1′-biphenyl]-2-yl)cyclopropane-1- carbonyl]pyrrolidin-3-yl}methanesulfonamide and rac-N-{(3S)-1-[(1R,2R)-2-(3′-chloro[1,1′- biphenyl]-2-yl)cyclopropane-1- carbonyl]pyrrolidin-3-yl}methanesulfonamide | 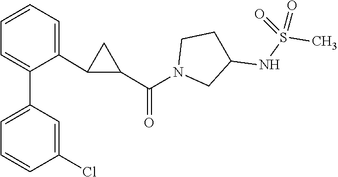 [SMILES: CS(=O)(=O)NC1CCN(C(=O)C2CC2c2ccccc2-c2cccc(Cl)c2)C1] | 419.2 |
| 80 | a mixture of rac-N-{(3R)-1-[(1R,2R)-2-(2′,5′- difluoro[1,1′-biphenyl]-2-yl)cyclopropane-1- carbonyl]pyrrolidin-3-yl}methanesulfonamide and rac-N-{(3S)-1-[(1R,2R)-2-(2′,5′- difluoro[1,1′-biphenyl]-2-yl)cyclopropane-1- carbonyl]pyrrolidin-3-yl}methanesulfonamide | 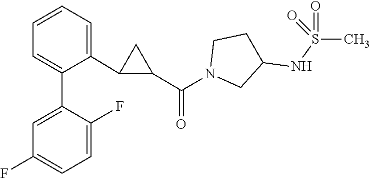 [SMILES: CS(=O)(=O)NC1CCN(C(=O)C2CC2c2ccccc2-c2cc(F)ccc2F)C1] | 421.2 |
| 81 | a mixture of rac-N-{(3R)-1-[(1R,2R)-2-(2′,3′- difluoro[1,1′-biphenyl]-2-yl)cyclopropane-1- carbonyl]pyrrolidin-3-yl}methanesulfonamide and rac-N-{(3S)-1-[(1R,2R)-2-(2′,3′- difluoro[1,1′-biphenyl]-2-yl)cyclopropane-1- carbonyl]pyrrolidin-3-yl}methanesulfonamide | 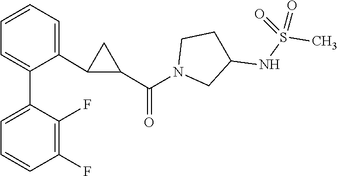 [SMILES: CS(=O)(=O)NC1CCN(C(=O)C2CC2c2ccccc2-c2cccc(F)c2F)C1] | 421.2 |
| 82 | a mixture of rac-N-{(3R)-1-[(1R,2R)-2-(3′,4′- difluoro[1,1′-biphenyl]-2-yl)cyclopropane-1- carbonyl]pyrrolidin-3-yl}methanesulfonamide and rac-N-{(3S)-1-[(1R,2R)-2-(3′,4′- difluoro[1,1′-biphenyl]-2-yl)cyclopropane-1- carbonyl]pyrrolidin-3-yl}methanesulfonamide | 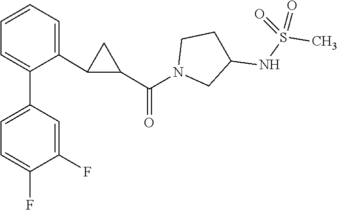 [SMILES: CS(=O)(=O)NC1CCN(C(=O)C2CC2c2ccccc2-c2ccc(F)c(F)c2)C1] | 421.2 |
| 83 | a mixture of rac-N-{(3R)-1-[(1R,2R)-2-(3′- methyl[1,1-biphenyl]-2-yl)cyclopropane-1- carbonyl]pyrrolidin-3-yl}methanesulfonamide and rac-N-{(3S)-1-[(1R,2R)-2-(3′-methyl[1,1′- biphenyl]-2-yl)cyclopropane-1- carbonyl]pyrrolidin-3-yl}methanesulfonamide | 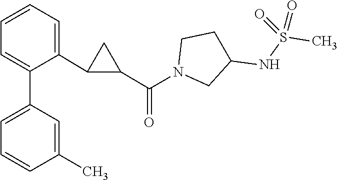 [SMILES: Cc1cccc(-c2ccccc2C2CC2C(=O)N2CCC(NS(C)(=O)=O)C2)c1] | 399.2 |
| 84 | a mixture of rac-N-{(3R)-1-[(1R,2R)-2-(3′,5′- difluoro[1,1′-biphenyl]-2-yl)cyclopropane-1- carbonyl]pyrrolidin-3-yl}methanesulfonamide and rac-N-{(3S)-1-[(1R,2R)-2-(3′,5′- difluoro[1,1′-biphenyl]-2-yl)cyclopropane-1- carbonyl]pyrrolidin-3-yl}methanesulfonamide | 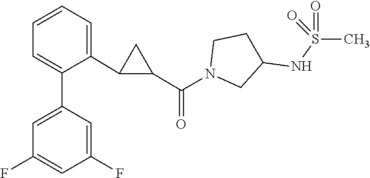 [SMILES: CS(=O)(=O)NC1CCN(C(=O)C2CC2c2ccccc2-c2cc(F)cc(F)c2)C1] | 421.2 |
| 85 | a mixture of rac-N-{(3R)-1-[(1R,2R)-2-(2′- chloro[1,1′-biphenyl]-2-yl)cyclopropane-1- carbonyl]pyrrolidin-3-yl}methanesulfonamide and rac-N-{(3S)-1-[(1R,2R)-2-(2′-chloro[1,1′- biphenyl]-2-yl)cyclopropane-1- carbonyl]pyrrolidin-3-yl}methanesulfonamide | 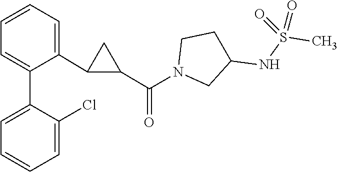 [SMILES: CS(=O)(=O)NC1CCN(C(=O)C2CC2c2ccccc2-c2ccccc2Cl)C1] | 419.2 |
| 86 | a mixture of rac-N-{(3R)-1-[(1R,2R)-2-(5- fluoro[1,1′-biphenyl]-2-yl)cyclopropane-1- carbonyl]piperidin-3-yl}methanesulfonamide and rac-N-{(3S)-1-[(1R,2R)-2-(5-fluoro[1,1′- biphenyl]-2-yl)cyclopropane-1- carbonyl]piperidin-3-yl}methanesulfonamide | 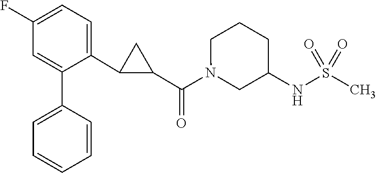 [SMILES: CS(=O)(=O)NC1CCCN(C(=O)C2CC2c2ccc(F)cc2-c2ccccc2)C1] | 417.2 |
| 87 | a mixture of rac-N-{(4R)-1-[(1R,2R)-2-(5- fluoro[1,1′-biphenyl]-2-yl)cyclopropane-1- carbonyl]azepan-4-yl}methanesulfonamide and rac-N-{(4S)-1-[(1R,2R)-2-(5-fluoro[1,1′- biphenyl]-2-yl)cyclopropane-1- carbonyl]azepan-4-yl}methanesulfonamide | 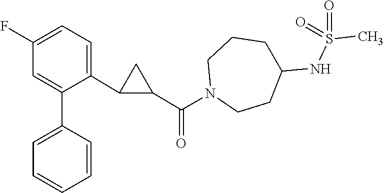 [SMILES: CS(=O)(=O)NC1CCCN(C(=O)C2CC2c2ccc(F)cc2-c2ccccc2)CC1] | 431.3 |
| 88 | a mixture of rac-N-{(4R)-1-[(1R,2R)-2-(6- methyl[1,1′-biphenyl]-2-yl)cyclopropane-1- carbonyl]azepan-4-yl}methanesulfonamide and rac-N-{(4S)-1-[(1R,2R)-2-(6-methyl[1,1′- biphenyl]-2-yl)cyclopropane-1- carbonyl]azepan-4-yl}methanesulfonamide | 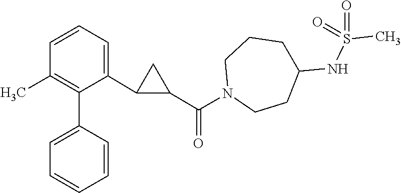 | 427.3 |
| 89 | a mixture of rac-N-{(3R)-1-[(1R,2R)-2-(6- methyl[1,1′-biphenyl]-2-yl)cyclopropane-1- carbonyl]piperidin-3-yl}methanesulfonamide and rac-N-{(3S)-1-[(1R,2R)-2-(6-methyl[1,1′- biphenyl]-2-yl)cyclopropane-1- carbonyl]piperidin-3-yl}methanesulfonamide | 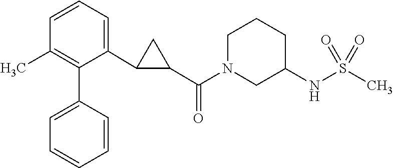 | 413.3 |
| 90 | a mixture of rac-N-{(4R)-1-[(1R,2R)-2-(4- methyl[1,1′-biphenyl]-2-yl)cyclopropane-1- carbonyl]azepan-4-yl}methanesulfonamide and rac-N-{(4S)-1-[(1R,2R)-2-(4-methyl[1,1′- biphenyl]-2-yl)cyclopropane-1- carbonyl]azepan-4-yl}methanesulfonamide | 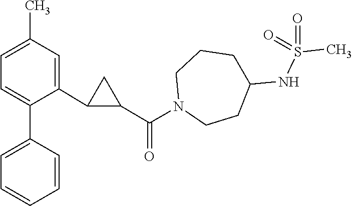 [SMILES: Cc1ccc(-c2ccccc2)c(C2CC2C(=O)N2CCCC(NS(C)(=O)=O)CC2)c1] | 427.3 |
| 91 | a mixture of rac-N-{(3R)-1-[(1R,2R)-2-(6- fluoro[1,1′-biphenyl]-2-yl)cyclopropane-1- carbonyl]piperidin-3-yl}methanesulfonamide and rac-N-{(3S)-1-[(1R,2R)-2-(6-fluoro[1,1′- biphenyl]-2-yl)cyclopropane-1- carbonyl]piperidin-3-yl}methanesulfonamide | 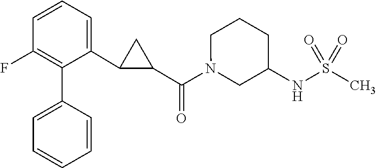 [SMILES: CS(=O)(=O)NC1CCCN(C(=O)C2CC2c2cccc(F)c2-c2ccccc2)C1] | 417.2 |
| 92 | a mixture of rac-N-{(3R)-1-[(1R,2R)-2-(3- fluoro[1,1′-biphenyl]-2-yl)cyclopropane-1- carbonyl]piperidin-3-yl}methanesulfonamide and rac-N-{(3S)-1-[(1R,2R)-2-(3-fluoro[1,1′- biphenyl]-2-yl)cyclopropane-1- carbonyl]piperidin-3-yl}methanesulfonamide | 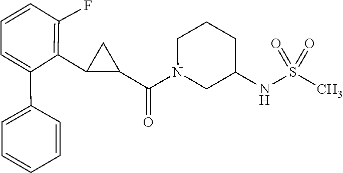 [SMILES: CS(=O)(=O)NC1CCCN(C(=O)C2CC2c2c(F)cccc2-c2ccccc2)C1] | 417.3 |
| 93 | a mixture of rac-N-{(4R)-1-[(1R,2R)-2-(6- fluoro[1,1′-biphenyl]-2-yl)cyclopropane-1- carbonyl]azepan-4-yl}methanesulfonamide and rac-N-{(4S)-1-[(1R,2R)-2-(6-fluoro[1,1′- biphenyl]-2-yl)cyclopropane-1- carbonyl]azepan-4-yl}methanesulfonamide | 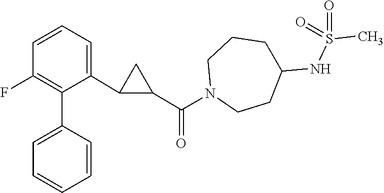 [SMILES: CS(=O)(=O)NC1CCCN(C(=O)C2CC2c2cccc(F)c2-c2ccccc2)CC1] | 431.2 |
| 94 | a mixture of rac-N-{(4R)-1-[(1R,2R)-2-(4- fluoro[1,1′-biphenyl]-2-yl)cyclopropane-1- carbonyl]azepan-4-yl}methanesulfonamide and rac-N-{(4S)-1-[(1R,2R)-2-(4-fluoro[1,1′- biphenyl]-2-yl)cyclopropane-1- carbonyl]azepan-4-yl}methanesulfonamide | 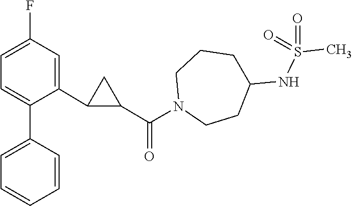 [SMILES: CS(=O)(=O)NC1CCCN(C(=O)C2CC2c2cc(F)ccc2-c2ccccc2)CC1] | 431.2 |
| 95 | a mixture of rac-N-{(4R)-1-[(1R,2R)-2-(3- fluoro[1,1′-biphenyl]-2-yl)cyclopropane-1- carbonyl]azepan-4-yl}methanesulfonamide and rac-N-{(4S)-1-[(1R,2R)-2-(3-fluoro[1,1′- biphenyl]-2-yl)cyclopropane-1- carbonyl]azepan-4-yl}methanesulfonamide | 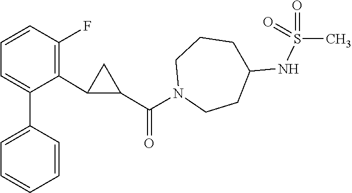 [SMILES: CS(=O)(=O)NC1CCCN(C(=O)C2CC2c2c(F)cccc2-c2ccccc2)CC1] | 431.2 |
| 96 | a mixture of rac-N-{(4R)-1-[(1R,2R)-2-(2′,4,6′- trifluoro[1,1′-biphenyl]-2-yl)cyclopropane-1- carbonyl]azepan-4-yl}methanesulfonamide and rac-N-{(4S)-1-[(1R,2R)-2-(2′,4,6′- trifluoro[1,1′-biphenyl]-2-yl)cyclopropane-1- carbonyl]azepan-4-yl}methanesulfonamide | 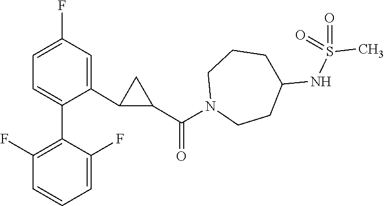 [SMILES: CS(=O)(=O)NC1CCCN(C(=O)C2CC2c2cc(F)ccc2-c2c(F)cccc2F)CC1] | 467.2 |
| 97 | a mixture of rac-N-{(3R)-1-[(1R,2R)-2-(2′,4,6′- trifluoro[1,1′-biphenyl]-2-yl)cyclopropane-1- carbonyl]pyrrolidin-3-yl}methanesulfonamide and rac-N-{(3S)-1-[(1R,2R)-2-(2′,4,6′- trifluoro[1,1′-biphenyl]-2-yl)cyclopropane-1- carbonyl]pyrrolidin-3-yl}methanesulfonamide | 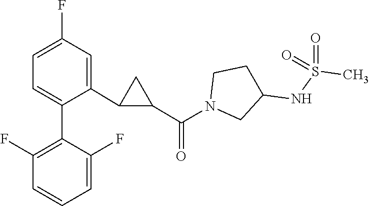 [SMILES: CS(=O)(=O)NC1CCN(C(=O)C2CC2c2cc(F)ccc2-c2c(F)cccc2F)C1] | 439.1 |
| 98 | a mixture of rac-N-{(3R)-1-[(1R,2R)-2-(2′,4,6′- trifluoro[1,1′-biphenyl]-2-yl)cyclopropane-1- carbonyl]piperidin-3-yl}methanesulfonamide and rac-N-{(3S)-1-[(1R,2R)-2-(2′,4,6′- trifluoro[1,1′-biphenyl]-2-yl)cyclopropane-1- carbonyl]piperidin-3-yl}methanesulfonamide | 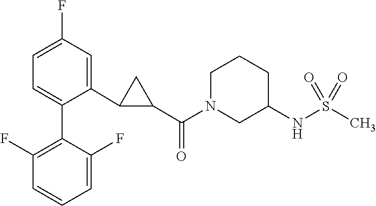 [SMILES: CS(=O)(=O)NC1CCCN(C(=O)C2CC2c2cc(F)ccc2-c2c(F)cccc2F)C1] | 453.1 |
| 99 | a mixture of rac-N-{(3R)-1-[(1R,2R)-2-[2- (pyridin-2-yl)phenyl]cyclopropane-1- carbonyl}piperidin-3-yl]methanesulfonamide and rac-N-[(3S)-1-{(1R,2R)-2-[2-(pyridin-2- yl)phenyl]cyclopropane-1-carbonyl}piperidin- 3-yl]methanesulfonamide | 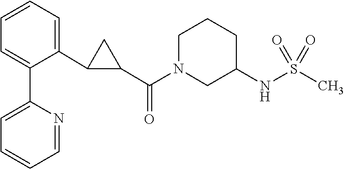 [SMILES: CS(=O)(=O)NC1CCCN(C(=O)C2CC2c2ccccc2-c2ccccn2)C1] | 400.3 |
| 100 | a mixture of rac-N-{(4R)-1-[(1R,2R)-2-(5- methyl[1,1′-biphenyl]-2-yl)cyclopropane-1- carbonyl]azepan-4-yl}methanesulfonamide and rac-N-{(4S)-1-[(1R,2R)-2-(5-methyl[1,1′- biphenyl]-2-yl)cyclopropane-1- carbonyl]azepan-4-yl}methanesulfonamide | 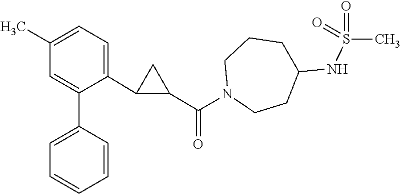 | 427.2 |
| 101 | a mixture of rac-N-{(3R)-1-[(1R,2R)-2-(5- methyl[1,1′-biphenyl]-2-yl)cyclopropane-1- carbonyl]piperidin-3-yl}methanesulfonamide and rac-N-{(3S)-1-[(1R,2R)-2-(5-methyl[1,1′- biphenyl]-2-yl)cyclopropane-1- carbonyl]piperidin-3-yl}methanesulfonamide | 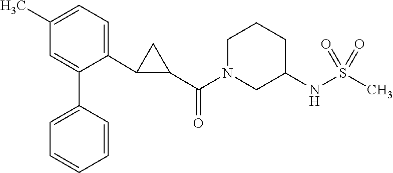 | 413.2 |
| 102 | a mixture of rac-N-{(3R)-1-[(1R,2R)-2-([1,1′- biphenyl]-2-yl)-2-methylcyclopropane-1- carbonyl]pyrrolidin-3-yl}methanesulfonamide and rac-N-{(3S)-1-[(1R,2R)-2-([1,1′- biphenyl]-2-yl)-2-methylcyclopropane-1- carbonyl]pyrrolidin-3-yl}methanesulfonamide | 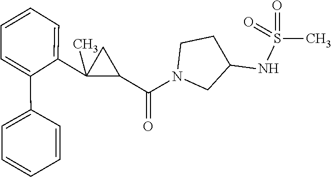 [SMILES: CC1(c2ccccc2-c2ccccc2)CC1C(=O)N1CCC(NS(C)(=O)=O)C1] | 399.2 |
| 103 | a mixture of rac-N-{(3R)-1-[(1R,2R)-2-([1,1′- biphenyl]-2-yl)-2-methylcyclopropane-1- carbonyl]piperidin-3-yl}methanesulfonamide and rac-N-{(3R)-1-[(1R,2R)-2-([1,1′- biphenyl]-2-yl)-2-methylcyclopropane-1- carbonyl]piperidin-3-yl}methanesulfonamide | 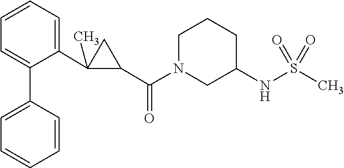 [SMILES: CC1(c2ccccc2-c2ccccc2)CC1C(=O)N1CCCC(NS(C)(=O)=O)C1] | 413.3 |
| 104 | a mixture of rac-N-{(4R)-1-[(1R,2R)-2-([1,1′- biphenyl]-2-yl)-2-methylcyclopropane-1- carbonyl]azepan-4-yl}methanesulfonamide and rac-N-{(4S)-1-[(1R,2R)-2-([1,1′- biphenyl-2-yl)-2-methylcyclopropane-1- carbonyl]azepan-4-yl}methanesulfonamide | 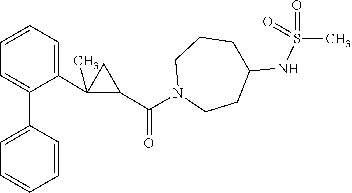 [SMILES: CC1(c2ccccc2-c2ccccc2)CC1C(=O)N1CCCC(NS(C)(=O)=O)CC1] | 427.2 |
| 105 | a mixture of rac-N-{(3R)-1-[(1R,2R)-2-(2′,6- difluoro[1,1′-biphenyl]-2-yl)cyclopropane-1- carbonyl]piperidin-3-yl}methanesulfonamide and rac-N-{(3S)-1-[(1R,2R)-2-(2′,6- difluoro[1,1′-biphenyl]-2-yl)cyclopropane-1- carbonyl]piperidin-3-yl}methanesulfonamide | 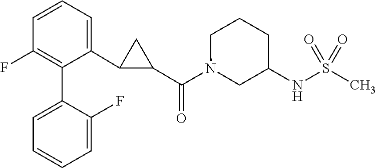 [SMILES: CS(=O)(=O)NC1CCCN(C(=O)C2CC2c2cccc(F)c2-c2ccccc2F)C1] | 435.2 |
| 106 | a mixture of rac-N-{(4R)-1-[(1R,2R)-2-(2′,6- difluoro[1,1′-biphenyl]-2-yl)cyclopropane-1- carbonyl]azepan-4-yl}methanesulfonamide and rac-N-{(4S)-1-[(1R,2R)-2-(2′,6- difluoro[1,1′-biphenyl]-2-yl)cyclopropane-1- carbonyl]azepan-4-yl}methanesulfonamide | 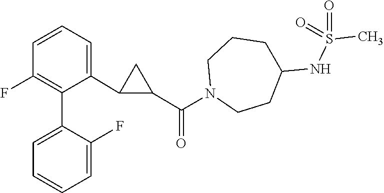 [SMILES: CS(=O)(=O)NC1CCCN(C(=O)C2CC2c2cccc(F)c2-c2ccccc2F)CC1] | 449.2 |
| 107 | rac-N-({1-[(1R,2R)-2-(2′,6′-difluoro[1,1′- biphenyl]-2-yl)cyclopropane-1- carbonyl]azetidin-3- yl}methyl)methanesulfonamide | 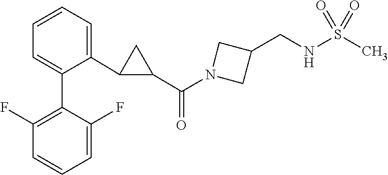 [SMILES: CS(=O)(=O)NCC1CN(C(=O)C2CC2c2ccccc2-c2c(F)cccc2F)C1] | 421.2 |
| 108 | rac-N-({1-[(1R,2R)-2-(2′,6′-difluoro[1,1′- biphenyl]-2-yl)cyclopropane-1- carbonyl]piperidin-4- yl}methyl)methanesulfonamide | 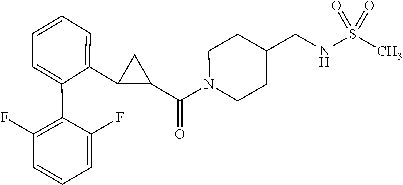 [SMILES: CS(=O)(=O)NCC1CCN(C(=O)C2CC2c2ccccc2-c2c(F)cccc2F)CC1] | 449.2 |
| 109 | a mixture of rac-N-{(1R,5S,6R)-3-[(1R,2R)-2- (2′,6-difluoro[1,1′-biphenyl]-2- yl)cyclopropane-1-carbonyl]-3- azabicyclo[3.1.0]hexan-6- yl}methanesulfonamide and rac-N- {(1S,5R,6S)-3-[(1R,2R)-2-(2′,6-difluoro[1,1′- biphenyl]-2-yl)cyclopropane-1-carbonyl]-3- azabicyclo[3.1.0]hexan-6- yl}methanesulfonamide | 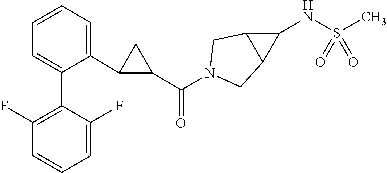 [SMILES: CS(=O)(=O)NC1C2CN(C(=O)C3CC3c3ccccc3-c3c(F)cccc3F)CC21] | 433.2 |
| 110 | a mixture of rac-N-{(4R)-1-[(1R,2R)-2-(3- methyl[1,1′-biphenyl]-2-yl)cyclopropane-1- carbonyl]azepan-4-yl}methanesulfonamide and rac-N-{(4S)-1-[(1R,2R)-2-(3-methyl[1,1′- biphenyl]-2-yl)cyclopropane-1- carbonyl]azepan-4-yl}methanesulfonamide | 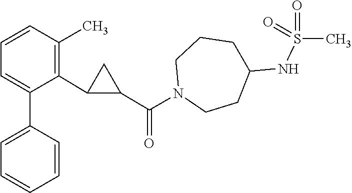 [SMILES: Cc1cccc(-c2ccccc2)c1C1CC1C(=O)N1CCCC(NS(C)(=O)=O)CC1] | 427.2 |
| 111 | rac-N-{8-[(1R,2R)-2-(2′,6′-difluoro[1,1′- biphenyl]-2-yl)cyclopropane-1-carbonyl]-8- azabicyclo[3.2.1]octan-3- yl}methanesulfonamide | 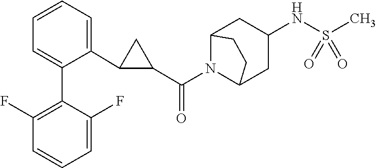 [SMILES: CS(=O)(=O)NC1CC2CCC(C1)N2C(=O)C1CC1c1ccccc1-c1c(F)cccc1F] | 461.2 |
| 112 | a mixture of rac-N-{(4R)-1-[(1R,2R)-2-(2′,6′- difluoro[1,1′-biphenyl]-2-yl)cyclopropane-1- carbonyl]azepan-4-yl}ethanesulfonamide and rac-N-{(4S)-1-[(1R,2R)-2-(2′,6-difluoro[1,1′- biphenyl]-2-yl)cyclopropane-1- carbonyl]azepan-4-yl}ethanesulfonamide | 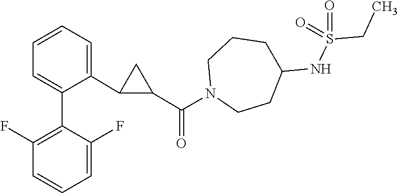 [SMILES: CCS(=O)(=O)NC1CCCN(C(=O)C2CC2c2ccccc2-c2c(F)cccc2F)CC1] | 463.2 |
| 113 | a mixture of rac-N′-{(3R)-1-[(1R,2R)-2-(2′,6′- difluoro[1,1′-biphenyl]-2-yl)cyclopropane-1- carbonyl]pyrrolidin-3-yl}-N,N-dimethylsulfuric diamide and rac-N′-{(3S)-1-[(1R,2R)-2-(2′,6- difluoro[1,1′-biphenyl]-2-yl)cyclopropane-1- carbonyl]pyrrolidin-3-yl}-N,N-dimethylsulfuric diamide | 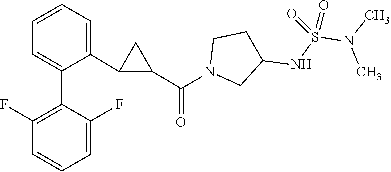 [SMILES: CN(C)S(=O)(=O)NC1CCN(C(=O)C2CC2c2ccccc2-c2c(F)cccc2F)C1] | 450.2 |
| 114 | a mixture of rac-N′-{(4R)-1-[(1R,2R)-2-(2′,6′- difluoro[1,1′-biphenyl]-2-yl)cyclopropane-1- carbonyl]azepan-4-yl}-N,N-dimethylsulfuric diamide and rac-N′-{(4S)-1-[(1R,2R)-2-(2′,6′- difluoro[1,1′-biphenyl]-2-yl)cyclopropane-1- carbonyl]azepan-4-yl}-N,N-dimethylsulfuric diamide | 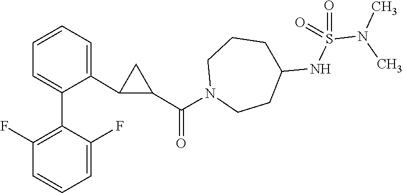 [SMILES: CN(C)S(=O)(=O)NC1CCCN(C(=O)C2CC2c2ccccc2-c2c(F)cccc2F)CC1] | 478.2 |
| 115 | rac-N-{2-[(1R,2R)-2-(2′,6′-difluoro[1,1′- biphenyl]-2-yl)cyclopropane-1-carbonyl]-2- azaspiro[3.3]heptan-6-yl}methanesulfonamide | 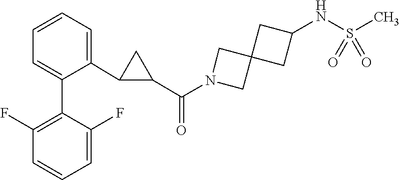 [SMILES: CS(=O)(=O)NC1CC2(C1)CN(C(=O)C1CC1c1ccccc1-c1c(F)cccc1F)C2] | 447.2 |
| 116 | a mixture of rac-N-{(4R)-1-[(1R,2R)-2-(2- cyclopentylphenyl)cyclopropane-1- carbonyl]azepan-4-yl}methanesulfonamide and rac-N-{(4S)-1-[(1R,2R)-2-(2- cyclopentylphenyl)cyclopropane-1- carbonyl]azepan-4-yl}methanesulfonamide | 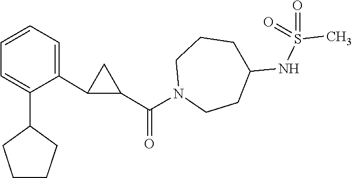 [SMILES: CS(=O)(=O)NC1CCCN(C(=O)C2CC2c2ccccc2C2CCCC2)CC1] | 405.2 |
| 117 | a mixture of rac-N-{(4R)-1-[(1R,2R)-2-(2- cyclobutylphenyl)cyclopropane-1- carbonyl]azepan-4-yl}methanesulfonamide and rac-N-{(4S)-1-[(1R,2R)-2-(2- cyclobutylphenyl)cyclopropane-1- carbonyl]azepan-4-yl}methanesulfonamide | 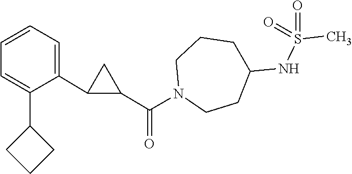 [SMILES: CS(=O)(=O)NC1CCCN(C(=O)C2CC2c2ccccc2C2CCC2)CC1] | 391.3 |
| 118 | a mixture of rac-N-{(4R)-1-[(1R,2R)-2-(2- cyclohexylphenyl)cyclopropane-1- carbonyl]azepan-4-yl}methanesulfonamide and rac-N-{(4S)-1-[(1R,2R)-2-(2- cyclohexylphenyl)cyclopropane-1- carbonyl]azepan-4-yl}methanesulfonamide | 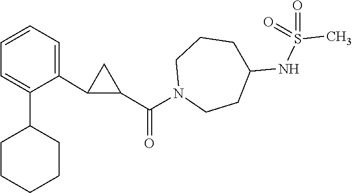 [SMILES: CS(=O)(=O)NC1CCCN(C(=O)C2CC2c2ccccc2C2CCCCC2)CC1] | 419.3 |
| 119 | a mixture of rac-N-{(3R)-1-[(1R,2R)-2-(2- cyclohexylphenyl)cyclopropane-1- carbonyl]piperidin-3-yl}methanesulfonamide and rac-N-{(3S)-1-[(1R,2R)-2-(2- cyclohexylphenyl)cyclopropane-1- carbonyl]piperidin-3-yl}methanesulfonamide | 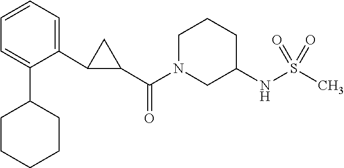 [SMILES: CS(=O)(=O)NC1CCCN(C(=O)C2CC2c2ccccc2C2CCCCC2)C1] | 405.3 |
| 120 | a mixture of rac-N-{(3R)-1-[(1R,2R)-2-(2- cyclopentylphenyl)cyclopropane-1- carbonyl]piperidin-3-yl}methanesulfonamide and rac-N-{(3S)-1-[(1R,2R)-2-(2- cyclopentylphenyl)cyclopropane-1- carbonyl]piperidin-3-yl}methanesulfonamide | 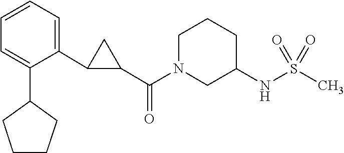 [SMILES: CS(=O)(=O)NC1CCCN(C(=O)C2CC2c2ccccc2C2CCCC2)C1] | 391.2 |
| 121 | a mixture of rac-N-{(3R)-1-[(1R,2R)-2-(2- cyclohexylphenyl)cyclopropane-1- carbonyl]pyrrolidin-3-yl}methanesulfonamide and rac-N-{(3S)-1-[(1R,2R)-2-(2- cyclohexylphenyl)cyclopropane-1- carbonyl]pyrrolidin-3-yl}methanesulfonamide | 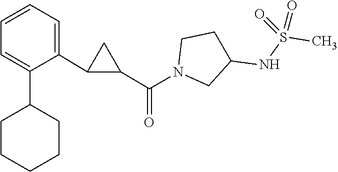 [SMILES: CS(=O)(=O)NC1CCN(C(=O)C2CC2c2ccccc2C2CCCCC2)C1] | 391.2 |
| 122 | a mixture of rac-N-{(3R)-1-[(1R,2R)-2-(2- cyclopentylphenyl)cyclopropane-1- carbonyl]pyrrolidin-3-yl}methanesulfonamide and rac-N-{(3S)-1-[(1R,2R)-2-(2- cyclopentylphenyl)cyclopropane-1- carbonyl]pyrrolidin-3-yl}methanesulfonamide | 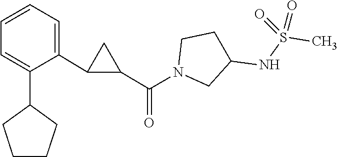 [SMILES: CS(=O)(=O)NC1CCN(C(=O)C2CC2c2ccccc2C2CCCC2)C1] | 377.2 |
| 123 | a mixture of rac-N-{(3R)-1-[(1R,2R)-2-(2′,6′- difluoro[1,1′-biphenyl]-2-yl)cyclopropane-1- carbonyl]piperidin-3- yl}cyclopropanesulfonamide and rac-N-{(3S)- 1-[(1R,2R)-2-(2′,6′-difluoro[1,1′-biphenyl]-2- yl)cyclopropane-1-carbonyl]piperidin-3- yl}cyclopropanesulfonamide | 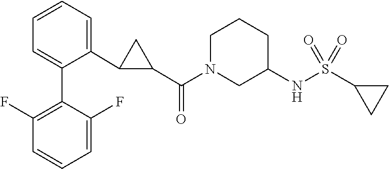 [SMILES: O=C(C1CC1c1ccccc1-c1c(F)cccc1F)N1CCCC(NS(=O)(=O)C2CC2)C1] | 461.2 |
| 124 | a mixture of rac-N-{(1S,5R)-3-[(1R,2R)-2- (2′,6′-difluoro[1,1′-biphenyl]-2- yl)cyclopropane-1-carbonyl]-3- azabicyclo[3.1.0]hexan-1- yl}methanesulfonamide and rac-N-{(1R,5S)-3- [(1R,2R)-2-(2′,6′-difluoro[1,1′-biphenyl]-2- yl)cyclopropane-1-carbonyl]-3- azabicyclo[3.1.0]hexan-1- yl}methanesulfonamide | 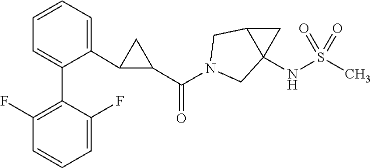 [SMILES: CS(=O)(=O)NC12CC1CN(C(=O)C1CC1c1ccccc1-c1c(F)cccc1F)C2] | 433.2 |
| 125 | a mixture of rac-N-{(3R)-1-[(1R,2R)-2-(2′- chloro-6-fluoro[1,1′-biphenyl]-2- yl)cyclopropane-1-carbonyl]pyrrolidin-3- yl}methanesulfonamide and rac-N-{(3S)-1- [(1R,2R)-2-(2′-chloro-6-fluoro[1,1′-biphenyl]- 2-yl)cyclopropane-1-carbonyl]pyrrolidin-3- yl}methanesulfonamide | 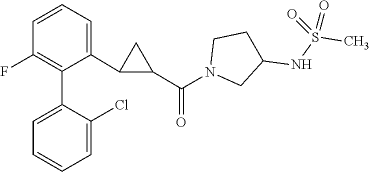 [SMILES: CS(=O)(=O)NC1CCN(C(=O)C2CC2c2cccc(F)c2-c2ccccc2Cl)C1] | 437.2 |
| 126 | a mixture of rac-N-{(3R)-1-[(1R,2R)-2-(2′- chloro-3-fluoro[1,1′-biphenyl]-2- yl)cyclopropane-1-carbonyl]pyrrolidin-3- yl}methanesulfonamide and rac-N-{(3S)-1- [(1R,2R)-2-(2′-chloro-3-fluoro[1,1′-biphenyl]- 2-yl)cyclopropane-1-carbonyl]pyrrolidin-3- yl}methanesulfonamide | 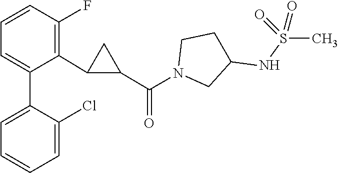 [SMILES: CS(=O)(=O)NC1CCN(C(=O)C2CC2c2c(F)cccc2-c2ccccc2Cl)C1] | 437.3 |
| 127 | a mixture of rac-N-({(2R)-1-[(1R,2R)-2-(2′,6′- difluoro[1,1′-biphenyl]-2-yl)cyclopropane-1- carbonyl]azetidin-2- yl}methyl)methanesulfonamide and rac-N- ({(2S)-1-[(1R,2R)-2-(2′,6′-difluoro[1,1′- biphenyl]-2-yl)cyclopropane-1- carbonyl]azetidin-2- yl}methyl)methanesulfonamide | 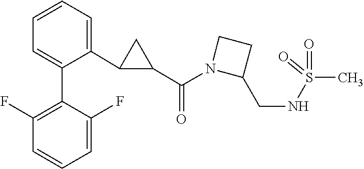 [SMILES: CS(=O)(=O)NCC1CCN1C(=O)C1CC1c1ccccc1-c1c(F)cccc1F] | 421.3 |
| 128 | a mixture of rac-N-({(3R)-1-[(1R,2R)-2-(2′,6′- difluoro[1,1′-biphenyl]-2-yl)cyclopropane-1- carbonyl]piperidin-3- yl}methyl)methanesulfonamide and rac-N- ({(3S)-1-[(1R,2R)-2-(2′,6′-difluoro[1,1′- biphenyl]-2-yl)cyclopropane-1- carbonyl]piperidin-3- yl}methyl)methanesulfonamide | 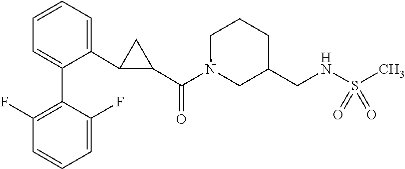 [SMILES: CS(=O)(=O)NCC1CCCN(C(=O)C2CC2c2ccccc2-c2c(F)cccc2F)C1] | 449.3 |
| 129 | a mixture of rac-N-{(3R)-1-[(1R,2R)-2-(2′,6′- difluoro[1,1′-biphenyl]-2-yl)cyclopropane-1- carbonyl]-4,4-difluoropiperidin-3- yl}methanesulfonamide and rac-N-{(3S)-1- [(1R,2R)-2-(2′,6′-difluoro[1,1′-biphenyl]-2- yl)cyclopropane-1-carbonyl]-4,4- difluoropiperidin-3-yl}methanesulfonamide | 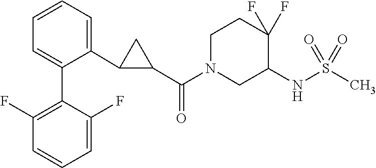 [SMILES: CS(=O)(=O)NC1CN(C(=O)C2CC2c2ccccc2-c2c(F)cccc2F)CCC1(F)F] | 471.3 |
| 130 | a mixture of rac-N-{(3R)-1-[(1R,2R)-2-(2′,6′- difluoro[1,1′-biphenyl]-2-yl)cyclopropane-1- carbonyl]-4,4-difluoropyrrolidin-3- yl}methanesulfonamide and rac-N-{(3S)-1- [(1R,2R)-2-(2′,6′-difluoro[1,1′-biphenyl]-2- yl)cyclopropane-1-carbonyl]-4,4- difluoropyrrolidin-3-yl}methanesulfonamide | 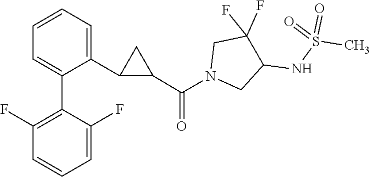 [SMILES: CS(=O)(=O)NC1CN(C(=O)C2CC2c2ccccc2-c2c(F)cccc2F)CC1(F)F] | 455.2 |
| 131 | a mixture of rac-N-({(2R)-1-[(1R,2R)-2-(2′,6′- difluoro[1,1′-biphenyl]-2-yl)cyclopropane-1- carbonyl]piperidin-2- yl}methyl)methanesulfonamide and rac-N- ({(2S)-1-[(1R,2R)-2-(2′,6′-difluoro[1,1′- biphenyl]-2-yl)cyclopropane-1- carbonyl]piperidin-2- yl}methyl)methanesulfonamide | 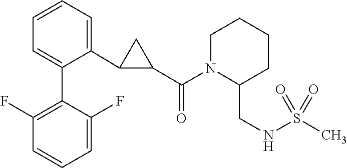 [SMILES: CS(=O)(=O)NCC1CCCCN1C(=O)C1CC1c1ccccc1-c1c(F)cccc1F] | 449.3 |
| 132 | a mixture of rac-N-{(3R)-1-[(1S,2S)-2-([1,1′- biphenyl]-2-yl)-2-fluorocyclopropane-1- carbonyl]pyrrolidin-3-yl}methanesulfonamide and rac-N-{(3S)-1-[(1S,2S)-2-([1,1′- biphenyl]-2-yl)-2-fluorocyclopropane-1- carbonyl]pyrrolidin-3-yl}methanesulfonamide | 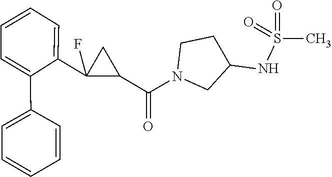 [SMILES: CS(=O)(=O)NC1CCN(C(=O)C2CC2(F)c2ccccc2-c2ccccc2)C1] | 403.2 |
| 134 | a mixture of rac-N-{(3R)-1-[(1R,2R)-2-(2′- chloro-5-fluoro[1,1′-biphenyl]-2- yl)cyclopropane-1-carbonyl]pyrrolidin-3- yl}methanesulfonamide and rac-N-{(3S)-1- [(1R,2R)-2-(2′-chloro-5-fluoro[1,1′-biphenyl]- 2-yl)cyclopropane-1-carbonyl]pyrrolidin-3- yl}methanesulfonamide | 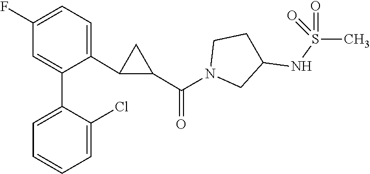 [SMILES: CS(=O)(=O)NC1CCN(C(=O)C2CC2c2ccc(F)cc2-c2ccccc2Cl)C1] | 437.1 |
| 135 | a mixture of rac-N-{(3R)-1-[(1R,2R)-2-(2′,5,6′- trifluoro[1,1′-biphenyl]-2-yl)cyclopropane-1- carbonyl]pyrrolidin-3-yl}methanesulfonamide and rac-N-{(3S)-1-[(1R,2R)-2-(2′,5,6′- trifluoro[1,1′-biphenyl]-2-yl)cyclopropane-1- carbonyl]pyrrolidin-3-yl}methanesulfonamide | 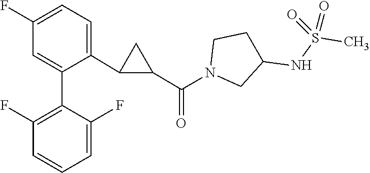 [SMILES: CS(=O)(=O)NC1CCN(C(=O)C2CC2c2ccc(F)cc2-c2c(F)cccc2F)C1] | 439.2 |
| 136 | a mixture of rac-N-{(3R)-1-[(1R,2R)-2-(2′,6′- difluoro-5-methyl[1,1′-biphenyl]-2- yl)cyclopropane-1-carbonyl]pyrrolidin-3- yl}methanesulfonamide and rac-N-{(3S)-1- [(1R,2R)-2-(2′,6′-difluoro-5-methyl[1,1′- biphenyl]-2-yl)cyclopropane-1- carbonyl]pyrrolidin-3-yl}methanesulfonamide | 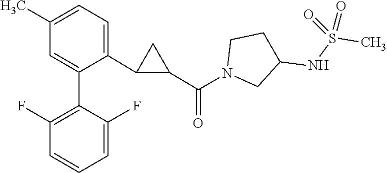 | 435.2 |
| 137 | a mixture of rac-N-{(3S,4R)-1-[(1R,2R)-2- (2′,6′-difluoro[1,1′-biphenyl]-2- yl)cyclopropane-1-carbonyl]-4- fluoropyrrolidin-3-yl}methanesulfonamide and rac-N-{(3R,4S)-1-[(1R,2R)-2-(2′,6′- difluoro[1,1′-biphenyl]-2-yl)cyclopropane-1- carbonyl]-4-fluoropyrrolidin-3- yl}methanesulfonamide | 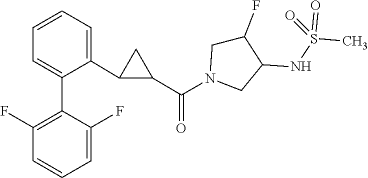 [SMILES: CS(=O)(=O)NC1CN(C(=O)C2CC2c2ccccc2-c2c(F)cccc2F)CC1F] | 439.2 |
| 138 | a mixture of rac-N-{(3R)-1-[(1R,2R)-2-(5- chloro[1,1′-biphenyl]-2-yl)cyclopropane-1- carbonyl]pyrrolidin-3-yl}methanesulfonamide and rac-N-{(3S)-1-[(1R,2R)-2-(5-chloro[1,1′- biphenyl]-2-yl)cyclopropane-1- carbonyl]pyrrolidin-3-yl}methanesulfonamide | 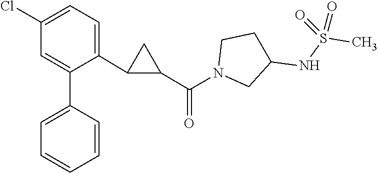 [SMILES: CS(=O)(=O)NC1CCN(C(=O)C2CC2c2ccc(Cl)cc2-c2ccccc2)C1] | 419.2 |
| 140 | a mixture of rac-N-{(3R)-1-[(1R,2R)-2-(6- chloro[1,1′-biphenyl]-2-yl)cyclopropane-1- carbonyl]piperidin-3-yl}methanesulfonamide and rac-N-{(3S)-1-[(1R,2R)-2-(6-chloro[1,1′- biphenyl]-2-yl)cyclopropane-1- carbonyl]piperidin-3-yl}methanesulfonamide | 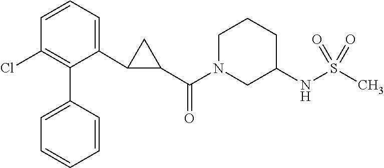 [SMILES: CS(=O)(=O)NC1CCCN(C(=O)C2CC2c2cccc(Cl)c2-c2ccccc2)C1] | 433.2 |
| 141 | a mixture of rac-N-{(4R)-1-[(1R,2R)-2-(6- chloro[1,1′-biphenyl]-2-yl)cyclopropane-1- carbonyl]azepan-4-yl}methanesulfonamide and rac-N-{(4S)-1-[(1R,2R)-2-(6-chloro[1,1′- biphenyl]-2-yl)cyclopropane-1- carbonyl]azepan-4-yl}methanesulfonamide | 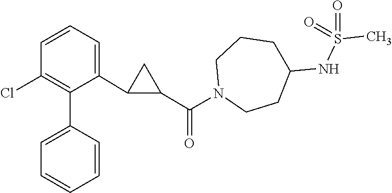 [SMILES: CS(=O)(=O)NC1CCCN(C(=O)C2CC2c2cccc(Cl)c2-c2ccccc2)CC1] | 447.2 |
| 142 | a mixture of rac-N-{(3R)-1-[(1R,2R)-2-(5- chloro[1,1′-biphenyl]-2-yl)cyclopropane-1- carbonyl]piperidin-3-yl}methanesulfonamide and rac-N-{(3S)-1-[(1R,2R)-2-(5-chloro[1,1′- biphenyl]-2-yl)cyclopropane-1- carbonyl]piperidin-3-yl}methanesulfonamide | 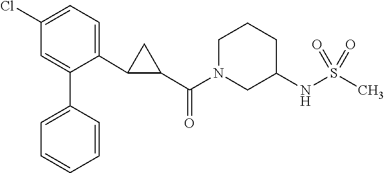 [SMILES: CS(=O)(=O)NC1CCCN(C(=O)C2CC2c2ccc(Cl)cc2-c2ccccc2)C1] | 433.2 |
| 143 | a mixture of rac-N-{(4R)-1-[(1R,2R)-2-(5- chloro[1,1′-biphenyl]-2-yl)cyclopropane-1- carbonyl]azepan-4-yl}methanesulfonamide and rac-N-{(4S)-1-[(1R,2R)-2-(5-chloro[1,1′- biphenyl]-2-yl)cyclopropane-1- carbonyl]azepan-4-yl}methanesulfonamide | 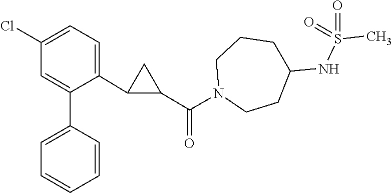 [SMILES: CS(=O)(=O)NC1CCCN(C(=O)C2CC2c2ccc(Cl)cc2-c2ccccc2)CC1] | 447.2 |
| 144 | a mixture of rac-N-{(3R)-1-[(1S,2S)-2-([1,1′- biphenyl]-2-yl)-2-fluorocyclopropane-1- carbonyl]piperidin-3-yl}methanesulfonamide and rac-N-{(3S)-1-[(1S,2S)-2-([1,1′- biphenyl]-2-yl)-2-fluorocyclopropane-1- carbonyl]piperidin-3-yl}methanesulfonamide | 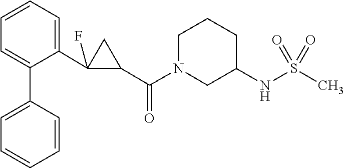 [SMILES: CS(=O)(=O)NC1CCCN(C(=O)C2CC2(F)c2ccccc2-c2ccccc2)C1] | 417.3 |
| 145 | a mixture of rac-N-{(3R)-1-[(1R,2R)-2-(2′- chloro-5-methyl[1,1′-biphenyl]-2- yl)cyclopropane-1-carbonyl]pyrrolidin-3- yl}methanesulfonamide and rac-N-{(3S)-1- [(1R,2R)-2-(2′-chloro-5-methyl[1,1-biphenyl]- 2-yl)cyclopropane-1-carbonyl]pyrrolidin-3- yl}methanesulfonamide | 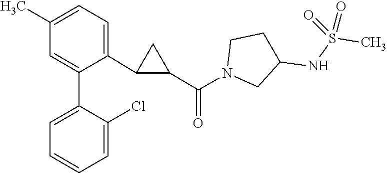 | 433.2 |
| 146 | a mixture of rac-N-{(3R)-1-[(1R,2R)-2-(3- phenylthiophen-2-yl)cyclopropane-1- carbonyl]piperidin-3-yl}methanesulfonamide and rac-{(3S)-1-[(1R,2R)-2-(3- phenylthiophen-2-yl)cyclopropane-1- carbonyl]piperidin-3-yl}methanesulfonamide | 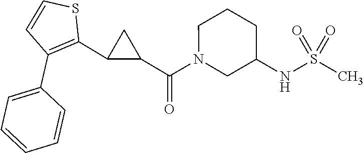 [SMILES: CS(=O)(=O)NC1CCCN(C(=O)C2CC2c2sccc2-c2ccccc2)C1] | 405.2 |
| 147 | a mixture of rac-N-{(4R)-1-[(1R,2R)-2-(2- phenylthiophen-3-yl)cyclopropane-1- carbonyl]azepan-4-yl}methanesulfonamide and rac-N-{(4S)-1-[(1R,2R)-2-(2- phenylthiophen-3-yl)cyclopropane-1- carbonyl]azepan-4-yl}methanesulfonamide | 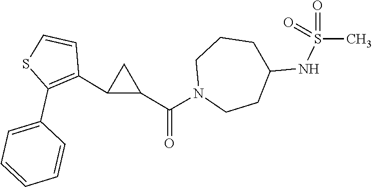 [SMILES: CS(=O)(=O)NC1CCCN(C(=O)C2CC2c2ccsc2-c2ccccc2)CC1] | 419.3 |
| 148 | a mixture of rac-N-{(4R)-1-[(1R,2R)-2-(3- phenylthiophen-2-yl)cyclopropane-1- carbonyl]azepan-4-yl}methanesulfonamide and rac-N-{(4S)-1-[(1R,2R)-2-(3- phenylthiophen-2-yl)cyclopropane-1- carbonyl]azepan-4-yl}methanesulfonamide | 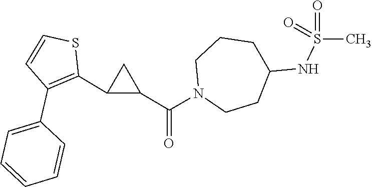 [SMILES: CS(=O)(=O)NC1CCCN(C(=O)C2CC2c2sccc2-c2ccccc2)CC1] | 419.2 |
| 149 | a mixture of rac-N-{(3R)-1-[(1R,2R)-2-(3- phenylthiophen-2-yl)cyclopropane-1- carbonyl]pyrrolidin-3-yl}methanesulfonamide and rac-N-{(3S)-1-[(1R,2R)-2-(3- phenylthiophen-2-yl)cyclopropane-1- carbonyl]pyrrolidin-3-yl}methanesulfonamide | 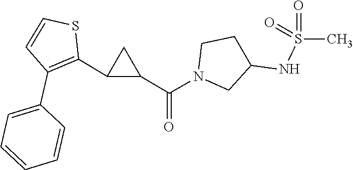 [SMILES: CS(=O)(=O)NC1CCN(C(=O)C2CC2c2sccc2-c2ccccc2)C1] | 391.3 |
| 150 | a mixture of rac-N-{(3R)-1-[(1R,2R)-2-(2- phenylthiophen-3-yl)cyclopropane-1- carbonyl]pyrrolidin-3-yl}methanesulfonamide and rac-N-{(3S)-1-[(1R,2R)-2-(2- phenylthiophen-3-yl)cyclopropane-1- carbonyl]pyrrolidin-3-yl}methanesulfonamide | 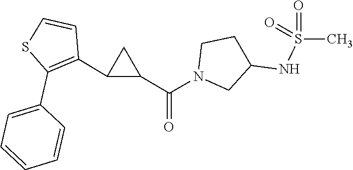 [SMILES: CS(=O)(=O)NC1CCN(C(=O)C2CC2c2ccsc2-c2ccccc2)C1] | 391.2 |
| 151 | a mixture of rac-N-{(3R)-1-[(1R,2R)-2-(5- phenylthiophen-2-yl)cyclopropane-1- carbonyl]piperidin-3-yl}methanesulfonamide and rac-N-{(3S)-1-[(1R,2R)-2-(5- phenylthiophen-2-yl)cyclopropane-1- carbonyl]piperidin-3-yl}methanesulfonamide | 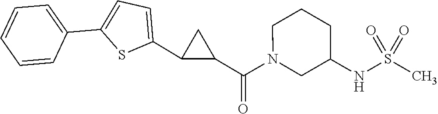 [SMILES: CS(=O)(=O)NC1CCCN(C(=O)C2CC2c2ccc(-c3ccccc3)s2)C1] | 405.3 |
| 152 | a mixture of rac-N-{(3R)-1-[(1R,2R)-2-(2′,5,6′- trifluoro[1,1′-biphenyl]-2-yl)cyclopropane-1- carbonyl]piperidin-3-yl}methanesulfonamide and rac-N-{(3S)-1-[(1R,2R)-2-(2′,5,6′- trifluoro[1,1′-biphenyl]-2-yl)cyclopropane-1- carbonyl]piperidin-3-yl}methanesulfonamide | 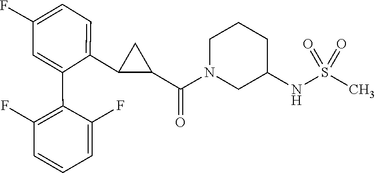 [SMILES: CS(=O)(=O)NC1CCCN(C(=O)C2CC2c2ccc(F)cc2-c2c(F)cccc2F)C1] | 453.2 |
| 153 | a mixture of rac-N-{(4R)-1-[(1R,2R)-2-(2′,5,6′- trifluoro[1,1′-biphenyl]-2-yl)cyclopropane-1- carbonyl]azepan-4-yl}methanesulfonamide and rac-N-{(4S)-1-[(1R,2R)-2-(2′,5,6′- trifluoro[1,1′-biphenyl]-2-yl)cyclopropane-1- carbonyl]azepan-4-yl}methanesulfonamide | 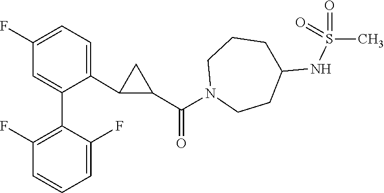 [SMILES: CS(=O)(=O)NC1CCCN(C(=O)C2CC2c2ccc(F)cc2-c2c(F)cccc2F)CC1] | 467.3 |
| 154 | a mixture of rac-N-{(4R)-1-[(1R,2R)-2-(4- phenylthiophen-2-yl)cyclopropane-1- carbonyl]azepan-4-yl}methanesulfonamide and rac-N-{(4S)-1-[(1R,2R)-2-(4- phenylthiophen-2-yl)cyclopropane-1- carbonyl]azepan-4-yl}methanesulfonamide | 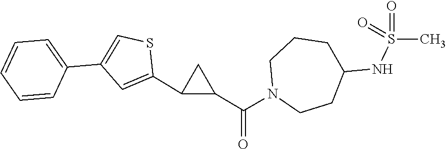 [SMILES: CS(=O)(=O)NC1CCCN(C(=O)C2CC2c2cc(-c3ccccc3)cs2)CC1] | 419.3 |
| 155 | a mixture of rac-N-{(4R)-1-[(1R,2R)-2-(3- chloro[1,1′-biphenyl]-2-yl)cyclopropane-1- carbonyl]azepan-4-yl}methanesulfonamide and rac-N-{(4S)-1-[(1R,2R)-2-(3-chloro[1,1′- biphenyl]-2-yl)cyclopropane-1- carbonyl]azepan-4-yl}methanesulfonamide | 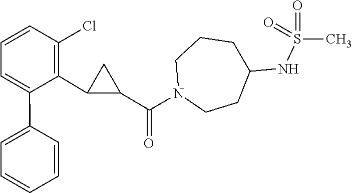 [SMILES: CS(=O)(=O)NC1CCCN(C(=O)C2CC2c2c(Cl)cccc2-c2ccccc2)CC1] | 447.3 |
| 156 | a mixture of rac-N-{(3R)-1-[(1R,2R)-2-(3- chloro[1,1′-biphenyl]-2-yl)cyclopropane-1- carbonyl]piperidin-3-yl}methanesulfonamide and rac-N-{(3S)-1-[(1R,2R)-2-(3-chloro[1,1′- biphenyl]-2-yl)cyclopropane-1- carbonyl]piperidin-3-yl}methanesulfonamide | 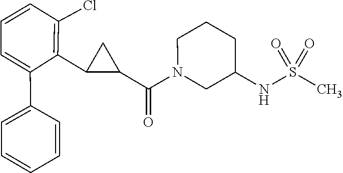 [SMILES: CS(=O)(=O)NC1CCCN(C(=O)C2CC2c2c(Cl)cccc2-c2ccccc2)C1] | 433.3 |
| 157 | a mixture of rac-N-{(3R)-1-[(1R,2R)-2-(3- chloro[1,1′-biphenyl]-2-yl)cyclopropane-1- carbonyl]pyrrolidin-3-yl}methanesulfonamide and rac-N-{(3S)-1-[(1R,2R)-2-(3-chloro[1,1′- biphenyl]-2-yl)cyclopropane-1- carbonyl]pyrrolidin-3-yl}methanesulfonamide | 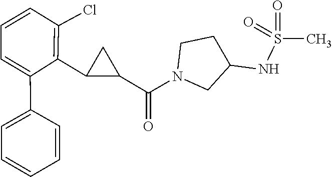 [SMILES: CS(=O)(=O)NC1CCN(C(=O)C2CC2c2c(Cl)cccc2-c2ccccc2)C1] | 419.3 |
| 158 | a mixture of rac-N-{(3S,5R)-1-[(1R,2R)-2- (2′,6′-difluoro[1,1′-biphenyl]-2- yl)cyclopropane-1-carbonyl]-5- methylpiperidin-3-yl}methanesulfonamide and rac-N-{(3R,5S)-1-[(1R,2R)-2-(2′,6′- difluoro[1,1′-biphenyl]-2-yl)cyclopropane-1- carbonyl]-5-methylpiperidin-3- yl}methanesulfonamide and rac-N-{(3S,5S)-1- [(1R,2R)-2-(2′,6′-difluoro[1,1′-biphenyl]-2- yl)cyclopropane-1-carbonyl]-5- methylpiperidin-3-yl}methanesulfonamide | 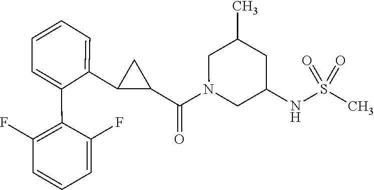 [SMILES: CC1CC(NS(C)(=O)=O)CN(C(=O)C2CC2c2ccccc2-c2c(F)cccc2F)C1] | 449.3 |
| 159 | a mixture of rac-N-{(3R)-1-[(1R,2R)-2-(2′,6′- difluoro[1,1′-biphenyl]-2-yl)cyclopropane-1- carbonyl]azepan-3-yl}ethanesulfonamide and rac-N-{(3S)-1-[(1R,2R)-2-(2′,6′-difluoro[1,1′- biphenyl]-2-yl)cyclopropane-1- carbonyl]azepan-3-yl}ethanesulfonamide | 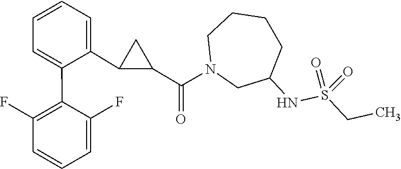 [SMILES: CCS(=O)(=O)NC1CCCCN(C(=O)C2CC2c2ccccc2-c2c(F)cccc2F)C1] | 463.3 |
| 160 | a mixture of rac-N-{(4R)-1-[(1R,2R)-2-([1,1′- biphenyl]-3-yl)cyclopropane-1- carbonyl]azepan-4-yl}methanesulfonamide and rac-N-{(4S)-1-[(1R,2R)-2-([1,1′- biphenyl]-3-yl)cyclopropane-1- carbonyl]azepan-4-yl}methanesulfonamide | 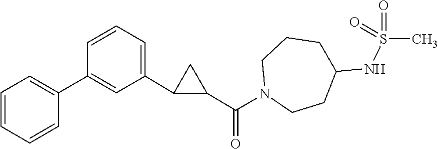 [SMILES: CS(=O)(=O)NC1CCCN(C(=O)C2CC2c2cccc(-c3ccccc3)c2)CC1] | 413.4 |
| 161 | a mixture of rac-N-{(3R)-1-[(1R,2R)-2-(2′,6′- difluoro[1,1′-biphenyl]-2-yl)cyclopropane-1- carbonyl]azepan-3- yl}cyclopropanesulfonamide and rac-N-{(3S)- 1-[(1R,2R)-2-(2′,6′-difluoro[1,1′-biphenyl]-2- yl)cyclopropane-1-carbonyl]azepan-3- yl}cyclopropanesulfonamide | 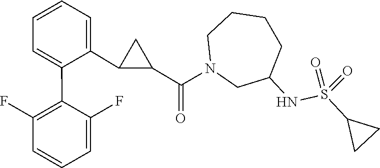 [SMILES: O=C(C1CC1c1ccccc1-c1c(F)cccc1F)N1CCCCC(NS(=O)(=O)C2CC2)C1] | 475.3 |
| 162 | a mixture of rac-N′-{(3R)-1-[(1R,2R)-2-(2′,6′- difluoro[1,1′-biphenyl]-2-yl)cyclopropane-1- carbonyl]azepan-3-yl}-N,N-dimethylsulfuric diamide and rac-N′-{(3S)-1-[(1R,2R)-2-(2′,6′- difluoro[1,1′-biphenyl]-2-yl)cyclopropane-1- carbonyl]azepan-3-yl}-N,N-dimethylsulfuric diamide | 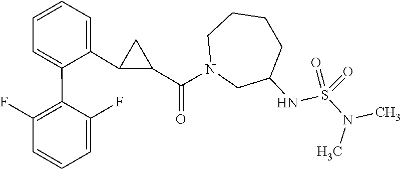 [SMILES: CN(C)S(=O)(=O)NC1CCCCN(C(=O)C2CC2c2ccccc2-c2c(F)cccc2F)C1] | 478.3 |
| 163 | a mixture of rac-N-{(3R)-1-[(1R,2R)-2-([1,1′- biphenyl]-3-yl)cyclopropane-1- carbonyl]piperidin-3-yl}methanesulfonamide and rac-N-{(3S)-1-[(1R,2R)-2-([1,1′- biphenyl]-3-yl)cyclopropane-1- carbonyl]piperidin-3-yl}methanesulfonamide | 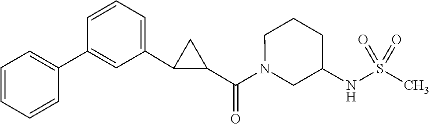 [SMILES: CS(=O)(=O)NC1CCCN(C(=O)C2CC2c2cccc(-c3ccccc3)c2)C1] | 399.3 |
| 164 | a mixture of rac-N′-{(3R)-1-[(1R,2R)-2-(2′,6′- difluoro[1,1′-biphenyl]-2-yl)cyclopropane-1- carbonyl]piperidin-3-yl}-N,N-dimethylsulfuric diamide and rac-N′-{(3S)-1-[(1R,2R)-2-(2′,6′- difluoro[1,1′-biphenyl]-2-yl)cyclopropane-1- carbonyl]piperidin-3-yl}-N,N-dimethylsulfuric diamide | 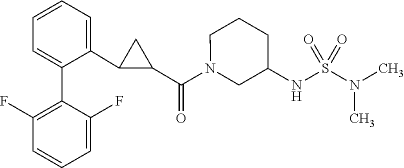 [SMILES: CN(C)S(=O)(=O)NC1CCCN(C(=O)C2CC2c2ccccc2-c2c(F)cccc2F)C1] | 464.3 |
| 165 | a mixture of rac-N-{(3R)-1-[(1R,2S)-2-([1,1′- biphenyl]-2-yl)-1-methylcyclopropane-1- carbonyl]pyrrolidin-3-yl}methanesulfonamide and rac-N-{(3S)-1-[(1R,2S)-2-([1,1′- biphenyl]-2-yl)-1-methylcyclopropane-1- carbonyl]pyrrolidin-3-yl}methanesulfonamide | 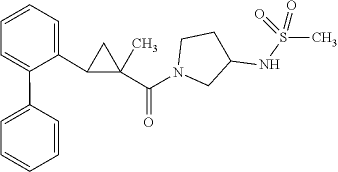 [SMILES: CC1(C(=O)N2CCC(NS(C)(=O)=O)C2)CC1c1ccccc1-c1ccccc1] | 399.3 |
| 166 | a mixture of rac-N-{(3R)-1-[(1R,2R)-2-(2′,6′- difluoro-5-methyl[1,1′-biphenyl]-2- yl)cyclopropane-1-carbonyl]piperidin-3- yl}methanesulfonamide and rac-N-{(3S)-1- [(1R,2R)-2-(2′,6′-difluoro-5-methyl[1,1′- biphenyl]-2-yl)cyclopropane-1- carbonyl]piperidin-3-yl}methanesulfonamide | 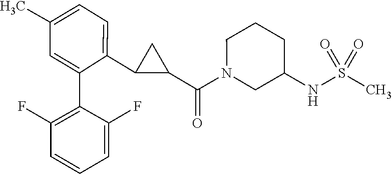 | 449.3 |
| 167 | a mixture of rac-N-{(4R)-1-[(1R,2R)-2-(2′,6′- difluoro-5-methyl[1,1′-biphenyl]-2- yl)cyclopropane-1-carbonyl]azepan-4- yl}methanesulfonamide and rac-N-{(4S)-1- [(1R,2R)-2-(2′,6′-difluoro-5-methyl[1,1′- biphenyl]-2-yl)cyclopropane-1- carbonyl]azepan-4-yl}methanesulfonamide | 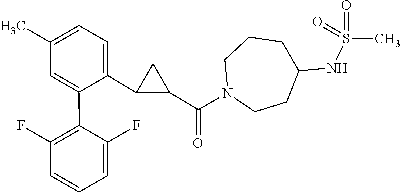 | 463.3 |
| 168 | a mixture of rac-N-[(3R)-1-{(1R,2R)-2-[5- (difluoromethyl)[1,1′-biphenyl]-2- yl]cyclopropane-1-carbonyl}piperidin-3- yl]methanesulfonamide and rac-N-[(3S)-1- {(1R,2R)-2-[5-(difluoromethyl)[1,1′-biphenyl]- 2-yl]cyclopropane-1-carbonyl}piperidin-3- yl]methanesulfonamide | 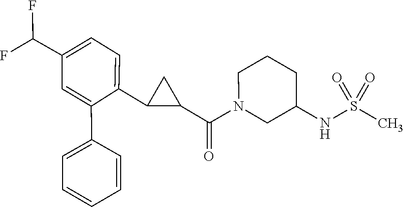 [SMILES: CS(=O)(=O)NC1CCCN(C(=O)C2CC2c2ccc(C(F)F)cc2-c2ccccc2)C1] | 449.3 |
| 169 | a mixture of rac-N-[(4R)-1-{(1R,2R)-2-[5- (difluoromethyl)[1,1′-biphenyl]-2- yl]cyclopropane-1-carbonyl}azepan-4- yl]methanesulfonamide and rac-N-[(4S)-1- {(1R,2R)-2-[5-(difluoromethyl)[1,1′-biphenyl]- 2-yl]cyclopropane-1-carbonyl}azepan-4- yl]methanesulfonamide | 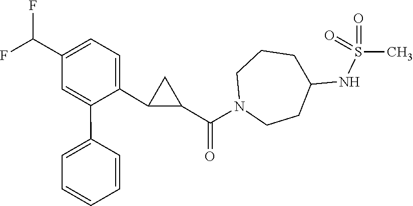 [SMILES: CS(=O)(=O)NC1CCCN(C(=O)C2CC2c2ccc(C(F)F)cc2-c2ccccc2)CC1] | 463.3 |
| 170 | a mixture of rac-N-[(3R)-1-{(1R,2R)-2-[5- (difluoromethyl)[1,1′-biphenyl]-2- yl]cyclopropane-1-carbonyl}pyrrolidin-3- yl]methanesulfonamide and rac-[(3S)-1- {(1R,2R)-2-[5-(difluoromethyl)[1,1′-biphenyl]- 2-yl]cyclopropane-1-carbonyl}pyrrolidin-3- yl]methanesulfonamide | 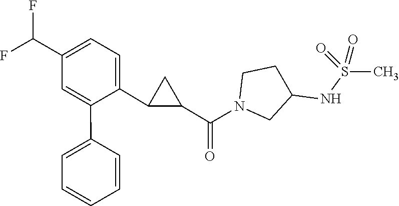 [SMILES: CS(=O)(=O)NC1CCN(C(=O)C2CC2c2ccc(C(F)F)cc2-c2ccccc2)C1] | 435.3 |
| 171 | a mixture of rac-N-{(3R)-1-[(1R,2R)-2-(5- phenylthiophen-2-yl)cyclopropane-1- carbonyl]pyrrolidin-3-yl}methanesulfonamide and rac-N-{(3S)-1-[(1R,2R)-2-(5- phenylthiophen-2-yl)cyclopropane-1- carbonyl]pyrrolidin-3-yl}methanesulfonamide | 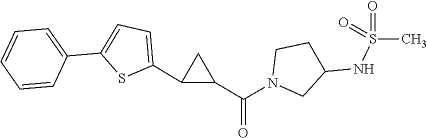 [SMILES: CS(=O)(=O)NC1CCN(C(=O)C2CC2c2ccc(-c3ccccc3)s2)C1] | 391.1 |
| 172 | a mixture of rac-N-{(2R,4R)-1-[(1R,2R)-2- (2′,6′-difluoro[1,1′-biphenyl]-2- yl)cyclopropane-1-carbonyl]-2-methylazepan- 4-yl}methanesulfonamide and rac-N-{(2S,4S)- 1-[(1R,2R)-2-(2′,6′-difluoro[1,1′-biphenyl]-2- yl)cyclopropane-1-carbonyl]-2-methylazepan- 4-yl}methanesulfonamide | 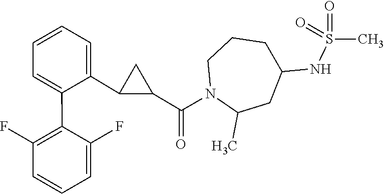 [SMILES: CC1CC(NS(C)(=O)=O)CCCN1C(=O)C1CC1c1ccccc1-c1c(F)cccc1F] | 463.2 |
| 173 | a mixture of rac-N-{(3R)-1-[(1R,2R)-2-(4- phenylthiophen-2-yl)cyclopropane-1- carbonyl]pyrrolidin-3-yl}methanesulfonamide and rac-N-{(3S)-1-[(1R,2R)-2-(4- phenylthiophen-2-yl)cyclopropane-1- carbonyl]pyrrolidin-3-yl}methanesulfonamide | 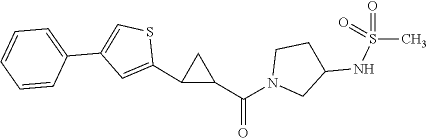 [SMILES: CS(=O)(=O)NC1CCN(C(=O)C2CC2c2cc(-c3ccccc3)cs2)C1] | 391.1 |
| 174 | a mixture of rac-N-{(3R)-1-[(1R,2R)-2-(4- phenylthiophen-2-yl)cyclopropane-1- carbonyl]piperidin-3-yl}methanesulfonamide and rac-N-{(3S)-1-[(1R,2R)-2-(4- phenylthiophen-2-yl)cyclopropane-1- carbonyl]piperidin-3-yl}methanesulfonamide | 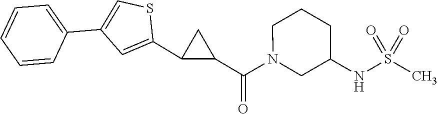 [SMILES: CS(=O)(=O)NC1CCCN(C(=O)C2CC2c2cc(-c3ccccc3)cs2)C1] | 405.1 |
| 175 | a mixture of rac-N-{(4R)-1-[(1R,2R)-2-(3- fluoro-5-methyl[1,1′-biphenyl]-2- yl)cyclopropane-1-carbonyl]azepan-4- yl}methanesulfonamide and rac-N-{(4S)-1- [(1R,2R)-2-(3-fluoro-5-methyl[1,1′-biphenyl]- 2-yl)cyclopropane-1-carbonyl]azepan-4- yl}methanesulfonamide | 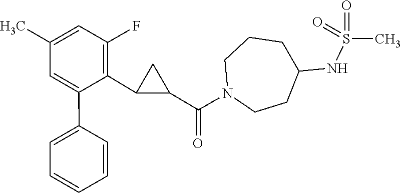 | 445.2 |
| 176 | a mixture of rac-N-{(3R)-1-[(1R,2R)-2-(3- fluoro-5-methyl[1,1′-biphenyl]-2- yl)cyclopropane-1-carbonyl]piperidin-3- yl}methanesulfonamide and rac-N-{(3S)-1- [(1R,2R)-2-(3-fluoro-5-methyl[1,1′-biphenyl]- 2-yl)cyclopropane-1-carbonyl]piperidin-3- yl}methanesulfonamide | 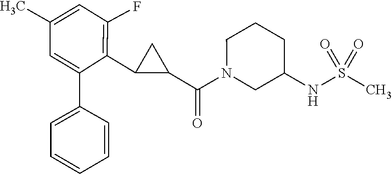 | 431.3 |
| 177 | a mixture of rac-N-{(3R)-1-[(1R,2R)-2-(3- fluoro-5-methyl[1,1′-biphenyl]-2- yl)cyclopropane-1-carbonyl]pyrrolidin-3- yl}methanesulfonamide and rac-N-{(3S)-1- [(1R,2R)-2-(3-fluoro-5-methyl[1,1′-biphenyl]- 2-yl)cyclopropane-1-carbonyl]pyrrolidin-3- yl}methanesulfonamide | 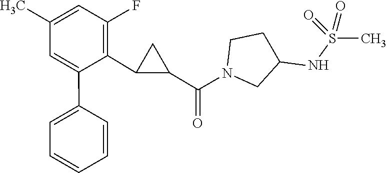 | 417.2 |
| 178 | a mixture of rac-N-{(3R)-1-[(1R,2R)-2-(5- methoxy[1,1′-biphenyl]-2-yl)cyclopropane-1- carbonyl]piperidin-3-yl}methanesulfonamide and rac-N-{(3S)-1-[(1R,2R)-2-(5- methoxy[1,1′-biphenyl]-2-yl)cyclopropane-1- carbonyl]piperidin-3-yl}methanesulfonamide | 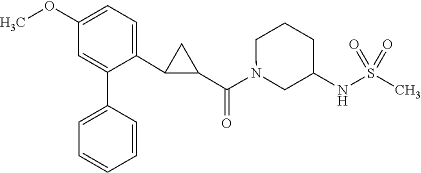 | 429.2 |
| 179 | a mixture of rac-N-{(3R)-1-[(1R,2R)-2-(5- methoxy[1,1′-biphenyl]-2-yl)cyclopropane-1- carbonyl]azepan-3-yl}methanesulfonamide and rac-N-{(3S)-1-[(1R,2R)-2-(5- methoxy[1,1′-biphenyl]-2-yl)cyclopropane-1- carbonyl]azepan-3-yl}methanesulfonamide | 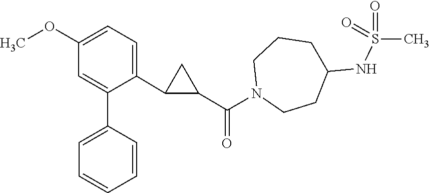 | 443.3 |
| 180 | N-{1-[2-(2′,6′-difluoro[1,1′-biphenyl]-2- yl)cyclopropane-1-carbonyl]-4,4- difluoropyrrolidin-3-yl}methanesulfonamide optical isomer | 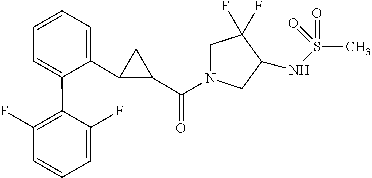 [SMILES: CS(=O)(=O)NC1CN(C(=O)C2CC2c2ccccc2-c2c(F)cccc2F)CC1(F)F] | 455.2 |
| 181 | a mixture of rac-N-{(4R)-1-[(1R,2R,3R)-2- ([1,1′-biphenyl]-2-yl)-3-methylcyclopropane- 1-carbonyl]azepan-4-yl}methanesulfonamide and rac-N-{(4S)-1-[(1R,2R,3R)-2-([1,1′- biphenyl]-2-yl)-3-methylcyclopropane-1- carbonyl]azepan-4-yl}methanesulfonamide | 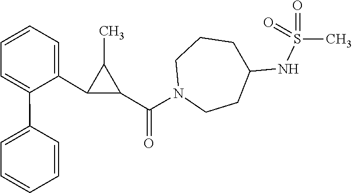 [SMILES: CC1C(C(=O)N2CCCC(NS(C)(=O)=O)CC2)C1c1ccccc1-c1ccccc1] | 427.3 |
| 182 | a mixture of rac-N-{(4R)-1-[(1R,2R,3S)-2- ([1,1′-biphenyl]-2-yl)-3-methylcyclopropane- 1-carbonyl]azepan-4-yl}methanesulfonamide and rac-N-{(4S)-1-[(1R,2R,3S)-2-([1,1′- biphenyl]-2-yl)-3-methylcyclopropane-1- carbonyl]azepan-4-yl}methanesulfonamide | 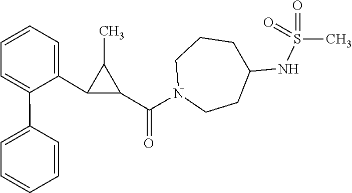 [SMILES: CC1C(C(=O)N2CCCC(NS(C)(=O)=O)CC2)C1c1ccccc1-c1ccccc1] | 427.2 |
| 183 | a mixture of N-{(3R)-1-[(1R,2R)-2-(2′,6′- difluoro[1,1′-biphenyl]-2-yl)cyclopropane-1- carbonyl]azepan-3-yl}methanesulfonamide and N-{(3R)-1-[(1S,2S)-2-(2′,6′-difluoro[1,1′- biphenyl]-2-yl)cyclopropane-1- carbonyl]azepan-3-yl}methanesulfonamide | 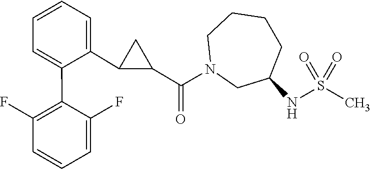 [SMILES: CS(=O)(=O)N[C@@H]1CCCCN(C(=O)C2CC2c2ccccc2-c2c(F)cccc2F)C1] | 449.2 |
| 184 | a mixture of N-{(3S)-1-[(1R,2R)-2-(2′,6′- difluoro[1,1′-biphenyl]-2-yl)cyclopropane-1- carbonyl]azepan-3-yl}methanesulfonamide and N-{(3S)-1-[(1S,2S)-2-(2′,6′-difluoro[1,1′- biphenyl]-2-yl)cyclopropane-1- carbonyl]azepan-3-yl}methanesulfonamide | 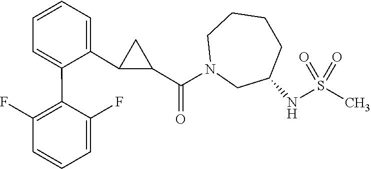 [SMILES: CS(=O)(=O)N[C@H]1CCCCN(C(=O)C2CC2c2ccccc2-c2c(F)cccc2F)C1] | 449.2 |
| 185 | a mixture of rac-N-{(3R,4R)-1-[(1R,2R)-2- (2′,6′-difluoro[1,1′-biphenyl]-2- yl)cyclopropane-1-carbonyl]-4- methylpiperidin-3-yl}methanesulfonamide and rac-N-{(3S,4S)-1-[(1R,2R)-2-(2′,6′- difluoro[1,1′-biphenyl]-2-yl)cyclopropane-1- carbonyl]-4-methylpiperidin-3- yl}methanesulfonamide | 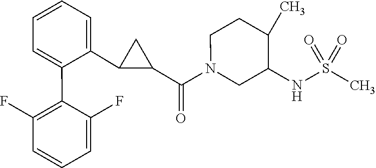 [SMILES: CC1CCN(C(=O)C2CC2c2ccccc2-c2c(F)cccc2F)CC1NS(C)(=O)=O] | 449.2 |
| 186 | a mixture of rac-N-{(4S,5R)-1-[(1R,2R)-2- (2′,6′-difluoro[1,1′-biphenyl]-2- yl)cyclopropane-1-carbonyl]-5-methylazepan- 4-yl}methanesulfonamide and rac-N-{(4R,5S)- 1-[(1R,2R)-2-(2′,6′-difluoro[1,1′-biphenyl]-2- yl)cyclopropane-1-carbonyl]-5-methylazepan- 4-yl}methanesulfonamide and rac-N-{(4S,5S)- 1-[(1R,2R)-2-(2′,6′-difluoro[1,1′-biphenyl]-2- yl)cyclopropane-1-carbonyl]-5-methylazepan- 4-yl}methanesulfonamide | 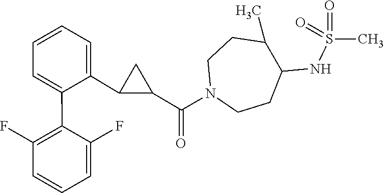 [SMILES: CC1CCN(C(=O)C2CC2c2ccccc2-c2c(F)cccc2F)CCC1NS(C)(=O)=O] | 463.2 |
| 187 | a mixture of rac-N-[(3R)-1-{(1R,2R)-2-[2- (piperidin-1-yl)phenyl]cyclopropane-1- carbonyl}pyrrolidin-3-yl]methanesulfonamide and rac-N-[(3S)-1-{(1R,2R)-2-[2-(piperidin-1- yl)phenyl]cyclopropane-1-carbonyl}pyrrolidin- 3-yl]methanesulfonamide | 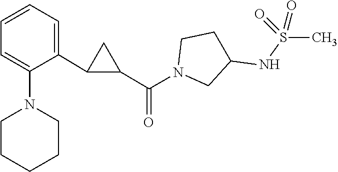 [SMILES: CS(=O)(=O)NC1CCN(C(=O)C2CC2c2ccccc2N2CCCCC2)C1] | 392.2 |
| 188 | a mixture of rac-N-{(3R)-1-[(1R,2R)-2-(2′,3- difluoro[1,1′-biphenyl]-2-yl)cyclopropane-1- carbonyl]pyrrolidin-3-yl}methanesulfonamide and rac-N-{(3S)-1-[(1R,2R)-2-(2′,3- difluoro[1,1′-biphenyl]-2-yl)cyclopropane-1- carbonyl]pyrrolidin-3-yl}methanesulfonamide | 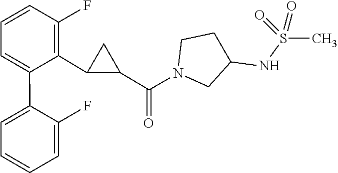 [SMILES: CS(=O)(=O)NC1CCN(C(=O)C2CC2c2c(F)cccc2-c2ccccc2F)C1] | 421.2 |
| 189 | a mixture of rac-N-[(3R)-1-{(1R,2R)-2-[3- (2,6-difluorophenyl)pyridin-2-yl]cyclopropane- 1-carbonyl}piperidin-3-yl]methanesulfonamide and rac-N-[(3S)-1-{(1R,2R)-2-[3-(2,6- difluorophenyl)pyridin-2-yl]cyclopropane-1- carbonyl}piperidin-3-yl]methanesulfonamide | 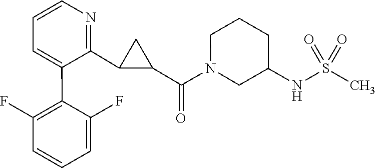 [SMILES: CS(=O)(=O)NC1CCCN(C(=O)C2CC2c2ncccc2-c2c(F)cccc2F)C1] | 436.3 |
| 190 | a mixture of rac-N-{(4R)-1-[(1R,2R)-2-([1,1′- biphenyl]-4-yl)cyclopropane-1- carbonyl]azepan-4-yl}methanesulfonamide and rac-N-{(4S)-1-[(1R,2R)-2-([1,1′- biphenyl]-4-yl)cyclopropane-1- carbonyl]azepan-4-yl}methanesulfonamide | 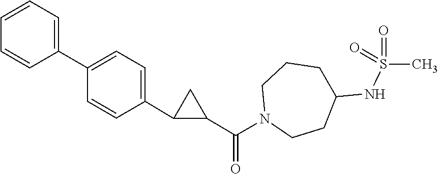 [SMILES: CS(=O)(=O)NC1CCCN(C(=O)C2CC2c2ccc(-c3ccccc3)cc2)CC1] | 413.3 |
| 191 | a mixture of rac-N-{(3R)-1-[(1R,2R)-2-([1,1′- biphenyl]-4-yl)cyclopropane-1- carbonyl]piperidin-3-yl}methanesulfonamide and rac-N-{(3S)-1-[(1R,2R)-2-([1,1′- biphenyl]-4-yl)cyclopropane-1- carbonyl]piperidin-3-yl}methanesulfonamide | 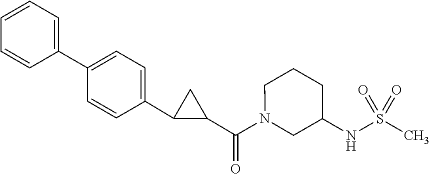 [SMILES: CS(=O)(=O)NC1CCCN(C(=O)C2CC2c2ccc(-c3ccccc3)cc2)C1] | 399.2 |
| 192 | a mixture of N-{(3S)-1-[(1R,2R)-2-(2′,6,6′- trifluoro[1,1′-biphenyl]-2-yl)cyclopropane-1- carbonyl]pyrrolidin-3-yl}methanesulfonamide and N-{(3S)-1-[(1S,2S)-2-(2′,6,6′- trifluoro[1,1′-biphenyl]-2-yl)cyclopropane-1- carbonyl]pyrrolidin-3-yl}methanesulfonamide | 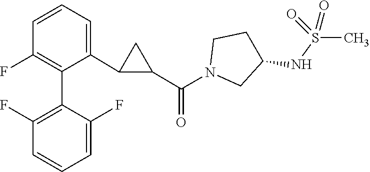 [SMILES: CS(=O)(=O)N[C@H]1CCN(C(=O)C2CC2c2cccc(F)c2-c2c(F)cccc2F)C1] | 439.1 |
| 193 | a mixture of N-{(3S)-1-[(1R,2R)-2-(2′,6,6′- trifluoro[1,1′-biphenyl]-2-yl)cyclopropane-1- carbonyl]piperidin-3-yl}methanesulfonamide and N-{(3S)-1-[(1S,2S)-2-(2′,6,6′- trifluoro[1,1′-biphenyl]-2-yl)cyclopropane-1- carbonyl]piperidin-3-yl}methanesulfonamide | 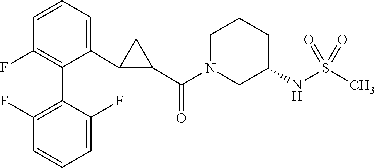 [SMILES: CS(=O)(=O)N[C@H]1CCCN(C(=O)C2CC2c2cccc(F)c2-c2c(F)cccc2F)C1] | 453.2 |
| 194 | a mixture of N-{(4S)-1-[(1R,2R)-2-(2′,6,6′- trifluoro[1,1′-biphenyl]-2-yl)cyclopropane-1- carbonyl]azepan-4-yl}methanesulfonamide and N-{(4S)-1-[(1S,2S)-2-(2′,6,6′- trifluoro[1,1′-biphenyl]-2-yl)cyclopropane-1- carbonyl]azepan-4-yl}methanesulfonamide | 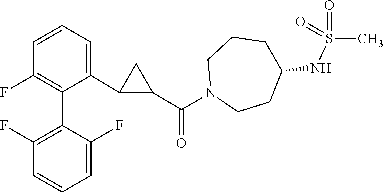 [SMILES: CS(=O)(=O)N[C@H]1CCCN(C(=O)C2CC2c2cccc(F)c2-c2c(F)cccc2F)CC1] | 467.2 |
| 195 | N-{(3S)-1-[(1R,2R)-2-(2′,3,6′-trifluoro[1,1′- biphenyl]-2-yl)cyclopropane-1- carbonyl]pyrrolidin-3-yl}methanesulfonamide, or N-{(3S)-1-[(1S,2S)-2-(2′,3,6′-trifluoro[1,1′- biphenyl]-2-yl)cyclopropane-1- carbonyl]pyrrolidin-3-yl}methanesulfonamide | 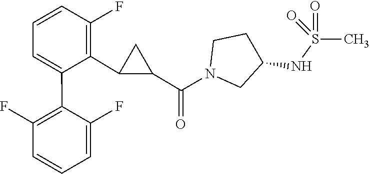 [SMILES: CS(=O)(=O)N[C@H]1CCN(C(=O)C2CC2c2c(F)cccc2-c2c(F)cccc2F)C1] | 439.2 |
| 196 | N-{(3S)-1-[(1R,2R)-2-(2′,3,6′-trifluoro[1,1′- biphenyl]-2-yl)cyclopropane-1- carbonyl]piperidin-3-yl}methanesulfonamide, or N-{(3S)-1-[(1S,2S)-2-(2′,3,6′-trifluoro[1,1′- biphenyl]-2-yl)cyclopropane-1- carbonyl]piperidin-3-yl}methanesulfonamide | 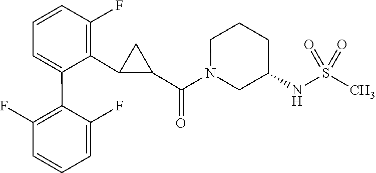 [SMILES: CS(=O)(=O)N[C@H]1CCCN(C(=O)C2CC2c2c(F)cccc2-c2c(F)cccc2F)C1] | 453.2 |
| 197 | a mixture of rac-N-[(3R)-1-{(1R,2R)-2-[6- (difluoromethyl)[1,1′-biphenyl]-2- yl]cyclopropane-1-carbonyl}piperidin-3- yl]methanesulfonamide and rac-N-[(3S)-1- {(1R,2R)-2-[6-(difluoromethyl)[1,1′-biphenyl]- 2-yl]cyclopropane-1-carbonyl}piperidin-3- yl]methanesulfonamide | 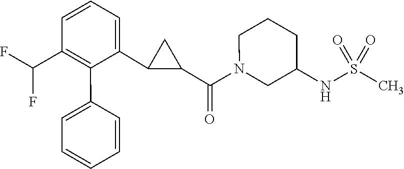 [SMILES: CS(=O)(=O)NC1CCCN(C(=O)C2CC2c2cccc(C(F)F)c2-c2ccccc2)C1] | 449.2 |
| 198 | a mixture of N-{(3S)-1-[(1R,2R)-2-(2′,6′- difluoro[1,1′-biphenyl]-2-yl)-2- fluorocyclopropane-1-carbonyl]pyrrolidin-3- yl}methanesulfonamide and N-{(3S)-1- [(1S,2S)-2-(2′,6′-difluoro[1,1′-biphenyl]-2-yl)- 2-fluorocyclopropane-1-carbonyl]pyrrolidin-3- yl}methanesulfonamide | 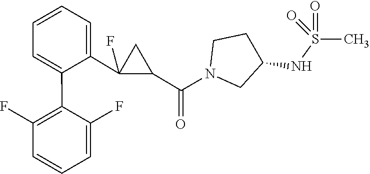 [SMILES: CS(=O)(=O)N[C@H]1CCN(C(=O)C2CC2(F)c2ccccc2-c2c(F)cccc2F)C1] | 439.3 |
| 199 | a mixture of N-{(3S)-1-[(1R,2R)-2-(2′,6′- difluoro[1,1′-biphenyl]-2-yl)-2- fluorocyclopropane-1-carbonyl]piperidin-3- yl}methanesulfonamide and N-{(3S)-1- [(1S,2S)-2-(2′,6′-difluoro[1,1′-biphenyl]-2-yl)- 2-fluorocyclopropane-1-carbonyl]piperidin-3- yl}methanesulfonamide | 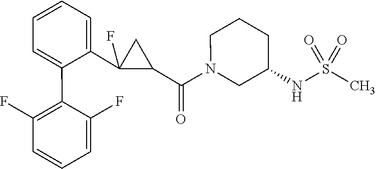 [SMILES: CS(=O)(=O)N[C@H]1CCCN(C(=O)C2CC2(F)c2ccccc2-c2c(F)cccc2F)C1] | 453.3 |
| 200 | N-{(3S)-1-[(1R,2R)-2-(2′,5,6′-tifluoro[1,1′- biphenyl]-2-yl)cyclopropane-1- carbonyl]pyrrolidin-3-yl}methanesulfonamide, or N-{(3S)-1-[(1S,2S)-2-(2′,5,6′-trifluoro[1,1′- biphenyl]-2-yl)cyclopropane-1- carbonyl]pyrrolidin-3-yl}methanesulfonamide | 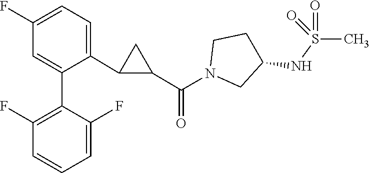 [SMILES: CS(=O)(=O)N[C@H]1CCN(C(=O)C2CC2c2ccc(F)cc2-c2c(F)cccc2F)C1] | 439.2 |
| 201 | N-{(3S)-1-[(1R,2R)-2-(2′,5,6′-trifluoro[1,1′- biphenyl]-2-yl)cyclopropane-1- carbonyl]piperidin-3-yl}methanesulfonamide, or N-{(3S)-1-[(1S,2S)-2-(2′,5,6′-trifluoro[1,1′- biphenyl]-2-yl)cyclopropane-1- carbonyl]piperidin-3-yl}methanesulfonamide | 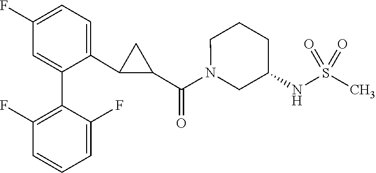 [SMILES: CS(=O)(=O)N[C@H]1CCCN(C(=O)C2CC2c2ccc(F)cc2-c2c(F)cccc2F)C1] | 453.3 |
| 202 | a mixture of rac-N-{(3S)-1-[(1R,2R)-2-(2′,6′- difluoro[1,1′-biphenyl]-2-yl)cyclopropane-1- carbonyl]pyrrolidin-3-yl}-1- fluoromethanesulfonamide and N-{(3S)-1- [(1S,2S)-2-(2′,6′-difluoro[1,1′-biphenyl]-2- yl)cyclopropane-1-carbonyl]pyrrolidin-3-yl}-1- fluoromethanesulfonamide | 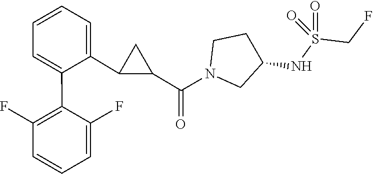 [SMILES: O=C(C1CC1c1ccccc1-c1c(F)cccc1F)N1CC[C@H](NS(=O)(=O)CF)C1] | 439.2 |
| 203 | a mixture of rac-N-({(2R)-1-[(1R,2R)-2-(2′,6′- difluoro[1,1′-biphenyl]-2-yl)cyclopropane-1- carbonyl]azetidin-2- yl}methyl)ethanesulfonamide and rac-N- ({(2S)-1-[(1R,2R)-2-(2′,6′-difluoro[1,1′- biphenyl]-2-yl)cyclopropane-1- carbonyl]azetidin-2- yl}methyl)ethanesulfonamide | 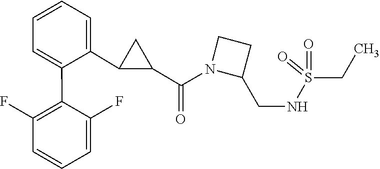 [SMILES: CCS(=O)(=O)NCC1CCN1C(=O)C1CC1c1ccccc1-c1c(F)cccc1F] | 435.3 |
| 204 | a mixture of rac-N′-({(2R)-1-[(1R,2R)-2-(2′,6′- difluoro[1,1′-biphenyl]-2-yl)cyclopropane-1- carbonyl]azetidin-2-yl}methyl)-N,N- dimethylsulfuric diamide and rac-N′-({(2S)-1- [(1R,2R)-2-(2′,6′-difluoro[1,1′-biphenyl]-2- yl)cyclopropane-1-carbonyl]azetidin-2- yl}methyl)-N,N-dimethysulfuric diamide | 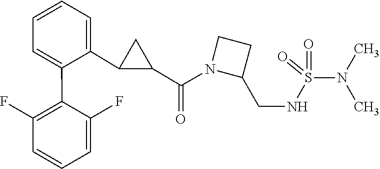 [SMILES: CN(C)S(=O)(=O)NCC1CCN1C(=O)C1CC1c1ccccc1-c1c(F)cccc1F] | 450.3 |
| 205 | a mixture of rac-N-({(2R)-1-[(1R,2R)-2-(2′,6′- difluoro[1,1′-biphenyl]-2-yl)cyclopropane-1- carbonyl]azetidin-2- yl}methyl)cyclopropanesulfonamide and rac- N-({(2S)-1-[(1R,2R)-2-(2′,6′-difluoro[1,1′- biphenyl]-2-yl)cyclopropane-1- carbonyl]azetidin-2- yl}methyl)cyclopropanesulfonamide | 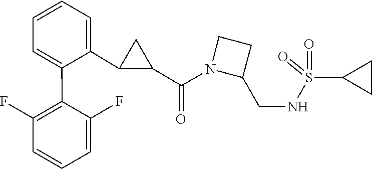 [SMILES: O=C(C1CC1c1ccccc1-c1c(F)cccc1F)N1CCC1CNS(=O)(=O)C1CC1] | 447.3 |
| 206 | a mixture of rac-N-[(4R)-1-{(1R,2R)-2-[4- (2,6-difluorophenyl)pyridin-3-yl]cyclopropane- 1-carbonyl}azepan-4-yl]methanesulfonamide and rac-N-[(4S)-1-{(1R,2R)-2-[4-(2,6- difluorophenyl)pyridin-3-yl]cyclopropane-1- carbonyl}azepan-4-yl]methanesulfonamide | 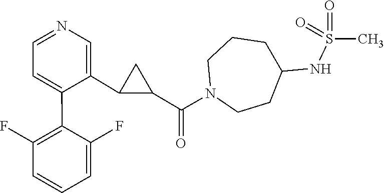 [SMILES: CS(=O)(=O)NC1CCCN(C(=O)C2CC2c2cnccc2-c2c(F)cccc2F)CC1] | 450.4 |
| 207 | a mixture of N-{(3S)-1-[(1R,2R)-2-(2′,6′- difluoro[1,1′-biphenyl]-2-yl)cyclopropane-1- carbonyl]piperidin-3-yl}-1- fluoromethanesulfonamide and N-{(3S)-1- [(1S,2S)-2-(2′,6′-difluoro[1,1′-biphenyl]-2- yl)cyclopropane-1-carbonyl]piperidin-3-yl}-1- fluoromethanesulfonamide | 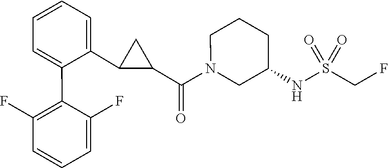 [SMILES: O=C(C1CC1c1ccccc1-c1c(F)cccc1F)N1CCC[C@H](NS(=O)(=O)CF)C1] | 453.3 |
| 208 | a mixture of rac-N-{(3R)-1-[(1R,2R)-2-(2′,5- difluoro[1,1′-biphenyl]-2-yl)cyclopropane-1- carbonyl]pyrrolidin-3-yl}methanesulfonamide and rac-N-{(3S)-1-[(1R,2R)-2-(2′,5- difluoro[1,1′-biphenyl]-2-yl)cyclopropane-1- carbonyl]pyrrolidin-3-yl}methanesulfonamide | 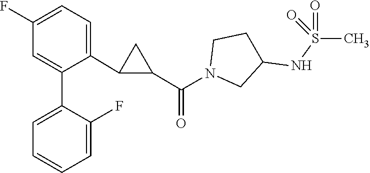 [SMILES: CS(=O)(=O)NC1CCN(C(=O)C2CC2c2ccc(F)cc2-c2ccccc2F)C1] | 421.3 |
| 209 | a mixture of rac-N-{(3R)-1-[(1R,2R)-2-(2′- fluoro-5-methyl[1,1′-biphenyl]-2- yl)cyclopropane-1-carbonyl]pyrrolidin-3- yl}methanesulfonamide and rac-N-{(3S)-1- [(1R,2R)-2-(2′-fluoro-5-methyl[1,1′-biphenyl]- 2-yl)cyclopropane-1-carbonyl]pyrrolidin-3- yl}methanesulfonamide | 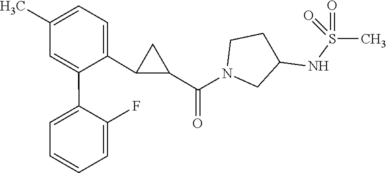 | 417.3 |
| 210 | N-{1-[2-(2′,3,6′-trifluoro[1,1′-biphenyl]-2- yl)cyclopropane-1-carbonyl]pyrrolidin-3- yl}methanesulfonamide optical isomer | 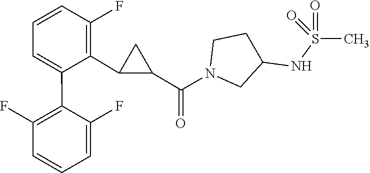 [SMILES: CS(=O)(=O)NC1CCN(C(=O)C2CC2c2c(F)cccc2-c2c(F)cccc2F)C1] | 439.3 |
| 211 | N-{1-[2-(2′-3,6′-trifluoro[1,1′-biphenyl]-2- yl)cyclopropane-1-carbonyl]piperidin-3- yl}methanesulfonamide optical isomer | 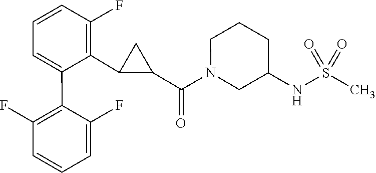 [SMILES: CS(=O)(=O)NC1CCCN(C(=O)C2CC2c2c(F)cccc2-c2c(F)cccc2F)C1] | 453.3 |
| 212 | a mixture of rac-N-{(4R)-1-[(1R,2R)-2-(2′,6′- difluoro[1,1′-biphenyl]-2-yl)cyclopropane-1- carbonyl]-5,5-difluoroazepan-4- yl}methanesulfonamide and rac-N-{(4S)-1- [(1R,2R)-2-(2′,6′-difluoro[1,1′-biphenyl]-2- yl)cyclopropane-1-carbonyl]-5,5- difluoroazepan-4-yl}methanesulfonamide | 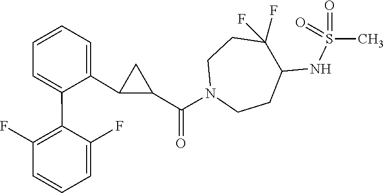 [SMILES: CS(=O)(=O)NC1CCN(C(=O)C2CC2c2ccccc2-c2c(F)cccc2F)CCC1(F)F] | 485.3 |
| 213 | a mixture of 1-bromo-N-{(3S)-1-[(1R,2R)-2- (2′,6′-difluoro[1,1′-biphenyl]-2- yl)cyclopropane-1-carbonyl]piperidin-3- yl}methanesulfonamide and 1-bromo-N-{(3S)- 1-[(1S,2S)-2-(2′,6′-difluoro[1,1′-biphenyl]-2- yl)cyclopropane-1-carbonyl]piperidin-3- yl}methanesulfonamide | 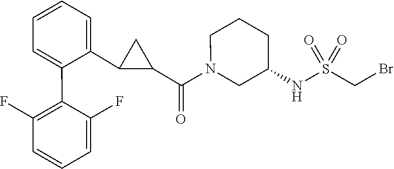 [SMILES: O=C(C1CC1c1ccccc1-c1c(F)cccc1F)N1CCC[C@H](NS(=O)(=O)CBr)C1] | 513.2 |
| 214 | a mixture of N-{(3S)-1-[(1R,2R)-2-(2′,6′- difluoro[1,1′-biphenyl]-2-yl)cyclopropane-1- carbonyl]piperidin-3-yl}-1- methoxymethanesulfonamide and N-{(3S)-1- [(1S,2S)-2-(2′,6′-difluoro[1,1′-biphenyl]-2- yl)cyclopropane-1-carbonyl]piperidin-3-yl}-1- methoxymethanesulfonamide | 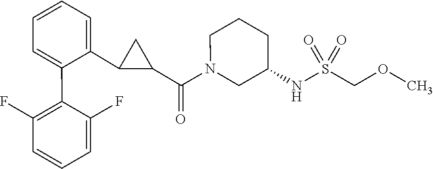 [SMILES: COCS(=O)(=O)N[C@H]1CCCN(C(=O)C2CC2c2ccccc2-c2c(F)cccc2F)C1] | 465.3 |
| 215 | N-{(3R)-1-[(1R,2R)-2-(2′,6′-difluoro[1,1′- biphenyl]-2-yl)cyclopropane-1-carbonyl]-4,4- difluoropiperidin-3-yl}methanesulfonamide, or N-{(3R)-1-[(1S,2S)-2-(2′,6′-difluoro[1,1′- biphenyl]-2-yl)cyclopropane-1-carbonyl]-4,4- difluoropiperidin-3-yl}methanesulfonamide | 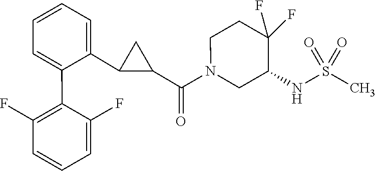 [SMILES: CS(=O)(=O)N[C@@H]1CN(C(=O)C2CC2c2ccccc2-c2c(F)cccc2F)CCC1(F)F] | 471.3 |
| 216 | N-{(3R,4S)-1-[(1R,2R)-2-(2′,6′-difluoro[1,1′- biphenyl]-2-yl)cyclopropane-1-carbonyl]-4- fluoropiperidin-3-yl}methanesulfonamide, or N-{(3R,4S)-1-[(1S,2S)-2-(2′,6′-difluoro[1,1′- biphenyl]-2-yl)cyclopropane-1-carbonyl]-4- fluoropiperidin-3-yl}methanesulfonamide | 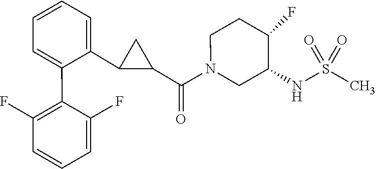 [SMILES: CS(=O)(=O)N[C@@H]1CN(C(=O)C2CC2c2ccccc2-c2c(F)cccc2F)CC[C@@H]1F] | 453.3 |
| 217 | a mixture of rac-N-{(3S,4R)-1-[(1R,2R)-2- (2′,6′-difluoro[1,1′-biphenyl]-2- yl)cyclopropane-1-carbonyl]-3-methylazepan- 4-yl}methanesulfonamide and rac-N-{(3R,4S)- 1-[(1R,2R)-2-(2′,6′-difluoro[1,1′-biphenyl]-2- yl)cyclopropane-1-carbonyl]-3-methylazepan- 4-yl}methanesulfonamide and rac-N-{(3S,4S)- 1-[(1R,2R)-2-(2′,6′-difluoro[1,1′-biphenyl]-2- yl)cyclopropane-1-carbonyl]-3-methylazepan- 4-yl}methanesulfonamide | 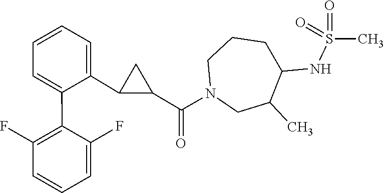 [SMILES: CC1CN(C(=O)C2CC2c2ccccc2-c2c(F)cccc2F)CCCC1NS(C)(=O)=O] | 463.3 |
| 218 | a mixture of rac-N-({(3R)-1-[(1R,2R)-2-(2′,6′- difluoro[1,1′-biphenyl]-2-yl)cyclopropane-1- carbonyl]-3-fluoropyrrolidin-3- yl}methyl)methanesulfonamide and rac-N- ({(3S)-1-[(1R,2R)-2-(2′,6′-difluoro[1,1′- biphenyl]-2-yl)cyclopropane-1-carbonyl]-3- fluoropyrrolidin-3- yl}methyl)methanesulfonamide | 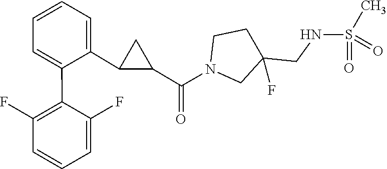 [SMILES: CS(=O)(=O)NCC1(F)CCN(C(=O)C2CC2c2ccccc2-c2c(F)cccc2F)C1] | 453.3 |
| 219 | a mixture of rac-N-{(6R)-1-[(1R,2R)-2-(2′,6′- difluoro[1,1′-biphenyl]-2-yl)cyclopropane-1- carbonyl]-1,4-oxazepan-6- yl}methanesulfonamide and rac-N-{(6S)-4- [(1R,2R)-2-(2′,6′-difluoro[1,1′-biphenyl]-2- yl)cyclopropane-1-carbonyl]-1,4-oxazepan-6- yl}methanesulfonamide | 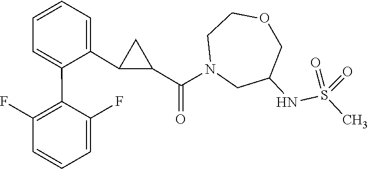 [SMILES: CS(=O)(=O)NC1COCCN(C(=O)C2CC2c2ccccc2-c2c(F)cccc2F)C1] | 451.3 |
| 220 | a mixture of rac-N-[(4R)-1-{(1R,2R)-2-[3-(2- fluorophenyl)thiophen-2-yl]cyclopropane-1- carbonyl}azepan-4-yl]methanesulfonamide and rac-N-[(4S)-1-{(1R,2R)-2-[3-(2- fluorophenyl)thiophen-2-yl]cyclopropane-1- carbonyl}azepan-4-yl]methanesulfonamide | 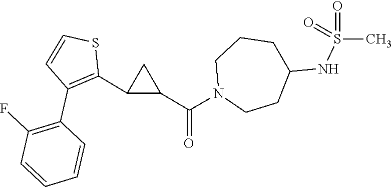 [SMILES: CS(=O)(=O)NC1CCCN(C(=O)C2CC2c2sccc2-c2ccccc2F)CC1] | 437.3 |
| 221 | a mixture of rac-N-[(4R)-1-{(1R,2R)-2-[3- (2,6-difluorophenyl)thiophen-2- yl]cyclopropane-1-carbonyl}azepan-4- yl]methanesulfonamide and rac-N-[(4S)-1- {(1R,2R)-2-[3-(2,6-difluorophenyl)thiophen-2- yl]cyclopropane-1-carbonyl}azepan-4- yl]methanesulfonamide | 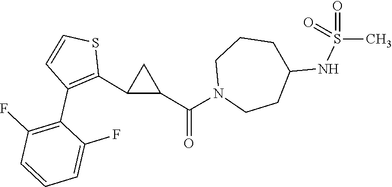 [SMILES: CS(=O)(=O)NC1CCCN(C(=O)C2CC2c2sccc2-c2c(F)cccc2F)CC1] | 455.3 |
| 222 | a mixture of rac-N-[(3R)-1-{(1R,2R)-2-[3-(2- fluorophenyl)thiophen-2-yl]cyclopropane-1- carbonyl}piperidin-3-yl]methanesulfonamide and rac-N-[(3S)-1-{(1R,2R)-2-[3-(2- fluorophenyl)thiophen-2-yl]cyclopropane-1- carbonyl}piperidin-3-yl]methanesulfonamide | 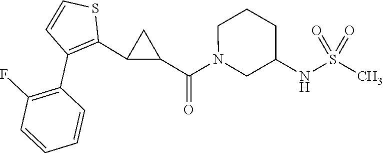 [SMILES: CS(=O)(=O)NC1CCCN(C(=O)C2CC2c2sccc2-c2ccccc2F)C1] | 423.3 |
| 223 | a mixture of rac-N-[(3R)-1-{(1R,2R)-2-[3- (2,6-difluorophenyl)thiophen-2- yl]cyclopropane-1-carbonyl}piperidin-3- yl]methanesulfonamide and rac-N-[(3S)-1- {(1R,2R)-2-[3-(2,6-difluorophenyl)thiophen-2- yl]cyclopropane-1-carbonyl}piperidin-3- yl]methanesulfonamide | 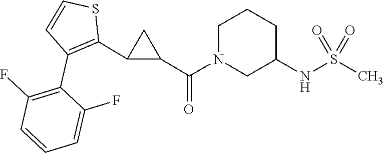 [SMILES: CS(=O)(=O)NC1CCCN(C(=O)C2CC2c2sccc2-c2c(F)cccc2F)C1] | 441.3 |
| 224 | a mixture of rac-N-[(3R)-1-{(1R,2R)-2-[4- (2,6-difluorophenyl)thiophen-2- yl]cyclopropane-1-carbonyl}pyrrolidin-3- yl]methanesulfonamide and rac-N-[(3S)-1- {(1R,2R)-2-[4-(2,6-difluorophenyl)thiophen-2- yl]cyclopropane-1-carbonyl}pyrrolidin-3- yl]methanesulfonamide | 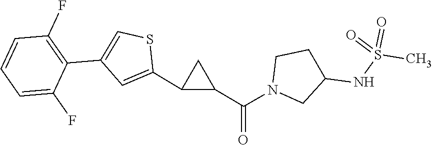 [SMILES: CS(=O)(=O)NC1CCN(C(=O)C2CC2c2cc(-c3c(F)cccc3F)cs2)C1] | 427.3 |
| 225 | a mixture of rac-N-{(4R)-1-[(1R,2R)-2-(5- fluoro-6-methyl[1,1′-biphenyl]-2- yl)cyclopropane-1-carbonyl]azepan-4- yl}methanesulfonamide and rac-N-{(4S)-1- [(1R,2R)-2-(5-fluoro-6-methyl[1,1′-biphenyl]- 2-yl)cyclopropane-1-carbonyl]azepan-4- yl}methanesulfonamide | 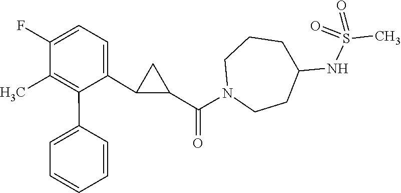 [SMILES: Cc1c(F)ccc(C2CC2C(=O)N2CCCC(NS(C)(=O)=O)CC2)c1-c1ccccc1] | 445.4 |
| 226 | N-{4,4-difluoro-1-[2-(2′,3,6′-trifluoro[1,1′- biphenyl]-2-yl)cyclopropane-1- carbonyl]pyrrolidin-3-yl}methanesulfonamide optical isomer | 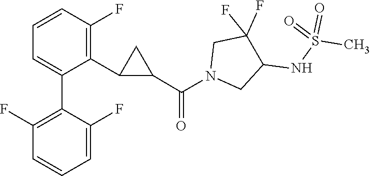 [SMILES: CS(=O)(=O)NC1CN(C(=O)C2CC2c2c(F)cccc2-c2c(F)cccc2F)CC1(F)F] | 475.2 |
| 227 | a mixture of N-{(3S)-1-[(1R,2R)-2-(2′,6′- difluoro[1,1′-biphenyl]-2-yl)cyclopropane-1- carbonyl]pyrrolidin-3-yl}-1- methoxymethanesulfonamide and N-{(3S)-1- [(1S,2S)-2-(2′,6′-difluoro[1,1′-biphenyl]-2- yl)cyclopropane-1-carbonyl]pyrrolidin-3-yl}-1- methoxymethanesulfonamide | 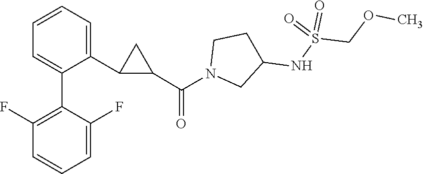 [SMILES: COCS(=O)(=O)NC1CCN(C(=O)C2CC2c2ccccc2-c2c(F)cccc2F)C1] | 451.3 |
| 228 | a mixture of rac-N-[(3R)-1-{(1R,2R)-2-[5- (difluoromethyl)-2′,6′-difluoro[1,1′-biphenyl]- 2-yl]cyclopropane-1-carbonyl}pyrrolidin-3- yl]methanesulfonamide and rac-N-[(3S)-1- {(1R,2R)-2-[5-(difluoromethyl)-2′,6′- difluoro[1,1′-biphenyl]-2-yl]cyclopropane-1- carbony}pyrrolidin-3-yl]methanesulfonamide | 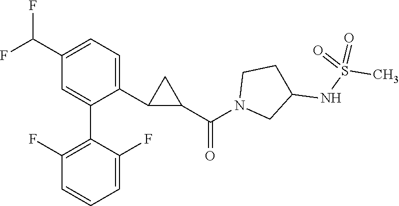 [SMILES: CS(=O)(=O)NC1CCN(C(=O)C2CC2c2ccc(C(F)F)cc2-c2c(F)cccc2F)C1] | 471.3 |
| 229 | a mixture of rac-N-[(4R)-1-{(1R,2R)-2-[5- (difluoromethyl)-2′,6′-difluoro[1,1′-biphenyl]- 2-yl]cyclopropane-1-carbonyl}azepan-4- yl]methanesulfonamide and rac-N-[(4S)-1- {(1R,2R)-2-[5-(difluoromethyl)-2′,6′- difluoro[1,1′-biphenyl]-2-yl]cyclopropane-1- carbonyl}azepan-4-yl]methanesulfonamide | 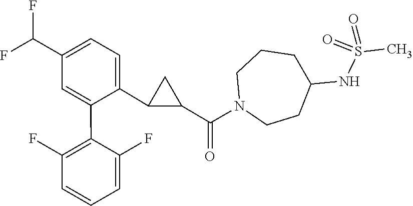 [SMILES: CS(=O)(=O)NC1CCCN(C(=O)C2CC2c2ccc(C(F)F)cc2-c2c(F)cccc2F)CC1] | 499.3 |
| 230 | a mixture of rac-N-[(3R)-1-{(1R,2R)-2-[5- (difluoromethyl)-2′,6′-difluoro[1,1′-biphenyl]- 2-yl]cyclopropane-1-carbonyl}piperidin-3- yl]methanesulfonamide and rac-N-[(3S)-1- {(1R,2R)-2-[5-(difluoromethyl)-2′,6′- difluoro[1,1′-biphenyl]-2-yl]cyclopropane-1- carbonyl}piperidin-3-yl]methanesulfonamide | 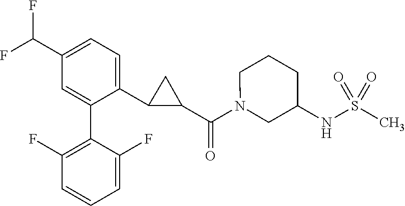 [SMILES: CS(=O)(=O)NC1CCCN(C(=O)C2CC2c2ccc(C(F)F)cc2-c2c(F)cccc2F)C1] | 485.3 |
| 232 | N-{(3S,5R)-1-[(1R,2R)-2-(2′,6′-difluoro[1,1′- biphenyl]-2-yl)cyclopropane-1-carbonyl]-5- fluoropiperidin-3-yl}methanesulfonamide | 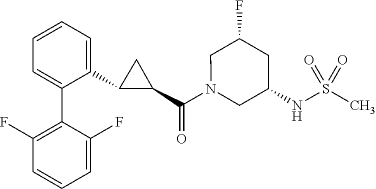 [SMILES: CS(=O)(=O)N[C@H]1C[C@@H](F)CN(C(=O)[C@@H]2C[C@H]2c2ccccc2-c2c(F)cccc2F)C1] | 453.2 |
| 233 | a mixture of rac-N-[(3R)-1-{(1R,2R)-2-[3- (2,6-difluorophenyl)thiophen-2- yl]cyclopropane-1-carbonyl}azepan-3- yl]methanesulfonamide and rac-N-[(3S)-1- {(1R,2R)-2-[3-(2,6-difluorophenyl)thiophen-2- yl]cyclopropane-1-carbonyl}azepan-3- yl]methanesulfonamide | 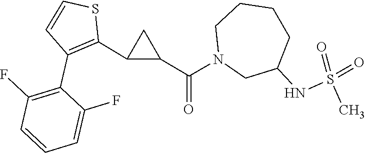 [SMILES: CS(=O)(=O)NC1CCCCN(C(=O)C2CC2c2sccc2-c2c(F)cccc2F)C1] | 455.3 |
| 234 | a mixture of rac-N-[(3R)-1-{(1R,2R)-2-[5-(2- fluorophenyl)thiophen-2-yl]cyclopropane-1- carbonyl}pyrrolidin-3-yl]methanesulfonamide and rac-N-[(3S)-1-{(1R,2R)-2-[5-(2- fluorophenyl)thiophen-2-yl]cyclopropane-1- carbonyl}pyrrolidin-3-yl]methanesulfonamide | 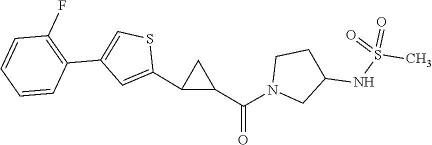 [SMILES: CS(=O)(=O)NC1CCN(C(=O)C2CC2c2cc(-c3ccccc3F)cs2)C1] | 409.1 |
| 235 | a mixture of rac-N-[(3R)-1-{(1R,2R)-2-[3- (2,6-difluorophenyl)thiophen-2- yl]cyclopropane-1-carbonyl}pyrrolidin-3- yl]methanesulfonamide and rac-N-[(3S)-1- {(1R,2R)-2-[3-(2,6-difluorophenyl)thiophen-2- yl]cyclopropane-1-carbonyl}pyrrolidin-3- yl]methanesulfonamide | 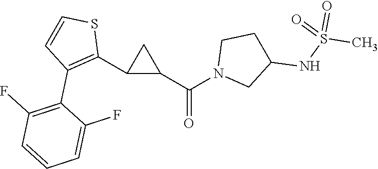 [SMILES: CS(=O)(=O)NC1CCN(C(=O)C2CC2c2sccc2-c2c(F)cccc2F)C1] | 427.3 |
| 236 | a mixture of rac-N-{(4R)-1-[(1R,2R)-2-(2- phenyl-1,3-thiazol-5-yl)cyclopropane-1- carbonyl]azepan-4-yl}methanesulfonamide and rac-N-{(4S)-1-[(1R,2R)-2-(2-phenyl-1,3- thiazol-5-yl)cyclopropane-1-carbonyl]azepan- 4-yl}methanesulfonamide | 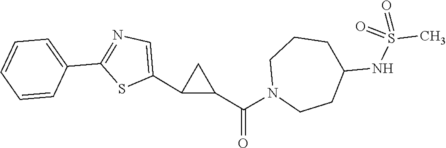 [SMILES: CS(=O)(=O)NC1CCCN(C(=O)C2CC2c2cnc(-c3ccccc3)s2)CC1] | 420.3 |
| 237 | a mixture of rac-N-{(1S,5R)-3-[(1R,2R)-2- (2′,6′-difluoro[1,1′-biphenyl]-2- yl)cyclopropane-1-carbonyl]-3- azabicyclo[3.1.0]hexan-1- yl}ethanesulfonamide and rac-N-{(1R,5S)-3- [(1R,2R)-2-(2′,6′-difluoro[1,1′-biphenyl]-2- yl)cyclopropane-1-carbonyl]-3- azabicyclo[3.1.0]hexan-1- yl}ethanesulfonamide | 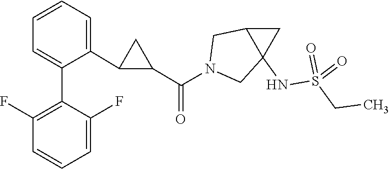 [SMILES: CCS(=O)(=O)NC12CC1CN(C(=O)C1CC1c1ccccc1-c1c(F)cccc1F)C2] | 447.2 |
| 238 | N-2-[(2-(2′,6′-difluoro[1,1′-biphenyl]-2- yl)cyclopropane-1- carbonyl]octahydrocyclopenta[c]pyrrol-4- yl}methanesulfonamide optical isomer | 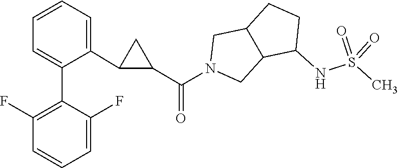 [SMILES: CS(=O)(=O)NC1CCC2CN(C(=O)C3CC3c3ccccc3-c3c(F)cccc3F)CC21] | 461.2 |
| 239 | N-{(3S)-1-[(1R,2R)-2-(2′,6′-difluoro[1,1′- biphenyl]-2-yl)cyclopropane-1- carbonyl]piperidin-3-yl}ethanesulfonamide, or N-{(3S)-1-[(1S,2S)-2-(2′,6′-difluoro[1,1′- biphenyl]-2-cyclopropane-1- carbonyl]piperidin-3-yl}ethanesulfonamide | 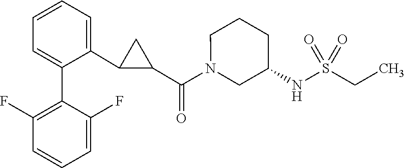 [SMILES: CCS(=O)(=O)N[C@H]1CCCN(C(=O)C2CC2c2ccccc2-c2c(F)cccc2F)C1] | 449.2 |
| 240 | N-{(3R)-1-[(1R,2R)-2-(2′,6′-difluoro[1,1′- biphenyl]-2-yl)cyclopropane-1-carbonyl]-4,4- difluoropiperidin-3-yl}ethanesulfonamide | 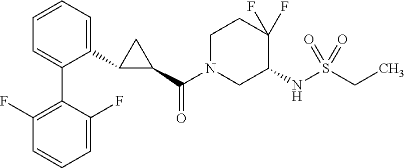 [SMILES: CCS(=O)(=O)N[C@@H]1CN(C(=O)[C@@H]2C[C@H]2c2ccccc2-c2c(F)cccc2F)CCC1(F)F] | 485.3 |
| 241 | N-{(3R,4S)-1-[(1R,2R)-2-(2′,6′-difluoro[1,1′- biphenyl]-2-yl)cyclopropane-1-carbonyl]-4- fluoropyrrolidin-3-yl}methanesulfonamide | 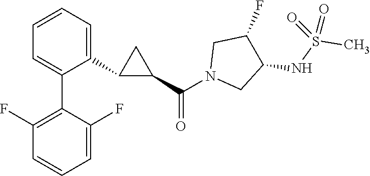 [SMILES: CS(=O)(=O)N[C@@H]1CN(C(=O)[C@@H]2C[C@H]2c2ccccc2-c2c(F)cccc2F)C[C@@H]1F] | 439.1 |
| 242 | a mixture of rac-N-{(4R)-1-[(1R,2R)-2-(2′- fluoro-6-methyl[1,1′-biphenyl]-2- yl)cyclopropane-1-carbonyl]azepan-4- yl}methanesulfonamide and rac-N-{(4S)-1- [(1R,2R)-2-(2′-fluoro-6-methyl[1,1′-biphenyl]- 2-yl)cyclopropane-1-carbonyl]azepan-4- yl}methanesulfonamide | 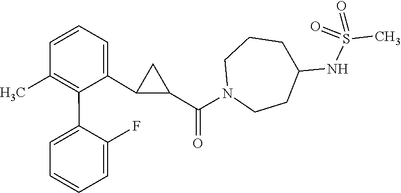 | 445.2 |
| 243 | a mixture of rac-N-{(3R)-1-[(1R,2R)-2-(5- phenyl-1,3-thiazol-2-yl)cyclopropane-1- carbonyl]piperidin-3-yl}methanesulfonamide and rac-N-{(3S)-1-[(1R,2R)-2-(5-phenyl-1,3- thiazol-2-yl)cyclopropane-1- carbonyl]piperidin-3-yl}methanesulfonamide | 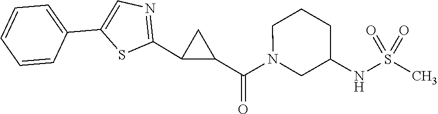 [SMILES: CS(=O)(=O)NC1CCCN(C(=O)C2CC2c2ncc(-c3ccccc3)s2)C1] | 406.1 |
| 244 | a mixture of rac-N-[(3R)-1-{(1R,2R)-2-[4-(2- fluorophenyl)thiophen-2-yl]cyclopropane-1- carbonyl}pyrrolidin-3-yl]methanesulfonamide and rac-N-[(3S)-1-{(1R,2R)-2-[4-(2- fluorophenyl)thiophen-2-yl]cyclopropane-1- carbonyl}pyrrolidin-3-yl]methanesulfonamide | 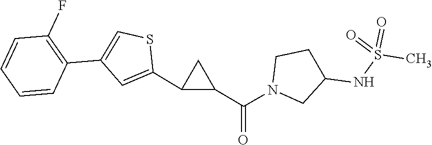 [SMILES: CS(=O)(=O)NC1CCN(C(=O)C2CC2c2cc(-c3ccccc3F)cs2)C1] | 409.2 |
| 245 | N-{4,4-difluoro-1-[2-(2′,5,6′-trifluoro[1,1′- biphenyl]-2-yl)cyclopropane-1- carbonyl]pyrrolidin-3-yl}methanesulfonamide optical isomer | 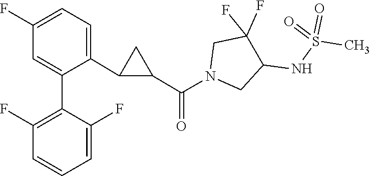 [SMILES: CS(=O)(=O)NC1CN(C(=O)C2CC2c2ccc(F)cc2-c2c(F)cccc2F)CC1(F)F] | 473.2 |
| 246 | N-{(4S)-1-[(1R,2S)-2-(2′,6′-difluoro[1,1′- biphenyl]-2-yl)-1-methylcyclopropane-1- carbonyl]azepan-4-yl}methanesulfonamide, or N-{(4S)-1-[(1S,2R)-2-(2′,6′-difluoro[1,1′- biphenyl]-2-yl)-1-methylcyclopropane-1- carbonyl]azepan-4-yl}methanesulfonamide | 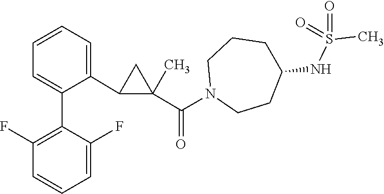 [SMILES: CC1(C(=O)N2CCC[C@H](NS(C)(=O)=O)CC2)CC1c1ccccc1-c1c(F)cccc1F] | 463.3 |
| 247 | N-{(3S)-1-[(1R,2S)-2-(2′,6′-difluoro[1,1′- biphenyl]-2-yl)-1-methylcyclopropane-1- carbonyl]pyrrolidin-3-yl}methanesulfonamide, or N-{(3S)-1-[(1S,2R)-2-(2′,6′-difluoro[1,1′- biphenyl]-2-yl)-1-methylcyclopropane-1- carbonyl]pyrrolidin-3-yl}methanesulfonamide | 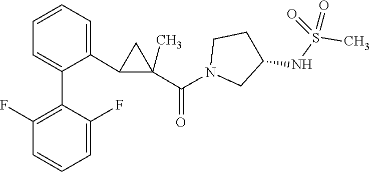 [SMILES: CC1(C(=O)N2CC[C@H](NS(C)(=O)=O)C2)CC1c1ccccc1-c1c(F)cccc1F] | 435.4 |
| 248 | N-{1-[2-(2′,6′-difluoro[1,1′-biphenyl]-2-yl)-1- methylcyclopropane-1-carbonyl]-4,4- difluoropyrrolidin-3-yl}methanesulfonamide optical isomer | 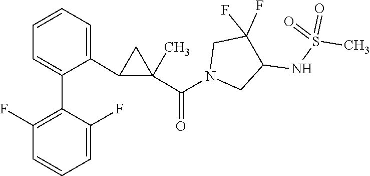 [SMILES: CC1(C(=O)N2CC(NS(C)(=O)=O)C(F)(F)C2)CC1c1ccccc1-c1c(F)cccc1F] | 471.3 |
| 249 | N-{(6R)-4-[(1R,2R)-2-(2′,6′-difluoro[1,1′- biphenyl]-2-yl)cyclopropane-1-carbonyl]-1,4- oxazepan-6-yl}methanesulfonamide, or N- {(6S)-4-[(1R,2R)-2-(2′,6′-difluoro[1,1′- biphenyl]-2-yl)cyclopropane-1-carbonyl]-1,4- oxazepan-6-yl}methanesulfonamide | 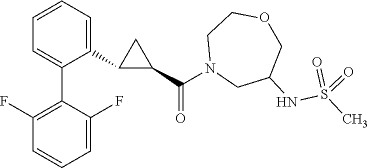 [SMILES: CS(=O)(=O)NC1COCCN(C(=O)[C@@H]2C[C@H]2c2ccccc2-c2c(F)cccc2F)C1] | 451.3 |
| 250 | N′-({1-[2-(2′,6′-difluoro[1,1′-biphenyl]-2- yl)cyclopropane-1-carbonyl]azetidin-2- yl}methyl)-N,N-dimethylsulfuric diamide optical isomer | 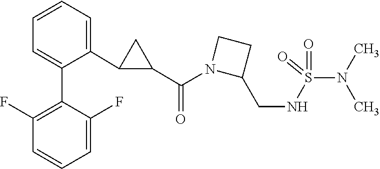 [SMILES: CN(C)S(=O)(=O)NCC1CCN1C(=O)C1CC1c1ccccc1-c1c(F)cccc1F] | 450.4 |
| 251 | N′-({1-[2-(2′,6′-difluoro[1,1′-biphenyl]-2- yl)cyclopropane-1-carbonyl]azetidin-2- yl}methyl)-N,N-dimethylsulfuric diamide optical isomer | 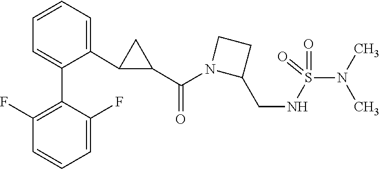 [SMILES: CN(C)S(=O)(=O)NCC1CCN1C(=O)C1CC1c1ccccc1-c1c(F)cccc1F] | 450.3 |
| 252 | a mixture of rac-N-{(3R)-1-[(1R,2R)-2-(5- fluoro-6-methyl[1,1′-biphenyl]-2- yl)cyclopropane-1-carbonyl]piperidin-3- yl}methanesulfonamide and rac-N-{(3S)-1- [(1R,2R)-2-(5-fluoro-6-methyl[1,1′-biphenyl]- 2-yl)cyclopropane-1-carbonyl]piperidin-3- yl}methanesulfonamide | 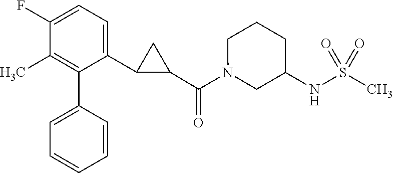 | 431.3 |
| 253 | a mixture of rac-N-{(3R)-1-[(1R,2R)-2-(6- fluoro-5-methyl[1,1′-biphenyl]-2- yl)cyclopropane-1-carbonyl]pyrrolidin-3- yl}methanesulfonamide and rac-N-{(3S)-1- [(1R,2R)-2-(6-fluoro-5-methyl[1,1′-biphenyl]- 2-yl)cyclopropane-1-carbonyl]pyrrolidin-3- yl}methanesulfonamide | 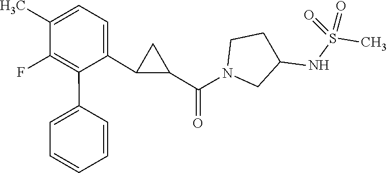 | 417.2 |
| 254 | a mixture of N-[(3S)-1-{(1R,2R)-2-[2-(3- fluoropyridin-2-yl)phenyl]cyclopropane-1- carbonyl}pyrrolidin-3-yl]methanesulfonamide and N-[(3S)-1-{(1S,2S)-2-[2-(3-fluoropyridin- 2-yl)phenyl]cyclopropane-1- carbonyl}pyrrolidin-3-yl]methanesulfonamide | 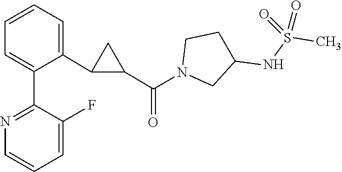 [SMILES: CS(=O)(=O)NC1CCN(C(=O)C2CC2c2ccccc2-c2ncccc2F)C1] | 404.3 |
| 255 | a mixture of N-[(3S)-1-{(1R,2R)-2-[2-(3- fluoropyridin-2-yl)phenyl]cyclopropane-1- carbonyl}piperidin-3-yl]methanesulfonamide and N-[(3S)-1-{(1S,2S)-2-[2-(3-fluoropyridin- 2-yl)phenyl]cyclopropane-1- carbonyl}piperidin-3-yl]methanesulfonamide | 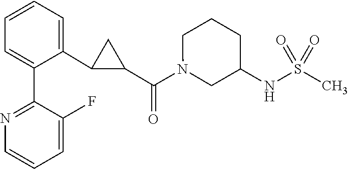 [SMILES: CS(=O)(=O)NC1CCCN(C(=O)C2CC2c2ccccc2-c2ncccc2F)C1] | 418.3 |
| 256 | a mixture of N-[(4S)-1-{(1R,2R)-2-[2-(3- fluoropyridin-2-yl)phenyl]cyclopropane-1- carbonyl}azepan-4-yl]methanesulfonamide and N-[(4S)-1-{(1S,2S)-2-[2-(3-fluoropyridin- 2-yl)phenyl]cyclopropane-1-carbonyl}azepan- 4-yl]methanesulfonamide | 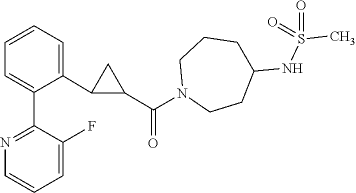 [SMILES: CS(=O)(=O)NC1CCCN(C(=O)C2CC2c2ccccc2-c2ncccc2F)CC1] | 432.3 |
| 257 | a mixture of N-{(6R)-1-[(1R,2R)-2-(2′,6′- difluoro[1,1′-biphenyl]-2-yl)cyclopropane-1- carbonyl]-1,4-oxazepan-6- yl}ethanesulfonamide and N-{(6S)-4-[(1R,2R)- 2-(2′,6′-difluoro[1,1′-biphenyl]-2- yl)cyclopropane-1-carbonyl]-1,4-oxazepan-6- yl}ethanesulfonamide | 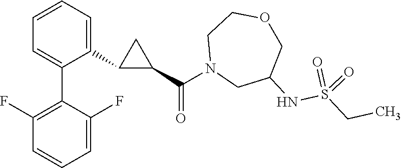 [SMILES: CCS(=O)(=O)NC1COCCN(C(=O)[C@@H]2C[C@H]2c2ccccc2-c2c(F)cccc2F)C1] | 465.3 |
| 258 | a mixture of N-{(6R)-4-[(1R,2R)-2-(2′,6′- difluoro[1,1′-biphenyl]-2-yl)cyclopropane-1- carbonyl]-1,4-oxazepan-6- yl}ethanesulfonamide and N-{(6S)-4-[(1R,2R)- 2-(2′,6′-difluoro[1,1′-biphenyl]-2- yl)cyclopropane-1-carbonyl]-1,4-oxazepan-6- yl}ethanesulfonamide | 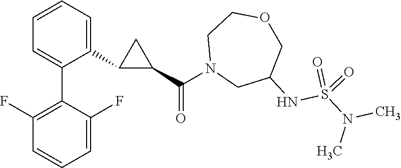 [SMILES: CN(C)S(=O)(=O)NC1COCCN(C(=O)[C@@H]2C[C@H]2c2ccccc2-c2c(F)cccc2F)C1] | 480.3 |
| 259 | N′-({(2S)-1-[(1R,2R)-2-(2′,6′-difluoro[1,1′- biphenyl]-2-yl)cyclopropane-1- carbonyl]azetidin-2-yl}methyl)-N,N- dimethylsulfuric diamide | 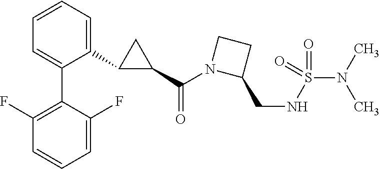 [SMILES: CN(C)S(=O)(=O)NC[C@@H]1CCN1C(=O)[C@@H]1C[C@H]1c1ccccc1-c1c(F)cccc1F] | 450.2 |
| 260 | N-{(2R,3S)-1-[(1R,2R)-2-(2′,6′-difluoro[1,1′- biphenyl]-2-yl)cyclopropane-1-carbonyl]-2- methylpyrrolidin-3-yl}methanesulfonamide | 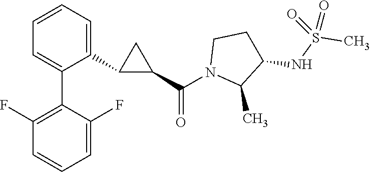 [SMILES: C[C@@H]1[C@@H](NS(C)(=O)=O)CCN1C(=O)[C@@H]1C[C@H]1c1ccccc1-c1c(F)cccc1F] | 435.3 |
| 261 | a mixture of N-{(2R,3R)-1-[(1R,2R)-2-(2′,6′- difluoro[1,1′-biphenyl]-2-yl)cyclopropane-1- carbonyl]-2-methylpyrrolidin-3- yl}methanesulfonamide and N-{(2S,3S)-1- [(1R,2R)-2-(2′,6′-difluoro[1,1′-biphenyl]-2- yl)cyclopropane-1-carbonyl]-2- methylpyrrolidin-3-yl}methanesulfonamide | 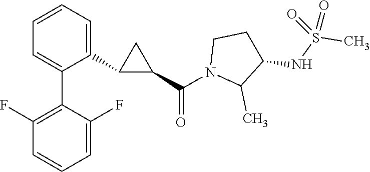 [SMILES: CC1[C@@H](NS(C)(=O)=O)CCN1C(=O)[C@@H]1C[C@H]1c1ccccc1-c1c(F)cccc1F] | 435.3 |
| 262 | N-{(3S,5S)-1-[(1R,2R)-2-(2′,6′-difluoro[1,1′- biphenyl]-2-yl)cyclopropane-1-carbonyl]-5- methylpyrrolidin-3-yl}methanesulfonamide | 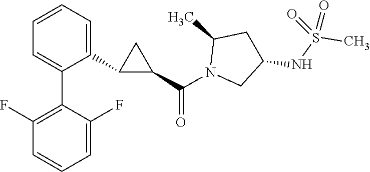 [SMILES: C[C@H]1C[C@H](NS(C)(=O)=O)CN1C(=O)[C@@H]1C[C@H]1c1ccccc1-c1c(F)cccc1F] | 435.2 |
| 263 | N-{(3S,5R)-1-[(1R,2R)-2-(2′,6′-difluoro[1,1′- biphenyl]-2-yl)cyclopropane-1-carbonyl]-5- methylpyrrolidin-3-yl}methanesulfonamide | 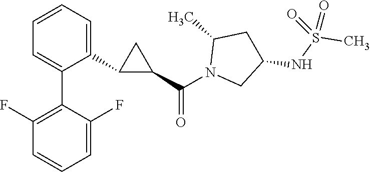 [SMILES: C[C@@H]1C[C@H](NS(C)(=O)=O)CN1C(=O)[C@@H]1C[C@H]1c1ccccc1-c1c(F)cccc1F] | 435.2 |
| 264 | N-{(3S,4R)-1-[(1R,2R)-2-(2′,6′-difluoro[1,1′- biphenyl]-2-yl)cyclopropane-1-carbonyl]-4- methylpyrrolidin-3-yl}methanesulfonamide | 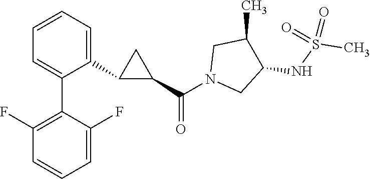 [SMILES: C[C@@H]1CN(C(=O)[C@@H]2C[C@H]2c2ccccc2-c2c(F)cccc2F)C[C@H]1NS(C)(=O)=O] | 435.3 |
| 265 | N-{(3S,4S)-1-[(1R,2R)-2-(2′,6′-difluoro[1,1′- biphenyl]-2-yl)cyclopropane-1-carbonyl]-4- methylpyrrolidin-3-yl}methanesulfonamide | 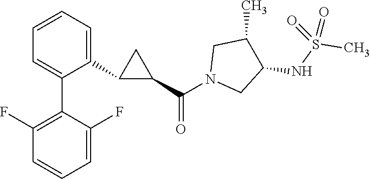 [SMILES: C[C@H]1CN(C(=O)[C@@H]2C[C@H]2c2ccccc2-c2c(F)cccc2F)C[C@H]1NS(C)(=O)=O] | 435.2 |
| 266 | a mixture of rac-N-[(4R)-1-{(1R,2R)-2-[4- (2,6-difluorophenyl)-6-methylpyridin-3- yl]cyclopropane-1-carbonyl}azepan-4- yl]methanesulfonamide and rac-N-[(4S)-1- {(1R,2R)-2-[4-(2,6-difluorophenyl)-6- methylpyridin-3-yl]cyclopropane-1- carbonyl}azepan-4-yl]methanesulfonamide | 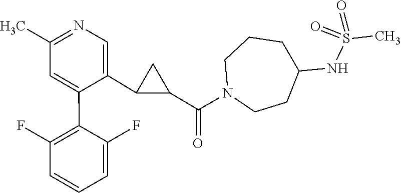 | 464.2 |
| 267 | a mixture of rac-N-[(3R)-1-{(1R,2R)-2-[4- (2,6-difluorophenyl)-6-methylpyridin-3- yl]cyclopropane-1-carbonyl}piperidin-3- yl]methanesulfonamide and rac-N-[(3S)-1- {(1R,2R)-2-[4-(2,6-difluorophenyl)-6- methylpyridin-3-yl]cyclopropane-1- carbonyl}piperidin-3-yl]methanesulfonamide | 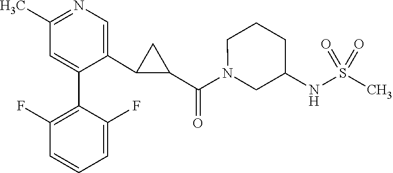 | 450.2 |
| 268 | a mixture of rac-N-[(3R)-1-{(1R,2R)-2-[4- (2,6-difluorophenyl)-6-methylpyridin-3- yl]cyclopropane-1-carbonyl}pyrrolidin-3- yl]methanesulfonamide and rac-N-[(3S)-1- {(1R,2R)-2-[4-(2,6-difluorophenyl)-6- methylpyridin-3-yl]cyclopropane-1- carbonyl}pyrrolidin-3-yl]methanesulfonamide | 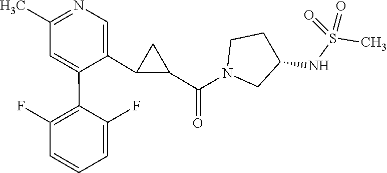 | 436.2 |
| 269 | N-{(3R,4R)-1-[(1R,2R)-2-(2′,6′-difluoro[1,1′- biphenyl]-2-yl)cyclopropane-1-carbonyl]-4- hydroxypiperidin-3-yl}methanesulfonamide | 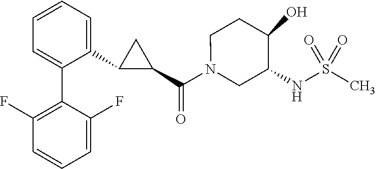 [SMILES: CS(=O)(=O)N[C@@H]1CN(C(=O)[C@@H]2C[C@H]2c2ccccc2-c2c(F)cccc2F)CC[C@H]1O] | 451.2 |
| 270 | N-{(3R,4S)-1-[(1R,2R)-2-(2′,6′-difluoro[1,1′- biphenyl]-2-yl)cyclopropane-1-carbonyl]-4- hydroxypiperidin-3-yl}methanesulfonamide | 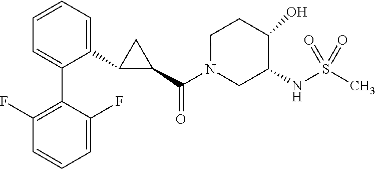 [SMILES: CS(=O)(=O)N[C@@H]1CN(C(=O)[C@@H]2C[C@H]2c2ccccc2-c2c(F)cccc2F)CC[C@@H]1O] | 451.2 |
| 271 | N-{(3S)-1-[(1R,2R)-2-(2′,6′-difluoro[1,1′- biphenyl]-2-yl)cyclopropane-1-carbonyl]-5,5- difluoropiperidin-3-yl}methanesulfonamide | 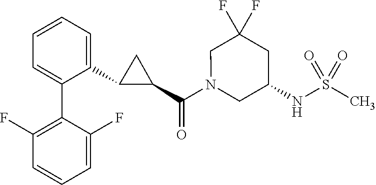 [SMILES: CS(=O)(=O)N[C@@H]1CN(C(=O)[C@@H]2C[C@H]2c2ccccc2-c2c(F)cccc2F)CC(F)(F)C1] | 469.2 |
| 272 | N-{3-[2-(2′,6′-difluoro[1,1′-biphenyl]-2- yl)cyclopropane-1-carbonyl]-3- azabicyclo[3.1.0]hexan-1- yl}methanesulfonamide optical isomer | 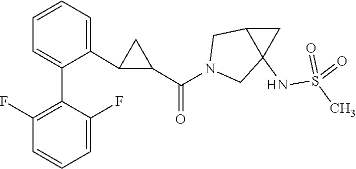 [SMILES: CS(=O)(=O)NC12CC1CN(C(=O)C1CC1c1ccccc1-c1c(F)cccc1F)C2] | 433.2 |
| 273 | N-{3-[2-(2′,6′-difluoro[1,1′-biphenyl]-2- yl)cyclopropane-1-carbonyl]-3- azabicyclo[3.1.0]hexan-1- yl}methanesulfonamide optical isomer | 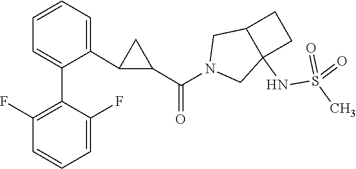 [SMILES: CS(=O)(=O)NC12CCC1CN(C(=O)C1CC1c1ccccc1-c1c(F)cccc1F)C2] | 447.2 |
| 274 | N-{(3S)-1-[(1R,2S)-2-(2′,6′-difluoro[1,1′- biphenyl]-2-yl)-1-methylcyclopropane-1- carbonyl]azepan-3-yl}methanesulfonamide, or N-{(3S)-1-[(1S,2R)-2-(2′,6′-difluoro[1,1′- biphenyl]-2-yl)-1-methylcyclopropane-1- carbonyl]azepan-3-yl}methanesulfonamide | 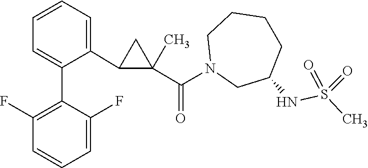 [SMILES: CC1(C(=O)N2CCCC[C@H](NS(C)(=O)=O)C2)CC1c1ccccc1-c1c(F)cccc1F] | 463.2 |
| 275 | a mixture of rac-N-({(3R)-1-[(1R,2R)-2-(2′,6′- difluoro[1,1′-biphenyl]-2-yl)cyclopropane-1- carbonyl]-4,4-difluoropiperidin-3- yl}methyl)methanesulfonamide and rac-N- ({(3S)-1-[(1R,2R)-2-(2′,6′-difluoro[1,1′- biphenyl]-2-yl)cyclopropane-1-carbonyl]-4,4- difluoropiperidin-3- yl}methyl)methanesulfonamide | 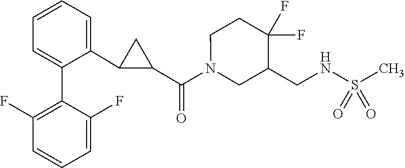 [SMILES: CS(=O)(=O)NCC1CN(C(=O)C2CC2c2ccccc2-c2c(F)cccc2F)CCC1(F)F] | 485.1 |
| 276 | N-({1-[2-(2′,6′-difluoro[1,1′-biphenyl]-2- yl)cyclopropane-1-carbonyl]azetidin-2- yl}methyl)methylsulfonamide optical isomer | 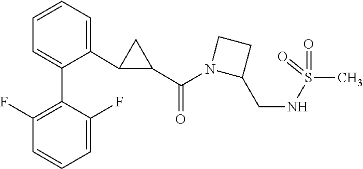 [SMILES: CS(=O)(=O)NCC1CCN1C(=O)C1CC1c1ccccc1-c1c(F)cccc1F] | 421.2 |
| 277 | a mixture of rac-N-[(3R)-1-{(1R,2R)-2-[2- (2,6-difluorophenyl)-1,3-thiazol-5- yl]cyclopropane-1-carbonyl}pyrrolidin-3- yl]methanesulfonamide and rac-N-[(3S)-1- {(1R,2R)-2-[2-(2,6-difluorophenyl)-1,3- thiazol-5-yl]cyclopropane-1- carbonyl}pyrrolidin-3-yl]methanesulfonamide | 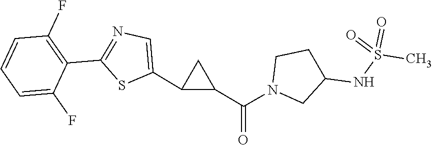 [SMILES: CS(=O)(=O)NC1CCN(C(=O)C2CC2c2cnc(-c3c(F)cccc3F)s2)C1] | 428.1 |
| 278 | a mixture of rac-N-[(3R)-1-{(1R,2R)-2-[5- (2,6-difluorophenyl)thiophen-2- yl]cyclopropane-1-carbonyl}pyrrolidin-3- yl]methanesulfonamide and rac-N-[(3S)-1- {(1R,2R)-2-[5-(2,6-difluorophenyl)thiophen-2- yl]cyclopropane-1-carbonyl}pyrrolidin-3- yl]methanesulfonamide | 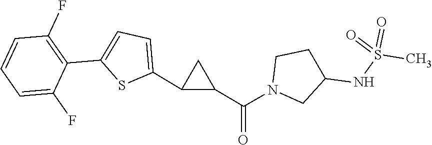 [SMILES: CS(=O)(=O)NC1CCN(C(=O)C2CC2c2ccc(-c3c(F)cccc3F)s2)C1] | 427.2 |
| 279 | N-{(3R,4R)-1-[(1R,2R)-2-(2′,6′-difluoro[1,1′- biphenyl]-2-yl)cyclopropane-1-carbonyl]-4- fluoropyrrolidin-3-yl}methanesulfonamide | 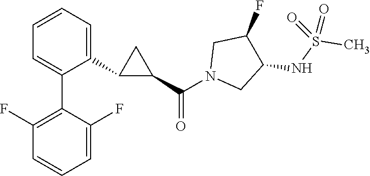 [SMILES: CS(=O)(=O)N[C@@H]1CN(C(=O)[C@@H]2C[C@H]2c2ccccc2-c2c(F)cccc2F)C[C@H]1F] | 439.2 |
| 280 | N-{(3R,4S)-1-[(1R,2R)-2-(2′,6′-difluoro[1,1′- biphenyl]-2-yl)cyclopropane-1-carbonyl]-4- methoxypyrrolidin-3-yl}methanesulfonamide | 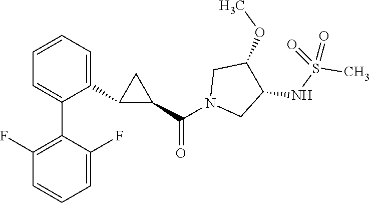 [SMILES: CO[C@H]1CN(C(=O)[C@@H]2C[C@H]2c2ccccc2-c2c(F)cccc2F)C[C@H]1NS(C)(=O)=O] | 451.2 |
| 281 | N′-{(3S,5S)-1-[(1R,2R)-2-(2′,6′-difluoro[1,1′- biphenyl]-2-yl)cyclopropane-1-carbonyl]-5- methylpyrrolidin-3-yl}-N,N-dimethylsulfuric diamide | 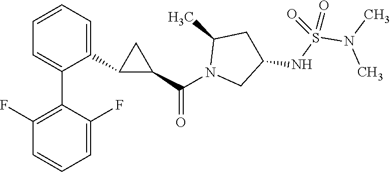 [SMILES: C[C@H]1C[C@H](NS(=O)(=O)N(C)C)CN1C(=O)[C@@H]1C[C@H]1c1ccccc1-c1c(F)cccc1F] | 464.2 |
| 282 | N-({(2R)-4-[(1R,2R)-2-(2′,6′-difluoro[1,1′- biphenyl]-2-yl)cyclopropane-1- carbonyl]morpholin-2- yl}methyl)methanesulfonamide | 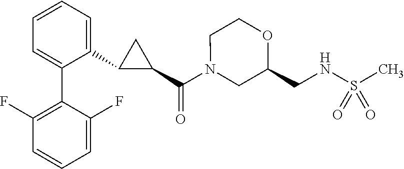 [SMILES: CS(=O)(=O)NC[C@H]1CN(C(=O)[C@@H]2C[C@H]2c2ccccc2-c2c(F)cccc2F)CCO1] | 451.2 |
| 283 | N-({(2S)-4-[(1R,2R)-2-(2′,6′-difluoro[1,1′- biphenyl]-2-yl)cyclopropane-1- carbonyl]morpholin-2- yl}methyl)methanesulfonamide | 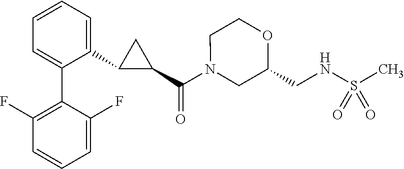 [SMILES: CS(=O)(=O)NC[C@@H]1CN(C(=O)[C@@H]2C[C@H]2c2ccccc2-c2c(F)cccc2F)CCO1] | 451.3 |
| 284 | N′-({(2R)-4-[(1R,2R)-2-(2′,6′-difluoro[1,1′- biphenyl]-2-yl)cyclopropane-1- carbonyl]morpholin-2-yl}methyl)-N,N- dimethylsulfuric diamide | 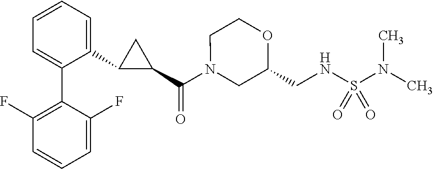 [SMILES: CN(C)S(=O)(=O)NC[C@@H]1CN(C(=O)[C@@H]2C[C@H]2c2ccccc2-c2c(F)cccc2F)CCO1] | 480.2 |
| 285 | a mixture of rac-N-({(3R)-1-[(1R,2R)-2-(2′,6′- difluoro[1,1′-biphenyl]-2-yl)cyclopropane-1- carbonyl]-4,4-difluoropyrrolidin-3- yl}methyl)methanesulfonamide and rac-N- ({(3S)-1-[(1R,2R)-2-(2′,6′-difluoro[1,1′- biphenyl]-2-yl)cyclopropane-1-carbonyl]-4,4- difluoropyrrolidin-3- yl}methyl)methanesulfonamide | 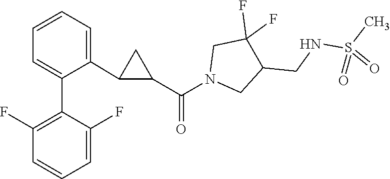 [SMILES: CS(=O)(=O)NCC1CN(C(=O)C2CC2c2ccccc2-c2c(F)cccc2F)CC1(F)F] | 471.1 |
| 286 | a mixture of N-{(3R)-1-[(1R,2R)-2-(2′,6′- difluoro[1,1′-biphenyl]-2-yl)cyclopropane-1- carbonyl]-3-methylpyrrolidin-3- yl}methanesulfonamide and N-{(3S)-1- [(1R,2R)-2-(2′,6′-difluoro[1,1′-biphenyl]-2- yl)cyclopropane-1-carbonyl]-3- methylpyrrolidin-3-yl}methanesulfonamide | 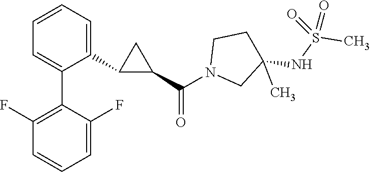 [SMILES: C[C@]1(NS(C)(=O)=O)CCN(C(=O)[C@@H]2C[C@H]2c2ccccc2-c2c(F)cccc2F)C1] | 435.3 |
| 287 | a mixture of N-{(3S)-1-[(1R,2R)-2-(2′,6′- difluoro[1,1′-biphenyl]-2-yl)-2,2- dimethylcyclopropane-1-carbonyl]pyrrolidin-3- yl}methanesulfonamide and N-{(3S)-1- [(1S,3S)-3-(2′,6′-difluoro[1,1′-biphenyl]-2-yl)- 2,2-dimethylcyclopropane-1- carbonyl]pyrrolidin-3-yl}methanesulfonamide | 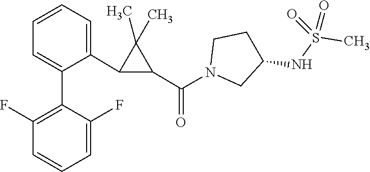 [SMILES: CC1(C)C(C(=O)N2CC[C@H](NS(C)(=O)=O)C2)C1c1ccccc1-c1c(F)cccc1F] | 449.2 |
| 288 | a mixture of N-{(4S)-1-[(1R,2R)-2-(2′,6′- difluoro[1,1′-biphenyl]-2-yl)-2,2- dimethylcyclopropane-1-carbonyl]azepan-4- yl}methanesulfonamide and N-{(4S)-1- [(1S,3S)-3-(2′,6′-difluoro[1,1′-biphenyl]-2-yl)- 2,2-dimethylcyclopropane-1-carbonyl]azepan- 4-yl}methanesulfonamide | 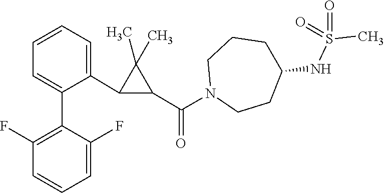 [SMILES: CC1(C)C(C(=O)N2CCC[C@H](NS(C)(=O)=O)CC2)C1c1ccccc1-c1c(F)cccc1F] | 477.3 |
| 289 | N′-{(3S)-1-[(1R,2R)-2-(2′,6′-difluoro[1,1′- biphenyl]-2-yl)cyclopropane-1- carbonyl]pyrrolidin-3-yl}-N,N-dimethylsulfuric diamide | 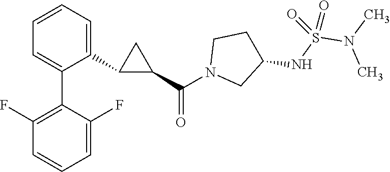 [SMILES: CN(C)S(=O)(=O)N[C@H]1CCN(C(=O)[C@@H]2C[C@H]2c2ccccc2-c2c(F)cccc2F)C1] | 450.3 |
| 290 | a mixture of N-{(4S)-1-[(1R,2R)-2-(2′,6′- difluoro[1,1′-biphenyl]-2-yl)-2- fluorocyclopropane-1-carbonyl]azepan-4- yl}methanesulfonamide and N-{(4S)-1- [(1S,2S)-2-(2′,6′-difluoro[1,1′-biphenyl]-2-yl)- 2-fluorocyclopropane-1-carbonyl]azepan-4- yl}methanesulfonamide | 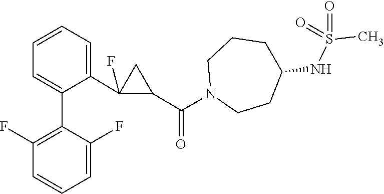 [SMILES: CS(=O)(=O)N[C@H]1CCCN(C(=O)C2CC2(F)c2ccccc2-c2c(F)cccc2F)CC1] | 467.2 |
| 291 | a mixture of N-{(3S)-1-[(1R,2R)-2-(2′,6′- difluoro[1,1′-biphenyl]-2-yl)-2- fluorocyclopropane-1-carbonyl]azepan-3- yl}methanesulfonamide and N-{(3S)-1- [(1S,2S)-2-(2′,6′-difluoro[1,1′-biphenyl]-2-yl)- 2-fluorocyclopropane-1-carbonyl]azepan-3- yl}methanesulfonamide | 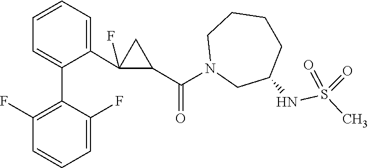 [SMILES: CS(=O)(=O)N[C@H]1CCCCN(C(=O)C2CC2(F)c2ccccc2-c2c(F)cccc2F)C1] | 467.2 |
| 292 | a mixture of rac-N-[(4R)-1-{(1R,2R)-2-[3- (2,6-difluorophenyl)pyridin-2-yl]cyclopropane- 1-carbonyl}azepan-4-yl]methanesulfonamide and rac-N-[(4S)-1-{(1R,2R)-2-[3-(2,6- difluorophenyl)pyridin-2-yl]cyclopropane-1- carbonyl}azepan-4-yl]methanesulfonamide | 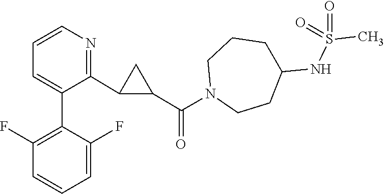 [SMILES: CS(=O)(=O)NC1CCCN(C(=O)C2CC2c2ncccc2-c2c(F)cccc2F)CC1] | 450.2 |
| 293 | a mixture of rac-N-[(4R)-1-{(1R,2R)-2-[4- (2,6-difluorophenyl)-2-methyl-1,3-thiazol-5- yl]cyclopropane-1-carbonyl}azepan-4- yl]methanesulfonamide and rac-N-[(4S)-1- {(1R,2R)-2-[4-(2,6-difluorophenyl)-2-methyl- 1,3-thiazol-5-yl]cyclopropane-1- carbonyl}azepan-4-yl]methanesulfonamide | 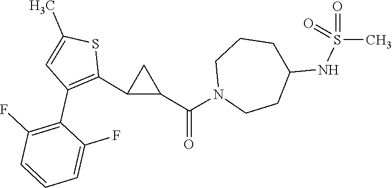 [SMILES: Cc1cc(-c2c(F)cccc2F)c(C2CC2C(=O)N2CCCC(NS(C)(=O)=O)CC2)s1] | 470.1 |
| 294 | N-{(6S)-4-[(1R,2R)-2-(2′,6′-difluoro[1,1′- biphenyl]-2-yl)cyclopropane-1-carbonyl]-1,4- oxazepan-6-yl}ethanesulfonamide, or N-{(6R)- 4-[(1R,2R)-2-(2′,6′-difluoro[1,1′-biphenyl]-2- yl)cyclopropane-1-carbonyl]-1,4-oxazepan-6- yl}ethanesulfonamide | 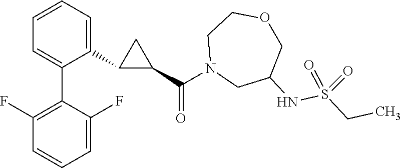 [SMILES: CCS(=O)(=O)NC1COCCN(C(=O)[C@@H]2C[C@H]2c2ccccc2-c2c(F)cccc2F)C1] | 465.3 |
| 295 | N′-{(6S)-4-[(1R,2R)-2-(2′,6′-difluoro[1,1′- biphenyl]-2-yl)cyclopropane-1-carbonyl]-1,4- oxazepan-6-yl}-N,N-dimethylsulfuric diamide, or N′-{(6R)-4-[(1R,2R)-2-(2′,6′-difluoro[1,1′- biphenyl]-2-yl)cyclopropane-1-carbonyl]-1,4- oxazepan-6-yl}-N,N-dimethylsulfuric diamide | 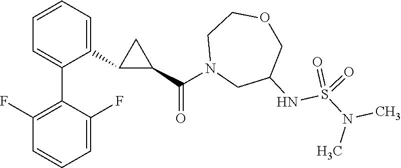 [SMILES: CN(C)S(=O)(=O)NC1COCCN(C(=O)[C@@H]2C[C@H]2c2ccccc2-c2c(F)cccc2F)C1] | 480.3 |
| 296 | N-{(2R,3R)-1-[(1R,2R)-2-(2′,6′-difluoro[1,1′- biphenyl]-2-yl)cyclopropane-1-carbonyl]-2- methylpyrrolidin-3-yl}methanesulfonamide, or N-{(2S,3S)-1-[(1R,2R)-2-(2′,6′-difluoro[1,1′- biphenyl]-2-yl)cyclopropane-1-carbonyl]-2- methylpyrrolidin-3-yl}methanesulfonamide | 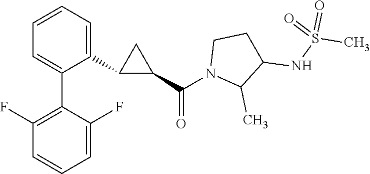 [SMILES: CC1C(NS(C)(=O)=O)CCN1C(=O)[C@@H]1C[C@H]1c1ccccc1-c1c(F)cccc1F] | 435.2 |
| 297 | N-{(2R,3R)-1-[(1R,2R)-2-(2′,6′-difluoro[1,1′- biphenyl]-2-yl)cyclopropane-1-carbonyl]-2- methylpyrrolidin-3-yl}methanesulfonamide, or N-{(2S,3S)-1-[(1R,2R)-2-(2′,6′-difluoro[1,1′- biphenyl]-2-yl)cyclopropane-1-carbonyl]-2- methylpyrrolidin-3-yl}methanesulfonamide | 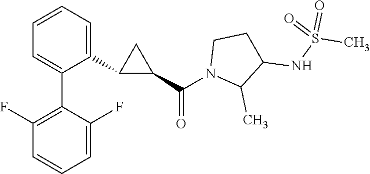 [SMILES: CC1C(NS(C)(=O)=O)CCN1C(=O)[C@@H]1C[C@H]1c1ccccc1-c1c(F)cccc1F] | 435.2 |
| 298 | N′-({(2R)-1-[(1R,2R)-2-(2′,6′-difluoro[1,1′- biphenyl]-2-yl)cyclopropane-1- carbonyl]azetidin-2-yl}methyl)-N,N- dimethylsulfuric diamide | 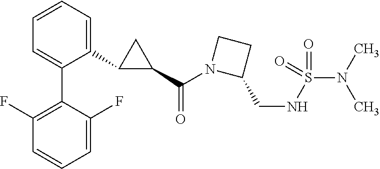 [SMILES: CN(C)S(=O)(=O)NC[C@H]1CCN1C(=O)[C@@H]1C[C@H]1c1ccccc1-c1c(F)cccc1F] | 450.2 |
| 299 | N′-({(2S)-4-[(1R,2R)-2-(2′,6′-difluoro[1,1′- biphenyl]-2-yl)cyclopropane-1- carbonyl]morpholin-2-yl}methyl)-N,N- dimethylsulfuric diamide | 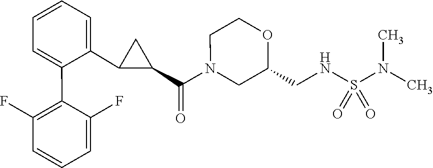 [SMILES: CN(C)S(=O)(=O)NC[C@@H]1CN(C(=O)[C@@H]2CC2c2ccccc2-c2c(F)cccc2F)CCO1] | 480.3 |
| 300 | N-{(3S,6R)-4-[(1R,2R)-2-(2′,6′-difluoro[1,1′- biphenyl]-2-yl)cyclopropane-1-carbonyl]-3- methyl-1,4-oxazepan-6- yl}methanesulfonamide, or N-{(3S,6S)-4- [(1R,2R)-2-(2′,6′-difluoro[1,1′-biphenyl]-2- yl)cyclopropane-1-carbonyl]-3-methyl-1,4- oxazepan-6-yl}methanesulfonamide | 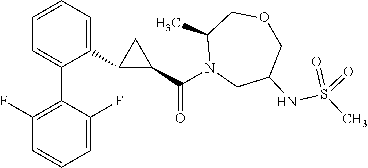 [SMILES: C[C@H]1COCC(NS(C)(=O)=O)CN1C(=O)[C@@H]1C[C@H]1c1ccccc1-c1c(F)cccc1F] | 465.2 |
| 301 | N-{(3S,6R)-4-[(1R,2R)-2-(2′,6′-difluoro[1,1′- biphenyl]-2-yl)cyclopropane-1-carbonyl]-3- methyl-1,4-oxazepan-6- yl}methanesulfonamide, or N-{(3S,6S)-4- [(1R,2R)-2-(2′,6′-difluoro[1,1′-biphenyl]-2- yl)cyclopropane-1-carbonyl]-3-methyl-1,4- oxazepan-6-yl}methanesulfonamide | 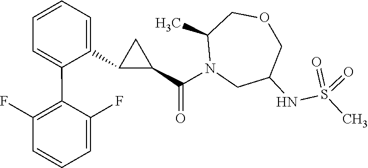 [SMILES: C[C@H]1COCC(NS(C)(=O)=O)CN1C(=O)[C@@H]1C[C@H]1c1ccccc1-c1c(F)cccc1F] | 465.2 |
| 302 | N′-{(3S)-1-[(1R,2R)-2-(2′,6′-difluoro[1,1′- biphenyl]-2-yl)cyclopropane-1- carbonyl]piperidin-3-yl}-N,N-dimethylsulfuric diamide | 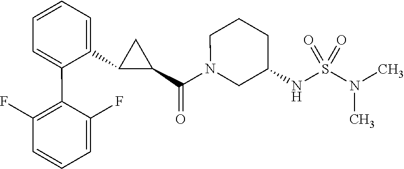 [SMILES: CN(C)S(=O)(=O)N[C@H]1CCCN(C(=O)[C@@H]2C[C@H]2c2ccccc2-c2c(F)cccc2F)C1] | 464.2 |
| 303 | a mixture of N-{(3S)-1-[(1R,2R,3S)-2-(2′,6′- difluoro[1,1′-biphenyl]-2-yl)-3- methylcyclopropane-1-carbonyl]pyrrolidin-3- yl}methanesulfonamide and N-{(3S)-1- [(1S,2S,3R)-2-(2′,6′-difluoro[1,1′-biphenyl]-2- yl)-3-methylcyclopropane-1- carbonyl]pyrrolidin-3-yl}methanesulfonamide | 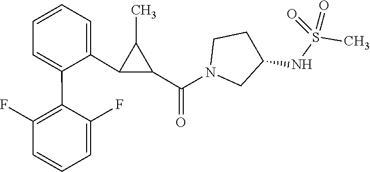 [SMILES: CC1C(C(=O)N2CC[C@H](NS(C)(=O)=O)C2)C1c1ccccc1-c1c(F)cccc1F] | 435.2 |
| 304 | N-{(3S)-1-[(1S,2S)-2-(2′,6′-difluoro[1,1′- biphenyl]-2-yl)-2-fluorocyclopropane-1- carbonyl]pyrrolidin-3-yl}methanesulfonamide, or N-{(3S)-1-[(1R,2R)-2-(2′,6′-difluoro[1,1′- biphenyl]-2-yl)-2-fluorocyclopropane-1- carbonyl]pyrrolidin-3-yl}methanesulfonamide | 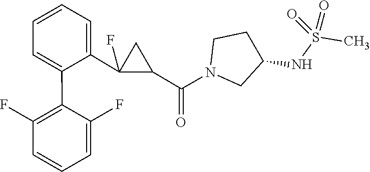 [SMILES: CS(=O)(=O)N[C@H]1CCN(C(=O)C2CC2(F)c2ccccc2-c2c(F)cccc2F)C1] | 439.3 |
| 305 | a mixture of N-{(3S)-1-[(1R,2R,3S)-2-(2′,6′- difluoro[1,1′-biphenyl]-2-yl)-3- methylcyclopropane-1-carbonyl]piperidin-3- yl}methanesulfonamide and N-{(3S)-1- [(1S,2S,3R)-2-(2′,6′-difluoro[1,1′-biphenyl]-2- yl)-3-methylcyclopropane-1- carbonyl]piperidin-3-yl}methanesulfonamide | 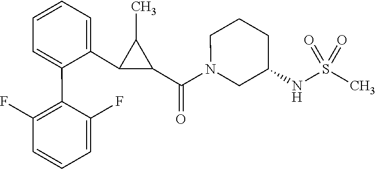 [SMILES: CC1C(C(=O)N2CCC[C@H](NS(C)(=O)=O)C2)C1c1ccccc1-c1c(F)cccc1F] | 449.2 |
| 306 | N-{(3S)-1-[(1S,2S)-2-(2′,6′-difluoro[1,1′- biphenyl]-2-yl)-2-fluorocyclopropane-1- carbonyl]piperidin-3-yl}methanesulfonamide, or N-{(3S)-1-[(1R,2R)-2-(2′,6′-difluoro[1,1′- biphenyl]-2-yl)-2-fluorocyclopropane-1- carbonyl]piperidin-3-yl}methanesulfonamide | 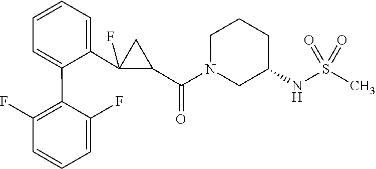 [SMILES: CS(=O)(=O)N[C@H]1CCCN(C(=O)C2CC2(F)c2ccccc2-c2c(F)cccc2F)C1] | 453.3 |
| 307 | N-{(3S)-1-[(1R,2R)-2-(2′,3,6′-trifluoro[1,1′- biphenyl]-2-yl)cyclopropane-1- carbonyl]piperidin-3-yl}ethanesulfonamide | 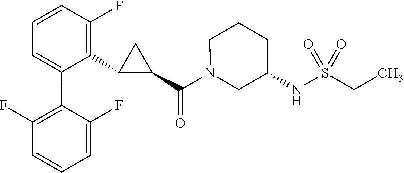 [SMILES: CCS(=O)(=O)N[C@H]1CCCN(C(=O)[C@@H]2C[C@H]2c2c(F)cccc2-c2c(F)cccc2F)C1] | 467.2 |
| 308 | a mixture of N-{(4S)-1-[(1R,2R,3S)-2-(2′,6′- difluoro[1,1′-biphenyl]-2-yl)-3- methylcyclopropane-1-carbonyl]azepan-4- yl}methanesulfonamide and N-{(4S)-1- [(1S,2S,3R)-2-(2′,6′-difluoro[1,1′-biphenyl]-2- yl)-3-methylcyclopropane-1-carbonyl]azepan- 4-yl}methanesulfonamide | 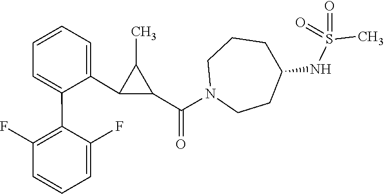 [SMILES: CC1C(C(=O)N2CCC[C@H](NS(C)(=O)=O)CC2)C1c1ccccc1-c1c(F)cccc1F] | 463.2 |
| 309 | N,N-dimethyl-N′-{(3S)-1-[(1R,2R)-2-(2′,3,6′- trifluoro[1,1′-biphenyl]-2-yl)cyclopropane-1- carbonyl]pyrrolidin-3-yl}sulfuric diamide | 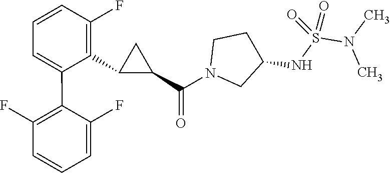 [SMILES: CN(C)S(=O)(=O)N[C@H]1CCN(C(=O)[C@@H]2C[C@H]2c2c(F)cccc2-c2c(F)cccc2F)C1] | 468.3 |
| 310 | a mixture of N-{(3R)-1-[(1R,2R)-2-(2′,6′- difluoro[1,1′-biphenyl]-2-yl)cyclopropane-1- carbonyl]-3-methylpiperidin-3- yl}methanesulfonamide and N-{(3S)-1- [(1R,2R)-2-(2′,6′-difluoro[1,1′-biphenyl]-2- yl)cyclopropane-1-carbonyl]-3- methylpiperidin-3-yl}methanesulfonamide | 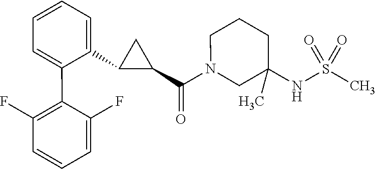 [SMILES: CC1(NS(C)(=O)=O)CCCN(C(=O)[C@@H]2C[C@H]2c2ccccc2-c2c(F)cccc2F)C1] | 449.2 |
| 311 | a mixture of N-{(3S)-1-[(1R,2R,3S)-2-(2′,6′- difluoro[1,1′-biphenyl]-2-yl)-3- methylcyclopropane-1-carbonyl]azepan-3- yl}methanesulfonamide and N-{(3S)-1- [(1S,2S,3R)-2-(2′,6′-difluoro[1,1′-biphenyl]-2- yl)-3-methylcyclopropane-1-carbonyl]azepan- 3-yl}methanesulfonamide | 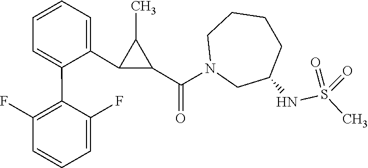 [SMILES: CC1C(C(=O)N2CCCC[C@H](NS(C)(=O)=O)C2)C1c1ccccc1-c1c(F)cccc1F] | 463.2 |
| 312 | N,N-dimethyl-N′-{(3S)-1-[(1R,2R)-2-(2′,3,6′- trifluoro[1,1′-biphenyl]-2-yl)cyclopropane-1- carbonyl]piperidin-3-yl}sulfuric diamide | 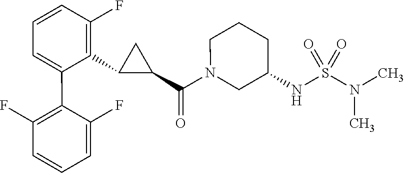 [SMILES: CN(C)S(=O)(=O)N[C@H]1CCCN(C(=O)[C@@H]2C[C@H]2c2c(F)cccc2-c2c(F)cccc2F)C1] | 482.2 |
| 313 | a mixture of N-{(4R)-1-[(1R,2R)-2-(2′,6′- difluoro[1,1′-biphenyl]-2-yl)cyclopropane-1- carbonyl]-4-methylazepan-4- yl}methanesulfonamide and N-{(4S)-1- [(1R,2R)-2-(2′,6′-difluoro[1,1′-biphenyl]-2- yl)cyclopropane-1-carbonyl]-4-methylazepan- 4-yl}methanesulfonamide | 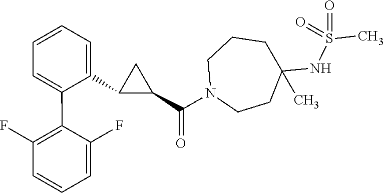 [SMILES: CC1(NS(C)(=O)=O)CCCN(C(=O)[C@@H]2C[C@H]2c2ccccc2-c2c(F)cccc2F)CC1] | 463.2 |
| 314 | N,N-dimethyl-N′-({(2S)-1-[(1R,2R)-2-(2′,3,6′- trifluoro[1,1′-biphenyl]-2-yl)cyclopropane-1- carbonyl]azetidin-2-yl}methyl)sulfuric diamide, or N,N-dimethyl-N′-({(2S)-1- [(1S,2S)-2-(2′,3,6′-trifluoro[1,1′-biphenyl]-2- yl)cyclopropane-1-carbonyl]azetidin-2- yl}methyl)sulfuric diamide | 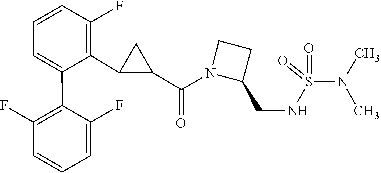 [SMILES: CN(C)S(=O)(=O)NC[C@@H]1CCN1C(=O)C1CC1c1c(F)cccc1-c1c(F)cccc1F] | 468.2 |
| 315 | N-{(3S,5S)-1-[(1R,2S)-2-(2′,6′-difluoro[1,1′- biphenyl]-2-yl)-1-methylcyclopropane-1- carbonyl]-5-methylpyrrolidin-3- yl}methanesulfonamide, or N-{(3S,5S)-1- [(1S,2R)-2-(2′,6′-difluoro[1,1′-biphenyl]-2-yl)- 1-methylcyclopropane-1-carbonyl]-5- methylpyrrolidin-3-yl}methanesulfonamide | 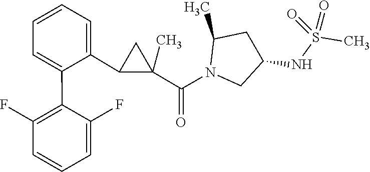 [SMILES: C[C@H]1C[C@H](NS(C)(=O)=O)CN1C(=O)C1(C)CC1c1ccccc1-c1c(F)cccc1F] | 449.2 |
| 316 | a mixture of rac-N-{(3S)-1-[(1R,2S)-1-cyano-2- (2′,6′-difluoro[1,1′-biphenyl]-2- yl)cyclopropane-1-carbonyl]pyrrolidin-3- yl}methanesulfonamide and N-{(3S)-1- [(1S,2R)-1-cyano-2-(2′,6′-difluoro[1,1′- biphenyl]-2-yl)cyclopropane-1- carbonyl]pyrrolidin-3-yl}methanesulfonamide | 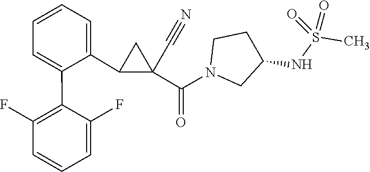 [SMILES: CS(=O)(=O)N[C@H]1CCN(C(=O)C2(C#N)CC2c2ccccc2-c2c(F)cccc2F)C1] | 444.2 |
| 317 | a mixture of rac-N-{(4R)-1-[(1R,2S)-1-cyano- 2-(2′,6′-difluoro[1,1′-biphenyl]-2- yl)cyclopropane-1-carbonyl]azepan-4- yl}methanesulfonamide and rac-N-{(4S)-1- [(1R,2S)-1-cyano-2-(2′,6′-difluoro[1,1′- biphenyl]-2-yl)cyclopropane-1- carbonyl]azepan-4-yl}methanesulfonamide | 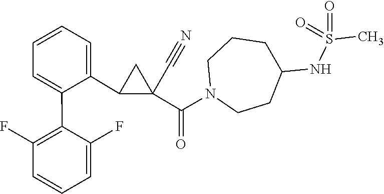 [SMILES: CS(=O)(=O)NC1CCCN(C(=O)C2(C#N)CC2c2ccccc2-c2c(F)cccc2F)CC1] | 474.2 |
| 318 | N-{(3S)-1-[(1R,2R)-2-(2′,6′-difluoro-5- methyl[1,1′-biphenyl]-2-yl)cyclopropane-1- carbonyl]pyrrolidin-3-yl}methanesulfonamide, or N-{(3S)-1-[(1S,2S)-2-(2′,6′-difluoro-5- methyl[1,1′-biphenyl]-2-yl)cyclopropane-1- carbonyl]pyrrolidin-3-yl}methanesulfonamide | 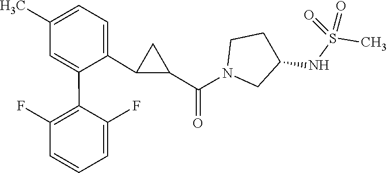 | 435.2 |
| 319 | N-{(3S,5S)-1-[(1R,2R)-2-(2′,6′-difluoro[1,1′- biphenyl]-2-yl)cyclopropane-1-carbonyl]-5- hydroxypiperidin-3-yl}methanesulfonamide | 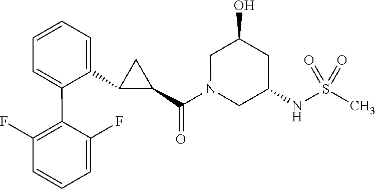 [SMILES: CS(=O)(=O)N[C@H]1C[C@H](O)CN(C(=O)[C@@H]2C[C@H]2c2ccccc2-c2c(F)cccc2F)C1] | 451.3 |
| 320 | a mixture of N-{(3S)-1-[(1R,2R)-2-(2′,6′- difluoro[1,1′-biphenyl]-2-yl)-2,2- difluorocyclopropane-1-carbonyl]pyrrolidin-3- yl}methanesulfonamide and N-{(3S)-1- [(1S,3S)-3-(2′,6′-difluoro[1,1′-biphenyl]-2-yl)- 2,2-difluorocyclopropane-1- carbonyl]pyrrolidin-3-yl}methanesulfonamide | 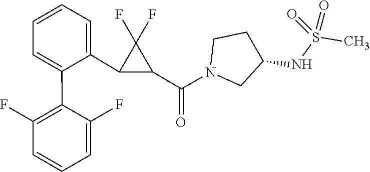 [SMILES: CS(=O)(=O)N[C@H]1CCN(C(=O)C2C(c3ccccc3-c3c(F)cccc3F)C2(F)F)C1] | 457.2 |
| 321 | a mixture of rac-N-[(3R)-1-{(1R,2R)-2-[4- (2,6-difluorophenyl)thiophen-3- yl]cyclopropane-1-carbonyl}pyrrolidin- 3-yl]methanesulfonamide and rac-N- [(3R)-1-{(1R,2R)-2-[4-(2,6- difluorophenyl)thiophen-3- yl]cyclopropane-1-carbonyl}pyrrolidin- 3-yl]methanesulfonamide | 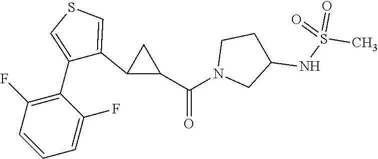 [SMILES: CS(=O)(=O)NC1CCN(C(=O)C2CC2c2cscc2-c2c(F)cccc2F)C1] | 427.2 |
| 322 | N-{(3R,4R)-1-[(1R,2R)-2-(2′,6′- difluoro[1,1′-biphenyl]-2- yl)cyclopropane-1-carbonyl]-4- hydroxypyrrolidin-3- yl}methanesulfonamide | 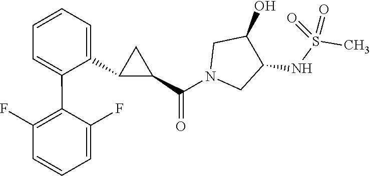 [SMILES: CS(=O)(=O)N[C@@H]1CN(C(=O)[C@@H]2C[C@H]2c2ccccc2-c2c(F)cccc2F)C[C@H]1O] | 437.2 |
| 323 | N-{(3R,4R)-1-[(1R,2R)-2-(2′,6′- difluoro[1,1′-biphenyl]-2- yl)cyclopropane-1-carbonyl]-4- methoxypyrrolidin-3- yl}methanesulfonamide | 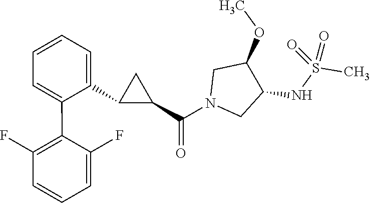 [SMILES: CO[C@@H]1CN(C(=O)[C@@H]2C[C@H]2c2ccccc2-c2c(F)cccc2F)C[C@H]1NS(C)(=O)=O] | 451.2 |
| 324 | N-{(3S)-1-[(1R,2R)-2-(2′,6′-difluoro[1,1′- biphenyl]-2-yl)-1-fluorocyclopropane- 1-carbonyl]pyrrolidin-3- yl}methanesulfonamide, or N-{(3S)-1- [(1S,2S)-2-(2′,6′-difluoro[1,1′-biphenyl]- 2-yl)-1-fluorocyclopropane-1- carbonyl]pyrrolidin-3- yl}methanesulfonamide | 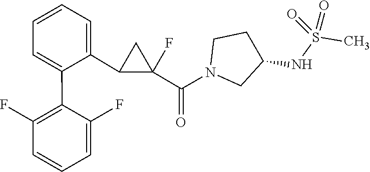 [SMILES: CS(=O)(=O)N[C@H]1CCN(C(=O)C2(F)CC2c2ccccc2-c2c(F)cccc2F)C1] | 439.1 |
| 325 | N-{(3S)-1-[(1R,2R)-2-(2′,6′-difluoro[1,1′- biphenyl]-2-yl)-1-fluorocyclopropane- 1-carbonyl]piperidin-3- yl}methanesulfonamide, or N-{(3S)-1- [(1S,2S)-2-(2′,6′-difluoro[1,1′-biphenyl]- 2-yl)-1-fluorocyclopropane-1- carbonyl]piperidin-3- yl}methanesulfonamide | 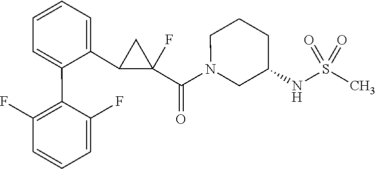 [SMILES: CS(=O)(=O)N[C@H]1CCCN(C(=O)C2(F)CC2c2ccccc2-c2c(F)cccc2F)C1] | 453.2 |
| 326 | N-{(3S)-1-[(1R,2R)-2-(2′,6′-difluoro[1,1′- biphenyl]-2-yl)cyclopropane-1- carbonyl]pyrrolidin-3-yl}azetidine-1- sulfonamide | 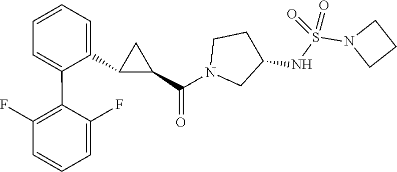 [SMILES: O=C([C@@H]1C[C@H]1c1ccccc1-c1c(F)cccc1F)N1CC[C@H](NS(=O)(=O)N2CCC2)C1] | 462.3 |
| 327 | N-({1-[2-(2′,6′-difluoro[1,1′-biphenyl]-2- yl)-1-methylcyclopropane-1- carbonyl]azetidin-2- yl}methyl)methanesulfonamide optical isomer | 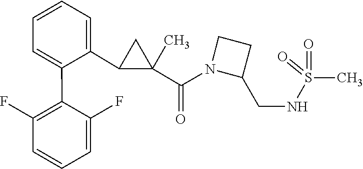 [SMILES: CC1(C(=O)N2CCC2CNS(C)(=O)=O)CC1c1ccccc1-c1c(F)cccc1F] | 435.2 |
| 328 | N-{(3S)-1-[(1R,3R)-3-(2′,6′-difluoro[1,1′- biphenyl]-2-yl)-2,2- difluorocyclopropane-1- carbonyl]pyrrolidin-3- yl}methanesulfonamide, or N-{(3S)-1- [(1R,3R)-3-(2′,6′-difluoro[1,1′-biphenyl]- 2-yl)-2,2-difluorocyclopropane-1- carbonyl]pyrrolidin-3- yl}methanesulfonamide | 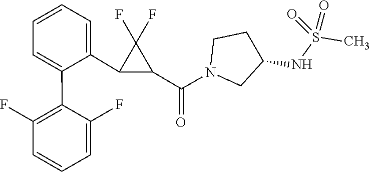 [SMILES: CS(=O)(=O)N[C@H]1CCN(C(=O)C2C(c3ccccc3-c3c(F)cccc3F)C2(F)F)C1] | 455.2 |
| 329 | a mixture of rac-N-[(3R)-1-{(1R,2R)-2-[4- (2,6-difluorophenyl)-2-methylthiophen- 3-yl]cyclopropane-1- carbonyl}pyrrolidin-3- yl]methanesulfonamide and rac-N- [(3S)-1-{(1R,2R)-2-[4-(2,6- difluorophenyl)-2-methylthiophen-3- yl]cyclopropane-1-carbonyl}pyrrolidin- 3-yl]methanesulfonamide | 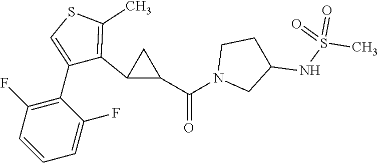 [SMILES: Cc1scc(-c2c(F)cccc2F)c1C1CC1C(=O)N1CCC(NS(C)(=O)=O)C1] | 441.2 |
| 330 | N-{(3S)-1-[(1R,3R)-3-(2′,6′-difluoro[1,1′- biphenyl]-2-yl)-2,2- difluorocyclopropane-1- carbonyl]piperidin-3- yl}methanesulfonamide, or N-{(3S)-1- [(1S,3S)-3-(2′,6′-difluoro[1,1′-biphenyl]- 2-yl)-2,2-difluorocyclopropane-1- carbonyl]piperidin-3- yl}methanesulfonamide | 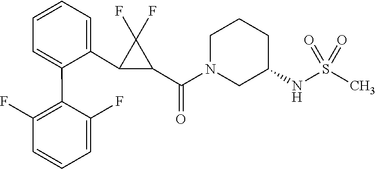 [SMILES: CS(=O)(=O)N[C@H]1CCCN(C(=O)C2C(c3ccccc3-c3c(F)cccc3F)C2(F)F)C1] | 471.2 |
| 331 | N-{(3R)-1-[(1S,2S)-2-(2′,6′-difluoro[1,1′- biphenyl]-2-yl)-2-fluorocyclopropane- 1-carbonyl]-4,4-difluoropyrrolidin-3- yl}methanesulfonamide, or N-{(3S)-1- [(1S,2S)-2-(2′,6′-difluoro[1,1′-biphenyl]- 2-yl)-2-fluorocyclopropane-1- carbonyl]-4,4-difluoropyrrolidin-3- yl}methanesulfonamide | 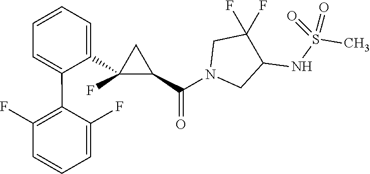 [SMILES: CS(=O)(=O)NC1CN(C(=O)[C@@H]2C[C@@]2(F)c2ccccc2-c2c(F)cccc2F)CC1(F)F] | 473.2 |
| 332 | N-{(3S,5S)-1-[(1S,2S)-2-(2′,6′- difluoro[1,1′-biphenyl]-2-yl)-2- fluorocyclopropane-1-carbonyl]-5- hydroxypiperidin-3- yl}methanesulfonamide | 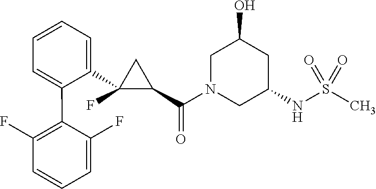 [SMILES: CS(=O)(=O)N[C@H]1C[C@H](O)CN(C(=O)[C@@H]2C[C@@]2(F)c2ccccc2-c2c(F)cccc2F)C1] | 469.2 |
| 333 | N-{(3R)-1-[(1S,2S)-2-(2′,6′-difluoro[1,1′- biphenyl]-2-yl)-2-fluorocyclopropane- 1-carbonyl]-4,4-difluoropiperidin-3- yl}methanesulfonamide | 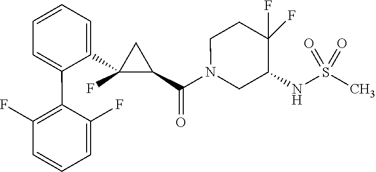 [SMILES: CS(=O)(=O)N[C@@H]1CN(C(=O)[C@@H]2C[C@@]2(F)c2ccccc2-c2c(F)cccc2F)CCC1(F)F] | 489.1 |
| 334 | N-({(2S)-1-[(1R,2R)-2-(2′,6′-difluoro[1,1′- biphenyl]-2-yl)cyclopropane-1- carbonyl]azetidin-2- yl}methyl)azetidine-1-sulfonamide | 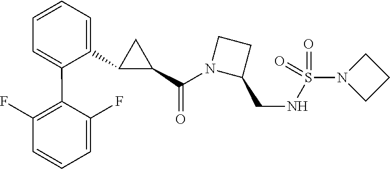 [SMILES: O=C([C@@H]1C[C@H]1c1ccccc1-c1c(F)cccc1F)N1CC[C@H]1CNS(=O)(=O)N1CCC1] | 462.3 |
| 335 | N-{(3S)-1-[(1R,2R)-2-(2′,6′-difluoro[1,1′- biphenyl]-2-yl)cyclopropane-1- carbonyl]pyrrolidin-3-yl}-N′- methylsulfuric diamide | 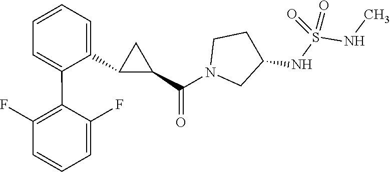 [SMILES: CNS(=O)(=O)N[C@H]1CCN(C(=O)[C@@H]2C[C@H]2c2ccccc2-c2c(F)cccc2F)C1] | 436.4 |
| 336 | a mixture of N-({(2R,4S)-1-[(1R,2R)-2- (2′,6′-difluoro[1,1′-biphenyl]-2- yl)cyclopropane-1-carbonyl]-4- methylazetidin-2- yl}methyl)methanesulfonamide and N- ({(2S,4R)-1-[(1R,2R)-2-(2′,6′- difluoro[1,1′-biphenyl]-2- yl)cyclopropane-1-carbonyl]-4- methylazetidin-2- yl}methyl)methanesulfonamide | 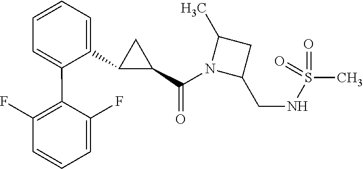 [SMILES: CC1CC(CNS(C)(=O)=O)N1C(=O)[C@@H]1C[C@H]1c1ccccc1-c1c(F)cccc1F] | 435.3 |
| 337 | N-({(2S)-4-[(1R,2R)-2-(2′,6′-difluoro[1,1′- biphenyl]-2-yl)cyclopropane-1- carbonyl]morpholin-2-yl}methyl)-N′- methylsulfuric diamide | 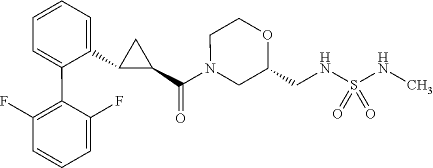 [SMILES: CNS(=O)(=O)NC[C@@H]1CN(C(=O)[C@@H]2C[C@H]2c2ccccc2-c2c(F)cccc2F)CCO1] | 466.3 |
| 338 | N-({(2S)-4-[(1R,2R)-2-(2′,6′-difluoro[1,1′- biphenyl]-2-yl)cyclopropane-1- carbonyl]morpholin-2- yl}methyl)azetidine-1-sulfonamide | 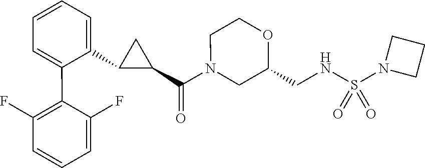 [SMILES: O=C([C@@H]1C[C@H]1c1ccccc1-c1c(F)cccc1F)N1CCO[C@H](CNS(=O)(=O)N2CCC2)C1] | 492.3 |
| 339 | N-({(2S)-1-[(1R,2R)-2-(2′,6′-difluoro[1,1′- biphenyl]-2-yl)cyclopropane-1- carbonyl]azetidin-2-yl}methyl)-N′- methylsulfuric diamide | 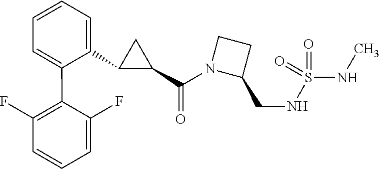 [SMILES: CNS(=O)(=O)NC[C@@H]1CCN1C(=O)[C@@H]1C[C@H]1c1ccccc1-c1c(F)cccc1F] | 436.3 |
| 340 | N-{(3aR,4R,6aS)-2-[(1R,2R)-2-(2′,6′- difluoro[1,1′-biphenyl]-2- yl)cyclopropane-1-carbonyl]-3a- fluorooctahydrocyclopenta[c]pyrrol-4- yl}methanesulfonamide, or N- {(3aS,4S,6aR)-2-[(1R,2R)-2-(2′,6′- difluoro[1,1′-biphenyl]-2- yl)cyclopropane-1-carbonyl]-3a- fluorooctahydrocyclopenta[c]pyrrol-4- yl}methanesulfonamide | 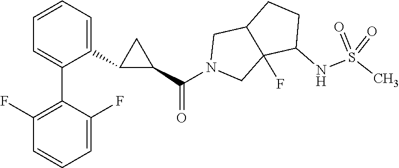 [SMILES: CS(=O)(=O)NC1CCC2CN(C(=O)[C@@H]3C[C@H]3c3ccccc3-c3c(F)cccc3F)CC21F] | 479.3 |
| 341 | N-{(3S,5R)-1-[(1S,2S)-2-(2′,6′- difluoro[1,1′-biphenyl]-2-yl)-2- fluorocyclopropane-1-carbonyl]-5- hydroxypiperidin-3- yl}methanesulfonamide | 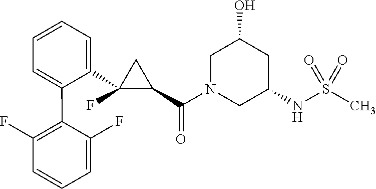 [SMILES: CS(=O)(=O)N[C@H]1C[C@@H](O)CN(C(=O)[C@@H]2C[C@@]2(F)c2ccccc2-c2c(F)cccc2F)C1] | 469.2 |
| 342 | N-[(3R)-1-[(1R,2R)-2-(2′,6′-difluoro[1,1′- biphenyl]-2-yl)cyclopropane-1- carbonyl]-3-(fluoromethyl)pyrrolidin-3- yl]methanesulfonamide, or N-[(3S)-1- [(1R,2R)-2-(2′,6′-difluoro[1,1′-biphenyl]- 2-yl)cyclopropane-1-carbonyl]-3- (fluoromethyl)pyrrolidin-3- yl]methanesulfonamide | 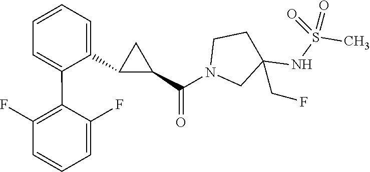 [SMILES: CS(=O)(=O)NC1(CF)CCN(C(=O)[C@@H]2C[C@H]2c2ccccc2-c2c(F)cccc2F)C1] | 453.2 |
| 343 | N-({(2S)-1-[(1R,2R)-2-(2′,6′-difluoro[1,1′- biphenyl]-2-yl)cyclopropane-1- carbonyl]-3,3-dimethylazetidin-2- yl}methyl)methanesulfonamide | 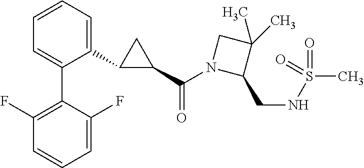 [SMILES: CC1(C)CN(C(=O)[C@@H]2C[C@H]2c2ccccc2-c2c(F)cccc2F)[C@@H]1CNS(C)(=O)=O] | 449.3 |
| 344 | N-{(3R)-1-[(1S,2S)-2-(2′,6′-difluoro[1,1′- biphenyl]-2-yl)-2-fluorocyclopropane- 1-carbonyl]-3-methylpyrrolidin-3- yl}methanesulfonamide, or N-{(3S)-1- [(1S,2S)-2-(2′,6′-difluoro[1,1′-biphenyl]- 2-yl)-2-fluorocyclopropane-1- carbonyl]-3-methylpyrrolidin-3- yl}methanesulfonamide | 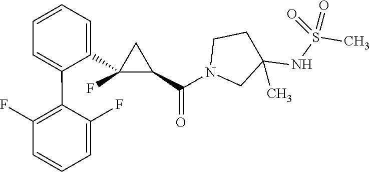 [SMILES: CC1(NS(C)(=O)=O)CCN(C(=O)[C@@H]2C[C@@]2(F)c2ccccc2-c2c(F)cccc2F)C1] | 453.3 |
| 345 | N-{2-[(1R,2R)-2-(2′,6′-difluoro[1,1′- biphenyl]-2-yl)cyclopropane-1- carbonyl]-2-azabicyclo[2.1.1]hexan-4- yl}methanesulfonamide | 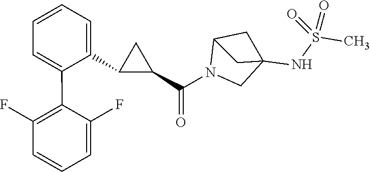 [SMILES: CS(=O)(=O)NC12CC(C1)N(C(=O)[C@@H]1C[C@H]1c1ccccc1-c1c(F)cccc1F)C2] | 433.1 |
| 346 | N-{(3S)-1-[(1R,2S)-2-(2′,6′-difluoro[1,1′- biphenyl]-2-yl)-1- (methoxymethyl)cyclopropane-1- carbonyl]pyrrolidin-3- yl}methanesulfonamide, or N-{(3S)-1- [(1S,2R)-2-(2′,6′-difluoro[1,1′-biphenyl]- 2-yl)-1-(methoxymethyl)cyclopropane- 1-carbonyl]pyrrolidin-3- yl}methanesulfonamide | 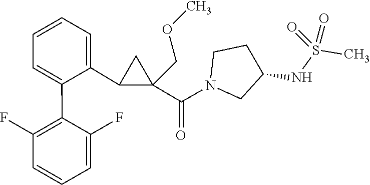 [SMILES: COCC1(C(=O)N2CC[C@H](NS(C)(=O)=O)C2)CC1c1ccccc1-c1c(F)cccc1F] | 465.0 |
| 347 | N-({(2S)-1-[(1R,2S)-2-(2′,6′-difluoro[1,1′- biphenyl]-2-yl)-1- (methoxymethyl)cyclopropane-1- carbonyl]azetidin-2- yl}methyl)methanesulfonamide, or N- ({(2S)-1-[(1S,2R)-2-(2′,6′-difluoro[1,1′- biphenyl]-2-yl)-1- (methoxymethyl)cyclopropane-1- carbonyl]azetidin-2- yl}methyl)methanesulfonamide | 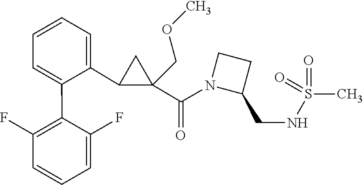 [SMILES: COCC1(C(=O)N2CC[C@H]2CNS(C)(=O)=O)CC1c1ccccc1-c1c(F)cccc1F] | 465.0 |
| 348 | N-{(3R,4R)-1-[(1S,2S)-2-(2′,6′- difluoro[1,1′-biphenyl]-2-yl)-2- fluorocyclopropane-1-carbonyl]-4- hydroxypiperidin-3- yl}methanesulfonamide | 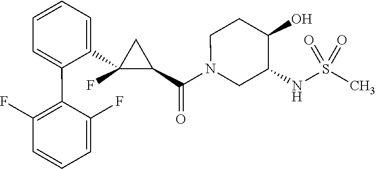 [SMILES: CS(=O)(=O)N[C@@H]1CN(C(=O)[C@@H]2C[C@@]2(F)c2ccccc2-c2c(F)cccc2F)CC[C@H]1O] | 469.0 |
| 349 | N-{(3S,5S)-1-[(1R,2R)-2-(2′,6′- difluoro[1,1′-biphenyl]-2- yl)cyclopropane-1-carbonyl]-5- methoxypiperidin-3- yl}methanesulfonamide | 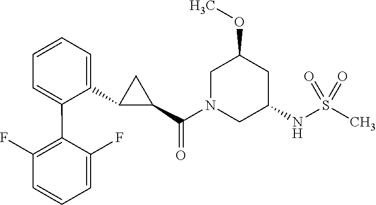 [SMILES: CO[C@H]1C[C@H](NS(C)(=O)=O)CN(C(=O)[C@@H]2C[C@H]2c2ccccc2-c2c(F)cccc2F)C1] | 465.1 |
| 350 | N-{(3R,4R)-1-[(1R,2R)-2-(2′,6′- difluoro[1,1′-biphenyl]-2- yl)cyclopropane-1-carbonyl]-4- methoxypiperidin-3- yl}methanesulfonamide | 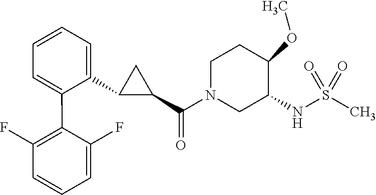 [SMILES: CO[C@@H]1CCN(C(=O)[C@@H]2C[C@H]2c2ccccc2-c2c(F)cccc2F)C[C@H]1NS(C)(=O)=O] | 465.0 |
| 351 | N-({(2R)-1-[(1S,2S)-2-(2′,6′-difluoro[1,1′- biphenyl]-2-yl)-2-fluorocyclopropane- 1-carbonyl]azetidin-2- yl}methyl)ethanesulfonamide, or N- ({(2S)-1-[(1S,2S)-2-(2′,6′-difluoro[1,1′- biphenyl]-2-yl)-2-fluorocyclopropane- 1-carbonyl]azetidin-2- yl}methyl)ethanesulfonamide | 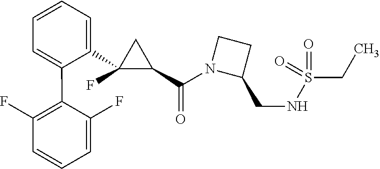 [SMILES: CCS(=O)(=O)NC[C@@H]1CCN1C(=O)[C@@H]1C[C@@]1(F)c1ccccc1-c1c(F)cccc1F] | 453.0 |
| 352 | N-({(2S)-1-[(1S,2S)-2-(2′,6′-difluoro[1,1′- biphenyl]-2-yl)-2-fluorocyclopropane- 1-carbonyl]azetidin-2- yl}methyl)methanesulfonamide | 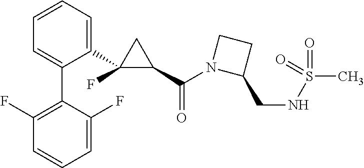 [SMILES: CS(=O)(=O)NC[C@@H]1CCN1C(=O)[C@@H]1C[C@@]1(F)c1ccccc1-c1c(F)cccc1F] | 439.2 |
| 353 | N-[(1S)-1-{(2S)-1-[(1S,2S)-2-(2′,6′- difluoro[1,1′-biphenyl]-2-yl)-2- fluorocyclopropane-1- carbonyl]azetidin-2- yl}ethyl]methanesulfonamide | 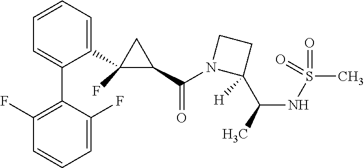 [SMILES: C[C@H](NS(C)(=O)=O)[C@@H]1CCN1C(=O)[C@@H]1C[C@@]1(F)c1ccccc1-c1c(F)cccc1F] | 453.0 |
| 355 | N-({(2S)-1-[(1R,2R)-2-(2′,6′-difluoro[1,1′- biphenyl]-2-yl)cyclopropane-1- carbonyl]azetidin-2- yl}methyl)methanesufonamide | 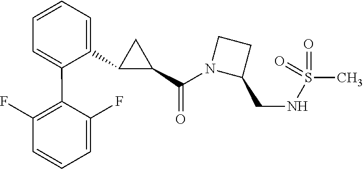 [SMILES: CS(=O)(=O)NC[C@@H]1CCN1C(=O)[C@@H]1C[C@H]1c1ccccc1-c1c(F)cccc1F] | 421.3 |
| 356 | N-({(2S)-1-[(1R,2S)-2-(2′,6′-difluoro[1,1′- biphenyl]-2-yl)-2-fluoro-1- methylcyclopropane-1- carbonyl]azetidin-2- yl}methyl)methanesulfonamide, or N- ({(2S)-1-[(1S,2R)-2-(2′,6′-difluoro[1,1′- biphenyl]-2-yl)-2-fluoro-1- methylcyclopropane-1- carbonyl]azetidin-2- yl}methyl)methanesulfonamide | 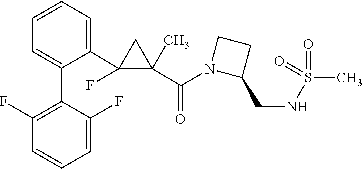 [SMILES: CC1(C(=O)N2CC[C@H]2CNS(C)(=O)=O)CC1(F)c1ccccc1-c1c(F)cccc1F] | 453.2 |
| 357 | N-({(2R,3R)-1-[(1S,2S)-2-(2′,6′- difluoro[1,1′-biphenyl]-2-yl)-2- fluorocyclopropane-1-carbonyl]-3- methylazetidin-2- yl}methyl)methanesulfonamide | 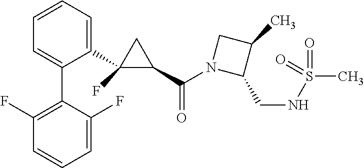 [SMILES: C[C@@H]1CN(C(=O)[C@@H]2C[C@@]2(F)c2ccccc2-c2c(F)cccc2F)[C@H]1CNS(C)(=O)=O] | 453.2 |
| 358 | N-({(2S,3R)-1-[(1S,2S)-2-(2′,6′- difluoro[1,1′-biphenyl]-2-yl)-2- fluorocyclopropane-1-carbonyl]-3- methylazetidin-2- yl}methyl)methanesulfonamide | 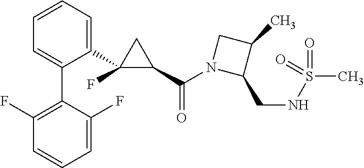 [SMILES: C[C@@H]1CN(C(=O)[C@@H]2C[C@@]2(F)c2ccccc2-c2c(F)cccc2F)[C@@H]1CNS(C)(=O)=O] | 453.1 |
| 359 | N-({2-[(1S,2S)-2-(2′,6′-difluoro[1,1′- biphenyl]-2-yl)-2-fluorocyclopropane- 1-carbonyl]-2-azabicyclo[2.1.1]hexan- 4-yl}methyl)methanesulfonamide | 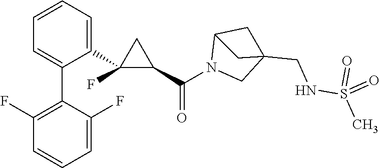 [SMILES: CS(=O)(=O)NCC12CC(C1)N(C(=O)[C@@H]1C[C@@]1(F)c1ccccc1-c1c(F)cccc1F)C2] | 465.0 |
| 360 | N-({(2S,3R)-1-[(1S,2S)-2-(2′,6′- difluoro[1,1′-biphenyl]-2-yl)-2- fluorocyclopropane-1-carbonyl]-3- methylazetidin-2- yl}methyl)ethanesulfonamide | 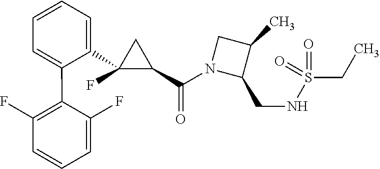 [SMILES: CCS(=O)(=O)NC[C@@H]1[C@H](C)CN1C(=O)[C@@H]1C[C@@]1(F)c1ccccc1-c1c(F)cccc1F] | 467.2 |
| 361 | N-[(1R)-1-{(2R)-1-[(1R,2R)-2-(2′,6′- difluoro[1,1′-biphenyl]-2- yl)cyclopropane-1-carbonyl]azetidin-2- yl}ethyl]ethanesulfonamide, or N-[(1S)- 1-{(2R)-1-[(1R,2R)-2-(2′,6′-difluoro[1,1′- biphenyl]-2-yl)cyclopropane-1- carbonyl]azetidin-2- yl}ethyl]ethanesulfonamide | 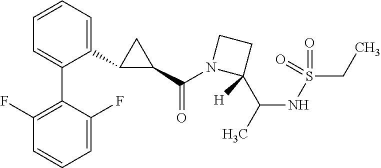 [SMILES: CCS(=O)(=O)NC(C)[C@H]1CCN1C(=O)[C@@H]1C[C@H]1c1ccccc1-c1c(F)cccc1F] | 449.2 |
| 362 | N-({(2S)-1-[(1R,2R)-2-(2′,6′-difluoro[1,1′- biphenyl]-2-yl)cyclopropane-1- carbonyl]azetidin-2- yl}methyl)ethanesulfonamide | 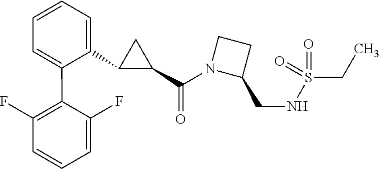 [SMILES: CCS(=O)(=O)NC[C@@H]1CCN1C(=O)[C@@H]1C[C@H]1c1ccccc1-c1c(F)cccc1F] | 435.2 |
| 363 | N-[(1R)-1-{(2S)-1-[(1S,2S)-2-(2′,6′- difluoro[1,1′-biphenyl]-2-yl)-2- fluorocyclopropane-1- carbonyl]azetidin-2- yl}ethyl]ethanesulfonamide, or N-[(1S)- 1-{(2S)-1-[(1S,2S)-2-(2′,6′-difluoro[1,1′- biphenyl]-2-yl)-2-fluorocyclopropane- 1-carbonyl]azetidin-2- yl}ethyl]ethanesulfonamide | 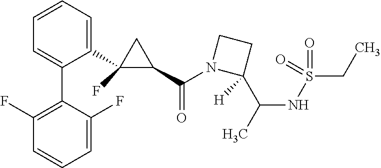 [SMILES: CCS(=O)(=O)NC(C)[C@@H]1CCN1C(=O)[C@@H]1C[C@@]1(F)c1ccccc1-c1c(F)cccc1F] | 467.2 |
| 364 | N-({(2S,6S)-4-[(1S,2S)-2-(2′,6′- difluoro[1,1′-biphenyl]-2-yl)-2- fluorocyclopropane-1-carbonyl]-6- methylmorpholin-2- yl}methyl)methanesulfonamide | 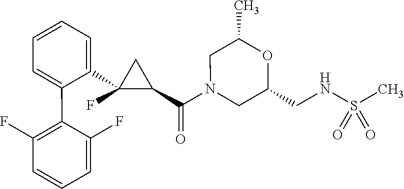 [SMILES: C[C@H]1CN(C(=O)[C@@H]2C[C@@]2(F)c2ccccc2-c2c(F)cccc2F)C[C@@H](CNS(C)(=O)=O)O1] | 483.2 |
| 365 | N-({(2S)-1-[(1R,2S)-2-(2′,6′-difluoro[1,1′- biphenyl]-2-yl)-1- (hydroxymethyl)cyclopropane-1- carbonyl]azetidin-2- yl}methyl)methanesulfonamide, or N- ({(2S)-1-[(1S,2R)-2-(2′,6′-difluoro[1,1′- biphenyl]-2-yl)-1- (hydroxymethyl)cyclopropane-1- carbonyl]azetidin-2- yl}methyl)methanesulfonamide | 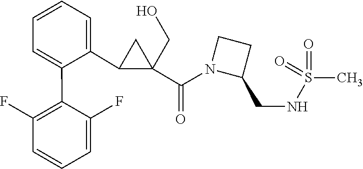 [SMILES: CS(=O)(=O)NC[C@@H]1CCN1C(=O)C1(CO)CC1c1ccccc1-c1c(F)cccc1F] | 451.0 |
| 366 | N-({(2S)-1-[(1R,2R)-2-(2′-chloro[1,1′- biphenyl]-2-yl)cyclopropane-1- carbonyl]azetidin-2- yl}methyl)methanesulfonamide | 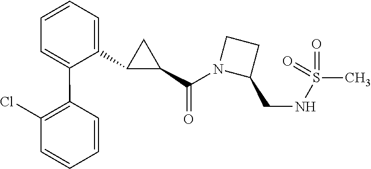 [SMILES: CS(=O)(=O)NC[C@@H]1CCN1C(=O)[C@@H]1C[C@H]1c1ccccc1-c1ccccc1Cl] | 419.1 |
| 367 | N-{[(2S)-1-{(1R,2R)-2-[4-(2,6- difluorophenyl)-2-fluorothiophen-3- yl]cyclopropane-1-carbonyl}azetidin-2- yl]methyl}methanesulfonamide, or N- {[(2S)-1-{(1S,2S)-2-[4-(2,6- difluorophenyl)-2-fluorothiophen-3- yl]cyclopropane-1-carbonyl}azetidin-2- yl]methyl}methanesulfonamide | 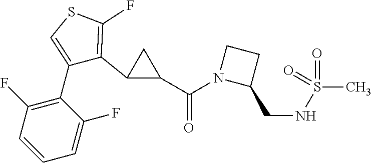 [SMILES: CS(=O)(=O)NC[C@@H]1CCN1C(=O)C1CC1c1c(-c2c(F)cccc2F)csc1F] | 445.0 |
| 368 | N-[(3S)-1-{(1R,2R)-2-[4-(2,6- difluorophenyl)-2-fluorothiophen-3- yl]cyclopropane-1-carbonyl}pyrrolidin- 3-yl]methanesulfonamide, or N-[(3S)-1- {(1S,2S)-2-[4-(2,6-difluorophenyl)-2- fluorothiophen-3-yl]cyclopropane-1- carbonyl}pyrrolidin-3- yl]methanesulfonamide | 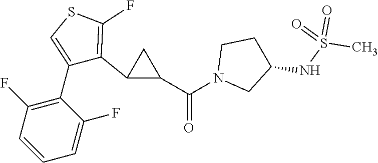 [SMILES: CS(=O)(=O)N[C@H]1CCN(C(=O)C2CC2c2c(-c3c(F)cccc3F)csc2F)C1] | 445.0 |
| 370 | N-({(2R)-1-[(1R,2R)-2-(2′,6′-difluoro[1,1′- biphenyl]-2-yl)cyclopropane-1- carbonyl]azetidin-2- yl}methyl)methanesulfonamide | 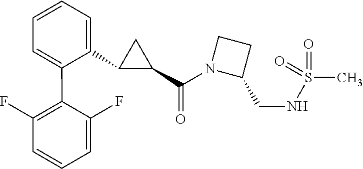 [SMILES: CS(=O)(=O)NC[C@H]1CCN1C(=O)[C@@H]1C[C@H]1c1ccccc1-c1c(F)cccc1F] | 421.1 |
| 371 | N-({(2R)-1-[(1S,2S)-2-(2′,6′-difluoro[1,1′- biphenyl]-2-yl)-2-fluorocyclopropane- 1-carbonyl]azetidin-2- yl}methyl)methanesulfonamide | 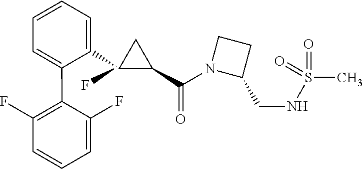 | 439.1 |
| 372 | N-({(2S,6R)-4-[(1S,2S)-2-(2′,6′- difluoro[1,1′-biphenyl]-2-yl)-2- fluorocyclopropane-1-carbonyl]-6- methylmorpholin-2- yl}methyl)methanesulfonamide | 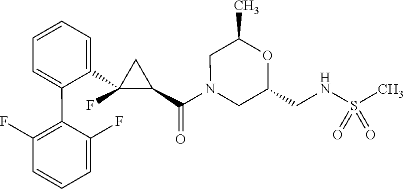 [SMILES: C[C@@H]1CN(C(=O)[C@@H]2C[C@@]2(F)c2ccccc2-c2c(F)cccc2F)C[C@@H](CNS(C)(=O)=O)O1] | 483.0 |
| 373 | N-[(3R)-1-[(1S,2S)-2-(2′,6′-difluoro[1,1′- biphenyl]-2-yl)-2-fluorocyclopropane- 1-carbonyl]-3- (methoxymethyl)pyrrolidin-3- yl]methanesulfonamide, or N-[(3S)-1- [(1S,2S)-2-(2′,6′-difluoro[1,1′-biphenyl]- 2-yl)-2-fluorocyclopropane-1- carbonyl]-3-(methoxymethyl)pyrrolidin- 3-yl]methanesulfonamide | 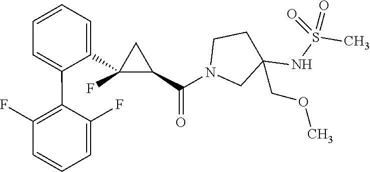 [SMILES: COCC1(NS(C)(=O)=O)CCN(C(=O)[C@@H]2C[C@@]2(F)c2ccccc2-c2c(F)cccc2F)C1] | 483.0 |
| 374 | N-({(2R,3R)-1-[(1S,2S)-2-(2′,6′- difluoro[1,1′-biphenyl]-2-yl)-2- fluorocyclopropane-1-carbonyl]-3- hydroxyazetidin-2- yl}methyl)ethanesulfonamide | 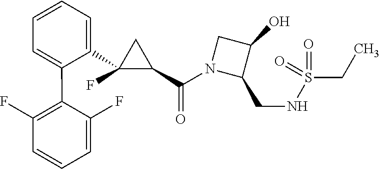 [SMILES: CCS(=O)(=O)NC[C@@H]1[C@H](O)CN1C(=O)[C@@H]1C[C@@]1(F)c1ccccc1-c1c(F)cccc1F] | 468.9 |
| 375 | N-{(3R)-1-[(1S,2S)-2-(2′,6′-difluoro[1,1′- biphenyl]-2-yl)-2-fluorocyclopropane- 1-carbonyl]-4,4-difluoro-3- methylpyrrolidin-3- yl}methanesulfonamide, or N-{(3S)-1- [(1S,2S)-2-(2′,6′-difluoro[1,1′-biphenyl]- 2-yl)-2-fluorocyclopropane-1- carbonyl]-4,4-difluoro-3- methylpyrrolidin-3- yl}methanesulfonamide | 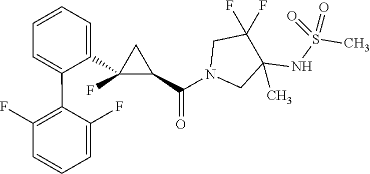 [SMILES: CC1(NS(C)(=O)=O)CN(C(=O)[C@@H]2C[C@@]2(F)c2ccccc2-c2c(F)cccc2F)CC1(F)F] | 489.2 |
| 376 | N-({(2R)-1-[(1R,2R)-2-(2′,6′-difluoro[1,1′- biphenyl]-2-yl)cyclopropane-1- carbonyl]-3,3-dimethylazetidin-2- yl}methyl)methanesulfonamide | 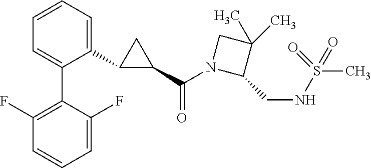 [SMILES: CC1(C)CN(C(=O)[C@@H]2C[C@H]2c2ccccc2-c2c(F)cccc2F)[C@H]1CNS(C)(=O)=O] | 449.3 |
| 377 | N-{(3aR,4S,6aS)-2-[(1S,2S)-2-(2′,6′- difluoro[1,1′-biphenyl]-2-yl)-2- fluorocyclopropane-1-carbonyl]-5,5- difluorooctahydrocyclopenta[c]pyrrol- 4-yl}methanesulfonamide, or N- {(3aS,4R,6aR)-2-[(1S,2S)-2-(2′,6′- difluoro[1,1′-biphenyl]-2-yl)-2- fluorocyclopropane-1-carbonyl]-5,5- difluorooctahydrocyclopenta[c]pyrrol- 4-yl}methanesulfonamide | 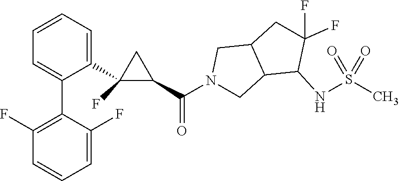 [SMILES: CS(=O)(=O)NC1C2CN(C(=O)[C@@H]3C[C@@]3(F)c3ccccc3-c3c(F)cccc3F)CC2CC1(F)F] | 515.2 |
| 378 | N-[(3R)-1-{(1R,2R)-2-[4-(2,6- difluorophenyl)-2-fluorothiophen-3- yl]cyclopropane-1-carbonyl}-4,4- difluoropiperidin-3- yl]methanesulfonamide, or N-[(3R)-1- {(1S,2S)-2-[4-(2,6-difluorophenyl)-2- fluorothiophen-3-yl]cyclopropane-1- carbonyl}-4,4-difluoropiperidin-3- yl]methanesulfonamide | 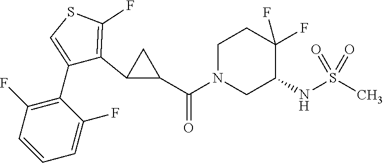 [SMILES: CS(=O)(=O)N[C@@H]1CN(C(=O)C2CC2c2c(-c3c(F)cccc3F)csc2F)CCC1(F)F] | 495.1 |
| 379 | N-[(1R)-1-{(2S)-1-[(1S,2S)-2-(2′,6′- difluoro[1,1′-biphenyl]-2-yl)-2- fluorocyclopropane-1- carbonyl]azetidin-2- yl}propyl]methanesulfonamide, or N- [(1S)-1-{(2S)-1-[(1S,2S)-2-(2′,6′- difluoro[1,1′-biphenyl]-2-yl)-2- fluorocyclopropane-1- carbonyl]azetidin-2- yl}propyl]methanesulfonamide | 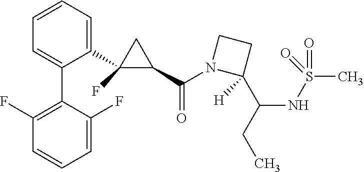 [SMILES: CCC(NS(C)(=O)=O)[C@@H]1CCN1C(=O)[C@@H]1C[C@@]1(F)c1ccccc1-c1c(F)cccc1F] | 466.9 |
| 380 | N-({(2S,3R)-1-[(1S,2S)-2-(2′,6′- difluoro[1,1′-biphenyl]-2-yl)-2- fluorocyclopropane-1-carbonyl]-3- ethylazetidin-2- yl}methyl)methanesulfonamide | 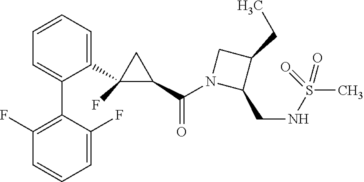 [SMILES: CC[C@@H]1CN(C(=O)[C@@H]2C[C@@]2(F)c2ccccc2-c2c(F)cccc2F)[C@@H]1CNS(C)(=O)=O] | 467.0 |
| 381 | N-({(2R,3R)-1-[(1S,2S)-2-(2′,6′- difluoro[1,1′-biphenyl]-2-yl)-2- fluorocyclopropane-1-carbonyl]-3- ethylazetidin-2- yl}methyl)methylsulfonamide | 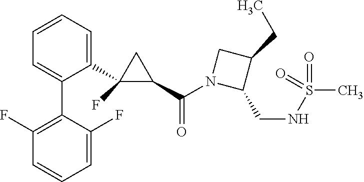 [SMILES: CC[C@@H]1CN(C(=O)[C@@H]2C[C@@]2(F)c2ccccc2-c2c(F)cccc2F)[C@H]1CNS(C)(=O)=O] | 467.0 |
| 382 | N-({(2R,3S)-1-[(1S,2S)-2-(2′,6′- difluoro[1,1′-biphenyl]-2-yl)-2- fluorocyclopropane-1-carbonyl]-3- hydroxyazetidin-2- yl}methyl)ethanesulfonamide | 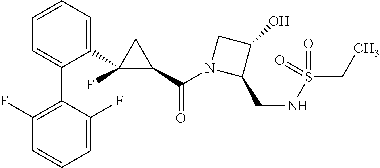 [SMILES: CCS(=O)(=O)NC[C@@H]1[C@@H](O)CN1C(=O)[C@@H]1C[C@@]1(F)c1ccccc1-c1c(F)cccc1F] | 468.9 |
| 383 | N-({(2R,3R)-1-[(1S,2S)-2-(2′,6′- difluoro[1,1′-biphenyl]-2-yl)-2- fluorocyclopropane-1-carbonyl]-3- methoxyazetidin-2- yl}methyl)methanesulfonamide | 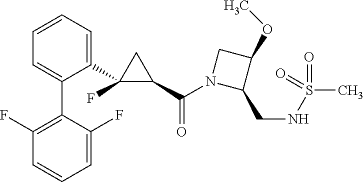 [SMILES: CO[C@@H]1CN(C(=O)[C@@H]2C[C@@]2(F)c2ccccc2-c2c(F)cccc2F)[C@@H]1CNS(C)(=O)=O] | 469.2 |
| 384 | N-{[(2S,3R)-1-[(1S,2S)-2-(2′,6′- difluoro[1,1′-biphenyl]-2-yl)-2- fluorocyclopropane-1-carbonyl]-3- (hydroxymethyl)azetidin-2- yl]methyl}methanesulfonamide | 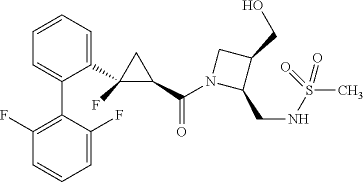 [SMILES: CS(=O)(=O)NC[C@@H]1[C@H](CO)CN1C(=O)[C@@H]1C[C@@]1(F)c1ccccc1-c1c(F)cccc1F] | 469.0 |
| 385 | N-[(1R)-1-{(2R)-1-[(1S,2S)-2-(2′,6′- difluoro[1,1′-biphenyl]-2-yl)-2- fluorocyclopropane-1- carbonyl]azetidin-2-yl}-2- hydroxyethyl]methanesulfonamide, or N-[(1R)-1-{(2S)-1-[(1S,2S)-2-(2′,6′- difluoro[1,1′-biphenyl]-2-yl)-2- fluorocyclopropane-1- carbonyl]azetidin-2-yl}-2- hydroxyethyl]methanesulfonamide | 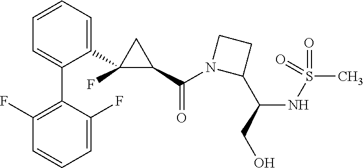 [SMILES: CS(=O)(=O)N[C@@H](CO)C1CCN1C(=O)[C@@H]1C[C@@]1(F)c1ccccc1-c1c(F)cccc1F] | 469.0 |
| 386 | N-({(2S,3R)-1-[(1S,2S)-2-fluoro-2-(2′,5,6′- trifluoro[1,1′-biphenyl]-2- yl)cyclopropane-1-carbonyl]-3- methylazetidin-2- yl}methyl)methanesulfonamide | 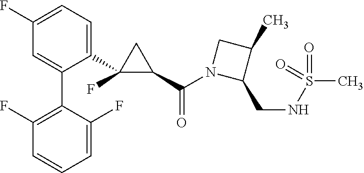 [SMILES: C[C@@H]1CN(C(=O)[C@@H]2C[C@@]2(F)c2ccc(F)cc2-c2c(F)cccc2F)[C@@H]1CNS(C)(=O)=O] | 471.1 |
| 387 | N-[(1S)-1-{(2S)-1-[(1S,2S)-2-(2′,6′- difluoro[1,1′-biphenyl]-2-yl)-2- fluorocyclopropane-1- carbonyl]azetidin-2- yl}ethyl]cyclopropanesulfonamide | 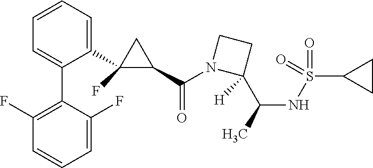 [SMILES: C[C@H](NS(=O)(=O)C1CC1)[C@@H]1CCN1C(=O)[C@@H]1C[C@@]1(F)c1ccccc1-c1c(F)cccc1F] | 479.1 |
| 388 | N-[(1S)-1-{(2S)-1-[(1S,2S)-2-(2′,6′- difluoro[1,1′-biphenyl]-2-yl)-2- fluorocyclopropane-1- carbonyl]azetidin-2-yl}ethyl]-1- methylcyclopropane-1-sulfonamide | 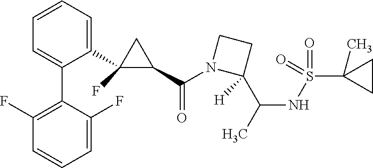 [SMILES: CC(NS(=O)(=O)C1(C)CC1)[C@@H]1CCN1C(=O)[C@@H]1C[C@@]1(F)c1ccccc1-c1c(F)cccc1F] | 493.0 |
| 389 | N-[(1S)-1-{(2S)-1-[(1S,2S)-2-(2′,6′- difluoro[1,1′-biphenyl]-2-yl)-2- fluorocyclopropane-1- carbonyl]azetidin-2-yl}ethyl]propane-2- sulfonamide | 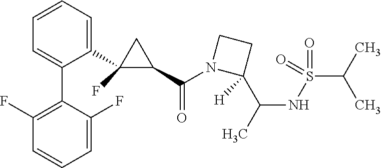 [SMILES: CC(NS(=O)(=O)C(C)C)[C@@H]1CCN1C(=O)[C@@H]1C[C@@]1(F)c1ccccc1-c1c(F)cccc1F] | 481.0 |
| 390 | N-({(2R,3R)-1-[(1S,2S)-2-(2′,6′- difluoro[1,1′-biphenyl]-2-yl)-2- fluorocyclopropane-1-carbonyl]-3- fluoroazetidin-2- yl}methyl)methanesulfonamide | 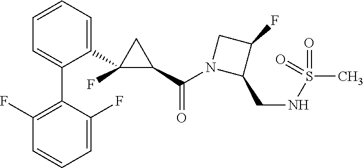 [SMILES: CS(=O)(=O)NC[C@@H]1[C@H](F)CN1C(=O)[C@@H]1C[C@@]1(F)c1ccccc1-c1c(F)cccc1F] | 457.0 |
| 391 | N-{[(2R,3R)-1-[(1S,2S)-2-(2′,6′- difluoro[1,1′-biphenyl]-2-yl)-2- fluorocyclopropane-1-carbonyl]-3- (hydroxymethyl)azetidin-2- yl]methyl}methanesulfonamide | 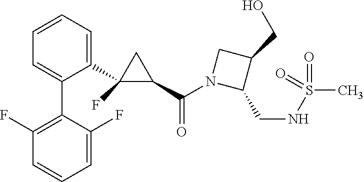 [SMILES: CS(=O)(=O)NC[C@H]1[C@H](CO)CN1C(=O)[C@@H]1C[C@@]1(F)c1ccccc1-c1c(F)cccc1F] | 469.0 |
| 392 | N-[(1R)-1-{2-[(1S,2S)-2-(2′,6′- difluoro[1,1′-biphenyl]-2-yl)-2- fluorocyclopropane-1-carbonyl]-2- azabicyclo[2.1.1]hexan-4- yl}ethyl]methanesulfonamide, or N- [(1S)-1-{2-[(1S,2S)-2-(2′,6′-difluoro[1,1′- biphenyl]-2-yl)-2-fluorocyclopropane- 1-carbonyl]-2-azabicyclo[2.1.1]hexan- 4-yl}ethyl]methanesulfonamide | 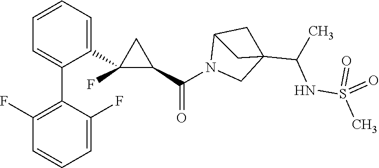 [SMILES: CC(NS(C)(=O)=O)C12CC(C1)N(C(=O)[C@@H]1C[C@@]1(F)c1ccccc1-c1c(F)cccc1F)C2] | 479.1 |
| 393 | N-({(2R,3R)-1-[(1S,2S)-2-(2′,6′- difluoro[1,1′-biphenyl]-2-yl)-2- fluorocyclopropane-1-carbonyl]-3- fluoroazetidin-2- yl}methyl)ethanesulfonamide | 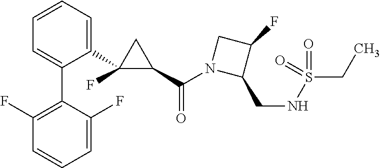 [SMILES: CCS(=O)(=O)NC[C@@H]1[C@H](F)CN1C(=O)[C@@H]1C[C@@]1(F)c1ccccc1-c1c(F)cccc1F] | 471.1 |
| 394 | N-[(R)-cyclopropyl{(2S)-1-[(1S,2S)-2- (2′,6′-difluoro[1,1′-biphenyl]-2-yl)-2- fluorocyclopropane-1- carbonyl]azetidin-2- yl}methyl]methanesulfonamide, or N- [(S)-cyclopropyl{(2S)-1-[(1S,2S)-2-(2′,6′- difluoro[1,1′-biphenyl]-2-yl)-2- fluorocyclopropane-1- carbonyl]azetidin-2- yl}methyl]methanesulfonamide | 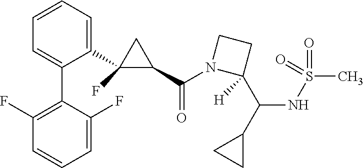 [SMILES: CS(=O)(=O)NC(C1CC1)[C@@H]1CCN1C(=O)[C@@H]1C[C@@]1(F)c1ccccc1-c1c(F)cccc1F] | 479.1 |
| 395 | N-({(2S,3R)-1-[(1R,2R)-2-fluoro-2- (2′,3,6′-trifluoro[1,1′-biphenyl]-2- yl)cyclopropane-1-carbonyl]-3- methylazetidin-2- yl}methyl)methanesulfonamide, or N- ({(2S,3R)-1-[(1S,2S)-2-fluoro-2-(2′,3,6′- trifluoro[1,1′-biphenyl]-2- yl)cyclopropane-1-carbonyl]-3- methylazetidin-2- yl}methyl)methanesulfonamide | 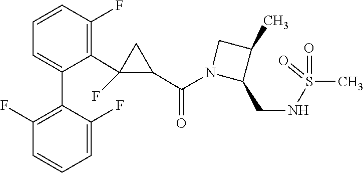 [SMILES: C[C@@H]1CN(C(=O)C2CC2(F)c2c(F)cccc2-c2c(F)cccc2F)[C@@H]1CNS(C)(=O)=O] | 471.2 |
| 396 | N-({(2S,3R)-1-[(1R,2R)-2-(2′,6′-difluoro- 5-methyl[1,1′-biphenyl]-2-yl)-2- fluorocyclopropane-1-carbonyl]-3- methylazetidin-2- yl}methyl)methanesulfonamide, or N- ({(2S,3R)-1-[(1S,2S)-2-(2′,6′-difluoro-5- methyl[1,1′-biphenyl]-2-yl)-2- fluorocyclopropane-1-carbonyl]-3- methylazetidin-2- yl}methyl)methanesulfonamide | 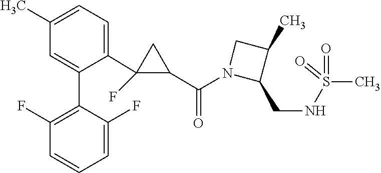 | 467.1 |
| 397 | N-{[(2S,3R)-1-[(1S,2S)-2-(2′,6′- difluoro[1,1′-biphenyl]-2-yl)-2- fluorocyclopropane-1-carbonyl]-3- (methoxymethyl)azetidin-2- yl]methyl}methanesulfonamide | 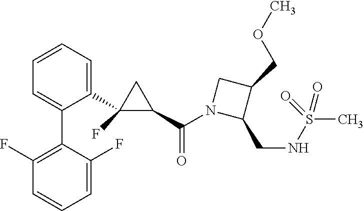 [SMILES: COC[C@@H]1CN(C(=O)[C@@H]2C[C@@]2(F)c2ccccc2-c2c(F)cccc2F)[C@@H]1CNS(C)(=O)=O] | 483.2 |
| 398 | N-{[(2S,3R)-1-[(1S,2S)-2-(2′,6′- difluoro[1,1′-biphenyl]-2-yl)-2- fluorocyclopropane-1-carbonyl]-3- (fluoromethyl)azetidin-2- yl]methyl}methanesulfonamide | 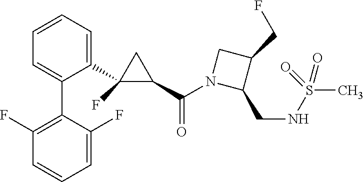 [SMILES: CS(=O)(=O)NC[C@@H]1[C@H](CF)CN1C(=O)[C@@H]1C[C@@]1(F)c1ccccc1-c1c(F)cccc1F] | 471.2 |
| 399 | N-{[(2R,3R)-1-[(1S,2S)-2-(2′,6′- difluoro[1,1′-biphenyl]-2-yl)-2- fluorocyclopropane-1-carbonyl]-3- (methoxymethyl)azetidin-2- yl]methyl}methanesulfonamide | 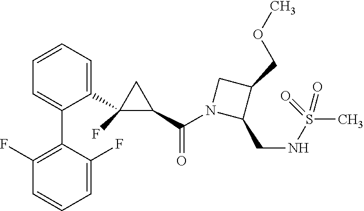 [SMILES: COC[C@@H]1CN(C(=O)[C@@H]2C[C@@]2(F)c2ccccc2-c2c(F)cccc2F)[C@@H]1CNS(C)(=O)=O] | 483.1 |
| 400 | N-{[(2R,3R)-1-[(1S,2S)-2-(2′,6′- difluoro[1,1′-biphenyl]-2-yl)-2- fluorocyclopropane-1-carbonyl]-3- (fluoromethyl)azetidin-2- yl]methyl}methanesulfonamide | 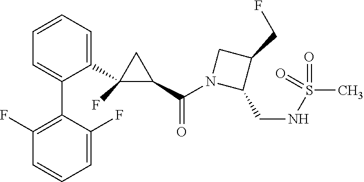 [SMILES: CS(=O)(=O)NC[C@H]1[C@H](CF)CN1C(=O)[C@@H]1C[C@@]1(F)c1ccccc1-c1c(F)cccc1F] | 471.2 |
| 401 | N-[(1R)-1-{(2S,3R)-1-[(1S,2S)-2-(2′,6′- difluoro[1,1′-biphenyl]-2-yl)-2- fluorocyclopropane-1-carbonyl]-3- methylazetidin-2- yl}ethyl]methanesulfonamide, or N- [(1S)-1-{(2S,3R)-1-[(1S,2S)-2-(2′,6′- difluoro[1,1′-biphenyl]-2-yl)-2- fluorocyclopropane-1-carbonyl]-3- methylazetidin-2- yl}ethyl]methanesulfonamide | 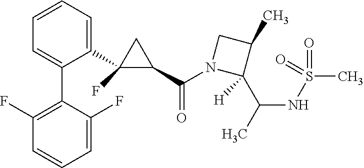 [SMILES: CC(NS(C)(=O)=O)[C@@H]1[C@H](C)CN1C(=O)[C@@H]1C[C@@]1(F)c1ccccc1-c1c(F)cccc1F] | 467.1 |
| 402 | N-[(1R)-1-{(2S)-1-[(1S,2S)-2-(2′,6′- difluoro[1,1′-biphenyl]-2-yl)-2- fluorocyclopropane-1- carbonyl]azetidin-2-yl}-2- methylpropyl]methanesulfonamide, or N-[(1R)-1-{(2S)-1-[(1S,2S)-2-(2′,6′- difluoro[1,1′-biphenyl]-2-yl)-2- fluorocyclopropane-1- carbonyl]azetidin-2-yl}-2- methylpropyl]methanesulfonamide | 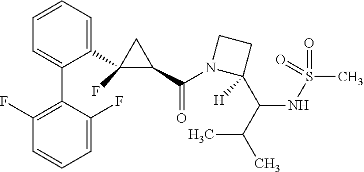 [SMILES: CC(C)C(NS(C)(=O)=O)[C@@H]1CCN1C(=O)[C@@H]1C[C@@]1(F)c1ccccc1-c1c(F)cccc1F] | 481.1 |
| 403 | N-({(2S)-1-[(1R,2R)-2-(2′,4′,6′- trifluoro[1,1′-biphenyl]-2- yl)cyclopropane-1-carbonyl]azetidin-2- yl}methyl)methanesulfonamide | 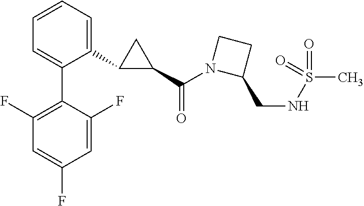 [SMILES: CS(=O)(=O)NC[C@@H]1CCN1C(=O)[C@@H]1C[C@H]1c1ccccc1-c1c(F)cc(F)cc1F] | 439.0 |
| 404 | a mixture of N-{[(2R,3R)-1-[(1S,2S)-2- (2′,6′-difluoro[1,1′-biphenyl]-2-yl)-2- fluorocyclopropane-1-carbonyl]-3- [(1R)-1-hydroxyethyl]azetidin-2- yl]methyl}methanesulfonamide and N- {[(2R,3R)-1-[(1S,2S)-2-(2′,6′- difluoro[1,1′-biphenyl]-2-yl)-2- fluorocyclopropane-1-carbonyl]-3- [(1S)-1-hydroxyethyl]azetidin-2- yl]methyl}methanesulfonamide | 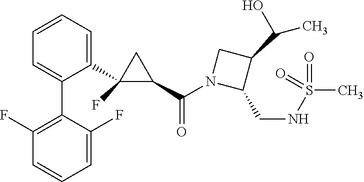 [SMILES: CC(O)[C@@H]1CN(C(=O)[C@@H]2C[C@@]2(F)c2ccccc2-c2c(F)cccc2F)[C@H]1CNS(C)(=O)=O] | 483.0 |
| 405 | N-{[(2R,3R)-1-[(1S,2S)-2-(2′,6′- difluoro[1,1′-biphenyl]-2-yl)-2- fluorocyclopropane-1-carbonyl]-3- (difluoromethyl)azetidin-2- yl]methyl}methanesulfonamide | 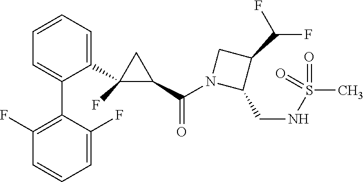 [SMILES: CS(=O)(=O)NC[C@H]1[C@H](C(F)F)CN1C(=O)[C@@H]1C[C@@]1(F)c1ccccc1-c1c(F)cccc1F] | 489.0 |
| 406 | N-({(2R,3R)-3-acetyl-1-[(1S,2S)-2-(2′,6′- difluoro[1,1′-biphenyl]-2-yl)-2- fluorocyclopropane-1- carbonyl]azetidin-2- yl}methyl)methanesulfonamide | 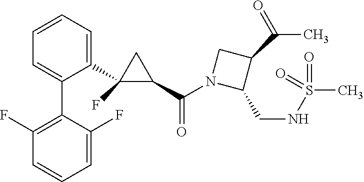 [SMILES: CC(=O)[C@@H]1CN(C(=O)[C@@H]2C[C@@]2(F)c2ccccc2-c2c(F)cccc2F)[C@H]1CNS(C)(=O)=O] | 481.0 |
| 407 | N-{[(2R,3R)-1-[(1S,2S)-2-(2′,6′- difluoro[1,1′-biphenyl]-2-yl)-2- fluorocyclopropane-1-carbonyl]-3-(2- hydroxypropan-2-yl)azetidin-2- yl]methyl}methanesulfonamide | 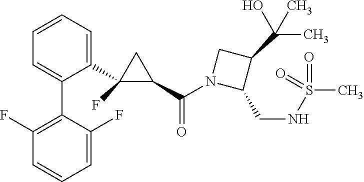 [SMILES: CC(C)(O)[C@@H]1CN(C(=O)[C@@H]2C[C@@]2(F)c2ccccc2-c2c(F)cccc2F)[C@H]1CNS(C)(=O)=O] | 497.0 |
| 408 | N-({(2S)-1-[(1R,2R)-2-(2′,3′,6′- trifluoro[1,1′-biphenyl]-2- yl)cyclopropane-1-carbonyl]azetidin-2- yl}methyl)methanesulfonamide | 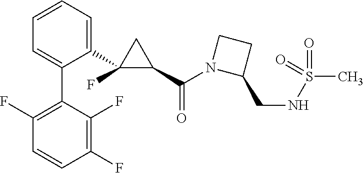 [SMILES: CS(=O)(=O)NC[C@@H]1CCN1C(=O)[C@@H]1C[C@@]1(F)c1ccccc1-c1c(F)ccc(F)c1F] | 439.0 |
| 409 | N-({(2R)-1-[(1S,2S)-2-(2′,6′-difluoro[1,1′- biphenyl]-2-yl)-2-fluorocyclopropane- 1-carbonyl]-3,3-difluoroazetidin-2- yl}methyl)methanesulfonamide, or N- ({(2S)-1-[(1S,2S)-2-(2′,6′-difluoro[1,1′- biphenyl]-2-yl)-2-fluorocyclopropane- 1-carbonyl]-3,3-difluoroazetidin-2- yl}methyl)methanesulfonamide | 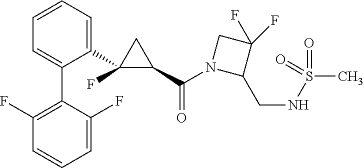 [SMILES: CS(=O)(=O)NCC1N(C(=O)[C@@H]2C[C@@]2(F)c2ccccc2-c2c(F)cccc2F)CC1(F)F] | 475.1 |
| 410 | N-({(2R,3R)-1-[(1S,2S)-2-(2′,6′-difluoro- 5-methyl[1,1′-biphenyl]-2-yl)-2- fluorocyclopropane-1-carbonyl]-3- fluoroazetidin-2- yl}methyl)ethanesulfonamide | 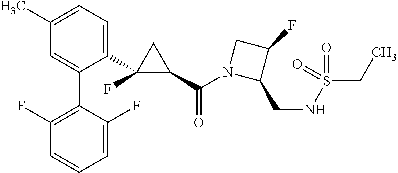 [SMILES: CCS(=O)(=O)NC[C@@H]1[C@H](F)CN1C(=O)[C@@H]1C[C@@]1(F)c1ccc(C)cc1-c1c(F)cccc1F] | 485.1 |
| 411 | N-({(2R,3R)-1-[(1S,2S)-2-(2′,6′-difluoro- 5-methyl[1,1′-biphenyl]-2-yl)-2- fluorocyclopropane-1-carbonyl]-3- fluoroazetidin-2- yl}methyl)methanesulfonamide | 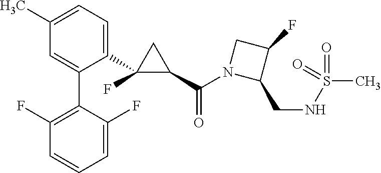 [SMILES: Cc1ccc([C@]2(F)C[C@H]2C(=O)N2C[C@@H](F)[C@H]2CNS(C)(=O)=O)c(-c2c(F)cccc2F)c1] | 471.1 |
| 412 | N-({(2R,3R)-3-fluoro-1-[(1S,2S)-2-fluoro- 2-(2′,5,6′-trifluoro[1,1′-biphenyl]-2- yl)cyclopropane-1-carbonyl]azetidin-2- yl}methyl)ethanesulfonamide | 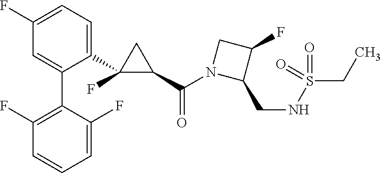 [SMILES: CCS(=O)(=O)NC[C@@H]1[C@H](F)CN1C(=O)[C@@H]1C[C@@]1(F)c1ccc(F)cc1-c1c(F)cccc1F] | 489.1 |
| 413 | N-({(2R,3R)-3-fluoro-1-[(1S,2S)-2-fluoro- 2-(2′,5,6′-trifluoro[1,1′-biphenyl]-2- yl)cyclopropane-1-carbonyl]azetidin-2- yl}methyl)methanesulfonamide | 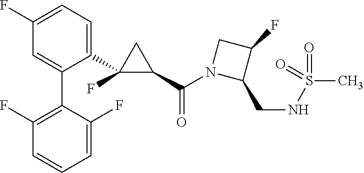 [SMILES: CS(=O)(=O)NC[C@@H]1[C@H](F)CN1C(=O)[C@@H]1C[C@@]1(F)c1ccc(F)cc1-c1c(F)cccc1F] | 475.1 |
| 414 | N-({(2R,3S)-1-[(1S,2S)-2-(2′,6′- difluoro[1,1′-biphenyl]-2-yl)-2- fluorocyclopropane-1-carbonyl]-3- fluoroazetidin-2- yl}methyl)ethanesulfonamide | 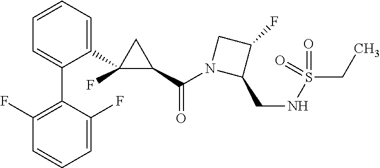 [SMILES: CCS(=O)(=O)NC[C@@H]1[C@@H](F)CN1C(=O)[C@@H]1C[C@@]1(F)c1ccccc1-c1c(F)cccc1F] | 471.1 |
| 415 | N-[(1R)-1-{(2R,3R)-1-[(1S,2S)-2-(2′,6′- difluoro[1,1′-biphenyl]-2-yl)-2- fluorocyclopropane-1-carbonyl]-3- fluoroazetidin-2- yl}ethyl]methanesulfonamide, or N- [(1S)-1-{(2R,3R)-1-[(1S,2S)-2-(2′,6′- difluoro[1,1′-biphenyl]-2-yl)-2- fluorocyclopropane-1-carbonyl]-3- fluoroazetidin-2- yl}ethyl]methanesulfonamide | 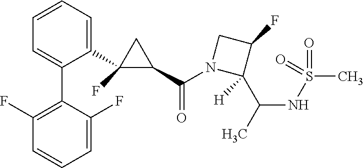 [SMILES: CC(NS(C)(=O)=O)[C@@H]1[C@H](F)CN1C(=O)[C@@H]1C[C@@]1(F)c1ccccc1-c1c(F)cccc1F] | 471.3 |
| 416 | N-[(1R)-1-{(2R,3R)-1-[(1S,2S)-2-(2′,6′- difluoro[1,1′-biphenyl]-2-yl)-2- fluorocyclopropane-1-carbonyl]-3- fluoroazetidin-2- yl}ethyl]ethanesulfonamide, or N-[(1S)- 1-{(2R,3R)-1-[(1S,2S)-2-(2′,6′- difluoro[1,1′-biphenyl]-2-yl)-2- fluorocyclopropane-1-carbonyl]-3- fluoroazetidin-2- yl}ethyl]ethanesulfonamide | 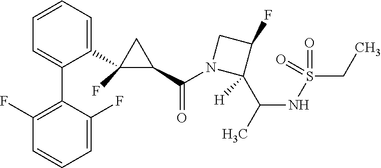 [SMILES: CCS(=O)(=O)NC(C)[C@@H]1[C@H](F)CN1C(=O)[C@@H]1C[C@@]1(F)c1ccccc1-c1c(F)cccc1F] | 485.2 |
| 417 | N-({(2S,3R)-1-[(1S,2S)-2-(2′,6′- difluoro[1,1′-biphenyl]-2-yl)-2- fluorocyclopropane-1-carbonyl]-3- fluoroazetidin-2- yl}methyl)ethanesulfonamide | 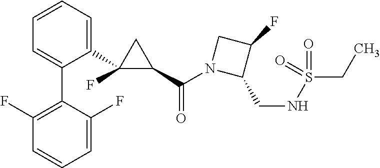 [SMILES: CCS(=O)(=O)NC[C@H]1[C@H](F)CN1C(=O)[C@@H]1C[C@@]1(F)c1ccccc1-c1c(F)cccc1F] | 471.1 |
| 418 | N-[(R)-{(2S)-1-[(1S,2S)-2-(2′,6′- difluoro[1,1′-biphenyl]-2-yl)-2- fluorocyclopropane-1- carbonyl]azetidin-2-yl}(1- hydroxycyclopropyl)methyl]methane- sulfonamide, or N-[(S)-{(2S)-1-[(1S,2S)-2- (2′,6′-difluoro[1,1′-biphenyl]-2-yl)-2- fluorocyclopropane-1-carbonyl] azetidin-2-yl}(1-hydroxycyclopropyl) methyl]methanesulfonamide | 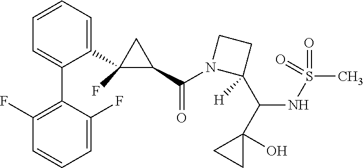 [SMILES: CS(=O)(=O)NC([C@@H]1CCN1C(=O)[C@@H]1C[C@@]1(F)c1ccccc1-c1c(F)cccc1F)C1(O)CC1] | 495.2 |
| 419 | N-[(1R)-1-{(2R)-1-[(1S,2S)-2-(2′,6′- difluoro[1,1′-biphenyl]-2-yl)-2- fluorocyclopropane-1- carbonyl]azetidin-2-yl}-2- methoxyethyl]methanesulfonamide, or N-[(1R)-1-{(2S)-1-[(1S,2S)-2-(2′,6′- difluoro[1,1′-biphenyl]-2-yl)-2- fluorocyclopropane-1- carbonyl]azetidin-2-yl}-2- methoxyethyl]methanesulfonamide | 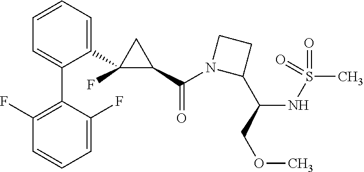 [SMILES: COC[C@H](NS(C)(=O)=O)C1CCN1C(=O)[C@@H]1C[C@@]1(F)c1ccccc1-c1c(F)cccc1F] | 483.3 |
| 420 | N-({(2R,3R)-1-[(1R,2R)-2-(5-chloro-2′,6′- difluoro[1,1′-biphenyl]-2-yl)-2- fluorocyclopropane-1-carbonyl]-3- fluoroazetidin-2- yl}methyl)ethanesulfonamide, or N- ({(2R,3R)-1-[(1S,2S)-2-(5-chloro-2′,6′- difluoro[1,1′-biphenyl]-2-yl)-2- fluorocyclopropane-1-carbonyl]-3- fluoroazetidin-2- yl}methyl)ethanesulfonamide | 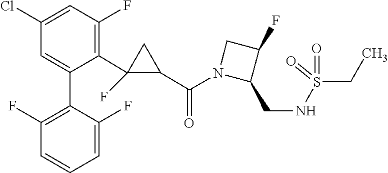 [SMILES: CCS(=O)(=O)NC[C@@H]1[C@H](F)CN1C(=O)C1CC1(F)c1c(F)cc(Cl)cc1-c1c(F)cccc1F] | 505.0 |
| 421 | a mixture of N-[(3R)-1-[(1S,2S)-2-(2′,6′- difluoro[1,1′-biphenyl]-2-yl)-2- fluorocyclopropane-1-carbonyl]-3-(3- methyl-1,2,4-oxadiazol-5-yl)pyrrolidin- 3-yl]methanesulfonamide and N-[(3S)- 1-[(1S,2S)-2-(2′,6′-difluoro[1,1′- biphenyl]-2-yl)-2-fluorocyclopropane- 1-carbonyl]-3-(3-methyl-1,2,4- oxadiazol-5-yl)pyrrolidin-3- yl]methanesulfonamide | 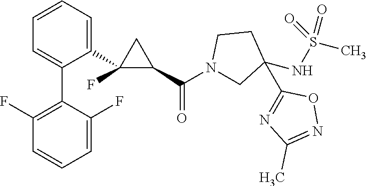 [SMILES: Cc1noc(C2(NS(C)(=O)=O)CCN(C(=O)[C@@H]3C[C@@]3(F)c3ccccc3-c3c(F)cccc3F)C2)n1] | 521.1 |
| 422 | a mixture of N-[(3R)-1-[(1S,2S)-2-(2′,6′- difluoro[1,1′-biphenyl]-2-yl)-2- fluorocyclopropane-1-carbonyl]-3-(5- methyl-1,3,4-oxadiazol-2-yl)pyrrolidin- 3-yl]methanesulfonamide and N-[(3S)- 1-[(1S,2S)-2-(2′,6′-difluoro[1,1′- biphenyl]-2-yl)-2-fluorocyclopropane- 1-carbonyl]-3-(5-methyl-1,3,4- oxadiazol-2-yl)pyrrolidin-3- yl]methanesulfonamide | 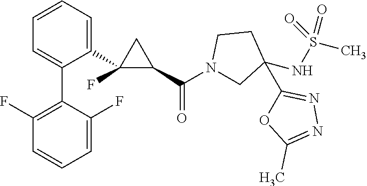 [SMILES: Cc1nnc(C2(NS(C)(=O)=O)CCN(C(=O)[C@@H]3C[C@@]3(F)c3ccccc3-c3c(F)cccc3F)C2)o1] | 521.1 |
| 423 | N-({(2R,3R)-3-fluoro-1-[(1S,2S)-2-fluoro- 2-(2′,5,6′-trifluoro[1,1′-biphenyl]-2- yl)cyclopropane-1-carbonyl]azetidin-2- yl}methyl)-N′-methylsulfuric diamide | 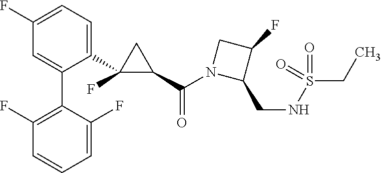 [SMILES: CCS(=O)(=O)NC[C@@H]1[C@H](F)CN1C(=O)[C@@H]1C[C@@]1(F)c1ccc(F)cc1-c1c(F)cccc1F] | 490.0 |
| 424 | N′-({(2R,3R)-3-fluoro-1-[(1S,2S)-2- fluoro-2-(2′,5,6′-trifluoro[1,1′- biphenyl]-2-yl)cyclopropane-1- carbonyl]azetidin-2-yl}methyl)-N,N- dimethylsulfuric diamide | 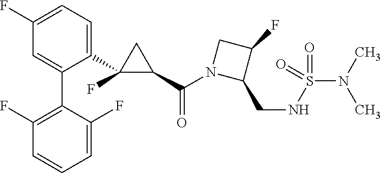 [SMILES: CN(C)S(=O)(=O)NC[C@@H]1[C@H](F)CN1C(=O)[C@@H]1C[C@@]1(F)c1ccc(F)cc1-c1c(F)cccc1F] | 504.1 |
| 425 | N-({(2R,3R)-3-fluoro-1-[(1S,2S)-2-fluoro- 2-(2′,5,6′-trifluoro[1,1′-biphenyl]-2- yl)cyclopropane-1-carbonyl]azetidin-2- yl}methyl)-N′-propan-2-ylsulfuric diamide | 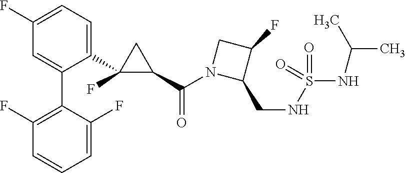 [SMILES: CC(C)NS(=O)(=O)NC[C@@H]1[C@H](F)CN1C(=O)[C@@H]1C[C@@]1(F)c1ccc(F)cc1-c1c(F)cccc1F] | 518.1 |
| 426 | N-({(2S,3S)-1-[(1S,2S)-2-(2′,6′- difluoro[1,1′-biphenyl]-2-yl)-2- fluorocyclopropane-1-carbonyl]-3- fluoroazetidin-2- yl}methyl)ethanesulfonamide | 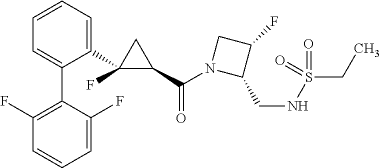 [SMILES: CCS(=O)(=O)NC[C@H]1[C@@H](F)CN1C(=O)[C@@H]1C[C@@]1(F)c1ccccc1-c1c(F)cccc1F] | 471.1 |
| 427 | N-ethyl-N′-({(2R,3R)-3-fluoro-1-[(1S,2S)- 2-fluoro-2-(2′,5,6′-trifluoro[1,1′- biphenyl]-2-yl)cyclopropane-1- carbonyl]azetidin-2-yl}methyl)sulfuric diamide | 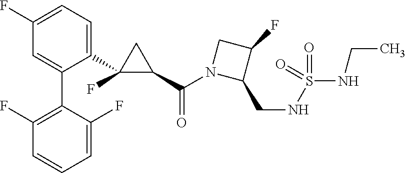 [SMILES: CCNS(=O)(=O)NC[C@@H]1[C@H](F)CN1C(=O)[C@@H]1C[C@@]1(F)c1ccc(F)cc1-c1c(F)cccc1F] | 504.1 |
| 428 | N-({(2R,3R)-1-[(1R,2R)-1-(cyanomethyl)- 2-(2′,6′-difluoro[1,1′-biphenyl]-2- yl)cyclopropane-1-carbonyl]-3- fluoroazetidin-2- yl}methyl)methanesulfonamide, or N- ({(2R,3R)-1-[(1S,2S)-1-(cyanomethyl)-2- (2′,6′-difluoro[1,1′-biphenyl]-2- yl)cyclopropane-1-carbonyl]-3- fluoroazetidin-2- yl}methyl)methanesulfonamide | 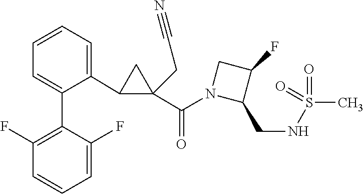 [SMILES: CS(=O)(=O)NC[C@@H]1[C@H](F)CN1C(=O)C1(CC#N)CC1c1ccccc1-c1c(F)cccc1F] | 478.1 |
| 429 | N-({(2R,3R)-3-fluoro-1-[(1S,2S)-2-fluoro- 2-(2′,5,6′-trifluoro[1,1′-biphenyl]-2- yl)cyclopropane-1-carbonyl]azetidin-2- yl}methyl)cyclopropanesulfonamide | 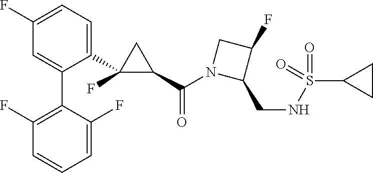 [SMILES: O=C([C@@H]1C[C@@]1(F)c1ccc(F)cc1-c1c(F)cccc1F)N1C[C@@H](F)[C@H]1CNS(=O)(=O)C1CC1] | 501.1 |
| 430 | 1-fluoro-N-({(2R,3R)-3-fluoro-1-[(1S,2S)- 2-fluoro-2-(2′,5,6′-trifluoro[1,1′- biphenyl]-2-yl)cyclopropane-1- carbonyl]azetidin-2- yl}methyl)methanesulfonamide | 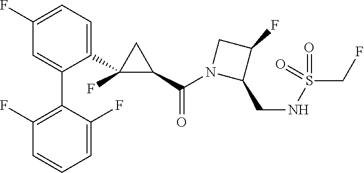 [SMILES: O=C([C@@H]1C[C@@]1(F)c1ccc(F)cc1-c1c(F)cccc1F)N1C[C@@H](F)[C@H]1CNS(=O)(=O)CF] | 493.0 |
| 431 | 1-bromo-N-({(2R,3R)-3-fluoro-1-[(1S,2S)- 2-fluoro-2-(2′,5,6′-trifluoro[1,1′- biphenyl]-2-yl)cyclopropane-1- carbonyl]azetidin-2- yl}methyl)methanesulfonamide | 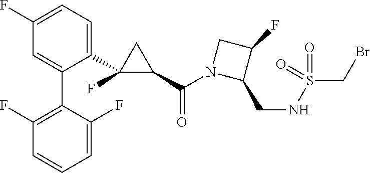 [SMILES: O=C([C@@H]1C[C@@]1(F)c1ccc(F)cc1-c1c(F)cccc1F)N1C[C@@H](F)[C@H]1CNS(=O)(=O)CBr] | 552.9 |
| 432 | N-({(2R,3R)-3-fluoro-1-[(1S,2S)-2-fluoro- 2-(2′,5,6′-trifluoro[1,1′-biphenyl]-2- yl)cyclopropane-1-carbonyl]azetidin-2- yl}methyl)-1- methoxymethanesulfonamide | 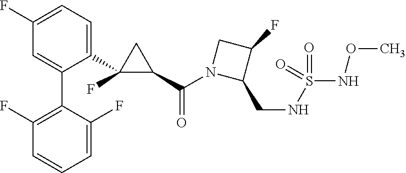 [SMILES: CONS(=O)(=O)NC[C@@H]1[C@H](F)CN1C(=O)[C@@H]1C[C@@]1(F)c1ccc(F)cc1-c1c(F)cccc1F] | 503.3 |
| 433 | N-({(2R,3R)-3-fluoro-1-[(1S,2S)-2-fluoro- 2-(2′,5,6′-trifluoro[1,1′-biphenyl]-2- yl)cyclopropane-1-carbonyl]azetidin-2- yl}methyl)propane-1-sulfonamide | 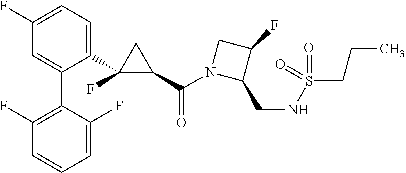 [SMILES: CCCS(=O)(=O)NC[C@@H]1[C@H](F)CN1C(=O)[C@@H]1C[C@@]1(F)c1ccc(F)cc1-c1c(F)cccc1F] | 503.1 |
| 434 | N-({(2S)-1-[(1R,2S)-1-(hydroxymethyl)-2- (2′,5,6′-trifluoro[1,1′-biphenyl]-2- yl)cyclopropane-1-carbonyl]azetidin-2- yl}methyl)methanesulfonamide, or N- ({(2S)-1-[(1S,2R)-1-(hydroxymethyl)-2- (2′,5,6′-trifluoro[1,1′-biphenyl]-2- yl)cyclopropane-1-carbonyl]azetidin-2- yl}methyl)methanesulfonamide | 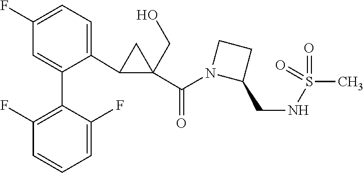 [SMILES: CS(=O)(=O)NC[C@@H]1CCN1C(=O)C1(CO)CC1c1ccc(F)cc1-c1c(F)cccc1F] | 469.1 |
| 435 | N-({(2S)-1-[(1R,2S)-1-(methoxymethyl)-2- (2′,5,6′-trifluoro[1,1′-biphenyl]-2- yl)cyclopropane-1-carbonyl]azetidin-2- yl}methyl)methanesulfonamide, or N- ({(2S)-1-[(1S,2R)-1-(methoxymethyl)-2- (2′,5,6′-trifluoro[1,1′-biphenyl]-2- yl)cyclopropane-1-carbonyl]azetidin-2- yl}methyl)methanesulfonamide | 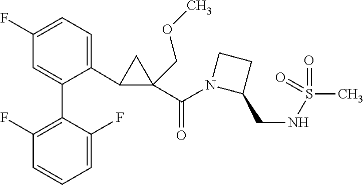 [SMILES: COCC1(C(=O)N2CC[C@H]2CNS(C)(=O)=O)CC1c1ccc(F)cc1-c1c(F)cccc1F] | 483.1 |
| 436 | N-({(2R,3R)-3-fluoro-1-[(1R,2S)-1- (methoxymethyl)-2-(2′,5,6′- trifluoro[1,1′-biphenyl]-2- yl)cyclopropane-1-carbonyl]azetidin-2- yl}methyl)methanesulfonamide, or N- ({(2R,3R)-3-fluoro-1-[(1S,2R)-1- (methoxymethyl)-2-(2′,5,6′- trifluoro[1,1′-biphenyl]-2- yl)cyclopropane-1-carbonyl]azetidin-2- yl}methyl)methanesulfonamide | 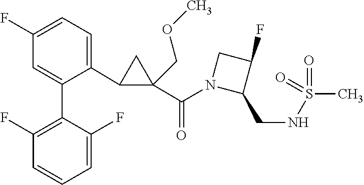 [SMILES: COCC1(C(=O)N2C[C@@H](F)[C@H]2CNS(C)(=O)=O)CC1c1ccc(F)cc1-c1c(F)cccc1F] | 501.1 |
| 437 | N-({(2R,3R)-3-fluoro-1-[(1R,2S)-1- (methoxymethyl)-2-(2′,5,6′- trifluoro[1,1′-biphenyl]-2- yl)cyclopropane-1-carbonyl]azetidin-2- yl}methyl)ethanesulfonamide, or N- ({(2R,3R)-3-fluoro-1-[(1S,2R)-1- (methoxymethyl)-2-(2′,5,6′- trifluoro[1,1′-biphenyl]-2- yl)cyclopropane-1-carbonyl]azetidin-2- yl}methyl)ethanesulfonamide | 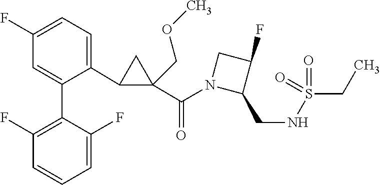 [SMILES: CCS(=O)(=O)NC[C@@H]1[C@H](F)CN1C(=O)C1(COC)CC1c1ccc(F)cc1-c1c(F)cccc1F] | 515.1 |
| 438 | N-({(2R,3R)-3-fluoro-1-[(1R,2S)-1- (methoxymethyl)-2-(2′,5,6′- trifluoro[1,1′-biphenyl]-2- yl)cyclopropane-1-carbonyl]azetidin-2- yl}methyl)-N′-methylsulfuric diamide, or N-({(2R,3R)-3-fluoro-1-[(1S,2R)-1- (methoxymethyl)-2-(2′,5,6′- trifluoro[1,1′-biphenyl]-2- yl)cyclopropane-1-carbonyl]azetidin-2- yl}methyl)-N′-methylsulfuric diamide | 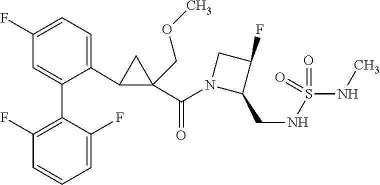 [SMILES: CNS(=O)(=O)NC[C@@H]1[C@H](F)CN1C(=O)C1(COC)CC1c1ccc(F)cc1-c1c(F)cccc1F] | 516.1 |
| 439 | N-({(2S,3R)-1-[(1R,2R)-2-fluoro-2-(2′- fluoro[1,1′-biphenyl]-2- yl)cyclopropane-1-carbonyl]-3- methylazetidin-2- yl}methyl)ethanesulfonamide, or N- ({(2S,3R)-1-[(1S,2S)-2-fluoro-2-(2′- fluoro[1,1′-biphenyl]-2- yl)cyclopropane-1-carbonyl]-3- methylazetidin-2- yl}methyl)ethanesulfonamide | 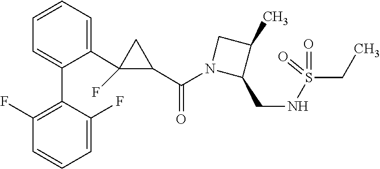 [SMILES: CCS(=O)(=O)NC[C@@H]1[C@H](C)CN1C(=O)C1CC1(F)c1ccccc1-c1c(F)cccc1F] | 449.1 |
| 440 | N-({(2R,3R)-1-[(1R,2R)-2-(2′-chloro-5- fluoro[1,1′-biphenyl]-2-yl)-2- fluorocyclopropane-1-carbonyl]-3- fluoroazetidin-2- yl}methyl)ethanesulfonamide, or N- ({(2R,3R)-1-[(1S,2S)-2-(2′-chloro-5- fluoro[1,1′-biphenyl]-2-yl)-2- fluorocyclopropane-1-carbonyl]-3- fluoroazetidin-2- yl}methyl)ethanesulfonamide | 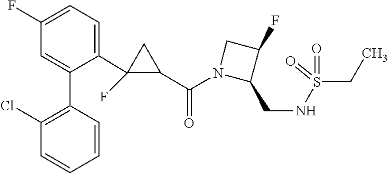 [SMILES: CCS(=O)(=O)NC[C@@H]1[C@H](F)CN1C(=O)C1CC1(F)c1ccc(F)cc1-c1ccccc1Cl] | 487.1 |
| 441 | N-({(2R,3R)-1-[(1R,2R)-2-(2′-chloro[1,1′- biphenyl]-2-yl)-2-fluorocyclopropane- 1-carbonyl]-3-fluoroazetidin-2- yl}methyl)ethanesulfonamide, or N- ({(2R,3R)-1-[(1S,2S)-2-(2′-chloro[1,1′- biphenyl]-2-yl)-2-fluorocyclopropane- 1-carbonyl]-3-fluoroazetidin-2- yl}methyl)ethanesulfonamide | 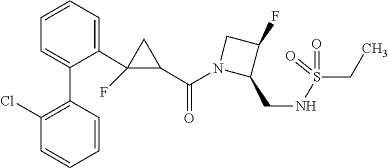 [SMILES: CCS(=O)(=O)NC[C@@H]1[C@H](F)CN1C(=O)C1CC1(F)c1ccccc1-c1ccccc1Cl] | 469.1 |
| 442 | N-({(2R,3R)-3-fluoro-1-[(1R,2R)-2-fluoro- 2-(2′-fluoro[1,1′-biphenyl]-2- yl)cyclopropane-1-carbonyl]azetidin-2- yl}methyl)ethanesulfonamide, or N- ({(2R,3R)-3-fluoro-1-[(1S,2S)-2-fluoro-2- (2′-fluoro[1,1′-biphenyl]-2- yl)cyclopropane-1-carbonyl]azetidin-2- yl}methyl)ethanesulfonamide | 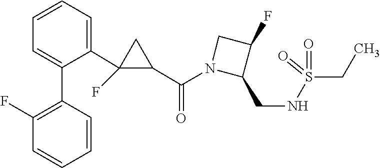 [SMILES: CCS(=O)(=O)NC[C@@H]1[C@H](F)CN1C(=O)C1CC1(F)c1ccccc1-c1ccccc1F] | 453.2 |
| 443 | N-({(2R,3R)-1-[(1S,2S)-2-(5-chloro-2′,6′- difluoro[1,1′-biphenyl]-2-yl)-2- fluorocyclopropane-1-carbonyl]-3- fluoroazetidin-2-yl}methyl)-N′- methylsulfuric diamide | 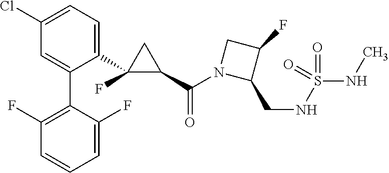 [SMILES: CNS(=O)(=O)NC[C@@H]1[C@H](F)CN1C(=O)[C@@H]1C[C@@]1(F)c1ccc(Cl)cc1-c1c(F)cccc1F] | 506.1 |
| 444 | N-({(2R,3R)-3-fluoro-1-[(1S,2S)-2-fluoro- 2-(2′,5,6′-trifluoro[1,1′-biphenyl]-2- yl)cyclopropane-1-carbonyl]azetidin-2- yl}methyl)propane-2-sulfonamide | 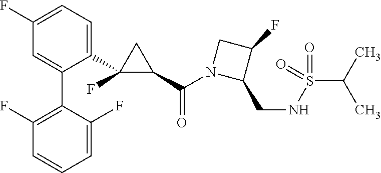 [SMILES: CC(C)S(=O)(=O)NC[C@@H]1[C@H](F)CN1C(=O)[C@@H]1C[C@@]1(F)c1ccc(F)cc1-c1c(F)cccc1F] | 503.1 |
| 445 | 1-cyclopropyl-N-({(2R,3R)-3-fluoro-1- [(1S,2S)-2-fluoro-2-(2′,5,6′- trifluoro[1,1′-biphenyl]-2- yl)cyclopropane-1-carbonyl]azetidin-2- yl}methyl)methanesulfonamide | 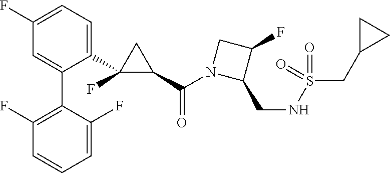 [SMILES: O=C([C@@H]1C[C@@]1(F)c1ccc(F)cc1-c1c(F)cccc1F)N1C[C@@H](F)[C@H]1CNS(=O)(=O)CC1CC1] | 515.1 |
| 446 | N-({(2R,3R)-3-fluoro-1-[(1S,2S)-2-fluoro- 2-(2′,5,6′-trifluoro[1,1′-biphenyl]-2- yl)cyclopropane-1-carbonyl]azetidin-2- yl}methyl)azetidine-1-sulfonamide | 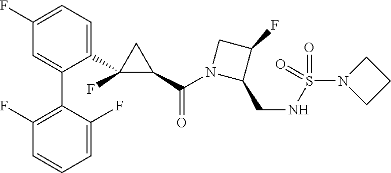 [SMILES: O=C([C@@H]1C[C@@]1(F)c1ccc(F)cc1-c1c(F)cccc1F)N1C[C@@H](F)[C@H]1CNS(=O)(=O)N1CCC1] | 516.1 |
| 447 | N-cyclopropyl-N′-({(2R,3R)-3-fluoro-1- [(1S,2S)-2-fluoro-2-(2′,5,6′- trifluoro[1,1′-biphenyl]-2- yl)cyclopropane-1-carbonyl]azetidin-2- yl}methyl)sulfuric diamide | 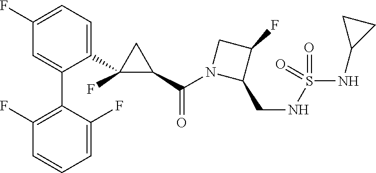 [SMILES: O=C([C@@H]1C[C@@]1(F)c1ccc(F)cc1-c1c(F)cccc1F)N1C[C@@H](F)[C@H]1CNS(=O)(=O)NC1CC1] | 516.1 |
| 448 | 1,1-difluoro-N-({(2R,3R)-3-fluoro-1- [(1S,2S)-2-fluoro-2-(2′,5,6′- trifluoro[1,1′-biphenyl]-2- yl)cyclopropane-1-carbonyl]azetidin-2- yl}methyl)methanesulfonamide | 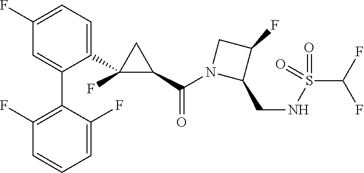 [SMILES: O=C([C@@H]1C[C@@]1(F)c1ccc(F)cc1-c1c(F)cccc1F)N1C[C@@H](F)[C@H]1CNS(=O)(=O)C(F)F] | 511.1 |
| 449 | 1,1,1-trifluoro-N-({(2R,3R)-3-fluoro-1- [(1S,2S)-2-fluoro-2-(2′,5,6′- trifluoro[1,1′-biphenyl]-2- yl)cyclopropane-1-carbonyl]azetidin-2- yl}methyl)methanesulfonamide | 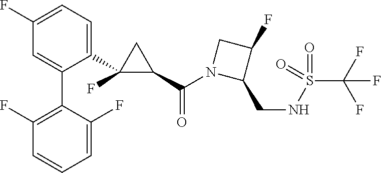 [SMILES: O=C([C@@H]1C[C@@]1(F)c1ccc(F)cc1-c1c(F)cccc1F)N1C[C@@H](F)[C@H]1CNS(=O)(=O)C(F)(F)F] | 527.1 |
| 450 | N-({(3R)-1-[(1S,2S)-2-(2′,6′-difluoro[1,1′- biphenyl]-2-yl)-2-fluorocyclopropane- 1-carbonyl]pyrrolidin-3- yl}methyl)methanesulfonamide, or N- ({(3S)-1-[(1S,2S)-2-(2′,6′-difluoro[1,1′- biphenyl]-2-yl)-2-fluorocyclopropane- 1-carbonyl]pyrrolidin-3- yl}methyl)methanesulfonamide | 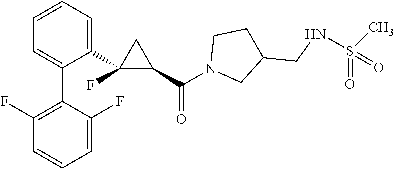 [SMILES: CS(=O)(=O)NCC1CCN(C(=O)[C@@H]2C[C@@]2(F)c2ccccc2-c2c(F)cccc2F)C1] | 453.2 |

### Experimental Example 1: Obtainment of Cell Stably Expressing Human Orexin Type 2 Receptor (hOX2R)

To obtain a cell clone stably expressing human orexin type 2 receptor, human orexin type 2 receptor cDNA was inserted into pcDNA3.1(+) plasmid vector (Invitrogen), and a plasmid DNA for expression of human orexin type 2 receptor (pcDNA3.1(+)/hOX2R) was cloned. The plasmid DNA was introduced into CHO-K1 cell by an electroporation method, and human orexin type 2 receptor expressing clone cells were obtained by limiting dilution method by using G418 drug resistance as a selection marker.

### Experimental Example 2: Measurement of Orexin Type 2 Receptor Agonist Activity

CHO cells forcibly expressing human OX2 receptor were seeded in each well of 384 well black transparent bottom plate (BD Falcon) at 10,000 cells/well, and cultured for one day in a 5% CO₂ incubator at 37° C. After removal of the medium in the cell plate, assay buffer A containing a calcium indicator (HBSS (Thermo Fisher Scientific), 20 mM HEPES (Thermo Fisher Scientific), 0.1% BSA (Sigma-Aldrich), 2.5 μg/mL Fluo-4 AM (DOJINDO Chemical), 0.08% Pluronic F127 (DOJINDO Chemical), 1.25 mM probenecid (DOJINDO Chemical)) was added at 30 μL/well. The plate was stood for 30 min in a 5% CO₂ incubator at 37° C., and further stood at room temperature for 30 min. A test compound prepared by diluting with assay buffer B (HBSS, 20 mM HEPES, 0.1% BSA) was added at 10 μL/well, and the fluorescence value was measured by FDSSμCELL (Hamamatsu Photonics K.K.) every one sec for 1 min, and thereafter every two sec for 1 min sec. The activity (%) of the test compound was calculated assuming that variation in the fluorescence value when DMSO was added instead of the test compound was 0%, and variation in the fluorescence value when orexin A (human) (PEPTIDE INSTITUTE, INC.) was added at the final concentration of 10 nM was 100%. The activity of each compound at the concentration of 3 μM was shown in Table 2. As is clear from the results, the compound of the present invention was shown to have an agonist activity on human orexin type 2 receptor.

TABLE 2

| Test compound | OX2R agonist activity (3 μM, %) |
|---|---|
| 3 | 81 |
| 4 | 102 |
| 10 | 80 |
| 12 | 66 |
| 15 | 92 |
| 23 | 105 |
| 24 | 95 |
| 25 | 93 |
| 26 | 104 |
| 27 | 98 |
| 28 | 103 |
| 31 | 101 |
| 32 | 102 |
| 37 | 101 |
| 38 | 106 |
| 39 | 102 |
| 40 | 77 |
| 41 | 97 |
| 43 | 92 |
| 51 | 75 |
| 52 | 65 |
| 54 | 79 |
| 56 | 85 |
| 59 | 80 |
| 65 | 83 |
| 66 | 52 |
| 67 | 89 |
| 68 | 85 |
| 72 | 81 |
| 74 | 84 |
| 80 | 66 |
| 81 | 70 |
| 85 | 68 |
| 87 | 81 |
| 89 | 64 |
| 95 | 61 |
| 96 | 62 |
| 98 | 56 |
| 105 | 60 |
| 106 | 63 |
| 112 | 101 |
| 113 | 100 |
| 114 | 102 |
| 123 | 100 |
| 124 | 104 |
| 126 | 89 |
| 127 | 57 |
| 128 | 89 |
| 129 | 101 |
| 130 | 87 |
| 134 | 74 |
| 135 | 111 |
| 136 | 110 |
| 137 | 59 |
| 145 | 94 |
| 152 | 95 |
| 153 | 100 |
| 158 | 106 |
| 159 | 101 |
| 161 | 99 |
| 162 | 106 |
| 164 | 104 |
| 166 | 110 |
| 167 | 96 |
| 180 | 95 |
| 183 | 78 |
| 184 | 108 |
| 185 | 57 |
| 186 | 101 |
| 188 | 86 |
| 189 | 67 |
| 192 | 95 |
| 193 | 98 |
| 194 | 100 |
| 195 | 99 |
| 196 | 101 |
| 198 | 101 |
| 199 | 104 |
| 200 | 103 |
| 201 | 101 |
| 202 | 106 |
| 203 | 94 |
| 204 | 96 |
| 205 | 78 |
| 206 | 87 |
| 207 | 101 |
| 208 | 78 |
| 209 | 95 |
| 210 | 94 |
| 211 | 105 |
| 212 | 91 |
| 213 | 100 |
| 214 | 95 |
| 215 | 98 |
| 216 | 99 |
| 217 | 83 |
| 219 | 96 |
| 220 | 79 |
| 221 | 107 |
| 223 | 81 |
| 226 | 104 |
| 227 | 85 |
| 228 | 90 |
| 229 | 64 |
| 230 | 91 |
| 232 | 103 |
| 233 | 80 |
| 235 | 89 |
| 237 | 99 |
| 238 | 98 |
| 239 | 97 |
| 240 | 100 |
| 241 | 103 |
| 245 | 103 |
| 246 | 78 |
| 247 | 100 |
| 248 | 98 |
| 249 | 105 |
| 250 | 104 |
| 251 | 104 |
| 256 | 52 |
| 257 | 98 |
| 258 | 97 |
| 259 | 98 |
| 261 | 88 |
| 262 | 109 |
| 263 | 98 |
| 264 | 97 |
| 265 | 86 |
| 266 | 87 |
| 267 | 87 |
| 268 | 80 |
| 269 | 100 |
| 270 | 64 |
| 271 | 87 |
| 272 | 99 |
| 273 | 98 |
| 274 | 86 |
| 275 | 57 |
| 276 | 101 |
| 279 | 87 |
| 281 | 96 |
| 282 | 87 |
| 283 | 100 |
| 284 | 94 |
| 285 | 63 |
| 286 | 96 |
| 287 | 96 |
| 288 | 96 |
| 289 | 99 |
| 290 | 98 |
| 291 | 93 |
| 294 | 104 |
| 295 | 100 |
| 297 | 101 |
| 298 | 100 |
| 299 | 90 |
| 302 | 97 |
| 303 | 99 |
| 304 | 93 |
| 305 | 85 |
| 306 | 93 |
| 307 | 100 |
| 308 | 100 |
| 309 | 97 |
| 310 | 100 |
| 311 | 98 |
| 312 | 100 |
| 313 | 97 |
| 314 | 99 |
| 315 | 91 |
| 316 | 88 |
| 318 | 96 |
| 319 | 101 |
| 320 | 101 |
| 321 | 95 |
| 323 | 96 |
| 324 | 96 |
| 325 | 96 |
| 326 | 89 |
| 327 | 100 |
| 328 | 94 |
| 329 | 91 |
| 330 | 91 |
| 331 | 96 |
| 332 | 95 |
| 333 | 99 |
| 334 | 96 |
| 335 | 96 |
| 337 | 100 |
| 338 | 98 |
| 339 | 103 |
| 340 | 94 |
| 341 | 101 |
| 342 | 101 |
| 344 | 95 |
| 346 | 100 |
| 347 | 95 |
| 348 | 97 |
| 349 | 97 |
| 350 | 102 |
| 351 | 101 |
| 352 | 96 |
| 353 | 102 |
| 355 | 94 |
| 356 | 84 |
| 357 | 96 |
| 358 | 96 |
| 359 | 94 |
| 360 | 103 |
| 361 | 90 |
| 362 | 89 |
| 363 | 98 |
| 364 | 88 |
| 365 | 103 |
| 367 | 93 |
| 368 | 97 |
| 370 | 84 |
| 371 | 103 |
| 372 | 95 |
| 373 | 95 |
| 374 | 98 |
| 375 | 92 |
| 377 | 93 |
| 378 | 103 |
| 379 | 86 |
| 380 | 92 |
| 381 | 90 |
| 382 | 96 |
| 383 | 72 |
| 384 | 99 |
| 385 | 95 |
| 386 | 98 |
| 387 | 99 |
| 388 | 98 |
| 389 | 97 |
| 390 | 98 |
| 391 | 97 |
| 392 | 68 |
| 393 | 104 |
| 394 | 106 |
| 395 | 99 |
| 396 | 104 |
| 397 | 84 |
| 398 | 97 |
| 399 | 95 |
| 400 | 99 |
| 401 | 79 |
| 403 | 83 |
| 404 | 95 |
| 405 | 96 |
| 406 | 50 |
| 408 | 73 |
| 409 | 100 |
| 410 | 103 |
| 411 | 95 |
| 412 | 111 |
| 413 | 109 |
| 414 | 101 |
| 415 | 96 |
| 416 | 97 |
| 417 | 98 |
| 418 | 99 |
| 419 | 104 |
| 420 | 97 |
| 421 | 88 |
| 422 | 75 |
| 423 | 102 |
| 424 | 98 |
| 425 | 72 |
| 426 | 104 |
| 427 | 96 |
| 428 | 53 |
| 429 | 99 |
| 430 | 92 |
| 431 | 95 |
| 432 | 105 |
| 433 | 95 |
| 434 | 96 |
| 435 | 101 |
| 436 | 99 |
| 437 | 99 |
| 438 | 101 |
| 439 | 107 |
| 440 | 91 |
| 441 | 90 |
| 442 | 100 |
| 443 | 100 |
| 444 | 96 |
| 445 | 76 |
| 446 | 100 |
| 447 | 90 |
| 448 | 98 |
| 449 | 94 |
| 450 | 96 |

### Experimental Example 3: X-Ray Crystallographic Analyses

The absolute stereochemistries of the compounds in Example 358, Example 411, Example 412, and Example 423 were determined using X-ray crystallographic analyses. All measurements were made on a Rigaku XtaLAB P200 diffractometer using multi-layer mirror monochromated Cu-Kα radiation. The structure was solved by direct methods with SHELXT-2018/2 and was refined using full-matrix least-squares on F2 with SHELXL-2018/3. All non-H atoms were refined with anisotropic displacement parameters.

### For Example 358

The structure was determined as N-({(2S,3R)-1-[(1S,2S)-2-(2′,6′-difluoro[1,1′-biphenyl]-2-yl)-2-fluorocyclopropane-1-carbonyl]-3-methylazetidin-2-yl}methyl)methanesulfonamide.

(confidence factor R₁=0.0267, wR₂=0.0732, Flack parameter χ=0.005(4))

### For Example 411

The structure was determined as N-({(2R,3R)-1-[(1S,2S)-2-(2′,6′-difluoro-5-methyl[1,1′-biphenyl]-2-yl)-2-fluorocyclopropane-1-carbonyl]-3-fluoroazetidin-2-yl}methyl)methanesulfonamide.

(confidence factor R₁=0.0702, wR₂=0.1823, Flack parameter χ=0.056(14))

### For Example 412

The structure was determined as N-({(2R,3R)-3-fluoro-1-[(1S,2S)-2-fluoro-2-(2′,5,6′-trifluoro[1,1′-biphenyl]-2-yl)cyclopropane-1-carbonyl]azetidin-2-yl}methyl)ethanesulfonamide.

(confidence factor R₁=0.0210, wR₂=0.0541, Flack parameter χ=0.000(4))

### For Example 423

The structure was determined as N-({(2R,3R)-3-fluoro-1-[(1S,2S)-2-fluoro-2-(2′,5,6′-trifluoro[1,1′-biphenyl]-2-yl)cyclopropane-1-carbonyl]azetidin-2-yl}methyl)-N′-methylsulfuric diamide.

(confidence factor R₁=0.0247, wR₂=0.0642, Flack parameter χ=0.000(8))

### Experimental Example 4: Evaluation of Wake-Promoting Effects in Cynomolgus Monkeys

The wake-promoting effects were evaluated by measuring the electroencephalogram (EEG) and electromyogram (EMG) in cynomolgus monkeys. Under isoflurane anesthesia (0.5-5%, Pfizer Japan Inc., Tokyo, Japan), male cynomolgus monkeys (2-3 years old, Hamri Co., Ltd., Ibaraki, Japan) were surgically implanted with radio-telemetry transmitters (L03-F3, Data Sciences International Inc., MN, USA). EEG electrodes were screwed into the skull at the parietal area. EMG electrodes were implanted on the cervical muscles. After the surgery, each monkey was given penicillin (100,000 units/head, i.m., Meiji Seika Pharma Co., Ltd., Tokyo, Japan), buprenorphine (0.02 mg/kg, i.m., Otsuka Pharmaceutical Co., Ltd., Tokyo, Japan) and prednisolone (1 mg/kg, s.c., Kyoritsu Seiyaku Co., Ltd., Tokyo, Japan) daily for one week. After at least a 1-month recovery period in home cages, the monkeys were habituated to the recording chamber placed in a soundproof room. EEG and EMG signals were recorded using the telemetry system (Ponemah software, Data Sciences International Inc., MN, USA) and the signals were analyzed using SleepSign software (Kissei Comtec Co., Ltd., Nagano, Japan). After confirming long sleep in dark phase in the experimental room, we used animals to examine the wake-promoting effect of compounds. Test compounds (10 mg/kg) suspended in 0.5% methylcellulose aqueous solution, or vehicle (i.e., 0.5% methylcellulose aqueous solution) was administered orally (p.o.) to monkeys at zeitgeber time 12 (ZT12) in a volume of 5 mL/kg body weight in pre-post design (n=2). EEG and EMG recordings were performed for 4 h after the compound administration. The time spent in wakefulness for 4 h after administration (% of vehicle treatment) was calculated by using SleepSign. The results are shown in Table 3.

TABLE 3

| | Wakefulness time |
|---|---|
| | (% of vehicle treatment) |
| Example No | (Mean) |
| EX353 | 397.50 |
| EX358 | 407.11 |
| EX393 | 731.96 |
| EX411 | 361.41 |
| EX412 | 573.33 |
| EX420 | 545.41 |
| EX423 | 391.59 |

As is clear from Table 3, compounds of the present invention increased the wakefulness time compared to the vehicle treatment group in cynomolgus monkeys. That is, these compounds were suggested to be potential therapeutics for narcolepsy.

### Formulation Example 1 (Production of Capsule)

| | | |
|---|---|---|
| 1) | compound of Example 1 | 30 mg |
| 2) | crystalline cellulose | 10 mg |
| 3) | lactose | 19 mg |
| 4) | magnesium stearate | 1 mg |

| | |
|---|---|
| total | 60 mg |

1), 2), 3) and 4) are mixed and filled in a gelatin capsule.

### Formulation Example 2 (Production of Tablet)

| | | |
|---|---|---|
| 1) | compound of Example 1 | 30 g |
| 2) | lactose | 50 g |
| 3) | cornstarch | 15 g |
| 4) | calcium carboxymethylcellulose | 44 g |
| 5) | magnesium stearate | 1 g |

| | |
|---|---|
| 1000 tablets | 140 g in total |

The total amount of 1), 2), 3) and 30 g of 4) are kneaded with water, vacuum dried and sieved. The sieved powder is mixed with 14 g of 4) and 1 g of 5), and the mixture is punched by a tableting machine. In this way, 1000 tablets containing 30 mg of the compound of Example 1 per tablet are obtained.

### INDUSTRIAL APPLICABILITY

The compound of the present invention has an orexin type 2 receptor agonist activity, and is useful as an agent for the prophylaxis or treatment of narcolepsy.

This application is based on patent application No. 2020-172765 filed on Oct. 13, 2020 in Japan, the contents of which are encompassed in full herein.

1. A compound represented by the formula: 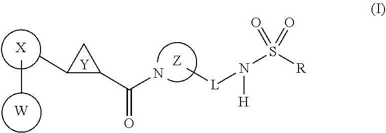
wherein
Ring W is an optionally further substituted ring;
Ring X is an optionally further substituted 5- or 6-membered aromatic ring;
Ring Y is an optionally further substituted cyclopropane ring;
Ring Z is an optionally further substituted nitrogen-containing heterocycle;
L is a bond, or an optionally substituted methylene; and
R is an optionally substituted C₁₋₆ alkyl group, an optionally substituted C₃₋₁₀ cycloalkyl group, or NR^{a}R^{b}; wherein
R^{a} is an optionally substituted C₁₋₆ alkyl group, or an optionally substituted C₃₋₁₀ cycloalkyl group;
R^{b} is a hydrogen atom, an optionally substituted C₁₋₆ alkyl group, or an optionally substituted C₃₋₁₀ cycloalkyl group; or
R^{a} and R^{b} in combination form an optionally further substituted nitrogen-containing heterocycle, together with adjacent nitrogen atom, or a salt thereof.

2. The compound or salt according to claim 1, wherein
Ring W is an optionally further substituted ring;
Ring X is an optionally further substituted 5- or 6-membered aromatic ring;
Ring Y is an optionally further substituted cyclopropane ring;
Ring Z is an optionally further substituted nitrogen-containing heterocycle;
L is a bond, or an optionally substituted methylene; and
R is an optionally substituted C₁₋₆ alkyl, an optionally substituted C₃₋₁₀ cycloalkyl, or an optionally substituted di-C₁₋₆ alkyl amine.

3. The compound or salt according to claim 1, wherein
Ring W is
(1) a 3- to 8-membered monocyclic non-aromatic heterocycle,
(2) a 5- to 6-membered monocyclic aromatic heterocycle optionally further substituted by 1 to 3 halogen atoms,
(3) a C₆₋₁₄ aromatic hydrocarbon ring optionally further substituted by 1 to 3 substituents selected from
(i) a halogen atom,
(ii) a C₁₋₆ alkyl group optionally substituted by 1 to 3 C₁₋₆ alkoxy groups, and
(iii) a C₁₋₆ alkoxy group, or
(4) a C₃₋₁₀ cycloalkane;
Ring X is
(1) a benzene ring optionally further substituted by 1 to 3 substituents selected from
(i) a halogen atom,
(ii) an optionally halogenated C₁₋₆ alkyl group, and
(iii) a C₁₋₆ alkoxy group, or
(2) a 5- or 6-membered monocyclic aromatic heterocycle optionally further substituted by 1 to 3 substituents selected from
(i) a halogen atom, and
(ii) C₁₋₆ alkyl groups;
Ring Y is a cyclopropane ring optionally further substituted by 1 to 3 substituents selected from
(1) a halogen atom,
(2) a cyano group, and
(3) a C₁₋₆ alkyl group, optionally substituted by 1 to 3 substituents selected from a C₁₋₆ alkoxy group, cyano, and hydroxy;
Ring Z is a 3- to 14-membered nitrogen-containing heterocycle optionally further substituted by 1 to 3 substituents selected from
(1) a halogen atom,
(2) a hydroxy group,
(3) a C₁₋₆ alkyl group, optionally substituted by 1 to 3 substituents selected from C₁₋₆ alkoxy group, halogen atom, and hydroxy,
(4) a C₁₋₆ alkoxy group,
(5) —C(O)—C₁₋₆ alkyl, and
(6) a 5-membered monocyclic aromatic heterocycle optionally substituted with a C₁₋₆ alkyl;
L is a bond or a methylene group optionally substituted with 1 substituent selected from
(1) a C₁₋₆ alkyl group further optionally substituted by 1 to 3 substituents selected from a C₁₋₆ alkoxy group, and hydroxy, and
(2) a C₃₋₆ cycloalkyl group further optionally substituted with a hydroxy group;
R is
(1) a C₁₋₆ alkyl group optionally substituted by 1 to 3 substituents selected from
(i) a halogen atom,
(ii) a C₁₋₆ alkoxy, and
(iii) a cyclopropyl group,
(2) a C₃₋₁₀ cycloalkyl group further optionally substituted with a C₁₋₆ alkyl group, or
(3) NR^{a}R^{b};
wherein
R^{a} is a C₁₋₆ alkyl group, or a C₃₋₁₀ cycloalkyl group;
R^{b} is a hydrogen atom, or a C₁₋₆ alkyl group; or
R^{a} and R^{b} in combination form a nitrogen-containing heterocycle, together with adjacent nitrogen atom.

4. The compound or salt according to claim 1 wherein
Ring W is a C₆₋₁₄ aromatic hydrocarbon ring optionally further substituted by 1 to 3 halogen atoms;
Ring X is a benzene ring optionally further substituted by 1 to 3 substituents selected from a halogen atom, and a C₁₋₆ alkyl group;
Ring Y is a cyclopropane ring optionally further substituted by 1 to 3 halogen atoms;
Ring Z is a 3- to 14-membered nitrogen-containing heterocycle ring optionally further substituted by 1 to 3 substituents selected from a halogen atom, a hydroxy group and a C₁₋₆ alkyl group;
L is a bond or a methylene group optionally substituted with a C₁₋₆ alkyl group;
R is
(1) a C₁₋₆ alkyl group, or
(2) NR^{a}R^{b};
wherein R^{a} is a C₁₋₆ alkyl group; and
R^{b} is a hydrogen atom, or a C₁₋₆ alkyl group.

5. The compound or salt according to claim 1, wherein
Ring W is a benzene ring optionally substituted by 1 to 3 halogen atoms;
Ring X is a benzene ring optionally further substituted by 1 to 3 substitutents selected from a halogen atom, and a C₁₋₆ alkyl group;
Ring Y is a cyclopropane ring optionally further substituted by 1 to 3 halogen atoms;
Ring Z is an azetidine ring optionally further substituted by 1 to 3 substitutents selected from a halogen atom and a C₁₋₆ alkyl group;
L is a methylene group optionally substituted with a C₁₋₆ alkyl group; and
R is a C₁₋₆ alkyl group or NR^{a}R^{b};
wherein R^{a} is a C₁₋₆ alkyl group; and
R^{b} is a hydrogen atom.

6. The compound or salt according to claim 1, wherein
Ring W is a benzene ring optionally substituted by 1 to 3 halogen atoms;
Ring X is a benzene ring optionally further substituted by 1 to 3 halogen atoms;
Ring Y is a cyclopropane ring optionally further substituted by 1 to 3 halogen atoms;
Ring Z is an azetidine ring optionally further substituted by 1 to 3 halogen atoms;
L is a methylene group;
R is a C₁₋₆ alkyl group, or NR^{a}R^{b};
wherein R^{a} is a C₁₋₆ alkyl group; and
R^{b} is a hydrogen atom.

7. A compound which is:
N-({(2R,3R)-3-fluoro-1-[(1S,2S)-2-fluoro-2-(2′,5,6′-trifluoro[1,1′-biphenyl]-2-yl)cyclopropane-1-carbonyl]azetidin-2-yl}methyl)ethanesulfonamide or a salt thereof.

8. A compound which is:
N-({(2R,3R)-3-fluoro-1-[(1S,2S)-2-fluoro-2-(2′,5,6′-trifluoro[1,1′-biphenyl]-2-yl)cyclopropane-1-carbonyl]azetidin-2-yl}methyl)-N′-methylsulfuric diamide or a salt thereof.

9. A compound which is:
N-({(2R,3R)-1-[(1S,2S)-2-(2′,6′-difluoro[1,1′-biphenyl]-2-yl)-2-fluorocyclopropane-1-carbonyl]-3-fluoroazetidin-2-yl}methyl)ethanesulfonamide or a salt thereof.

10. A compound of formula (II) 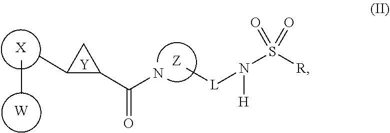
wherein:
Ring W is selected from benzene, 5- or 6-membered heteroaryl, 3-8-membered cycloalkyl, and 3-8-membered heterocyclyl, wherein each ring is optionally substituted by one, two, three, or four groups independently selected from halogen, (C₁-C₆)alkyl, and (C₁-C₆) alkoxy, wherein said (C₁-C₆)alkyl or (C₁-C₆)alkoxy is optionally substituted by one, two, three, or four halogen atoms;
Ring X is benzene or 5- or 6-membered heteroaryl, wherein each ring is optionally substituted with one, two, or three groups independently selected from halogen, (C₁-C₆)alkyl, and (C₁-C₆) alkoxy, wherein said (C₁-C₆)alkyl is optionally substituted by one, two, three, or four halogen atoms;
Ring Y is a cyclopropane ring optionally substituted by one, two, or three groups independently selected from halogen, (C₁-C₆)alkyl, (C₁-C₆) alkoxy, (C₁-C₆)alkyl-OH, (C₁-C₆)alkyl-O—(C₁-C₆)alkyl, (C₁-C₆)alkyl-CN, and —CN;
Ring Z is 3-9-membered nitrogen-containing monocyclic ring or 6-8-membered nitrogen-containing bicyclic ring, wherein each ring optionally contains one or two additional heteroatoms independently selected from oxygen, nitrogen, and sulfur; wherein said 3-9-membered nitrogen-containing monocyclic ring is optionally substituted by one, two, three, or four groups independently selected from halogen, (C₁-C₆)alkyl, halo(C₁-C₆)alkyl, (C₁-C₆) alkoxy, (C₁-C₆)alkyl-OH, (C₁-C₆)alkyl-O—(C₁-C₆)alkyl, —OH, and —C(O)—(C₁-C₆)alkyl and is also optionally substituted with a 5- or 6-membered heteroaryl ring optionally substituted with one, two, or three (C₁-C₆)alkyl groups; and wherein said 6-8-membered bicyclic ring is optionally substituted by one, two, three, or four groups independently selected from halogen, (C₁-C₆)alkyl, halo(C₁-C₆)alkyl, (C₁-C₆)alkoxy, (C₁-C₆)alkyl-OH, (C₁-C₆)alkyl-O—(C₁-C₆)alkyl, —OH, and —C(O)—(C₁-C₆)alkyl;
L is a bond, or —CH₂—, wherein said —CH₂— is optionally substituted by one or two groups independently selected from (C₁-C₆)alkyl, (C₁-C₆)alkoxy, and 3-6-membered cycloalkyl, wherein said (C₁-C₆)alkyl and 3-6-membered cycloalkyl ring are optionally substituted by one, two, or three groups independently selected from (C₁-C₆)alkyl, (C₁-C₆)alkoxy, and —OH; or, alternatively, wherein said —CH₂— is optionally substituted with two groups which, taken together with the carbon atom to which they are attached, form a 3-6 membered cycloalkyl or 3-6 membered heterocyclyl ring containing one or two oxygen atoms; and
R is (C₁-C₆)alkyl, —(C₁-C₆)alkyl-3-7 membered cycloalkyl, 3-7-membered cycloalkyl, (C₁-C₆)alkoxy, (C₁-C₆)alkyl-O—(C₁-C₆)alkyl, —NH₂, —NH((C₁-C₆)alkyl), —N((C₁-C₆)alkyl) 2, —NH(3-7-membered cycloalkyl ring), —N(3-7-membered cycloalkyl ring)₂, —N(3-7-membered cycloalkyl ring)(C₁-C₆)alkyl), and 3-7 membered heterocyclyl, wherein said (C₁-C₆)alkyl, —(C₁-C₆)alkyl-3-7 membered cycloalkyl, 3-7-membered cycloalkyl, (C₁-C₆) alkoxy, (C₁-C₆)alkyl-O—(C₁-C₆)alkyl, —NH((C₁-C₆)alkyl), N((C₁-C₆)alkyl)₂, —NH(3-7-membered cycloalkyl ring), —N(3-7-membered cycloalkyl ring)₂, —N(3-7-membered cycloalkyl ring)(C₁-C₆)alkyl, and 3-7-membered heterocyclyl are optionally substituted by one, two, or three groups independently selected from halogen and (C₁-C₆)alkyl,
or a pharmaceutically acceptable salt thereof.

11. A compound of Formula (II′) 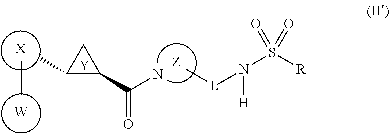
wherein:
Ring W is selected from benzene, 5- or 6-membered heteroaryl, 3-8-membered cycloalkyl, and 3-8-membered heterocyclyl, wherein each ring is optionally substituted by one, two, three, or four groups independently selected from halogen, (C₁-C₆)alkyl, and (C₁-C₆) alkoxy, wherein said (C₁-C₆)alkyl or (C₁-C₆)alkoxy is optionally substituted by one, two, three, or four halogen atoms;
Ring X is benzene or 5- or 6-membered heteroaryl, wherein each ring is optionally substituted with one, two, or three groups independently selected from halogen, (C₁-C₆)alkyl, and (C₁-C₆) alkoxy, wherein said (C₁-C₆)alkyl is optionally substituted by one, two, three, or four halogen atoms;
Ring Y is a cyclopropane ring optionally substituted by one, two, or three groups independently selected from halogen, (C₁-C₆)alkyl, (C₁-C₆) alkoxy, (C₁-C₆)alkyl-OH, (C₁-C₆)alkyl-O—(C₁-C₆)alkyl, (C₁-C₆)alkyl-CN, and —CN;
Ring Z is 3-9-membered nitrogen-containing monocyclic ring or 6-8-membered nitrogen-containing bicyclic ring, wherein each ring optionally contains one or two additional heteroatoms independently selected from oxygen, nitrogen, and sulfur; wherein said 3-9-membered nitrogen-containing monocyclic ring is optionally substituted by one, two, three, or four groups independently selected from halogen, (C₁-C₆)alkyl, halo(C₁-C₆)alkyl, (C₁-C₆) alkoxy, (C₁-C₆)alkyl-OH, (C₁-C₆)alkyl-O—(C₁-C₆)alkyl, —OH, and —C(O)—(C₁-C₆)alkyl and is also optionally substituted with a 5- or 6-membered heteroaryl ring optionally substituted with one, two, or three (C₁-C₆)alkyl groups; and wherein said 6-8-membered bicyclic ring is optionally substituted by one, two, three, or four groups independently selected from halogen, (C₁-C₆)alkyl, halo(C₁-C₆)alkyl, (C₁-C₆)alkoxy, (C₁-C₆)alkyl-OH, (C₁-C₆)alkyl-O—(C₁-C₆)alkyl, —OH, and —C(O)—(C₁-C₆)alkyl;
L is a bond, or —CH₂—, wherein said —CH₂— is optionally substituted by one or two groups independently selected from (C₁-C₆)alkyl, (C₁-C₆)alkoxy, and 3-6-membered cycloalkyl, wherein said (C₁-C₆)alkyl and 3-6-membered cycloalkyl ring are optionally substituted by one, two, or three groups independently selected from (C₁-C₆)alkyl, (C₁-C₆)alkoxy, and —OH; or, alternatively, wherein said —CH₂— is optionally substituted with two groups which, taken together with the carbon atom to which they are attached, form a 3-6 membered cycloalkyl or 3-6 membered heterocyclyl ring containing one or two oxygen atoms; and
R is (C₁-C₆)alkyl, —(C₁-C₆)alkyl-3-7 membered cycloalkyl, 3-7-membered cycloalkyl, (C₁-C₆)alkoxy, (C₁-C₆)alkyl-O—(C₁-C₆)alkyl, —NH₂, —NH((C₁-C₆)alkyl), —N((C₁-C₆)alkyl) 2, —NH(3-7-membered cycloalkyl ring), —N(3-7-membered cycloalkyl ring)₂, —N(3-7-membered cycloalkyl ring)(C₁-C₆)alkyl), and 3-7 membered heterocyclyl, wherein said (C₁-C₆)alkyl, —(C₁-C₆)alkyl-3-7 membered cycloalkyl, 3-7-membered cycloalkyl, (C₁-C₆) alkoxy, (C₁-C₆)alkyl-O—(C₁-C₆)alkyl, —NH((C₁-C₆)alkyl), N((C₁-C₆)alkyl)₂, —NH(3-7-membered cycloalkyl ring), —N(3-7-membered cycloalkyl ring)₂, —N(3-7-membered cycloalkyl ring)(C₁-C₆)alkyl, and 3-7-membered heterocyclyl are optionally substituted by one, two, or three groups independently selected from halogen and (C₁-C₆)alkyl,
or a pharmaceutically acceptable salt thereof.

12. The compound or pharmaceutically acceptable salt thereof according to claim 10 or 11, wherein
Ring W is benzene 5- or 6-membered heteroaryl, 3-8-membered cycloalkyl, and 6-membered heterocyclyl, wherein each ring is optionally substituted by one, two, three, or four groups independently selected from halogen, (C₁-C₆)alkyl, and (C₁-C₆)alkoxy, wherein said (C₁-C₆)alkyl or (C₁-C₆)alkoxy is optionally substituted by one, two, three, or four halogen atoms;
Ring X is benzene or 5- or 6-membered heteroaryl, wherein each ring is optionally substituted by one, two, or three groups independently selected from halogen, (C₁-C₆)alkyl, and (C₁-C₆)alkoxy, wherein said (C₁-C₆)alkyl is optionally substituted by one, two, three, or four halogen atoms;
Ring Y is a cyclopropane ring optionally substituted by one, two, or three groups independently selected from halogen, (C₁-C₆)alkyl, (C₁-C₆)alkyl-OH, (C₁-C₆)alkyl-O—(C₁-C₆)alkyl, (C₁-C₆)alkyl-CN, and —CN;
Ring Z is 3-9-membered nitrogen-containing monocyclic ring or 6-8-membered nitrogen-containing bicyclic ring, wherein each ring optionally contains one or two additional heteroatoms independently selected from oxygen, nitrogen, and sulfur; wherein said 3-9-membered nitrogen-containing monocyclic ring is optionally substituted by one, two, three, or four groups independently selected from halogen, (C₁-C₆)alkyl, halo(C₁-C₆)alkyl, ((C₁-C₆)alkyl)-OH, (C₁-C₆)alkoxy, (C₁-C₆)alkyl-O—(C₁-C₆)alkyl, —OH, and —C(O)—(C₁-C₆)alkyl and is also optionally substituted with a 5- or 6-membered heteroaryl ring optionally substituted with one, two, or three (C₁-C₆)alkyl groups; and wherein said 6-8-membered bicyclic ring is optionally substituted by one, two, three, or four groups independently selected from halogen, (C₁-C₆)alkyl, halo(C₁-C₆)alkyl, ((C₁-C₆)alkyl)-OH, (C₁-C₆)alkoxy, (C₁-C₆)alkyl-O—(C₁-C₆)alkyl, —OH, and —C(O)—(C₁-C₆)alkyl;
L is a bond or —CH₂—, wherein said —CH₂— is optionally substituted by one or two groups independently selected from (C₁-C₆)alkyl, (C₁-C₆)alkoxy, and 3-6-membered cycloalkyl, wherein said (C₁-C₆)alkyl and 3-6-membered cycloalkyl ring are optionally substituted by one, two, or three groups independently selected from (C₁-C₆)alkyl, (C₁-C₆)alkoxy, and —OH; and
R is (C₁-C₆)alkyl, —(C₁-C₆)alkyl-3-7-membered cycloalkyl, 3-7-membered cycloalkyl, (C₁-C₆)alkyl-O—(C₁-C₆)alkyl, —NH₂, —NH((C₁-C₆)alkyl), —N((C₁-C₆)alkyl)₂, —NH(3-7-membered cycloalkyl ring), —N(3-7-membered cycloalkyl ring)₂, —N(3-7-membered cycloalkyl ring)(C₁-C₆)alkyl), and 3-7-membered heterocyclyl, wherein said (C₁-C₆)alkyl, —(C₁-C₆)alkyl-3-7-membered cycloalkyl, 3-7-membered cycloalkyl, (C₁-C₆)alkoxy, (C₁-C₆)alkyl-O—(C₁-C₆)alkyl, —NH((C₁-C₆)alkyl), N((C₁-C₆)alkyl)₂, —NH(3-7-membered cycloalkyl ring), —N(3-7-membered cycloalkyl ring)₂, —N(3-7-membered cycloalkyl ring)(C₁-C₆)alkyl, and 3-7-membered heterocyclyl are optionally substituted by one, two, or three groups independently selected from halogen and (C₁-C₆)alkyl.

13. The compound or pharmaceutically acceptable salt thereof according to claim 10 or 11, wherein
Ring W is benzene, 5- or 6-membered heteroaryl, 4-6-membered cycloalkyl, and 6-membered heterocyclyl, wherein each ring is optionally substituted by one, two, three, or four groups independently selected from halogen, (C₁-C₆)alkyl, and (C₁-C₆)alkoxy, wherein said (C₁-C₆)alkyl or (C₁-C₆)alkoxy is optionally substituted by one, two, three, or four halogen atoms;
Ring X is benzene or 5- or 6-membered heteroaryl, wherein each ring is optionally substituted by one, two, or three groups independently selected from halogen, (C₁-C₆)alkyl, and (C₁-C₆) alkoxy, wherein said (C₁-C₆)alkyl is optionally substituted by one, two, three, or four halogen atoms;
Ring Y is a cyclopropane ring optionally substituted by one, two, or three groups independently selected from halogen, (C₁-C₆)alkyl, (C₁-C₆)alkyl-OH, (C₁-C₆)alkyl-O—(C₁-C₆)alkyl, (C₁-C₆)alkyl-CN, and —CN;
Ring Z is 3-9-membered nitrogen-containing monocyclic ring or 6-8-membered nitrogen-containing bicyclic ring, wherein each ring optionally contains one or two additional heteroatoms independently selected from oxygen, nitrogen, and sulfur; wherein said 3-9-membered nitrogen-containing monocyclic ring is optionally substituted by one, two, or three groups independently selected from halogen, (C₁-C₆)alkyl, halo(C₁-C₆)alkyl, ((C₁-C₆)alkyl)-OH, (C₁-C₆)alkoxy, (C₁-C₆)alkyl-O—(C₁-C₆)alkyl, —OH, and —C(O)—(C₁-C₆)alkyl, and is also optionally substituted with a 5- or 6-membered heteroaryl ring optionally substituted with one, two, or three (C₁-C₆)alkyl groups; and wherein said 6-8-membered bicyclic ring is optionally substituted by one, two, three, or four groups independently selected from halogen, (C₁-C₆)alkyl, halo(C₁-C₆)alkyl, ((C₁-C₆)alkyl)-OH, (C₁-C₆)alkoxy, (C₁-C₆)alkyl-O—(C₁-C₆)alkyl, —OH, and —C(O)—(C₁-C₆)alkyl;
L is a bond or —CH₂—, wherein said —CH₂— is optionally substituted by one or two groups independently selected from (C₁-C₆)alkyl, (C₁-C₆)alkoxy, and 3-6-membered cycloalkyl, wherein said (C₁-C₆)alkyl and 3-6-membered cycloalkyl ring are optionally substituted by one, two, or three groups independently selected from (C₁-C₆)alkyl, (C₁-C₆) alkoxy, and —OH; and
R is (C₁-C₆)alkyl, —(C₁-C₆)alkyl-3-7-membered cycloalkyl, 3-7-membered cycloalkyl, (C₁-C₆)alkyl-O—(C₁-C₆)alkyl, —NH₂, —NH((C₁-C₆)alkyl), —N((C₁-C₆)alkyl)₂, —NH(3-7-membered cycloalkyl ring), —N(3-7-membered cycloalkyl ring)₂, —N(3-7-membered cycloalkyl ring)(C₁-C₆)alkyl), and 3-7-membered heterocyclyl, wherein said (C₁-C₆)alkyl, —(C₁-C₆)alkyl-3-7-membered cycloalkyl, 3-7-membered cycloalkyl, (C₁-C₆)alkoxy, (C₁-C₆)alkyl-O—(C₁-C₆)alkyl, —NH((C₁-C₆)alkyl), N((C₁-C₆)alkyl)₂, —NH(3-7-membered cycloalkyl ring), —N(3-7-membered cycloalkyl ring)₂, —N(3-7-membered cycloalkyl ring)(C₁-C₆)alkyl, and 3-7-membered heterocyclyl are optionally substituted by one, two, or three groups independently selected from halogen and (C₁-C₆)alkyl.

14. The compound or pharmaceutically acceptable salt thereof according to any one of claims 10 to 13, wherein Ring W is benzene optionally substituted by one, two, or three halogen atoms.

15. The compound or pharmaceutically acceptable salt according to any one of claims 10 to 14, wherein Ring W is benzene optionally substituted by one, two, or three halogen atoms independently selected from chloro and fluoro.

16. The compound or pharmaceutically acceptable salt thereof according to any one of claims 10 to 15, wherein Ring X is benzene optionally further substituted by one, two, or three groups independently selected from a halogen atom, and a (C₁-C₆)-alkyl group.

17. The compound or pharmaceutically acceptable salt thereof according to any one of claims 10 to 16, wherein Ring Y is a cyclopropane ring optionally substituted by one, two, or three halogen atoms.

18. The compound or pharmaceutically acceptable salt thereof according to any one of claims 10 to 17, wherein Ring Z is a 4- to 8-membered nitrogen-containing monocyclic ring optionally substituted by one, two, or three groups independently selected from a halogen atom and a (C₁-C₆)-alkyl group.

19. The compound or pharmaceutically acceptable salt according to any one of claims 10 to 18, wherein Ring Z is azetidine optionally substituted by one, two, or three groups independently selected from halogen atom and a (C₁-C₆)alkyl group.

20. The compound or pharmaceutically acceptable salt according to any one of claims 10 to 19, wherein Ring Z is azetidine optionally substituted by one fluoro or methyl group.

21. The compound or pharmaceutically acceptable salt thereof according to any one of claims 10 to 20, wherein L is a bond or —CH₂— optionally substituted with a C₁₋₆ alkyl group.

22. The compound or pharmaceutically acceptable salt thereof according to any one of claims 10 to 21, wherein R is (C₁-C₆)alkyl, —NH((C₁-C₆)alkyl), or —N((C₁-C₆)alkyl)₂.

23. A compound of Tables 1-1 to 1-80, or a pharmaceutically acceptable salt thereof.

24. The compound or pharmaceutically acceptable salt according to claim 10 or 11, wherein
Ring W is benzene optionally substituted by one, two, or three halogen atoms;
Ring X is benzene optionally substituted by one, two, or three groups independently selected from a halogen atom and a C₁₋₆ alkyl;
Ring Y is a cyclopropane ring optionally substituted by one, two, or three halogen atoms;
Ring Z is a 4- to 8-membered nitrogen-containing monocyclic ring optionally substituted by one, two, or three groups independently selected from a halogen atom and a (C₁-C₆) alkyl group;
L is a bond or a —CH₂— group optionally substituted by a (C₁-C₆)alkyl group; and
R is a (C₁-C₆)alkyl group, —NH((C₁-C₆)alkyl), or —N((C₁-C₆)alkyl)₂.

25. The compound or pharmaceutically acceptable salt according to claim 10 or 11, wherein
Ring W is benzene optionally substituted by one, two, or three halogen atoms;
Ring X is benzene optionally further substituted by one, two, or three groups independently selected from a halogen atom and a (C₁-C₆)alkyl group;
Ring Y is a cyclopropane ring optionally substituted by one, two, or three halogen atoms;
Ring Z is an azetidine ring optionally substituted by one, two, or three groups independently selected from a halogen atom and a (C₁-C₆)alkyl group;
L is —CH₂— optionally substituted by a (C₁-C₆)alkyl group; and
R is (C₁-C₆)alkyl or —NH((C₁-C₆)alkyl).

26. The compound or pharmaceutically acceptable salt according to claim 10 or 11, wherein
Ring W is benzene optionally substituted by one, two, or three halogen atoms;
Ring X is benzene optionally substituted by one, two, or three halogen atoms;
Ring Y is a cyclopropane ring optionally substituted by a one, two, or three halogen atoms;
Ring Z is azetidine optionally substituted by one, two, or three halogen atoms;
L is —CH₂—; and
R is a C₁₋₆ alkyl group, or —NH((C₁-C₆)alkyl).

27. A compound selected from
N-({(2R,3R)-1-[(1S,2S)-2-(2′,6′-difluoro [1,1′-biphenyl]-2-yl)-2-fluorocyclopropane-1-carbonyl]-3-fluoroazetidin-2-yl}methyl)ethanesulfonamide,
N-({(2R,3R)-3-fluoro-1-[(1S,2S)-2-fluoro-2-(2′,5,6′-trifluoro[1,1′-biphenyl]-2-yl)cyclopropane-1-carbonyl]azetidin-2-yl}methyl)ethanesulfonamide,
N-({(2R,3R)-3-fluoro-1-[(1S,2S)-2-fluoro-2-(2′,5,6′-trifluoro[1,1′-biphenyl]-2-yl)cyclopropane-1-carbonyl]azetidin-2-yl}methyl)-N′-methylsulfuric diamide,
N-[(1S)-1-{(2S)-1-[(1S,2S)-2-(2′,6′-difluoro[1,1′-biphenyl]-2-yl)-2-fluorocyclopropane-1-carbonyl]azetidin-2-yl}ethyl]methanesulfonamide,
N-({(2S,3R)-1-[(1S,2S)-2-(2′,6′-difluoro [1,1′-biphenyl]-2-yl)-2-fluorocyclopropane-1-carbonyl]-3-methylazetidin-2-yl}methyl)methanesulfonamide,
N-({(2R,3R)-1-[(1S,2S)-2-(2′,6′-difluoro-5-methyl[1,1′-biphenyl]-2-yl)-2-fluorocyclopropane-1-carbonyl]-3-fluoroazetidin-2-yl}methyl)methanesulfonamide,
N-({(2R,3R)-1-[(1R,2R)-2-(5-chloro-2′,6′-difluoro[1,1′-biphenyl]-2-yl)-2-fluorocyclopropane-1-carbonyl]-3-fluoroazetidin-2-yl}methyl)ethanesulfonamide, and
N-({(2R,3R)-1-[(1S,2S)-2-(5-chloro-2′,6′-difluoro[1,1′-biphenyl]-2-yl)-2-fluorocyclopropane-1-carbonyl]-3-fluoroazetidin-2-yl}methyl)ethanesulfonamide,
or a pharmaceutically acceptable salt thereof.

28. A medicament comprising the compound or salt according to any one of claims 1 to 27.

29. The medicament according to claim 28, which is an orexin type 2 receptor agonist.

30. The medicament according to claim 28, which is an agent for the prophylaxis or treatment of narcolepsy.

31. The compound or salt according to any one of claims 1 to 27 for use in the prophylaxis or treatment of narcolepsy.

32. A method of activating an orexin type 2 receptor in a mammal, which comprises administering an effective amount of the compound or salt according to any one of claims 1 to 27 to the mammal.

33. A method for the prophylaxis or treatment of narcolepsy in a mammal, which comprises administering an effective amount of the compound or salt according to any one of claims 1 to 27 to the mammal.

34. A method of treating an orexin-mediated disease or disorder in a mammal in need thereof comprising administering to the mammal a therapeutically effective amount of the compound or pharmaceutically acceptable salt thereof according to any one of claims 1 to 27.

35. The method according to claim 34, wherein the orexin-mediated disease or disorder is narcolepsy.

36. Use of the compound or salt according to any one of claims 1 to 27 for the manufacture of an agent for the prophylaxis or treatment of narcolepsy.

37. Use of the compound or pharmaceutically acceptable salt according to any one of claims 1 to 27 for the manufacture of a medicament in the treatment of an orexin-mediated disease or disorder.

38. A pharmaceutical composition comprising a compound or pharmaceutically acceptable salt thereof according to any one of claims 1 to 27.
